SEQUENCE LISTING (102 sequences)

SEQ ID NO: 1 - protein, 128 aa, synthetic construct
MAAAVEALYVVLEREGAILPRQEGFSGVYVFFSPINFVIPPMGAVMLSLRLRVCIPPGYFGRFLALTDVNQPDVFTESYIMTPDMTEELSVVLFNHGDQFFYGHAGMAVVRLMLIRVVFPVVRQASNV

SEQ ID NO: 2 - protein, 150 aa, synthetic construct
MTTSGVPFGMTLRPTRSRLSRRTPYSRDRLPPFETETRATILEDHPLLPECNTLTMHNAWTSPSPPVKQPQVGQQPVAQQLDSDMNLSELPGEFINITDERLARQETVWNITPKNMSVTHDMMLFKASRGERTVYSVCWEGGGRLNTRVL

SEQ ID NO: 3 - protein, 153 aa, synthetic construct
MRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 4 - protein, 448 aa, synthetic construct
QVQLVESGGGVVQPGRSLRLSCAASGFTFSVYGMNWVRQAPGKGLEWVAIIWYDGDNQYYADSVKGRFTISRDNSKNTLYLQMNGLRAEDTAVYYCARDLRTGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 214 aa, synthetic construct
EIVLTQSPDFQSVTPKEKVTITCRASQSIGSSLHWYQQKPDQSPKLLIKYASQSFSGVPSRFSGSGSGTDFTLTINSLEAEDAAAYYCHQSSSLPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 880 aa, synthetic construct
SERCDDWGLDTMRQIQVFEDEPARIKCPLFEHFLKFNYSTAHSAGLTLIWYWTRQDRDLEEPINFRLPENRISKEKDVLWFRPTLLNDTGNYTCMLRNTTYCSKVAFPLEVVQKDSCFNSPMKLPVHKLYIEYGIQRITCPNVDGYFPSSVKPTITWYMGCYKIQNFNNVIPEGMNLSFLIALISNNGNYTCVVTYPENGRTFHLTRTLTVKVVGSPKNAVPPVIHSPNDHVVYEKEPGEELLIPCTVYFSFLMDSRNEVWWTIDGKKPDDITIDVTINESISHSRTEDETRTQILSIKKVTSEDLKRSYVCHARSAKGEVAKAAKVKQKVPAPRYTVEKCKEREEKIILVSSANEIDVRPCPLNPNEHKGTITWYKDDSKTPVSTEQASRIHQHKEKLWFVPAKVEDSGHYYCVVRNSSYCLRIKISAKFVENEPNLCYNAQAIFKQKLPVAGDGGLVCPYMEFFKNENNELPKLQWYKDCKPLLLDNIHFSGVKDRLIVMNVAEKHRGNYTCHASYTYLGKQYPITRVIEFITLEENKPTRPVIVSPANETMEVDLGSQIQLICNVTGQLSDIAYWKWNGSVIDEDDPVLGEDYYSVENPANKRRSTLITVLNISEIESRFYKHPFTCFAKNTHGIDAAYIQLIYPVTNSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 130 aa, synthetic construct
MSTEEQSSSLRHHPYRRARLPRCEEETRASLTEQHPLLPDCDHADYHNTVTLDCEARLDDFSEDGFISITDPRLARQETVWIIDPKSSSRTNQNISLFKATRAERTVYTVKWAGGGRLTTRAGVKINKDT

SEQ ID NO: 8 - DNA, 306 bp, synthetic construct
  source 1..306: mol_type = other DNA
atggatggatggtgtcctgggagagaccgtcggacagaggaagaaaatttccgcaaaaaagaagagcattgcccggagctgcccccagggagtgcaaagagagcactgcccaccagcacaagctcctctccccagcaaaagaaaaaaccactcgatggagaatatttcacccttaagatccgtgggcgtgagcgcttcgagatgttccgagagctgaatgaggccttggaattaaaggatgcccgtgctgccgaggagtcaggagacagcagggctcactccagctacccgaagatagttagttag

SEQ ID NO: 9 - protein, 101 aa, synthetic construct
MDGWCPGRDRRTEEENFRKKEEHCPELPPGSAKRALPTSTSSSPQQKKKPLDGEYFTLKIRGRERFEMFRELNEALELKDARAAEESGDSRAHSSYPKIVS

SEQ ID NO: 10 - DNA, 387 bp, synthetic construct
  source 1..387: mol_type = other DNA
atggccgctgctgtggaagccctgtacgtggtgcttgaaagagagggcgccatcctgcctagacaagagggcttttctggcgtgtacgtgttcttcagccccatcaacttcgtgatccctccaatgggcgccgtgatgctgagcctgagactgagagtgtgtatccctcctggctacttcggccggtttctggccctgaccgatgtgaaccagcctgacgtgttcaccgagagctacatcatgacccctgacatgaccgaggaactgagcgtggtgctgttcaaccacggcgaccagttcttttatggccacgccggaatggccgtcgtgcggctgatgctgatcagagtggtgtttcccgtcgtccggcaggccagcaatgtttga

SEQ ID NO: 11 - DNA, 453 bp, synthetic construct
  source 1..453: mol_type = other DNA
atgaccaccagcggcgtgcccttcggcatgacactcagacctaccagaagccggctgagcagaagaaccccttacagcagagacaggctgcctccattcgagacagagacacgggccaccatcctggaagatcaccctctgctgcccgagtgtaacaccctgaccatgcacaacgcctggacaagcccatctcctccagtgaaacagccccaagtgggacagcagcctgttgctcagcagctggacagcgacatgaacctgtctgaactgcccggcgagttcatcaacatcaccgacgagagactggcccggcaagagacagtgtggaacatcacccctaagaacatgagcgtgacccacgacatgatgctgttcaaggccagcagaggcgagcggacagtgtacagcgtttgttgggaaggcggcggacggctgaataccagagtgctgtaa

SEQ ID NO: 12 - DNA, 885 bp, synthetic construct
  source 1..885: mol_type = other DNA
atgacaaccagcggcgtgcccttcggcatgacactgaggcctacaagaagccggctgagcagaagaaccccttacagcagagacagactgcctccattcgagacagagacacgggccaccatcctggaagatcaccctctgctgcccgagtgcaacaccctgacaatgcacaacgtgtcctacgtgcggggcctgccttgtagcgttggcttcacactgatccaagagtgggtcgtgccctgggacatggtgctgaccagagaggaactggtcatcctgcggaagtgtatgcacgtgtgcctgtgctgcgccaacatcgacatcatgaccagcatgatgatccacggctacgagagctgggccctgcactgtcactgttctagccctggcagcctgcagtgtattgctggtggacaggttctggccagctggttcagaatggtggtggacggcgccatgttcaaccagagattcatctggtacagagaggtggtcaactacaacatgcccaaagaagtgatgttcatgagcagcgttttcatgcggggcagacacctgatctacctgcggctttggtacgatggccacgtgggatctgtggtgcctgccatgagctttggctacagcgccctgcattgcggcatcctgaacaacatcgtggtgctgtgctgcagctactgcgccgatctgagcgagatcagagtgcggtgttgtgccagacggaccagacggctgatgctgagagccgtgcggatcattgccgaagagacaaccgccatgctgtactcttgccggaccgagagaagaaggcagcagttcatcagagccctgctccagcaccaccggcctatcctgatgcacgactacgacagcacccctatgtag

SEQ ID NO: 13 - protein, 294 aa, synthetic construct
MTTSGVPFGMTLRPTRSRLSRRTPYSRDRLPPFETETRATILEDHPLLPECNTLTMHNVSYVRGLPCSVGFTLIQEWVVPWDMVLTREELVILRKCMHVCLCCANIDIMTSMMIHGYESWALHCHCSSPGSLQCIAGGQVLASWFRMVVDGAMFNQRFIWYREVVNYNMPKEVMFMSSVFMRGRHLIYLRLWYDGHVGSVVPAMSFGYSALHCGILNNIVVLCCSYCADLSEIRVRCCARRTRRLMLRAVRIIAEETTAMLYSCRTERRRQQFIRALLQHHRPILMHDYDSTPM

SEQ ID NO: 14 - DNA, 1488 bp, synthetic construct
  source 1..1488: mol_type = other DNA
atggaaagacggaaccccagcgagagaggcgtgccagctggattttctggacacgccagcgtggaaagcggctgcgagacacaagaaagccctgccaccgtggtgttcagaccacctggcgataataccgatggcggagctgctgctgcagctggtggatctcaggcagcagctgcaggcgctgaacctatggaacctgagagcagacctggacctagcggcatgaatgtggtgcaggtcgccgagctgtatcccgagctgagaagaatcctgaccatcaccgaggatggccagggactgaagggcgtgaagagagaaagaggcgcctgcgaggccacagaggaagccagaaatctggccttcagcctgatgaccagacacagacccgagtgcatcaccttccagcagatcaaggacaactgcgccaacgagctggacctgctggcccagaagtacagcatcgagcagctgaccacctactggctgcaacccggcgacgatttcgaagaggccatcagagtgtacgccaaggtggccctcagacctgactgcaagtacaagatcagcaagctggtcaacatccggaactgctgctacatcagcggcaatggcgccgaggtggaaatcgacacagaggacagagtggccttccggtgcagcatgatcaacatgtggcctggcgtgctcggcatggatggcgtggtcattatgaacgtgcggttcacaggccccaacttcagcggcacagtgtttctggccaacaccaacctgatcctgcacggcgtgtccttctacggcttcaacaatacctgcgtggaagcctggaccgacgttcgcgttagaggctgcgccttctactgctgttggaagggcgtcgtgtgcagacccaagagcagagccagcatcaagaagtgcctgttcgagagatgcaccctgggcatcctgagcgagggcaacagcagagtcagacacaacgtggccagcgactgcggctgcttcatgctggttaagagcgtggccgtgatcaagcacaacatggtctgcggcaactgcgaggatagagccagccagatgctgacctgcagcgacggcaattgtcatctgctgaaaaccatccacgtggcctctcacagcagaaaggcctggcctgtgttcgagcacaatatcctgacacggtgctccctgcacctgggcaatagacggggagtgttcctgccttaccagtgcaacctgagccacaccaagatcctgctggaacccgagtccatgagcaaagtgaacctgaatggcgtgttcgacatgaccatgaagatctggaaagtgctgcgctacgacgagacacggaccagatgtagaccttgcgagtgtggcggcaagcacatcagaaaccagcctgtgatgctggacgtgaccgaggaactgaggcctgatcatctggtgctggcctgtaccagagccgagtttggcagctccgacgaggataccgat

SEQ ID NO: 15 - protein, 496 aa, synthetic construct
MERRNPSERGVPAGFSGHASVESGCETQESPATVVFRPPGDNTDGGAAAAAGGSQAAAAGAEPMEPESRPGPSGMNVVQVAELYPELRRILTITEDGQGLKGVKRERGACEATEEARNLAFSLMTRHRPECITFQQIKDNCANELDLLAQKYSIEQLTTYWLQPGDDFEEAIRVYAKVALRPDCKYKISKLVNIRNCCYISGNGAEVEIDTEDRVAFRCSMINMWPGVLGMDGVVIMNVRFTGPNFSGTVFLANTNLILHGVSFYGFNNTCVEAWTDVRVRGCAFYCCWKGVVCRPKSRASIKKCLFERCTLGILSEGNSRVRHNVASDCGCFMLVKSVAVIKHNMVCGNCEDRASQMLTCSDGNCHLLKTIHVASHSRKAWPVFEHNILTRCSLHLGNRRGVFLPYQCNLSHTKILLEPESMSKVNLNGVFDMTMKIWKVLRYDETRTRCRPCECGGKHIRNQPVMLDVTEELRPDHLVLACTRAEFGSSDEDTD

SEQ ID NO: 16 - DNA, 131 bp, synthetic construct
  source 1..131: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcact

SEQ ID NO: 17 - DNA, 796 bp, synthetic construct
  source 1..796: mol_type = other DNA
tgctttctctgaccagcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccac

SEQ ID NO: 18 - DNA, 514 bp, synthetic construct
  source 1..514: mol_type = other DNA
ccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctcccca

SEQ ID NO: 19 - DNA, 720 bp, synthetic construct
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 20 - DNA, 226 bp, synthetic construct
  source 1..226: mol_type = other DNA
actgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 21 - DNA, 258 bp, synthetic construct
  source 1..258: mol_type = other DNA
tagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtgg

SEQ ID NO: 22 - DNA, 120 bp, synthetic construct
  source 1..120: mol_type = other DNA
agtgatggagttggccactccctctctgcgcgctcgctcgctcactgaggccgggcgaccaaaggtcgcccgacgcccgggcggcctcagtgagcgagcgagcgcgcagctgcctgcagg

SEQ ID NO: 23 - DNA, 131 bp, synthetic construct; identical to SEQ ID NO: 16
  source 1..131: mol_type = other DNA

SEQ ID NO: 24 - DNA, 290 bp, synthetic construct
  source 1..290: mol_type = other DNA
agaggaaacgtgtgggtggggaggggtagtgggtgagggacccaggttcctgacacagacagactacacccagggaatgaagagcaagcgccatgttgaagccatcattaccattcacatccctcttattcctgcagctgcccctgctgggagtggggctgaacacgacaattctgacgcccaatgggaatgaagacaccacagctggtgggaaatctgggactggagggggctggtgagaagggtggctgtgggaaggggccgtacagagatctggtgcctgccactgg

SEQ ID NO: 25 - DNA, 154 bp, synthetic construct
  source 1..154: mol_type = other DNA
atttctttctgaccaccatgcccaccgacagcctgagcgtgagcaccctgcccctgcccgaggtgcagtgcttcgtgttcaacgtggagtacatgaactgcacctggaacagcagcagcgagccccagcccaccaatctgaccctgcactactg

SEQ ID NO: 26 - DNA, 185 bp, synthetic construct
  source 1..185: mol_type = other DNA
gtacaagaacagcgacaacgacaaggtgcagaagtgcagccactacctgttcagcgaggaaatcaccagcggctgccagctgcagaagaaagagatccacctgtaccagaccttcgtggtgcagctgcaggacccccgggagccccgcaggcaggccacccagatgctgaagctgcagaacctgg

SEQ ID NO: 27 - DNA, 140 bp, synthetic construct
  source 1..140: mol_type = other DNA
tgatcccctgggcccctgagaacctgacactgcacaagctgtccgagagccagctggaactgaactggaacaaccgcttcctgaaccactgcctggaacacctggtgcagtaccggaccgactgggaccacagctggacc

SEQ ID NO: 28 - DNA, 163 bp, synthetic construct
  source 1..163: mol_type = other DNA
gagcagagcgtggactaccggcacaagttcagcctgcccagcgtggacggccagaagcggtacaccttcagagtgcggagccggttcaaccccctgtgcggcagcgcccagcactggtccgagtggagccaccccatccactggggcagcaacaccagcaaag

SEQ ID NO: 29 - DNA, 97 bp, synthetic construct
  source 1..97: mol_type = other DNA
agaaccccttcctgttcgccctggaagccgtggtgatcagcgtgggcagcatgggcctgatcatctccctgctgtgcgtgtacttctggctggaacg

SEQ ID NO: 30 - DNA, 70 bp, synthetic construct
  source 1..70: mol_type = other DNA
gaccatgcccagaatccccaccctgaagaacctggaagatctggtgaccgagtaccacggcaacttcagc

SEQ ID NO: 31 - DNA, 186 bp, synthetic construct
  source 1..186: mol_type = other DNA
gcctggtccggcgtgagcaagggcctggccgagagcctgcagcccgactacagcgagcggctgtgcctggtgtccgagatcccccccaaaggcggagccctgggcgaaggccctggcgccagcccctgcaaccagcacagcccctactgggcccctccttgctacaccctgaagcccgagacctga

SEQ ID NO: 32 - DNA, 330 bp, synthetic construct
  source 1..330: mol_type = other DNA
tgaaccccaatcctctgacagaagaaccccagggtcctgtagccctaagtggtactaactttccttcattcaacccactgcgtctcatactcacctcaccccactgtggctgatttggaattttgtgcccccatgtaagcaccccttcatttggcattccccacttgagaattacccttttgccccgaacatgtttttcttctccctcagtctggcccttccttttcgcaggattcttcctccccccctctttccctcccttcctctttccatctaccctccgattgttcctgaaccgatgagaaataaagtttctgttgataatcatcg

SEQ ID NO: 33 - DNA, 226 bp, synthetic construct
  source 1..226: mol_type = other DNA
actgtgccttctagttgccagccatctgttgtttgcccctcccccgtgccttccttgaccctggaaggtgccactcccactgccctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 34 - DNA, 516 bp, synthetic construct
  source 1..516: mol_type = other DNA
ccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagg

SEQ ID NO: 35 - DNA, 720 bp, synthetic construct; identical to SEQ ID NO: 19
  source 1..720: mol_type = other DNA

SEQ ID NO: 36 - DNA, 244 bp, synthetic construct
  source 1..244: mol_type = other DNA
tgagatccagacatgataagatacattgatgagtttggacaaaccaaaactagaatgcagtgaaaaaaatgccttatttgtgaaatttgtgatgctattgccttatttgtaaccattataagctgcaataaacaagttaacaacaacaattgcattcattttatgtttcaggttcagggggaggtgtgggaggttttttaaagcaagtaaaacctctacaaatgtggtatggctgattatgatc

SEQ ID NO: 37 - DNA, 267 bp, synthetic construct
  source 1..267: mol_type = other DNA
tacaatcatgtgggcagaattgaaaagtggagtgggaagggcaagggggagggttccctgcctcacgctacttcttctttctttcttgtttgtttgtttctttctttcttttgaggcagggtctcactatgttgcctaggctggtctcaaactcctggcctctagtgatcctcctgcctcagcctttcaaagcaccaggattacagacatgagccaccgtgcttggcctcctccttctgaccatcatttctctttccctccctgcct

SEQ ID NO: 38 - DNA, 120 bp, synthetic construct; identical to SEQ ID NO: 22
  source 1..120: mol_type = other DNA

SEQ ID NO: 39 - protein, 130 aa, synthetic construct
MQTEDQSSLLRHHPYRRARLPRSDEETRASLTEQHPLLPDCDHADYHNTVTLDCEARLDDFSEDGFISITDPRLARQETVWIIDPKFSSRTNQNIPLFKATRAERIVYTVKWAGGGRLTTRAGVKINKDT

SEQ ID NO: 40 - protein, 130 aa, synthetic construct
MQTEDQSSLLRHHPYRRARLPRSDEETRASLTEQHPLLPDCDHAEYHNTVTLDCEARLDDFSEDGFISITDPRLARQETVWLIDTKSSSRANQNIPLFKATRAERIVYTVKWAGGGRLTTRAGVKINKDT

SEQ ID NO: 41 - protein, 130 aa, synthetic construct
MSTEEQSSSLRHHPYRRARLPRCEEETRASLTEQHPLLPDCDHADYHNTVTLDCEARLDDFSEDGFISITDPRLARQETVWIIDSKSSSRTNQNIPLFKATRAERIVYTVKWAGGGRLTTRAGVKINKDP

SEQ ID NO: 42 - protein, 130 aa, synthetic construct
MSTEEQSSSLRHHPYRRARLPRCDEETRASLTEQHPLLPDCDHADYHNTVTLDCEARLDDFSEDGFISITDPRLARQETVWIIDPKSSSRTNQNIFLFKATRAERIVYTVKWAGGGRLTTRAGVKINKDT

SEQ ID NO: 43 - protein, 141 aa, synthetic construct
MSGSNSIMTRLRARSTSCARHHPYTRAQLPRCEENETRASMTEDHPLLPDCDTMTMHSMTVIQTPESHPQQLDCESALKDYPDAFLSITDPRLARSETVWNVESKTMSISNGIQMFKAVRGERLVYSVKWEGGGKITTRIL

SEQ ID NO: 44 - protein, 130 aa, synthetic construct
MSTEEQSTSLRHHPYRRARLPRSEEETRASLTEQHPLLPDCDHAEYHNTVTLDCEARLEDFSEDGFISITDPRLARQETVWIIDTKSSSRTNQNIPLFKATRAERIVYTVKWAGGGRLTTRAGVKINKDT

SEQ ID NO: 45 - protein, 130 aa, synthetic construct
MSTEEQSSLLRHHPYRRARLPRCEEETRASLTEQHPLLPDCDHADYHNTVTLDCEARLDDFSEDGFISITDPRLARQETVWIIDTKSSSRSNNNIPLFKATRAERIVYTVKWAGGGRLTTRAGVKINKDS

SEQ ID NO: 46 - protein, 130 aa, synthetic construct
MSTEEQSSLLRHHPYRRARLPRCEEETRASLTEQHPLLPDCDHADYHNTVTLDCEARLEDFSEDGFISITDPRLARQETVWIIDTKSSSRTNQNIPLFKATRAERIVYTVKWAGGGRLTTRAGVKINKDT

SEQ ID NO: 47 - protein, 130 aa, synthetic construct
MSTEEQSSLLRHHPYRRARLPRCEQETRASLTEQHPLLPDCDHAEYHNTVTLDCEPRLEDFSEDGFISITDPRLARQETVWLIDTKTSSRTNQNIPLFKATRAERTVYTVKWAGGGRLTTRAGVKINKDT

SEQ ID NO: 48 - protein, 130 aa, synthetic construct
MQTEDQSSLLRHHPYRRARLPRCEEETRASLTEQHPLLPDCDHADYHNTVTLDCEARLEDFSEDGFISITDPRLARQETVWLIDTKSSSRTNQNIPLFKATRAERTVYTVKWAGGGRLTTRAGVKINKDT

SEQ ID NO: 49 - protein, 130 aa, synthetic construct
MSTEEQSSLLRHHPYRRARLPRCEEETRASLTEQHPLLPDCDHADYHNTVTLDCEARLDDFSEDGFISITDPRLARQETVWIIDPKSSSRTNQNISLFKATRAERIVYTVKWAGGGRLTTRAGVKINKDT

SEQ ID NO: 50 - protein, 130 aa, synthetic construct
MSTEEQSSLLRHHPYRRARLPRSDEETRASLTEQHPLLPDCDHADYHNTVTLDCEARLEDFSEDGFISITDPRLARQETVWIIDPKSSSRTNQNIPLFKATRAERIVYTVKWAGGGRLTTRAGVKINKDT

SEQ ID NO: 51 - protein, 141 aa, synthetic construct
MSGNNSIMTRLRARSTSCARHHPYTRAQLPRCEENETRASMTEDHPLLPDCDTMTMHSMTVIQTPESHPQQLDCESALKDYPDAFLCITDPRLARSETVWNVETKTMSISNGIQMFKAVRGERLVYSVKWEGGGKITTRIL

SEQ ID NO: 52 - protein, 130 aa, synthetic construct
MQTEIQSSSLRHHPYRRARLPRSDEETRASLTEQHPLLPDCDHADYHNTVTLDCEARLDDFSEDGFISITDPRLARQETVWIINPKSSSRTNENFPLFKATRAERIVYTVKWAGGGRMCTRAGVKINKDT

SEQ ID NO: 53 - protein, 130 aa, synthetic construct
MSTEEQSTLLRHHPYRRARLSRYDKETRASLTEQHPLLPDCDHADYHNTVTLDCEARLDDLSKDGFISITDPRLARQETVWIIDPKSSSRTNENFPLFKATRTERIVYTVKWAGGGRMCTRAGVKINKDT

SEQ ID NO: 54 - protein, 130 aa, synthetic construct
MQTEIQSSLLRHHPYRRARLPRSDEETRASLTEQHPLLPDCDHADYHNTVTLDCEARLDDFSEDGFISITDPRLARQETVWLIDTKSSSRANQNIPLFKATRAERIVYTVKWAGGGRLTTRAGVKINKDT

SEQ ID NO: 55 - protein, 130 aa, synthetic construct
MQTEIQSSSLRHHPYRRARLPRSDEETRASLTEQHPLLPDCDHADYHNTVTLDCEARLDDFSEDGFISITDPRLARQETVWIIDPKSNSRTNENISLFKATRAERTVYTVKWAGGGRLSTRAGVKINKDT

SEQ ID NO: 56 - protein, 130 aa, synthetic construct
MQTEIQSSSLRHHPYRRARLPRSDEETRASLTEQHPLLPDCDHADYHNTVTLDCEARLDDFSEDGFISITDPRLARQETVWIIDPKSSSRTNQNISLFKATRAERIVYTVKWAGGGRLTTRAGVKINKDT

SEQ ID NO: 57 - protein, 128 aa, synthetic construct
MAAAVEALYVVLEREGAILPRQEGFSGVYVFFSPINFVIPPMGAVMLPLRLRVCIPPGYFGRFLALTDVNQPDVFTESYIMTPDMTEELSVVLFNHGDQFFYGHAGMAVVRLMLIRVVFPVVRQASNV

SEQ ID NO: 58 - protein, 128 aa, synthetic construct
MAAAVEALYVVLEREGAILPRQEGFSGVYVFFSPINFVIPPMGAVMLSLRLRVCIPPGYFGRFLALTDVNQPDVFTESYIMTPDMIEELSVVLFNHGDQFFYGHAGMAVVRLMLIRVVFPVVRQASNV

SEQ ID NO: 59 - protein, 125 aa, synthetic construct
MADEALYVYLEGPGATLPEQQQRNNYIFYSPVPFTLYPRGVALLYLKLSIIIPRGYVGCFFSLTDANMSGLYASSRIIHAGHREELSVLLFNHNDRFYEGRAGDPVACLVMERLIFPPVRQATMI

SEQ ID NO: 60 - protein, 124 aa, synthetic construct
MDAQVLYVFLEGAGALLPVQKGSNYIFYAPANFVLHPHGVALLELRLSIVVPQGFIGRFFSLTDANVPGVYASSRIIHAGHREGLSVMLFNHNVSFYNGRAGDPVACLVLERVIYPPVRQASMV

SEQ ID NO: 61 - protein, 128 aa, synthetic construct
MAAAVEALYVVLEREGAILPRQEGFSGVYVFFSPINFVIPPMGAVMLSLRLRVCIPPGYFGRFLALTDVSQPDVFTESYIMTPDMTEELSVVLFNHGDQFFYGHAGMAVVRLMLIRVVFPVVRQASNV

SEQ ID NO: 62 - protein, 125 aa, synthetic construct
MADEALYVYLEGPGATLPEQQQRNNYIFYSPVPFTLYPRGVALLYLRLSIIIPRGYVGCFFSLTDANMSGLYASSRIIHAGHREELSVLLFNHDDRFYEGRAGDPVACLVMERLIYPPVRQATMI

SEQ ID NO: 63 - protein, 125 aa, synthetic construct
MAESLYAFIDSPGGIAPVQEGTSNRYTFFCPESFHIPPHGVVLLHLKVSVLVPTGYQGRFMALNDYHARDILTQSDVIFAGRRQELTVLLFNHTDRFLYVRKGHPVGTLLLERVIFPSVKIATLV

SEQ ID NO: 64 - protein, 125 aa, synthetic construct
MADEALYVYLEGPGATLPEQQQRNNYIFYSPVPFTLYPRGVALLYLRLSIIIPRGYIGCFLSLTDANMFGLYASSRIIHAGHREELSVLLFNHDDRFYEGRAGDPVACLVMERLIYPPVRQATLI

SEQ ID NO: 65 - protein, 126 aa, synthetic construct
MADEALYVYFRGPGATLPEQQQQRNNYIFYSPVPFTLYPRGVALLYLRLSIIIPRGYVGCFFSLTDANMSGLYASSRIIHAGHREELSVLLFNHDDRFYEGRAGDPVACLVMERLIYPPVRQATMI

SEQ ID NO: 66 - protein, 127 aa, synthetic construct
MAALQALYVYFKGPGAMLPEQEGYSNAYVLFSPANFVIPPHGVVLLYLHIAVDIPPGYLGTLFSLSDMNARGVFVGAETLYPGSRMELSVLLFNHSDVFCDVRAKQPVARLLLSRVIFPPVRQASLL

SEQ ID NO: 67 - protein, 125 aa, synthetic construct
MAESLYAFIDSPGGIAPVQEGTSNRYNFFCPQSFHIPPHGVVLLHLKVSVLVPTGYQGRFMALNDYHARDILTQSDVIFAGRRQELTVLLFNHTDRFLYVRKGHPVGTLLLERVIFPSVKIATLV

SEQ ID NO: 68 - protein, 123 aa, synthetic construct
MAEVLYVILEGPGARLPVQEGNNYIFYAPVDFTLHPRGVALLHLRLSIIVPRCYIGRFFSLTDTNTSGLYASSQIIFAAHQQPLSVMLFNHTDRFYEGRVGDPVACLVLERVIYPSVRQASMM

SEQ ID NO: 69 - protein, 125 aa, synthetic construct
MAESLYAFIDSPGGIAPVQEGSSNRYNFFCPESFHIPPHGVVLLHLRVSVLIPTGYQGRFMALNDYHARGILTQSDVIFAGRRHELTVLLFNHTDRFLYVREGHPVGTLLLERVIFPSVRLATLV

SEQ ID NO: 70 - protein, 125 aa, synthetic construct
MAESLYAFIDSPGGIAPVQEGTSNRYTFFCPESFHIPPHGVVLVHLRVSVLIPNGYQGRFMALNDYHSRGILTQSDVIFAGRRQELTVLLFNHTDRFLYVREGHPVGTLLLERVIFPSVRLATLV

SEQ ID NO: 71 - protein, 125 aa, synthetic construct
MAESLYAFIDSPGGIAPVQEGASNRYTFFCPESFHIPPHGVILLHLRVSVLVPTGYQGRFMALNDYHARGILTQSDVIFAGRRHDLSVLLFNHTDRFLYVREGHPVGTLLLERVIFPSVRLATLV

SEQ ID NO: 72 - protein, 125 aa, synthetic construct
MAESLYAFIDSPGGIAPVQEGASNRYIFFCPESFHIPPHGVILLHLRVSVLVPTGYQGRFMALNDYHARGILTQSDVIFAGRRHDLSVLLFNHTDRFLYVREGHPVGTLLLERVIFPSVRIATLV

SEQ ID NO: 73 - protein, 125 aa, synthetic construct
MADEALYVYLDGPGATLPEQQQRNNYIFYSPVPFTLYPRGVALLYLRLSIIIPRGYVGCFFSLTDANMSGLYASSRIIHAGHREELSVLLFNHDDRFYEGRAGDPVACLVMERLIYPPVRQATMI

SEQ ID NO: 74 - protein, 125 aa, synthetic construct
MAHEALYVYLEGPGATLPEQQQRNNYIFYSPVPFTLYPRGVALLYLRLSIIIPRGYIGCFLSLTDANMFGLYASSRIIHAGHREELSVLLFNHDDRFYEGRAGDPVACLVMERLIYPPVRQATLI

SEQ ID NO: 75 - protein, 125 aa, synthetic construct
MAESLYAFIDSPGGIAPVQEGASNRYIFFCPESFHIPPHGVILLHLRVSVMVPTGYQGRFMALNDYHARGILTQSDVIFAGRRHDLSVLLFNHTDRFLYVREGHPVGTLLLERVIFPSVRIATLV

SEQ ID NO: 76 - protein, 125 aa, synthetic construct
MAESLYAFIDSPGGIAPVQEGTSNRYDFFCPESFHIPPHGVVLLHLRVSVLIPTGYQGRFMALNDYHARGILTQSDVIFAGRRHELTVLLFNHTDRFLYVREGHPVGTLLLERVIFPSVRLATLV

SEQ ID NO: 77 - protein, 150 aa, synthetic construct
MTTSGVPFGMTLRPTRSRLSRRTPYSRDRLPPFETETRATILEDHPLLPECNTLTMHNAWTSPSPPVEQPQVGQQPVAQQLDSNMNLRELPGEFINITDERLARQETVWNITPKNMSVTHDMMLFKASRGERTVYSVCWEGGGRLNTRVL

SEQ ID NO: 78 - protein, 150 aa, synthetic construct
MTTSGVPFGMTLRPTRSRLSRRTPYSRDRLPPFETETRATILEDHPLLPECNTLTMHNAWTSPSPPVEQPQVGQQPVAQQLDSDMNLSELPGEFINITDERLARQETVWNITPKNMSVTHDMMLFKASRGERTVYSVCWEGGGRLNTRVL

SEQ ID NO: 79 - protein, 141 aa, synthetic construct
MSGSNSIMTRLRARSTSCARHHPYTRAQLPRCEENETRASMTEDHPLLPDCDTMTMHSMTVIQTPESHPQQLDCESALKDYPDAFLCITDPRLARSETVWNVESKTMSISNGIQMFKAVRGERLVYSVKWEGGGKITTRIL

SEQ ID NO: 80 - protein, 141 aa, synthetic construct
MSGNSSIMTRSRTRLALSRHHPYQPPATLPRCEETESRASLVEDHPVLPDCDTLSMHNITVIPITEDSPQLLNCEVQMQECPEGFISLTDPRLSRSETVWNVEIKTMSITNSIQMFKAVRGERIVYSMRWEGGGKITTRIL

SEQ ID NO: 81 - protein, 150 aa, synthetic construct
MTTSGVPFGMTLRPTRSRLSRRTPYSRDRLPPFETETRATILEDHPLLPECNTLTMHNAWTSPSPSVKRPQVGQQPVAQQLDSDMNLSELPGEFINITDERLARQETVWNITPKNMSVTHDMMLFKASRGERTVYSVCWEGGGRLNTRVL

SEQ ID NO: 82 - protein, 141 aa, synthetic construct
MSGSNSIMTRLRARSTSCARHHPYTRAQLPRCEENETRASMTEDHPLLPDCDTMTMHSMTVIQTPESHPQQLDCESALKDYPDAFLCITDPRLARFETVWNVESKTMSICNGIQMFKAVRGERLVYSVKWEGGGKITTRIL

SEQ ID NO: 83 - protein, 141 aa, synthetic construct
MSGSNSIMTRLRARSTSCARHHPYTRAQLPRCEENETRASMTEDHPLLPDCDTMTMHSMTVIQTPESQPQQLDCESALKDYPDAFLSITDPRLARSETVWNVESKTMSISNGIQMFKAVRGERLVYSVKWEGGGKITTRIL

SEQ ID NO: 84 - protein, 141 aa, synthetic construct
MSGSNSIMTRLCARSTSCARHHPYTRAQLPRCEENETRASMTEDHPLLPDCDTMTMHSMTVIQTPESHPQQLDCESALKDYPAGFLSITDPRLARYETVWNVESKTMSISNGIQMFKAVRGERLVYSVKWEGGGKITTRSL

SEQ ID NO: 85 - protein, 126 aa, synthetic construct
MQRNRRYPYRLAPYGKYPLPPCEKEMRASLFGPENSSVSECNSLTLHNVINMDLVLDGESYLSDCVGEGFVSIIDHRFARKETIWTVTPKNLSRNMHMQLFSAIKGERVVYKIKWEGGGSLTTRIV

SEQ ID NO: 86 - protein, 130 aa, synthetic construct
MQTEDQSSLLRHHPYRRARLPRSDEETRASLTEQHPLLPDCDHADYHNTVTLDCEARLDDFSEDGFISITDPRLARQETVWIIDPKLSSRTNQNIPLFKATRAERTVYTVKWAGGGRLTTRAGVKINKDT

SEQ ID NO: 87 - protein, 130 aa, synthetic construct
MQTEIQSSSLRHHPYRRARLPRSDEETRASLTEQHPLLPDCDHADYHNTVTLDCEARLDDFSEDGFISITDPRLARQETVWIIDPKSSSRTNQNISLFKATRAERTVYTVKWAGGGRLTTRAGVKINKDT

SEQ ID NO: 88 - protein, 130 aa, synthetic construct
MSTEEQSSSLRHHPYRRARLPRCEEETRASLTEQHPLLPDCDHADYHNTVTLDCEARLDDFSEDGFISITDPRLARQETVWIIDTKTSSRTNNNIPLFKATRAERIVYTVKWAGGGRLTTRAGVKINKDT

SEQ ID NO: 89 - DNA, 23 bp, synthetic construct
  source 1..23: mol_type = other DNA
ctgtgatcttcaagaccattgtg

SEQ ID NO: 90 - DNA, 21 bp, synthetic construct
  source 1..21: mol_type = other DNA
agtttgggtttgcttgtccag

SEQ ID NO: 91 - DNA, 24 bp, synthetic construct
  source 1..24: mol_type = other DNA
catctcactgtgtgtaaacatgac

SEQ ID NO: 92 - DNA, 23 bp, synthetic construct
  source 1..23: mol_type = other DNA
ccttggcaaaactgcaccttcac

SEQ ID NO: 93 - DNA, 24 bp, synthetic construct
  source 1..24: mol_type = other DNA
gattcaatgaggagacttgcctgg

SEQ ID NO: 94 - DNA, 25 bp, synthetic construct
  source 1..25: mol_type = other DNA
ctcactactctcaaatctgttctgg

SEQ ID NO: 95 - DNA, 21 bp, synthetic construct
  source 1..21: mol_type = other DNA
ggttgagtttaagccaatcca

SEQ ID NO: 96 - DNA, 20 bp, synthetic construct
  source 1..20: mol_type = other DNA
tgctgacctaggcttgatga

SEQ ID NO: 97 - DNA, 24 bp, synthetic construct
  source 1..24: mol_type = other DNA
cagcatgacagatttctaccactc

SEQ ID NO: 98 - DNA, 18 bp, synthetic construct
  source 1..18: mol_type = other DNA
ctcgcgcttccaggactg

SEQ ID NO: 99 - DNA, 23 bp, synthetic construct
  source 1..23: mol_type = other DNA
gcagaggaacctccagtctcagc

SEQ ID NO: 100 - DNA, 23 bp, synthetic construct
  source 1..23: mol_type = other DNA
gagagaggtactccttgaatgcc

SEQ ID NO: 101 - DNA, 23 bp, synthetic construct
  source 1..23: mol_type = other DNA
ctgacacctccaagtatcccaag

SEQ ID NO: 102 - DNA, 24 bp, synthetic construct
  source 1..24: mol_type = other DNA
gtcgtgtacagaagtacagaccgc

(19) United States
(12) Patent Application Publication
(10) Pub. No.: US 2024/0408145 A1
(43) Pub. Date: Dec. 12, 2024

(54) Gene Therapy

(71) Applicants: Ospedale San Raffaele S.r.l., Milan (IT); Fondazione Telethon ETS, Rome (IT)

(72) Inventors: Rafaella Di Micco, Milan (IT); Lucrezia Della Volpe, Milan (IT); Anastasia Conti, Milan (IT)

(21) Appl. No.: 18/702,592

(22) PCT Filed: Oct. 11, 2022
(86) PCT No.: PCT/EP2022/078295

§ 371 Date: Apr. 18, 2024

(30) Foreign Application Priority Data
Oct. 19, 2021 (GB) 2114972.9

(51) Int. Cl. A61K 35/28; C12N 5/0789; C12N 15/86

(52) U.S. Cl. CPC A61K 35/28; C12N 5/0647; C12N 15/86; C12N 2501/2301; C12N 2501/727; C12N 2510/00; C12N 2750/14143

(57) ABSTRACT

Use of one or more inhibitor(s) of senescence for increasing the survival and/or engraftment of haematopoietic cells, haematopoietic stem cells, haematopoietic progenitor cells and/or T cells in gene therapy.

### FIELD OF THE INVENTION

The present invention relates to the genetic modification of cells. More specifically, the present invention relates to the use of inhibitors to improve the efficiency of gene editing and to improve the survival and/or engraftment of haematopoietic stem cells and/or T cells which have been gene edited.

### BACKGROUND TO THE INVENTION

The haematopoietic system is a complex hierarchy of cells of different mature cell lineages. These include cells of the immune system that offer protection from pathogens, cells that carry oxygen through the body and cells involved in wound healing. All these mature cells are derived from a pool of haematopoietic stem cells (HSCs) that are capable of self-renewal and differentiation into any blood cell lineage. HSCs have the ability to replenish the entire haematopoietic system.

Haematopoietic cell transplantation (HCT) is a curative therapy for several inherited and acquired disorders. However, allogeneic HCT is limited by the poor availability of matched donors, the mortality associated with the allogeneic procedure, which is mostly related to graft-versus-host disease (GvHD), and infectious complications provoked by the profound and long-lasting state of immune dysfunction.

Adoptive immunotherapy with engineered cells (e.g. T-cells or NK cells) is a promising new clinical strategy. T-cells and NK cells engineered with e.g. transgenic T-cell receptors (TCRs) or chimeric antigen receptors (CARs) are promising cancer treatments.

Gene therapy approaches based on the transplantation of genetically modified autologous HSCs and/or T cells offer potentially improved safety and efficacy over allogeneic HCT and/or allogeneic adoptive immunotherapy. They are particularly relevant for patients lacking a matched donor.

The concept of stem cell gene therapy or adoptive immunotherapy is based on the genetic modification of a relatively small number of stem cells, T cells or NK cells. These modified stem cells persist long-term in the body by undergoing self-renewal, and generate large numbers of genetically “corrected” progeny. This ensures a continuous supply of corrected cells for the rest of the patient's lifetime. HSCs are particularly attractive targets for gene therapy since their genetic modification will be passed to all the blood cell lineages as they differentiate. Furthermore, HSCs can be easily and safely obtained, for example from bone marrow, mobilised peripheral blood and umbilical cord blood.

Efficient long-term gene modification of HSCs and their progeny (as well as T cells) requires a technology which permits stable integration of the corrective DNA into the genome, without affecting HSC function. Accordingly, the use of integrating recombinant viral systems such as γ-retroviruses, lentiviruses and spumaviruses has dominated this field (Chang, A. H. et al. (2007) Mol. Ther. 15: 445-456). Therapeutic benefits have already been achieved in γ-retrovirus-based clinical trials for Adenosine Deaminase Severe Combined Immunodeficiency (ADA-SCID; Aiuti, A. et al. (2009) N. Engl. J. Med. 360: 447-458), X-linked Severe Combined Immunodeficiency (SCID-X1; Hacein-Bey-Abina, S. et al. (2010) N. Engl. J. Med. 363: 355-364) and Wiskott-Aldrich syndrome (WAS; Boztug, K. et al. (2010) N. Engl. J. Med. 363: 1918-1927). In addition, lentiviruses have been employed as delivery vehicles in the treatment of X-linked adrenoleukodystrophy (ALD; Cartier, N. et al. (2009) Science 326: 818-823), and recently for metachromatic leukodystrophy (MLD; Biffi, A. et al. (2013) Science 341: 1233158) and WAS (Aiuti, A. et al. (2013) Science 341: 1233151).

In addition to the use of retro- and lentiviral-based vectors, vectors derived from other viruses, such as adenoviruses and adeno-associated viruses (AAV), may also be utilised for the modification of haematopoietic stem and progenitor cells.

The scope of genetic engineering has recently broadened from gene replacement to targeted gene editing using engineered nucleases, which enable precise sequence modification of a locus of interest. In other words, Hematopoietic Stem and/or Progenitor Cells (HSPCs) and/or T cells can be genetically modified to prevent or treat diseases by adding healthy genes (gene transfer) or by precisely repairing a genetic defect (gene editing). Gene editing applications encompass targeted disruption of a gene coding sequence, precise sequence substitution for in situ correction of mutations and targeted transgene insertion into a predetermined locus. Gene editing is based on the design of artificial endonucleases that target a double-strand break (DSB) or nick into the sequence of interest in the genome. Cells repair the DSB through two major mechanisms, Non-Homologous End-Joining (NHEJ) or Homology Directed Repair (HDR), although other repair mechanisms might be eventually exploited. NHEJ often creates small insertions or deletions (“indels”) at the target site that may disrupt the coding sequence of a gene, whereas HDR can be exploited to precisely introduce a novel sequence at the target site by providing an exogenous template DNA bearing homology to the sequences flanking the DSB.

Multiple platforms of artificial endonucleases can be used to target a locus of interest, including Zinc Finger Nucleases (ZFNs), TAL effector nucleases (TALENs) and the more recently developed RNA-based CRISPR/Cas9 nucleases. Viral vectors are the most efficient delivery vehicle for a DNA template, for example, the AAV6 vector is able to achieve a high transduction efficiency in human primary cells, such as HSPCs and T lymphocytes.

The success of HSPC gene therapies and adoptive immunotherapies critically depends on the capacity to genetically modify HSPCs and/or T cells without compromising their functional properties and vitality. Current protocols for gene transfer and gene editing require prolonged ex-vivo culture, high viral vector doses and nuclease-induced DNA DSBs, that activates the DNA Damage Response (DDR) pathway, leading to cell cycle arrest. Emerging data indicates that cellular detection of viral vectors employed in classical gene therapy settings, instead of eliciting innate immune mediated recognition of viral nucleic acids or proteins, unexpectedly triggers the DDR.

The DDR pathway is an evolutionary conserved set of actions converging on key decision-making factors such as the tumour suppressor p53 to enforce cell cycle arrest. The activation of the DDR pathway impairs the haematopoietic reconstitution of gene-modified cells by gene addition with lentiviral vectors upon transplantation (Piras, F. et al., 2017, EMBO Mol Med 9: 1198-1211). In line with this, the engagement of the p53 DDR signalling cascade was recently identified as a barrier to successful gene-editing procedures in HSPCs (Schiroli, G. et al., 2019, Cell Stem Cell 24: 551-565; and Conti, A. & Di Micco, R., 2018, Genome Med 10: 66). Even a single nuclease-induced DSB triggers a detectable, albeit transient, DDR in HSPCs. Unexpectedly, the concomitant exposure to nuclease-induced DSB and recombinant adeno-associated virus serotype 6 (rAAV6), which currently represents the preferred source for HDR template delivery during gene-editing, led to elevated DDR burden and prolonged HSPC proliferation arrest, in the absence of cell death, with consequent impairment in the post-transplant engraftment capacity of the HSPCs. An in-depth molecular characterization of DDR dynamics coupled to single-cell transcriptomic studies recently guided the development of an innovative strategy based on transient inhibition of p53 during the editing procedure that improves haematopoietic reconstitution, without aggravating chromosomal translocations or mutational burden (Schiroli, G. et al., 2019, Cell Stem Cell 24: 551-565; and WO 2020002380). This experimental evidence uncovers a previously uncharted interplay between viral vector sensing and the host cell DDR machinery and represents the first example of how temporary manipulation of DDR programs may impact on the biology of gene-corrected HSPCs.

Substantial difficulties remain with the methods employed for the genetic modification of HPSCs and T cells. In particular, the multiple hits of high vector doses required and prolonged ex vivo activation times associated with existing methods give rise to problems with survival of the transduced HSPCs and T cells during culture and potentially impact their biological properties. Furthermore, improvements in the engraftment of transduced cells will greatly benefit clinical applications.

### SUMMARY OF THE INVENTION

The present inventors have studied the inflammatory response caused by DDR activation due to the induction of double strand breaks (DSBs) of DNA by nucleases in gene editing/therapy engineering technologies. The present inventors have recently found that cellular detection of viral vectors employed in classical gene therapy settings unexpectedly triggers the DDR. The activation of the DDR pathway, via the induction of DSBs and cellular detection of viral vectors, impairs the haematopoietic reconstitution of gene-modified cells upon transplantation.

In addition to the above challenges associated with gene therapy and gene editing approaches in HSPCs and T cells, the present inventors hypothesise that current gene engineering protocols (which require prolonged activation in culture, high vector doses and nuclease-induced DSB) may inadvertently trigger the activation of a cellular senescence program in HSPCs with both cell-autonomous and paracrine short- and long-term consequences on engineered human haematopoiesis. This program may in turn hamper the proliferation of gene-modified cells and affect their clonal composition and dynamics of haematopoietic reconstitution upon transplantation, thus posing a real challenge to unlocking the full potential of gene therapy approaches. The impact of senescence on haematopoietic reconstitution may be further worsened when i) low numbers of ex-vivo gene corrected cells are available for transplantation, ii) HSPCs are collected from donors of older age and/or iii) the underlying disease pathophysiology is inherently linked to hematopoietic stress (e.g. Fanconi Anemia) or hyper-inflammation (e.g. Chronic Granulomatous Disease).

The present inventors have developed an improved protocol for culturing Hematopoietic Stem Cells (HSC), Progenitor Cell (HSPC) and T cells engineered with viral vectors (AAV or LV) for gene therapy and/or gene editing. The present inventors have surprisingly found that adding inhibitors of senescence, and in particular inhibitors targeting IL-1 and NF-kB signalling pathways, at the time of gene editing and/or transduction with viral vectors dampens the DDR-dependent inflammatory response and improves clonogenic potential and in vivo long-term reconstitution of gene edited and/or transduced cells. The present inventors also surprisingly found that pre-culturing cells with inhibitors of senescence (which decrease the percentage of senescent cells), and in particular inhibitors of p38 MAPK, improves the efficacy of gene editing and/or transduction.

Advantageously, the invention improves functionality of gene edited and/or transduced HSPCs and/or T cells by inhibiting senescence before gene editing/transduction and by inhibiting the DDR pathway due to DNA DSBs and its associated inflammatory response during gene editing/transduction.

Accordingly, in one aspect, the invention provides the use of one or more inhibitor(s) of senescence for increasing the survival and/or engraftment of haematopoietic cells, haematopoietic stem cells, haematopoietic progenitor cells and/or T cells in gene therapy.

In a further aspect, the invention provides the use of one or more inhibitor(s) of senescence for increasing the efficiency of gene editing of haematopoietic cells, haematopoietic stem cells, haematopoietic progenitor cells, and/or T cells.

In a further aspect, the invention provides one or more inhibitor(s) of senescence for use in haematopoietic cell gene therapy, haematopoietic stem cell gene therapy, haematopoietic progenitor cell gene therapy and/or T cell gene therapy.

In a further aspect, the invention provides one or more inhibitor(s) of senescence for use in gene therapy in increasing the survival and/or engraftment of haematopoietic cells, haematopoietic stem cells, haematopoietic progenitor cells and/or T cells.

In a further aspect, the invention provides the use of one or more inhibitor(s) of senescence for preserving or increasing the fitness of haematopoietic cells, haematopoietic stem cells, haematopoietic progenitor cells and/or T cells in gene therapy.

In a further aspect, the invention provides one or more inhibitor(s) of senescence for use in gene therapy in preserving or increasing the fitness of haematopoietic cells, haematopoietic stem cells, haematopoietic progenitor cells and/or T cells.

In some embodiments, the fitness is preserved or increased in gene edited haematopoietic cells, haematopoietic stem cells, haematopoietic progenitor cells and/or T cells.

Suitably, a combination of inhibitors of senescence are used. Suitably, each of the inhibitors of senescence within the combination are distinct inhibitors. For example, each of the inhibitors of senescence may target a different molecule, i.e. the inhibitors may not target the same molecule.

In some embodiments, the one or more inhibitor(s) of senescence are in the form of a composition or a kit.

In some embodiments, the inhibitors of senescence are in combination. Suitably, the inhibitors may be administered simultaneously, sequentially or separately.

In some embodiments, the one or more inhibitor(s) of senescence comprises or consists of an inhibitor of MAPK/ERK signalling, an IL-1 inhibitor and/or an NF-κB inhibitor. Suitably, the inhibitor of MAPK/ERK signalling is a MAPK inhibitor.

In some embodiments, the one or more inhibitor(s) of senescence comprises or consists of a MAPK inhibitor, an IL-1 inhibitor and/or an NF-κB inhibitor.

Accordingly, in one aspect, the invention provides the use of a MAPK inhibitor, an IL-1 inhibitor and/or an NF-κB inhibitor for increasing the survival and/or engraftment of haematopoietic cells, haematopoietic stem cells, haematopoietic progenitor cells and/or T cells in gene therapy.

In a further aspect, the invention provides the use of a MAPK inhibitor, an IL-1 inhibitor and/or an NF-κB inhibitor for increasing the efficiency of gene editing of haematopoietic cells, haematopoietic stem cells, haematopoietic progenitor cells, and/or T cells.

In a further aspect, the invention provides a MAPK inhibitor, an IL-1 inhibitor and/or an NF-κB inhibitor for use in haematopoietic cell gene therapy, haematopoietic stem cell gene therapy, haematopoietic progenitor cell gene therapy and/or T cell gene therapy.

In a further aspect, the invention provides a MAPK inhibitor, an IL-1 inhibitor and/or an NF-κB inhibitor for use in gene therapy in increasing the survival and/or engraftment of haematopoietic cells, haematopoietic stem cells, haematopoietic progenitor cells and/or T cells.

In some embodiments, the gene therapy comprises gene transfer.

In some embodiments, the gene therapy comprises gene editing.

In some embodiments, the one or more inhibitor(s) of senescence comprises or consists of a MAPK inhibitor. Suitably, a MAPK inhibitor is used.

In some embodiments, the one or more inhibitor(s) of senescence comprises or consists of an IL-1 inhibitor. Suitably, an IL-1 inhibitor is used.

In some embodiments, the one or more inhibitor(s) of senescence comprises or consists of an NF-κB inhibitor. Suitably, an NF-κB inhibitor is used.

In some embodiments, the one or more inhibitor(s) of senescence comprises or consists of a MAPK inhibitor and an IL-1 inhibitor. Suitably, a MAPK inhibitor and an IL-1 inhibitor are used.

In some embodiments, the one or more inhibitor(s) of senescence comprises or consists of a MAPK inhibitor and an NF-κB inhibitor. Suitably, a MAPK inhibitor and an NF-κB inhibitor are used.

In some embodiments, the one or more inhibitor(s) of senescence comprises or consists of an IL-1 inhibitor and an NF-κB inhibitor. Suitably, an IL-1 inhibitor and an NF-κB inhibitor are used.

In some embodiments, the one or more inhibitor(s) of senescence comprises or consists of a MAPK inhibitor, an IL-1 inhibitor and an NF-κB inhibitor. Suitably, a MAPK inhibitor, an IL-1 inhibitor and an NF-κB inhibitor are used.

In one embodiment, the inhibitor of MAPK/ERK signalling (e.g. a MAPK inhibitor), IL-1 inhibitor and/or NF-κB inhibitor are administered simultaneously, sequentially or separately.

In some embodiments, the IL-1 inhibitor and/or NF-κB inhibitor inhibits DDR-dependent inflammation. Suitably, the inhibition of DDR-dependent inflammation increases the survival and/or engraftment of haematopoietic cells, haematopoietic stem cells, haematopoietic progenitor cells and/or T cells in gene therapy. Suitably, the inhibition of DDR-dependent inflammation increases the efficiency of gene editing of haematopoietic cells, haematopoietic stem cells, haematopoietic progenitor cells, and/or T cells.

In one embodiment, at least 1%, 2%, 3%, 4%, 5%, 6%, 7%, 8%, 9%, 10%, 15%, 20%, 25%, 30%, 50%, 75% or 90%, preferably at least 70%, more cells survive in culture for a period of time (e.g. about 6 or 12 hours, or 1, 2, 3, 4, 5, 6, 7 or more days, preferably about 2 days) when the cells have been exposed to the inhibitor(s) rather than in its absence. Preferably, the period of time begins with thawing of the cells into the culture medium.

The present invention may allow the timing of the pretreatment/conditioning of cells with cytokines to prime the cells (otherwise quiescent) for transduction to be reduced. Standard timing for HSPCs is 3 days. This time may be reduced to less than 3 days, or less than 2 days, or less than 1 day, for example 0, 1 or 2 days.

In one embodiment, at least 1%, 2%, 3%, 4%, 5%, 6%, 7%, 8%, 9%, 10%, 15%, 20%, 25%, 30%, 40%, 50% or 75%, preferably at least 10%, more transplanted haematopoietic stem cells and/or haematopoietic progenitor cells and/or their descendant cells (e.g. graft-derived cells) engraft in a host subject when the cells have been exposed to the inhibitor(s) rather than in its absence.

In one embodiment, the cells are HSCs.

In one embodiment, the cells are HSPCs.

In one embodiment, the HSPCs are CD34⁺ cells.

In one embodiment, the population of haematopoietic stem and/or progenitor cells comprises, is enriched in or substantially consists of CD34⁺ cells. The population of cells may be further enriched for a particular sub-population of cells, for example CD34⁺CD38-cells. The population of cells may be further enriched for a particular sub-population of cells, for example CD34⁺CD133⁺ and CD90⁺ cells.

In some embodiments, the inhibitor of MAPK/ERK signalling is a MAP3K inhibitor, a MAK2K inhibitor, a MAPK inhibitor, preferably an MKK7 inhibitor, an MKK4 inhibitor, an MKK3/6 inhibitor, an MEK1/2 inhibitor, a JNK inhibitor, a p38 inhibitor or an ERK inhibitor.

In some embodiments, the MAPK inhibitor is an inhibitor of p38 phosphorylation, an inhibitor of JNK phosphorylation or an inhibitor of ERK phosphorylation, preferably an inhibitor of p38 phosphorylation.

In some embodiments, the MAPK inhibitor is a JNK inhibitor, a p38 inhibitor or an ERK inhibitor.

In some embodiments, the MAPK inhibitor is FR180204, SP600125, SB203580, SB202190, LY2228820, BIRB 796; SB203580 hydrochloride, SCIO 469 hydrochloride, TMCB, XMD 8-92, TCS JNK 6o, SU 3327, CC 401 dihydrochloride, or a derivative thereof.

In some embodiments, the MAPK inhibitor is FR180204, SP600125, SB203580 or a derivative thereof.

In some embodiments, the IL-1 inhibitor is an anti-IL-1a antibody, an anti-IL-1β antibody, an IL-1 antagonist, an IL-1 receptor antagonist, an IL-1a converting enzyme inhibitor, an IL-1β converting enzyme inhibitor, or a soluble decoy IL-1 receptor.

In some embodiments, the IL-1 inhibitor is anakinra, canakinumab, rilonacept, gevokizumab or a variant thereof.

In some embodiments, the IL-1 inhibitor is anakinra or a variant thereof.

In some embodiments, the NF-κB inhibitor is an IL-1 inhibitor, an IL-1 receptor inhibitor, a TLR4 inhibitor, a TAK1 inhibitor, an Akt inhibitor, an IKK inhibitor, an inhibitor of IκB phosphorylation, an inhibitor of IκB degradation, an inhibitor of the proteasome, an inhibitor of IκBα upregulation, an inhibitor of NF-κB nuclear translocation, an inhibitor of NF-κB expression, an inhibitor of NF-κB DNA binding, or an inhibitor of NF-κB transactivation.

In some embodiments, the NF-κB inhibitor is SC514 or a derivative thereof; anakinra, canakinumab, rilonacept, gevokizumab or a variant thereof; or metformin, apigenin, kaempferol, BAY 11-7082, or a derivative thereof.

In some embodiments, the NF-κB inhibitor is SC514 or a derivative thereof.

In some embodiments, the use further comprises the use of an agent which promotes homology directed DNA repair.

In some embodiments, the agent is an inhibitor of p53 activation, preferably wherein the inhibitor is an inhibitor of p53 phosphorylation, more preferably an inhibitor of p53 Serine 15 phosphorylation.

In some embodiments, the inhibitor of p53 activation is a p53 dominant negative peptide, an ataxia telangiectasia mutated (ATM) kinase inhibitor or an ataxia telangiectasia and Rad3-related protein (ATR) inhibitor.

In some embodiments, the inhibitor of p53 activation is pifithrin-α or a derivative thereof; KU-55933 or a derivative thereof; GSE56 or a variant thereof; KU-60019, BEZ235, wortmannin, CP-466722, Torin 2, CGK 733, KU-559403, AZD6738 or derivatives thereof; or an siRNA, shRNA, miRNA or antisense DNA/RNA, preferably wherein the inhibitor of p53 activation is GSE56 or a variant thereof.

In some embodiments, the inhibition of senescence (e.g. the inhibition of MAPK, inhibition of IL-1 and/or inhibition of NF-κB) in the haematopoietic cells, haematopoietic stem cells, haematopoietic progenitor cells and/or T cells is transient.

In one embodiment, the one or more inhibitor(s) of senescence is a transient inhibitor (e.g. has an inhibitory action lasting less than about 1, 2, 3, 4, 5, 6, 7 or 14 days), such as a reversible inhibitor. Preferably, the cells are exposed to the inhibitor(s) for about 1-48 or 1-24 hours, preferably 1-24 hours. The cells may be, for example, exposed to the inhibitor(s) prior to, at the same time as or after the viral vector and/or gene editing machinery.

In some embodiments, the inhibition of p53 activation in the haematopoietic cells, haematopoietic stem cells, haematopoietic progenitor cells and/or T cells is transient. In one embodiment, the inhibitor of p53 activation is a transient inhibitor (e.g. has an inhibitory action lasting less than about 1, 2, 3, 4, 5, 6, 7 or 14 days), such as a reversible inhibitor. Preferably, the cells are exposed to the inhibitor(s) for about 1-48 or 1-24 hours, preferably 1-24 hours. The cells may be, for example, exposed to the inhibitor(s) prior to, at the same time as or after the viral vector and/or gene editing machinery.

In some embodiments, the haematopoietic cells, haematopoietic stem cells, haematopoietic progenitor cells and/or T cells are exposed to the IL-1 inhibitor and/or NF-κB inhibitor prior to, at the same time as and/or after the gene editing machinery is introduced into the cell, preferably at the same time as the gene editing machinery is introduced to the cell.

In some embodiments, the inhibition of IL-1 and/or NF-κB occurs during gene editing of the haematopoietic cells, haematopoietic stem cells, haematopoietic progenitor cells and/or T cells.

In some embodiments, the haematopoietic cells, haematopoietic stem cells, haematopoietic progenitor cells and/or T cells are exposed to the MAPK inhibitor prior to the gene editing machinery being introduced to the cell.

In some embodiments, the MAPK inhibition occurs prior to and/or during gene editing of the haematopoietic cells, haematopoietic stem cells, haematopoietic progenitor cells and/or T cells.

In some embodiments, the haematopoietic cells, haematopoietic stem cells, haematopoietic progenitor cells and/or T cells are exposed to the inhibitor of p53 activation prior to, at the same time as and/or after the gene editing machinery is introduced into the cell.

In some embodiments, the inhibition of p53 occurs during gene editing of the haematopoietic cells, haematopoietic stem cells, haematopoietic progenitor cells and/or T cells.

In some embodiments, the one or more inhibitor(s) of senescence (e.g. the MAPK inhibitor, IL-1 inhibitor and/or NF-κB inhibitor) is added to the haematopoietic cell, haematopoietic stem cell, haematopoietic progenitor cells and/or T cells at a concentration of about 0.5-200 μM or about 0.1-200 ng/μl.

In one embodiment, the one or more inhibitor(s) of senescence (e.g. the MAPK inhibitor, IL-1 inhibitor and/or NF-κB inhibitor) is added to the haematopoietic cells, haematopoietic stem cells, haematopoietic progenitor cells and/or T cells (e.g. in an in vitro or ex vivo culture) at a concentration of about 0.5-200, 0.5-150, 0.5-100, 0.5-50, 0.5-40, 0.5-30, 0.5-20 or 0.5-15 μM, preferably about 0.5-30 μM, more preferably about 0.5-15 μM. In one embodiment, the one or more inhibitor(s) of senescence (e.g. the MAPK inhibitor, IL-1 inhibitor and/or NF-κB inhibitor) is added to the haematopoietic cells, haematopoietic stem cells, haematopoietic progenitor cells and/or T cells (e.g. in an in vitro or ex vivo culture) at a concentration of about 1-200, 1-150, 1-100, 1-50, 1-40, 1-30, 1-20 or 1-15 μM, preferably about 1-30 μM, more preferably about 1-15 μM. In another embodiment, the inhibitor(s) is added to the haematopoietic cells, haematopoietic stem cells, haematopoietic progenitor cells and/or T cells at a concentration of about 5-200, 5-150, 5-100, 5-50, 5-40, 5-30, 5-20 or 5-15 μM, preferably about 5-30 μM.

In one embodiment, the one or more inhibitor(s) of senescence (e.g. the MAPK inhibitor, IL-1 inhibitor and/or NF-κB inhibitor) is added to the haematopoietic cells, haematopoietic stem cells, haematopoietic progenitor cells and/or T cells (e.g. in an in vitro or ex vivo culture) at a concentration of about 0.5, 1, 2, 3, 4, 5, 6, 7, 8, 9, 10, 11, 12, 13, 14, 15, 20, 25, 30, 35, 40, 45, 50, 75, 100, 125, 150, 175 or 200 μM. In another embodiment, the one or more inhibitor(s) of senescence (e.g. the MAPK inhibitor, IL-1 inhibitor and/or NF-κB inhibitor) is added to the haematopoietic cells, haematopoietic stem cells, haematopoietic progenitor cells and/or T cells (e.g. in an in vitro or ex vivo culture) at a concentration of about 2 μM. In another embodiment, the one or more inhibitor(s) of senescence (e.g. the MAPK inhibitor, IL-1 inhibitor and/or NF-κB inhibitor) is added to the haematopoietic cells, haematopoietic stem cells, haematopoietic progenitor cells and/or T cells (e.g. in an in vitro or ex vivo culture) at a concentration of about 4 μM. In another embodiment, the one or more inhibitor(s) of senescence is added to the haematopoietic cells, haematopoietic stem cells, haematopoietic progenitor cells and/or T cells (e.g. in an in vitro or ex vivo culture) at a concentration of about 12 μM.

In one embodiment, the one or more inhibitor(s) of senescence (e.g. the MAPK inhibitor, IL-1 inhibitor and/or NF-κB inhibitor) is added to the haematopoietic cells, haematopoietic stem cells, haematopoietic progenitor cells and/or T cells (e.g. in an in vitro or ex vivo culture) at a concentration of about 0.1-200, 0.1-150, 0.1-100, 0.1-75, 0.1-60, 0.1-50, 0.1-25, 0.1-20, 0.1-15 or 0.1-10 ng/μl, preferably about 0.1-60 ng/μl. In another embodiment, the inhibitor(s) is added to the haematopoietic cells, haematopoietic stem cells, haematopoietic progenitor cells and/or T cells at a concentration of about 5-200, 5-150, 5-100, 5-75, 5-60, 5-50, 5-25, 5-20, 5-15 or 5-10 ng/μl, preferably about 5-60 ng/μl.

In one embodiment, the one or more inhibitor(s) of senescence (e.g. the MAPK inhibitor, IL-1 inhibitor and/or NF-κB inhibitor) is added to the haematopoietic cells, haematopoietic stem cells, haematopoietic progenitor cells and/or T cells (e.g. in an in vitro or ex vivo culture) at a concentration of about 0.1, 1, 2, 3, 4, 5, 6, 7, 8, 9, 10, 11, 12, 13, 14, 15, 20, 25, 30, 35, 40, 45, 50, 55, 60, 65, 70, 75, 80, 90, 100, 125, 150, 175, or 200 ng/μl, preferably about 50 ng/μl.

In some embodiments, the one or more inhibitor(s) of senescence (e.g. the MAPK inhibitor, IL-1 inhibitor and/or NF-κB inhibitor) is used in combination with at least one adenoviral protein or a nucleic acid sequence encoding therefor.

In some embodiments, the one or more inhibitor(s) of senescence (e.g. the MAPK inhibitor, IL-1 inhibitor and/or NF-κB inhibitor) further comprises at least one adenoviral protein.

In some embodiments, the one or more inhibitor(s) of senescence (e.g. the MAPK inhibitor, IL-1 inhibitor and/or NF-κB inhibitor) further comprises a nucleic acid sequence encoding at least one adenoviral protein.

In some embodiments, the adenoviral protein is expressed transiently in the haematopoietic cell, haematopoietic stem cell, haematopoietic progenitor cell and/or T cell, preferably wherein the transient expression occurs during gene editing of the haematopoietic cell, haematopoietic stem cell, haematopoietic progenitor cell and/or T cell.

In some embodiments, the target of the gene editing is selected from the group consisting of FANC-A, CD40L, RAG-1, IL-2RG, CYBA, CYBB, NCF1, NCF2, and NCF4. In one embodiment, the target of the gene editing is a gene mutated in chronic granulomatous disease or the gene mutated SCID, atypical SCID and Omenn syndrome, or Hyper IgM syndrome.

In a further aspect, the invention provides a method of gene editing a population of haematopoietic cells, haematopoietic stem cells, haematopoietic progenitor cells and/or T cells comprising the steps:
(a) contacting the population of haematopoietic cells, haematopoietic stem cells, haematopoietic progenitor cells and/or T cells with one or more inhibitor(s) of senescence;
(b) introducing gene editing machinery to the population of haematopoietic cells, haematopoietic stem cells, haematopoietic progenitor cells and/or T cells with one or more vectors; and
(c) editing the genome of said haematopoietic cells, haematopoietic stem cells, haematopoietic progenitor cells and/or T cells.

In a further aspect, the invention provides a method of gene editing a population of haematopoietic cells, haematopoietic stem cells, haematopoietic progenitor cells and/or T cells comprising the steps:
(a) contacting the population of haematopoietic cells, haematopoietic stem cells, haematopoietic progenitor cells and/or T cells with a MAPK inhibitor, an IL-1 inhibitor and/or an NF-κB inhibitor;
(b) introducing gene editing machinery to the population of haematopoietic cells, haematopoietic stem cells, haematopoietic progenitor cells and/or T cells with one or more vectors; and
(c) editing the genome of said haematopoietic cells, haematopoietic stem cells, haematopoietic progenitor cells and/or T cells.

In a further aspect, the invention provides a method of transducing a population of haematopoietic cells, haematopoietic stem cells, haematopoietic progenitor cells and/or T cells with a viral vector comprising the steps:
(a) contacting the population of haematopoietic cells, haematopoietic stem cells, haematopoietic progenitor cells and/or T cells with one or more inhibitor(s) of senescence; and
(b) transducing the population of haematopoietic cells, haematopoietic stem cells, haematopoietic progenitor cells and/or T cells with one or more viral vectors.

In a further aspect, the invention provides a method of transducing a population of haematopoietic cells, haematopoietic stem cells, haematopoietic progenitor cells and/or T cells with a viral vector comprising the steps:
(a) contacting the population of haematopoietic cells, haematopoietic stem cells, haematopoietic progenitor cells and/or T cells with a MAPK inhibitor, an IL-1 inhibitor and/or an NF-κB inhibitor; and
(b) transducing the population of haematopoietic cells, haematopoietic stem cells, haematopoietic progenitor cells and/or T cells with one or more viral vectors.

In some embodiments, the method increases the efficiency of transduction of the population of haematopoietic cells, haematopoietic stem cells, haematopoietic progenitor cells and/or T cells. The increased transduction efficiency may, for example, be an increased vector copy number per cell (for example, increased by at least 1.1 fold, 1.2 fold, 1.3 fold, 1.4 fold, 1.5 fold, 1.6 fold, 1.7 fold, 1.8 fold, 1.9 fold, 1.8 fold, 1.9 fold, 2.0 fold, 2.1 fold, 2.2 fold, 2.3 fold, 2.4 fold, 2.5 fold, 3 fold or more). The increased transduction efficiency may, for example, be an increased percentage of cells transduced (for example, increased by 5%, 10%, 15%, 20%, 25%, 30%, 40%, 50%, 60%, 70%, 80%, 90%, 100%, 200%, 300% or more).

In some embodiments, the steps of introducing gene editing machinery to the population of cells and of contacting the population of cells with one or more inhibitor(s) of senescence are carried out ex vivo or in vitro.

In some embodiments, the steps of transducing the population of cells and of contacting the population of cells with one or more inhibitor(s) of senescence are carried out ex vivo or in vitro.

In one embodiment, the cells are HSCs.

In one embodiment, the cells are HSPCs.

In one embodiment, the HSPCs are CD34⁺ cells.

In one embodiment, the population of haematopoietic stem and/or progenitor cells comprises, is enriched in or substantially consists of CD34⁺ cells. The population of cells may be further enriched for a particular sub-population of cells, for example CD34⁺CD38⁻ cells. The population of cells may be further enriched for a particular sub-population of cells, for example CD34⁺CD133⁺ and CD90⁺ cells.

In some embodiments, the one or more inhibitor(s) of senescence comprises or consists of a MAPK inhibitor, an IL-1 inhibitor and/or an NF-κB inhibitor.

In some embodiments, the one or more inhibitor(s) of senescence comprises or consists of a MAPK inhibitor.

In some embodiments, the one or more inhibitor(s) of senescence comprises or consists of an IL-1 inhibitor.

In some embodiments, the one or more inhibitor(s) of senescence comprises or consists of an NF-κB inhibitor.

In some embodiments, the one or more inhibitor(s) of senescence comprises or consists of a MAPK inhibitor and an IL-1 inhibitor.

In some embodiments, the one or more inhibitor(s) of senescence comprises or consists of a MAPK inhibitor and an NF-κB inhibitor.

In some embodiments, the one or more inhibitor(s) of senescence comprises or consists of an IL-1 inhibitor and an NF-κB inhibitor.

In some embodiments, the one or more inhibitor(s) of senescence comprises or consists of a MAPK inhibitor, an IL-1 inhibitor and an NF-κB inhibitor.

In some embodiments, the inhibitor of MAPK/ERK signalling is a MAP3K inhibitor, a MAK2K inhibitor, a MAPK inhibitor, preferably an MKK7 inhibitor, an MKK4 inhibitor, an MKK3/6 inhibitor, an MEK1/2 inhibitor, a JNK inhibitor, a p38 inhibitor or an ERK inhibitor.

In some embodiments, the MAPK inhibitor is an inhibitor of p38 phosphorylation, an inhibitor of JNK phosphorylation or an inhibitor of ERK phosphorylation, preferably an inhibitor of p38 phosphorylation.

In some embodiments, the MAPK inhibitor is a JNK inhibitor, a p38 inhibitor or an ERK inhibitor.

In some embodiments, the MAPK inhibitor is FR180204, SP600125, SB203580, SB202190, LY2228820, BIRB 796; SB203580 hydrochloride, SCIO 469 hydrochloride, TMCB, XMD 8-92, TCS JNK 6o, SU 3327, CC 401 dihydrochloride, or a derivative thereof.

In some embodiments, the MAPK inhibitor is FR180204, SP600125, SB203580 or a derivative thereof.

In some embodiments, the IL-1 inhibitor is an anti-IL-1α antibody, an anti-IL-1β antibody, an IL-1 antagonist, an IL-1 receptor antagonist, an IL-1α converting enzyme inhibitor, an IL-1β converting enzyme inhibitor, or a soluble decoy IL-1 receptor.

In some embodiments, the IL-1 inhibitor is anakinra, canakinumab, rilonacept, gevokizumab or a variant thereof.

In some embodiments, the IL-1 inhibitor is anakinra or a variant thereof.

In some embodiments, the NF-κB inhibitor is an IL-1 inhibitor, an IL-1 receptor inhibitor, a TLR4 inhibitor, a TAK1 inhibitor, an Akt inhibitor, an IKK inhibitor, an inhibitor of IκB phosphorylation, an inhibitor of IκB degradation, an inhibitor of the proteasome, an inhibitor of IκBα upregulation, an inhibitor of NF-κB nuclear translocation, an inhibitor of NF-κB expression, an inhibitor of NF-κB DNA binding, or an inhibitor of NF-κB transactivation.

In some embodiments, the NF-κB inhibitor is SC514 or a derivative thereof; anakinra, canakinumab, rilonacept, gevokizumab or a variant thereof; or metformin, apigenin, kaempferol, BAY 11-7082, or a derivative thereof.

In some embodiments, the NF-κB inhibitor is SC514 or a derivative thereof.

In one embodiment, the inhibitor of MAPK/ERK signalling (e.g. MAPK inhibitor), IL-1 inhibitor and/or NF-κB inhibitor are administered simultaneously, sequentially or separately.

In some embodiments, the population of haematopoietic cells, haematopoietic stem cells and/or haematopoietic progenitor cells is contacted with the inhibitor of MAPK/ERK signalling (e.g. the MAPK inhibitor) prior to or concurrently with the step of introducing gene editing machinery to said cells, preferably prior to the step of introducing gene editing machinery to said cells.

In some embodiments, the population of haematopoietic cells, haematopoietic stem cells and/or haematopoietic progenitor cells is contacted with the IL-1 inhibitor and/or NF-κB inhibitor prior to, concurrently with or following the step of introducing gene editing machinery to said cells.

In some embodiments, the population of haematopoietic cells, haematopoietic stem cells and/or haematopoietic progenitor cells is contacted with the inhibitor of MAPK/ERK signalling (e.g. the MAPK inhibitor) prior to or concurrently with the step of transducing said cells, preferably prior to the step of transducing said cells.

In some embodiments, the population of haematopoietic cells, haematopoietic stem cells and/or haematopoietic progenitor cells is contacted with the IL-1 inhibitor and/or inhibitor of NF-κB prior to, concurrently with or following the step of transducing said cells.

Thus, in one embodiment of the method of gene editing or the method of transducing a population of haematopoietic cells, haematopoietic stem cells, haematopoietic progenitor cells and/or T cells, steps (a) and (b) may be carried out simultaneously. In another embodiment, steps (a) and (b) are carried out sequentially, either step (a) before step (b) or step (b) before step (a).

In one embodiment, the haematopoietic cells, haematopoietic stem cells, haematopoietic progenitor cells and/or T cells are contacted with the one or more inhibitor(s) of senescence (e.g. the MAPK inhibitor, IL-1 inhibitor and/or NF-κB inhibitor, preferably with the MAPK inhibitor) are contacted with the inhibitor(s) about 15 minutes to about 72 hours; about 15 minutes to about 48 hours; or about 15 minutes to about 24 hours; about 15 minutes to about 4 hours; about 15 minutes to about 3 hours; about 15 minutes to about 2 hours; about 15 minutes to about 1 hour before transducing the population of cells with the viral vector and/or before introducing the gene editing machinery into the cell. In another embodiment, the cells are contacted with the inhibitor(s) about 1 hour to about 72 hours; about 1 hour to about 48 hours; or about 1 hour to about 24 hours before transducing the population of cells with the viral vector and/or before introducing the gene editing machinery into the cell. In another embodiment, the cells are contacted with the inhibitor(s) about 1-4 hours; 1-3 hours; or 1-2 hours before transducing the population of cells with the viral vector and/or before introducing the gene editing machinery into the cell.

In one embodiment, the haematopoietic cells, haematopoietic stem cells, haematopoietic cell progenitor cells and/or T cells are contacted with the one or more inhibitor(s) of senescence (e.g. the MAPK inhibitor, IL-1 inhibitor and/or NF-κB inhibitor, preferably with the MAPK inhibitor) about 15 minutes, 30 minutes, 1 hour, 1.5 hours, 2 hours, 2.5 hours, 3 hours, 3.5 hours, 4 hours, 12 hours, 24 hours, 36 hours, 48 hours, 60 hours, or 72 hours, preferably about 24 hours or 48 hours, before transducing the population of cells with the viral vector and/or before introducing the gene editing machinery into the cell.

In one embodiment, the haematopoietic cells, haematopoietic stem cells, haematopoietic cell progenitor cells and/or T cells are contacted with the one or more inhibitor(s) of senescence (e.g. the MAPK inhibitor, IL-1 inhibitor and/or NF-κB inhibitor, preferably the MAPK inhibitor) about 15 minutes, 30 minutes, 1 hour, 1.5 hours, 2 hours, 2.5 hours, 3 hours, 3.5 hours or 4 hours, preferably about 15 minutes, before transducing the population of cells with the viral vector and/or before introducing the gene editing machinery into the cell.

In one embodiment, the haematopoietic cells, haematopoietic stem cells, haematopoietic progenitor cells and/or T cells are contacted with the one or more inhibitor(s) of senescence (e.g. the MAPK inhibitor, IL-1 inhibitor and/or NF-κB inhibitor, preferably the IL-1 inhibitor and/or NF-κB inhibitor) about 15 minutes to about 4 hours; about 15 minutes to about 3 hours; or about 15 minutes to about 2 hours after transducing the population of cells with the one or more viral vectors and/or after introducing the gene editing machinery into the cell. In another embodiment, the cells are contacted with the inhibitor(s) about 1-4 hours; 1-3 hours; or 1-2 hours after transducing the population of cells with the viral vector and/or after introducing the gene editing machinery into the cell (e.g. after electroporating the cell).

In one embodiment, the haematopoietic cells, haematopoietic stem cells, haematopoietic cell progenitor cells and/or T cells are contacted with the one or more inhibitor(s) of senescence (e.g. the MAPK inhibitor, IL-1 inhibitor and/or NF-κB inhibitor, preferably the IL-1 inhibitor and/or NF-κB inhibitor) about 15 minutes, 30 minutes, 1 hour, 1.5 hours, 2 hours, 2.5 hours, 3 hours, 3.5 hours or 4 hours, preferably about 15 minutes, after transducing the population of cells with the viral vector and/or after introducing the gene editing machinery into the cell (e.g. after electroporating the cell).

Suitably, the inhibitor(s) may be active during gene editing.

Suitably, the inhibitor(s) may be active during transduction.

In one embodiment, the contacting step is performed for about 12-60 h, such as 24-60 h, 36-60 h or 42-54 h, preferably about 42-54 h, before the step of introducing the gene editing machinery and/or of transducing the viral vector is started. In one embodiment, the contacting step is for about 12, 18, 24, 30, 36, 42, 48, 54 or 60 h, preferably about 48 h, before the introducing step is started.

In one embodiment, the contacting step is performed for about 12-96 h, such as 12-60 h, 24-60 h, 36-60 h or 42-54 h, preferably about 42-54 h, after the step of introducing the gene editing machinery and/or of transducing the viral vector is started. In one embodiment, the contacting step is for about 12, 18, 24, 30, 36, 42, 48, 54, 60, 66, 72, 78, 84, 90, 96, 102 h, preferably about 48 h or about 96 h, after the introducing and/or transducing step is started.

In one embodiment, the contacting step is carried out about for about 12-96 h, such as 12-72 h, 12-60 h, 24-60 h, 36-60 h or 42-54 h, preferably about 72-96 h, after beginning culture of the population of haematopoietic cells, haematopoietic stem cells, haematopoietic progenitor cells and/or T cells (e.g. beginning culture after the cells are thawed from a frozen state). In one embodiment, the contacting step is carried out about 12, 18, 24, 30, 36, 42, 48, 54, 60, 66, 72, 78, 84, 90, 96, 102 h, preferably about 72 h, after beginning culture of the population of haematopoietic cells, haematopoietic stem cells, haematopoietic progenitor cells and/or T cells.

In one embodiment, the contacting step is carried out about 12-96 h, such as 12-60 h, 24-60 h, 36-60 h or 42-54 h, preferably about 72-96 h, after thawing the population of haematopoietic cells, haematopoietic stem cells, haematopoietic progenitor cells and or T cells (e.g. which had been stored in a frozen state). In one embodiment, the contacting step is carried out about 12, 18, 24, 30, 36, 42, 48, 54, 60, 66, 72, 78, 84, 90, 96 or 102 h, preferably about 72 h, after thawing the population of haematopoietic cells, haematopoietic stem cells, haematopoietic progenitor cells and/or T cells.

In some embodiments, the method further comprises the step of contacting the population of haematopoietic cells, haematopoietic stem cells, haematopoietic progenitor cells and/or T cells with an agent which promotes homology directed DNA repair, preferably wherein the agent is an inhibitor of p53 activation.

In some embodiments, the agent is an inhibitor of p53 activation, preferably wherein the inhibitor is an inhibitor of p53 phosphorylation, more preferably an inhibitor of p53 Serine 15 phosphorylation.

In some embodiments, the inhibitor of p53 activation is a p53 dominant negative peptide, an ataxia telangiectasia mutated (ATM) kinase inhibitor or an ataxia telangiectasia and Rad3-related protein (ATR) inhibitor.

In some embodiments, the inhibitor of p53 activation is pifithrin-α or a derivative thereof; KU-55933 or a derivative thereof; GSE56 or a variant thereof; KU-60019, BEZ235, wortmannin, CP-466722, Torin 2, CGK 733, KU-559403, AZD6738 or derivatives thereof; or an siRNA, shRNA, miRNA or antisense DNA/RNA, preferably wherein the inhibitor of p53 activation is GSE56 or a variant thereof.

In some embodiments, the one or more inhibitor(s) of senescence (e.g. the MAPK inhibitor, IL-1 inhibitor and/or NF-κB inhibitor) is added to the haematopoietic cell, haematopoietic stem cell, haematopoietic progenitor cells and/or T cells at a concentration of about 0.5-200 μM or about 0.1-200 ng/μl.

In one embodiment, the one or more inhibitor(s) of senescence (e.g. the MAPK inhibitor, IL-1 inhibitor and/or NF-κB inhibitor, preferably the IL-1 inhibitor and/or NF-κB inhibitor) is added to the haematopoietic cells, haematopoietic stem cells, haematopoietic progenitor cells and/or T cells (e.g. in an in vitro or ex vivo culture) at a concentration of about 0.5-200, 0.5-150, 0.5-100, 0.5-50, 0.5-40, 0.5-30, 0.5-20 or 0.5-15 μM, preferably about 0.5-30 μM, more preferably about 0.5-15 μM. In one embodiment, the one or more inhibitor(s) of senescence (e.g. the MAPK inhibitor, IL-1 inhibitor and/or NF-κB inhibitor, preferably the IL-1 inhibitor and/or NF-κB inhibitor) is added to the haematopoietic cells, haematopoietic stem cells, haematopoietic progenitor cells and/or T cells (e.g. in an in vitro or ex vivo culture) at a concentration of about 1-200, 1-150, 1-100, 1-50, 1-40, 1-30, 1-20 or 1-15 μM, preferably about 1-30 μM, more preferably about 1-15 μM. In another embodiment, the inhibitor(s) is added to the haematopoietic cells, haematopoietic stem cells, haematopoietic progenitor cells and/or T cells at a concentration of about 5-200, 5-150, 5-100, 5-50, 5-40, 5-30, 5-20 or 5-15 μM, preferably about 5-30 μM.

In one embodiment, the one or more inhibitor(s) of senescence (e.g. the MAPK inhibitor, IL-1 inhibitor and/or NF-κB inhibitor, preferably the IL-1 inhibitor and/or NF-κB inhibitor) is added to the haematopoietic cells, haematopoietic stem cells, haematopoietic progenitor cells and/or T cells (e.g. in an in vitro or ex vivo culture) at a concentration of about 0.5, 1, 2, 3, 4, 5, 6, 7, 8, 9, 10, 11, 12, 13, 14, 15, 20, 25, 30, 35, 40, 45, 50, 75, 100, 125, 150, 175 or 200 μM. In another embodiment, the one or more inhibitor(s) of senescence (e.g. the MAPK inhibitor, IL-1 inhibitor and/or NF-κB inhibitor, preferably the IL-1 inhibitor and/or NF-κB inhibitor) is added to the haematopoietic cells, haematopoietic stem cells, haematopoietic progenitor cells and/or T cells (e.g. in an in vitro or ex vivo culture) at a concentration of about 2 μM. In another embodiment, the one or more inhibitor(s) of senescence (e.g. the MAPK inhibitor, IL-1 inhibitor and/or NF-κB inhibitor, preferably the IL-1 inhibitor and/or NF-κB inhibitor) is added to the haematopoietic cells, haematopoietic stem cells, haematopoietic progenitor cells and/or T cells (e.g. in an in vitro or ex vivo culture) at a concentration of about 4 μM. In another embodiment, the one or more inhibitor(s) of senescence (e.g. the MAPK inhibitor, IL-1 inhibitor and/or NF-κB inhibitor, preferably the IL-1 inhibitor and/or NF-κB inhibitor) is added to the haematopoietic cells, haematopoietic stem cells, haematopoietic progenitor cells and/or T cells (e.g. in an in vitro or ex vivo culture) at a concentration of about 12 μM.

In one embodiment, the one or more inhibitor(s) of senescence (e.g. the MAPK inhibitor, IL-1 inhibitor and/or NF-κB inhibitor, preferably the IL-1 inhibitor and/or NF-κB inhibitor) is added to the haematopoietic cells, haematopoietic stem cells, haematopoietic progenitor cells and/or T cells (e.g. in an in vitro or ex vivo culture) at a concentration of about 0.1-200, 0.1-150, 0.1-100, 0.1-75, 0.1-60, 0.1-50, 0.1-25, 0.1-20, 0.1-15 or 0.1-10 ng/μl, preferably about 0.1-60 ng/μl. In another embodiment, the inhibitor(s) is added to the haematopoietic cells, haematopoietic stem cells, haematopoietic progenitor cells and/or T cells at a concentration of about 5-200, 5-150, 5-100, 5-75, 5-60, 5-50, 5-25, 5-20, 5-15 or 5-10 ng/μl, preferably about 5-60 ng/μl.

In one embodiment, the one or more inhibitor(s) of senescence (e.g. the MAPK inhibitor, IL-1 inhibitor and/or NF-κB inhibitor, preferably the IL-1 inhibitor and/or NF-κB inhibitor) inhibitor is added to the haematopoietic cells, haematopoietic stem cells, haematopoietic progenitor cells and/or T cells (e.g. in an in vitro or ex vivo culture) at a concentration of about 0.1, 1, 2, 3, 4, 5, 6, 7, 8, 9, 10, 11, 12, 13, 14, 15, 20, 25, 30, 35, 40, 45, 50, 55, 60, 65, 70, 75, 80, 90, 100, 125, 150, 175, or 200 ng/μl, preferably about 50 ng/μl.

In some embodiments, the method further comprises the step of contacting the population of haematopoietic cells, haematopoietic stem cells, haematopoietic progenitor cells and/or T cells with at least one adenoviral protein or a nucleic acid sequence encoding therefor.

In some embodiments, the inhibitor further comprises at least one adenoviral protein.

In some embodiments, the inhibitor further comprises a nucleic acid sequence encoding at least one adenoviral protein.

In some embodiments, the adenoviral protein is expressed transiently in the haematopoietic cell, haematopoietic stem cell, haematopoietic progenitor cell and/or T cell, preferably wherein the transient expression occurs during gene editing and/or transduction of the haematopoietic cell, haematopoietic stem cell, haematopoietic progenitor cell and/or T cell.

In some embodiments, the population of haematopoietic cells, haematopoietic stem cells, haematopoietic progenitor cells and/or T cells is obtained from mobilised peripheral blood, bone marrow or umbilical cord blood.

In some embodiments, the method includes a further step of enriching the population for haematopoietic stem and/or progenitor cells and/or T cells.

In some embodiments, the target of the gene editing is selected from the group consisting of FANC-A, CD40L, RAG-1, IL-2RG, CYBA, CYBB, NCF1, NCF2, and NCF4. In one embodiment, the target of the gene editing is a gene mutated in chronic granulomatous disease or the gene mutated SCID, atypical SCID and Omenn syndrome, or Hyper IgM syndrome.

In a further aspect, the invention provides a method of gene therapy comprising the steps:
(a) gene editing a population of haematopoietic cells, haematopoietic stem cells, haematopoietic progenitor cells and/or T cells according to the method of the invention; and
(b) administering the gene edited haematopoietic cells, haematopoietic stem cells, haematopoietic progenitor cells and/or T cells to a subject.

In some embodiments, the gene edited cells are administered to a subject as part of an autologous stem cell transplant procedure or an allogeneic stem cell transplant procedure.

In some embodiments, the method increases the efficiency of gene editing of the population of haematopoietic cells, haematopoietic stem cells, haematopoietic progenitor cells and/or T cells.

In some embodiments, the method increases the survival and/or engraftment of the population of haematopoietic cells, haematopoietic stem cells, haematopoietic progenitor cells and/or T cells.

In some embodiments, the method increases the efficiency of gene therapy.

In a further aspect, the invention provides a method of gene therapy comprising the steps:
(a) transducing a population of haematopoietic cells, haematopoietic stem cells, haematopoietic progenitor cells and/or T cells according to the method of the invention; and
(b) administering the transduced population of haematopoietic cells, haematopoietic stem cells, haematopoietic progenitor cells and/or T cells to a subject.

In some embodiments, step (b) comprises administering the transduced cells to a subject as part of an autologous stem cell transplant procedure or an allogeneic stem cell transplant procedure.

In some embodiments, the method increases the survival and/or engraftment of the population of haematopoietic cells, haematopoietic stem cells, haematopoietic progenitor cells and/or T cells.

In some embodiments, the method increases the efficiency of gene therapy. In some embodiments, the method increases the efficiency of transduction of the population of haematopoietic cells, haematopoietic stem cells, haematopoietic progenitor cells and/or T cells. The increased transduction efficiency may, for example, be an increased vector copy number per cell (for example, increased by at least 1.1 fold, 1.2 fold, 1.3 fold, 1.4 fold, 1.5 fold, 1.6 fold, 1.7 fold, 1.8 fold, 1.9 fold, 1.8 fold, 1.9 fold, 2.0 fold, 2.1 fold, 2.2 fold, 2.3 fold, 2.4 fold, 2.5 fold, 3 fold or more). The increased transduction efficiency may, for example, be an increased percentage of cells transduced (for example, increased by 5%, 10%, 15%, 20%, 25%, 30%, 40%, 50%, 60%, 70%, 80%, 90%, 100%, 200%, 300% or more).

The method of gene therapy may be, for example, a method of treatment of a disease selected from the group consisting of mucopolysaccharidosis type I (MPS-1), chronic granulomatous disorder, Fanconi anaemia (FA), sickle cell disease, metachromatic leukodystrophy (MLD), globoid cell leukodystrophy (GLD), GM2 gangliosidosis, thalassemia and cancer.

The method of gene therapy may be, for example, a method of treatment of diseases caused by Rag-1 mutations, e.g. SCID, atypical SCID and Omenn syndrome

In one embodiment, the subject is a mammalian subject, preferably a human subject.

In a further aspect, the invention provides a gene edited and/or transduced population of haematopoietic cells, haematopoietic stem cells, haematopoietic progenitor cells and/or T cells prepared according to the method of the invention.

In a further aspect, the invention provides a pharmaceutical composition comprising the population of gene edited and/or transduced haematopoietic cells, haematopoietic stem cells, haematopoietic progenitor cells and/or T cells of the invention.

In a further aspect, the invention provides the population of gene edited and/or transduced haematopoietic cells, haematopoietic stem cells, haematopoietic progenitor cells and/or T cells of the invention for use in therapy.

In some embodiments, the population is administered as part of an autologous stem cell transplant procedure or an allogeneic stem cell transplant procedure.

### BRIEF DESCRIPTION OF THE FIGURES

FIG. 1. A) Schematic representation of experimental design. B) Percentage of edited allele by HDR by digital droplets PCR (ddPCR). (HS/AAV6: n=20; HS/AAV6+ANAK: n=15). ns, P>0.05, Mann-Whitney test. C) Percentage of GFP+ cells within HSPC subpopulations, from the more primitive (CD90+) to the more differentiated (CD133−). n=8-9; ns, P>0.05, Mann-Whitney test. D) Relative expression of CDKN1A (p21) in HS/AAV6 edited HSPCs at 24 h and 96 post-electroporation (n=3, 2, 4, 5). ns, P>0.05, Mann-Whitney test. E) Number of colonies formed by HSPCs treated with −DSB and HS/AAV6 in presence or absence of anakinra at 24 h and 96 h post-electroporation. Kruskal-Wallis tests. F) Culture composition of edited HSPC different subpopulations at 24 h and 96 h post gene editing.

FIG. 2. A) GSEA on whole transcriptomic analysis 24 h and 96 h post gene editing in presence or absence of anakinra. B) GSEA lots of two of the most upregulated inflammatory pathways from Hallmark categories. C) Relative expression of IL1A, IL8, IL6 in HS/AAV6 edited HSPCs at 24 h and 96 post-electroporation (n=9, 2, 9, 3). Mann-Whitney tests. *p<0.05; **p<0.01; ***p<0.001; ****p<0.0001.

FIG. 3. A) Percentage of human CD45⁺ cells in peripheral blood (PB) of NSG mice transplanted with 3×10⁵ HSPCs electroporated with HS RNP+AAV6 in presence or absence of Anakinra (ANAK). Mean+SEM. ns, P>0.05, Linear Mixed Model (LME) at 18 weeks. B) Percentage of human edited cells in peripheral blood (PB) of NSG mice transplanted as indicated in A). *p<0.05; **p<0.01, Linear Mixed Model (LME) at 18 weeks. C) Percentage of human CD45⁺ cells in bone marrow (BM) of NSG mice transplanted with 3×10⁵ HSPCs electroporated with HS RNP+AAV6 in presence or absence of Anakinra (ANAK). Mean+SEM. ns, P>0.05, Linear Mixed Model (LME) at 18 weeks. ns, P>0.05, Kruskal-Wallis tests. D) Percentage of human edited cells in bone marrow (BM) of NSG mice transplanted as indicated in C). **p<0.01, Linear Mixed Model (LME) at 18 weeks. E) Percentage of edited subpopulations (HSPCs, myeloid and B cells) in bone marrow (BM) of NSG mice transplanted as indicated in C). **p<0.01, Linear Mixed Model (LME) at 18 weeks. F) Number of colonies formed by BM-derived CD34+ cells treated as in C). *p<0.05; **p<0.01, Kruskal-Wallis tests. G) Relative expression of IL8 and CXCL10 in HS/AAV6 BM-derived edited HSPCs at the indicated treatments. ns, P>0.05, *p≤0.05, Mann-Whitney test. H) Number of dominant unique BARs in human BM-derived cells at the end of the experiment (18 w) in the represented conditions. **p<0.01; ***p<0.001, Kruskal-Wallis tests. I) Number of dominant unique BARs in human PB cells at 8-9, 12 and 15 weeks post transplantation into NSG mice. *p<0.05, Mann-Whitney test.

FIG. 4. A) Quantification of immunofluorescence staining for nuclear NF-kB (>100 nuclei analysed) 24 h and 96 h upon gene editing at the represented conditions. B) Representative images of the quantification in A).

FIG. 5. A) Schematic representation of experimental design. B) Percentage of edited allele by HDR by digital droplets PCR (ddPCR). (n=20,11, 3,5). ns, P>0.05, *P<0.05, Mann-Whitney test. C) Relative expression of IL8, IL6 and CCL2 in edited in presence or absence of SC-514 or the combination of GSE56 and SC-514. D) Number of colonies formed by HSPCs treated 96 h post-electroporation. *P<0.05, Kruskal-Wallis tests. E) Percentage of human CD45⁺ cells in peripheral blood (PB) of NSG mice transplanted with 1.5×10⁵ HSPCs electroporated with HS RNP+AAV6 in presence of GSE56, SC-514 or GSE56+SC-514. Mean+SEM. **p<0.01; ****p<0.0001. Linear Mixed Model (LME) at 15 weeks. F) Percentage of human CD45+ cells in the bone marrow (BM) of NSG mice transplanted with 1.5×10⁵ HSPCs electroporated with HS RNP+AAV6 in presence of GSE56, SC-514 or GSE56+SC-514. Mean+SEM. *P<0.05, **p<0.01, Kruskal-Wallis test. G) Number of dominant unique BARs in human BM-derived cells at the end of the experiment (15 w). **p<0.01, Kruskal-Wallis test. H) Number of dominant unique BARs in human PB cells at 8-9, 12 and 15 weeks post transplantation into NSG mice. **p<0.01, Kruskal-Wallis test.

FIG. 6. A) Schematic representation of experimental design. B) Number of colonies formed by HSPCs treated with DMSO, 4 μM or 8 μM p38i at 24 h post-electroporation (+DSB (HS): n=7, 7, 3; HS/AAV6: n=10, 10, 5). Kruskal-Wallis tests. C) Quantification of cytosolic ROS detected by CM-H₂DCFDA and D) mitochondrial superoxide with MitoSOX in HS/AAV6 edited HSPCs at 24 h and 96 post-electroporation (C: n=3, 4, 3, 3; D: n=2). Mann-Whitney tests. *p<0.05; **p<0.01; ***p<0.001; ****p<0.0001.

FIG. 7. A) Schematic representation of experimental design. B) Percentage of human CD45⁺ cells in peripheral blood (PB) and C) in bone marrow (BM) of NSG mice transplanted with 1-1.5×10⁵ HSPCs pre-treated with DMSO or 4 μM p38i and then electroporated with HS RNP or HS RNP+AAV6. Mean+SEM. D) Number of dominant unique BARs in human BM-derived cells at the end of the experiment (15 w). Median. (n=4, 5). E) Schematic representation of experimental design. F) Number of colonies formed by CD34⁺ purified from BM at 15 weeks. HSPCs were treated with DMSO or with 4 μM p38i before electroporation (n=7, 10, 9, 8 from 2 independent experiments). G) Percentage of fluorescent Q-Galactosidase positive cells measured on CD34+ cells purified from BM at 15 weeks. Mann-Whitney test, *P<0.05; ***P<0.001; ****P<0.0001.

FIG. 8. A) Schematic representation of experimental design. B) GFP expression within different HSPCs subpopulation (CD34+CD133−; CD34+CD133+ and CD34+CD133+CD90+ cells) edited upon different treatments (DMSO; 4 μM ERKi; 2 μM JNKi) at 96 h post-editing. C) Number of colonies formed at 24 h and 96 h post-editing by HSPCs treated with DMSO; 4 μM p38i; 4 μM ERKi or with 2 μM JNKi before the electroporation with HS RNP or HS/AAV6.

FIG. 9. A) Schematic representation of experimental design. B) Quantification of mitochondrial superoxide with MitoSOX in CD3+ T cells purified from 2 different donors not edited or edited after the treatment with 4 μM or 10 μM p38i at 24 h post-editing.

FIG. 10. A) Schematic representation of experimental design. B) Percentage of p16+senescent cells 24 h and 96 h upon GE. (UT EL=cells experiencing the sole electroporation; HS/AAV6=gene edited cells, (n=1)). C) Percentage of SA-β-Gal+senescent cells 24 h and 96 h upon GE. Cell transductions were performed with increasing MOI of AAV6 vector (2,000, 10,000, 20,000) and the analysis was performed on both GFP− and GFP+ subfractions, where indicated (n=1). D) Number of colonies formed by HSPCs treated 24 and 96 h post gene editing at the indicated conditions (n=3). E) Percentage of SA-β-Gal+ senescent cells in colonies derived from edited cells 24 h and 96 h upon GE in GFP− and GFP+ subfractions (n=8). F, G, H) Percentage of human CD45+ cells in peripheral blood (PB) of NSG mice transplanted with edited HSPCs treated as indicated. I, J) Percentage of SA-β-Gal+ senescent cells in all human engrafted HSPCs (CD45+ cells, I) or BM-derived CD34+ cells (J) at the endpoint (15 weeks). Cell transductions were performed with increasing MOI of AAV6 vector (2,000, 10,000, 20,000) and the analysis was performed on both GFP− and GFP+ subfractions, where indicated (I: n=4, 5,5,5,3,4,4,4,5,5; J: I: n=4, 5,5,5,3,15,15,4,5,5). Mann-Whitney test. *p<0.05, **p<0.01.

FIG. 11. A) Schematic representation of experimental design. B) Percentage of edited allele by HDR by digital droplets PCR (ddPCR). (HS/AAV6 DMSO: n=1; HS/AAV6+ATMi: n=2). C) Percentage of p16+ senescent cells 24 h and 96 h upon GE. (UT EL=cells experiencing the sole electroporation; HS/AAV6=gene edited cells, (n=1)). C) Percentage of GFP+ cells within HSPC subpopulations, from the more primitive (CD90+) to the more differentiated (CD133−) (n=11, 4). D) Relative expression of IL1A and IL6 in HS/AAV6 edited HSPCs at 96 post-electroporation in presence or absence of ATMi (n=2). E) Number of colonies formed by HSPCs treated as indicated 24 and 96 h post GE. F) Percentage of human CD45+ cells in peripheral blood (PB) of NSG mice transplanted with edited HSPCs treated as indicated (n=25, 8). G) Percentage of human CD45+ cells in total BM from NSG mice transplanted with edited HSPCs 18 weeks post transplantation (n=7, 8). H) Percentage of SA-β-Gal+senescent cells in BM-derived CD34+ cells at the endpoint (18 weeks) (n=26, 20, 17, 17). Cell were edited ex-vivo in presence or absence of p53 inhibition with GSE56. I, J, K) Relative expression of p21, p16 and IL8 in BM-derived HSPCs 18 weeks post transplantation at the indicated conditions (n=5). L) Number of colonies formed by BM-derived HSPCs (n=13, 14). Mann-Whitney test. *p<0.05, **p<0.01, ***p<0.001.

FIG. 12. A) Schematic representation of experimental design. B) Percentage of edited allele by HDR by digital droplets PCR (ddPCR). (HS/AAV6: n=20; HS/AAV6+ANAK: n=15). C) Percentage of GFP+ cells within HSPC subpopulations, from the more primitive (CD90+) to the more differentiated (CD133−) (n=8, 9). D) Relative expression of CDKN1A (p21) in HS/AAV6 edited HSPCs at 24 h and 96 post-electroporation (24 h: n=3, 2, 4, 5; 96 h: n=3, 2, 4, 3). E) Number of colonies formed by HSPCs treated with −DSB and HS/AAV6 in presence or absence of anakinra at 24 h and 96 h post-electroporation (24 h: n=7, 2, 5, 5; 96 h: 3, 2, 5, 4). F) Culture composition of edited HSPC different subpopulations at 24 h and 96 h post gene editing (24 h: n=4, 2, 5, 3; 96 h: n=4, 2, 6, 5). Mann-Whitney test. ns, p>0.05, *p<0.05.

FIG. 13. A) GSEA on whole transcriptomic analysis 24 h and 96 h post gene editing in presence or absence of anakinra. B) Representative GSEA plots from two of the most upregulated inflammatory pathways from Hallmark categories. C) Relative expression of IL1A, IL8, IL6 in HS/AAV6 edited HSPCs in presence or absence of Anakinra 96 post-electroporation (IL1A: n=9, 2, 9, 3; IL8: n=42, 2, 10, 4; IL6: n=27, 2, 6, 3;). Mann-Whitney tests. *p<0.05, **p<0.01, ***p<0.001, ****p<0.0001.

FIG. 14. A) Quantification of immunofluorescence staining for nuclear NF-kB (>100 nuclei analysed per sample) 24 h and 96 h upon gene editing at the indicated conditions. B) Representative images of the quantification in A (24 h: n=6, 6, 3, 3, 5, 6; 24 h: n=6, 6, 3, 3, 6, 4). Mann-Whitney tests. ns, p>0.05, *p<0.05, **p<0.01.

FIG. 15. A) Percentage of human CD45+ cells in peripheral blood (PB) of NSG mice transplanted with 3×105 HSPCs electroporated with HS RNP+AAV6 in presence or absence of Anakinra (ANAK). Mean+SEM (n=17, 28). B) Percentage of human edited cells in peripheral blood (PB) of NSG mice transplanted as indicated in A (n=17, 28). C) Percentage of human CD45+ cells in bone marrow (BM) of NSG mice transplanted with 3×105 HSPCs electroporated with HS RNP+AAV6 in presence or absence of Anakinra (ANAK). Mean+SEM (n=16, 19). D) Percentage of human edited cells in bone marrow (BM) of NSG mice transplanted as indicated in C (n=16, 19). E) Percentage of edited subpopulations (HSPCs, myeloid and B cells) in bone marrow (BM) of NSG mice transplanted as indicated in C) (n=16, 19, 16, 19, 16, 19). F) Number of colonies formed by BM-derived CD34+ cells treated as in C (n=13, 20). G, H) Relative expression of IL8 (G, n=6) and CXCL10 (H, n=6) in HS/AAV6 BM-derived edited CD34+ cells at the indicated treatments. 1) Percentage of p16+senescent cells in BM-derived HSPC (n=26, 20, 19, 17). J) Number of dominant unique BARs in human BM cells at 15 weeks post transplantation into NSG mice (n=19, 15). K) Number of dominant unique BARs in human PB cells at 8-9, 12 and 15 weeks post transplantation into NSG mice (n=6, 9, 6, 10, 6, 9). A, B: Linear Mixed Model (LME) at 18 weeks. C-I: Mann-Whitney test; ns, p>0.05, *p<0.05, **p<0.01.

FIG. 16. A) Schematic representation of experimental design. B) Percentage of GFP+cells within HSPC subpopulations, from the more primitive (CD90+) to the more differentiated (CD133−) (n=8). C) Culture composition of edited HSPC different subpopulations at 24 h and 96 h post gene editing (n=6). D) Number of colonies formed by HSPCs treated with the sole electroporation (UT ELECTRO) and HS/AAV6 in presence or absence of Anakinra at 24 h and 96 h post-electroporation (24 h: n=7, 2, 5, 5; 96 h: 3, 2, 5, 4). E) Percentage of p16+senescent cells 24 h and 96 h upon GE in presence or absence of Anakinra within GFP− and GFP+subfractions (24 h: n=3; 96 h: n=3, 4, 4, 4, 4). F) Percentage of human CD45+ cells in bone marrow (BM) of NSG mice at 15 weeks transplanted with HSPCs edited as indicated (n=6, 5, 4). G) Percentage of human edited cells in bone marrow (BM) of NSG mice at 15 weeks transplanted as indicated (n=5, 4). H) Percentage p16+ senescent cells in BM-derived HSPC treated as indicated (n=6, 5, 4). Mann-Whitney test; ns, p>0.05, *p<0.05.

FIG. 17. A) Schematic representation of experimental design for 1-hit IDLV gene editing. B) Schematic representation of experimental design for 2-hit IDLV gene editing. C) Schematic representation of experimental design for AAV6 gene editing. D) Quantification of NBS1 positive cells and distribution of the percentage of foci bearing cells over time from HSPCs edited with the indicated treatments (n=5, 5, 2, 5). E) Quantification of immunofluorescence staining for nuclear NF-kB (>100 nuclei analysed per sample) 24, 96 and 168 h upon gene editing at the indicated conditions (24 h: n=2, 2, 2, 3, 3; 96 h: n=3; 168 h: n=3). F) Quantification of immunofluorescence staining for nuclear NF-kB (>100 nuclei analysed per sample) 24 and 96 h upon gene editing at the indicated conditions (24 h: n=2; 96 h: n=1). G) Number of colonies formed by HSPCs treated 24 and 96 h post gene editing at the indicated conditions (n=2). H) Percentage of GFP+ cells within HSPC subpopulations, from the more primitive (CD90+) to the more differentiated (CD133−) (n=5). 1) Apoptosis analysis performed at 24 h upon GE in HSPC subfractions in H. Early apoptosis: Annexin V+, 7AAD−; Late apoptosis: Annexin V+, 7AAD+; Necrosis: Annexin V−; 7AAD+ (n=5). J) Culture composition of edited HSPC different subpopulations at 24 h and 96 h post gene editing (n=5). Mann-Whitney tests. ns, p>0.05, *p<0.05, **p<0.01.

FIG. 18. A) Schematic representation of experimental design. B) Percentage of CB-derived edited allele by HDR by digital droplets PCR (ddPCR). (n=20, 11, 3,5). C) Relative expression of IL8, IL6 and CCL2 in HSPCs edited in presence or absence of SC-514 or the combination of GSE56 and SC-514 (n=2). D) Number of colonies formed by HSPCs treated 96 h post GE (n=16, 3, 3, 3). E) Percentage of GFP+ cells within HSPC subpopulations, from the more primitive (CD90+) to the more differentiated (CD133−) (n=2, 1). F) Number of colonies formed by HSPCs treated 24 and 96 h post GE (n=3). G) Percentage of p16+senescent cells 24 h and 96 h upon GE in presence or absence of SC514 within GFP− and GFP+subfractions (24 h: n=3; 96 h: n=3, 4, 4, 3, 3). Kruskall-Wallis tests. ns, p>0.05, *p<0.05.

FIG. 19. A) Percentage of human CD45+ cells in peripheral blood (PB) of NSG mice transplanted with 1.5×105 HSPCs electroporated with HS RNP+AAV6 in presence of GSE56, SC-514 or GSE56+SC-514 (n=5). B) Percentage of human CD45+ cells 15 weeks post transplantation in the bone marrow (BM) of NSG mice transplanted with HSPCs treated as indicated (n=8, 12, 9, 11). C) Number of colonies formed by BM-derived HSPCs 15 weeks upon transplantation treated as indicated (n=6). D) Number of dominant unique BARs in human BM-derived cells at the end of the experiment (15 w) (n=19, 12, 16, 11). E) Number of unique indels retrieved by on-target AAVS1 sequencing in human bone marrow cells of mice treated as indicated (n=5, 12, 6, 11). F, G) Percentage of p16 intracellular levels (F, n=26, 20, 12, 12, 15, 14, 13, 13) and SA-β-Gal+senescent cells (G, n=22, 22, 11, 11, 5, 5, 5, 5) in BM-derived CD34+ cells at the endpoint (15 weeks). A: Linear Mixed Model (LME) at 15 weeks. B-E: Kruskall Wallis test. F-G: Mann-Whitney test; ns, p>0.05, *p<0.05, **p<0.01, ***p<0.001, ****p<0.0001.

FIG. 20. A) Schematic representation of experimental design: HSPCs were treated with DMSO or with 4 μM p38i on days 1 and 2 post-thawing and analysed on days 1, 3, and 7. B) Quantification of Phospho-p38 in HSPCs at the indicated time points (n=5). C) Quantification of single and double-strand DNA breaks by alkaline comet assay on days 3 and 7 post thawing in DMSO or p38i treated cells (n=2. More than 100 cells per condition were analysed). D) Representative confocal images of γH2AX (green) and pRPA (red) foci and respective zoom in pictures. Nuclei were stained with DAPI. E) Quantification of γH2AX foci at day 3 and 7 (n=8, 3, 7, 4). F) Quantification of pRPA foci on days 3 and 7 (n=7, 3, 7, 3). G, H) Quantification of cytosolic ROS detected by CM-H2DCFDA (G) and mitochondrial superoxides with MitoSOX (H) in HSPCs on days 3 and 7 post thawing (G: n=3, 3, 5, 5, 5, 4; H: n=3, 3, 6, 6, 5, 4). Mann-Whitney tests. *p<0.05; **p<0.01.

FIG. 21. A) Schematic representation of experimental design: HSPCs were treated with DMSO, or with 4 μM or 8 μM p38i on days 1 and 2 post-thawing, and on day 3, electroporated with HS RNP or HS RNP+AAV6 on day 3. Subsequent analyses were performed 24 or 96 h post-electroporation. B) Percentage of edited alleles within CD34+ cells upon DMSO or p38i treatments measured by ddPCR (n=10, 10, 3). C) Number of colonies formed by HSPCs treated with DMSO, 4 μM or 8 μM p38i 24 h post-electroporation (+DSB (HS): n=7, 7, 3; HS/AAV6: n=10, 10, 5). D, E) Quantification of cytosolic ROS detected by CM-H2DCFDA (D) and mitochondrial superoxides with MitoSOX (E) in HS/AAV6 edited HSPCs 24 h and 96 post-electroporation (D: n=3, 4, 3, 3; E: n=2). F) Expression of the phosphorylated form of p38-MAPK within different subpopulations represented as median fluorescence intensity (MFI) at the indicated time points post gene editing. G) Percentage of edited alleles within CD34+CD133+CD45RA-CD90+ cells upon DMSO or p38i treatments measured by ddPCR (n=3). C: Kruskal-Wallis test. A-G: Mann Whitney test. *p<0.05; **p<0.01; ***p<0.001; ****p<0.0001.

FIG. 22. A) Schematic representation of experimental design: HSPCs were treated with DMSO or with 4 μM p38i on days 1 and 2 post-thawing and then electroporated with HS RNP or HS RNP+AAV6 on day 3. Transplantation in NSG mice was performed 24 h post-electroporation. B, C) Percentage of human CD45+ cells in peripheral blood (PB) (B) and in bone marrow (BM) (C) of NSG mice transplanted with 1-1.5×105 HSPCs pre-treated with DMSO or 4 μM p38i and then electroporated with HS RNP or HS RNP+AAV6. B: Mean±SEM; C: Median±SEM; (n=7, 10, 9, 8). D) Number of dominant unique BARs in human BM-derived cells at the end of the experiment (15 w). Median; (n=9, 8). E) Peripheral blood composition: percentage of CD19+, CD13+ or CD3+ cells within human CD45+ population. (n=7, 10, 9, 8). F) Schematic representation of the experimental design: at the end point BM-derived CD34+ cells were challenged in CFU-C in vitro assay. G) Number of colonies formed by CD34+purified from BM at 15 weeks (n=7, 10, 9, 8 from 2 independent experiments). H) Percentage of fluorescent SA-β-Galactosidase positive cells measured on BM-derived CD34+ at 15 weeks (n=5, 3, 5, 2). Mann-Whitney test, *p<0.05; **p<0.01; ***p<0.001; ****p<0.0001.

FIG. 23. A) Schematic representation of experimental design: HSPCs were treated with DMSO or 4 μM p38i on days 1 and 2 post-thawing and then electroporated (where indicated) with HS RNP+AAV6 on day 3. 24 h post-electroporation, HSC/MPP pool (CD34+CD133+CD45RA-CD90+) was sorted and seeded as single-cell in a differentiation medium. B) Percentage of uni-, bi-, and multi-lineage colonies was calculated based on marker expression and representation within single colonies. C) Percentage of colonies with the indicated lineage composition. (B, C: n=4; more than 500 colonies were analysed per donor and condition). D) Schematic representation of experimental design: HSPCs were treated with DMSO or 4 μM p38i on days 1 and 2 post-thawing and then electroporated with HS RNP+AAV6 on day 3. 24 h post-electroporation, HSC/MPP pool (CD34+CD133+CD45RA-CD90+GFP+ or GFP−) was sorted and processed for a single-cell RNA sequencing analysis. E) Umap (Uniform Manifold Approximation and Projection) plot comprising scRNA-seq data. Clusters and associated cell types are indicated by the indicated names and colors. F) Stacked bar plots showing the distribution of the identified clusters across samples. G) Heatmap showing Log FC values for differentially expressed genes (DEGs) between indicated samples.

FIG. 24. A) Schematic representation of the experimental design: HSPCs were treated with DMSO, 2 μM JNKi, or 4 μM ERKi on days 1 and 2 post-thawing and electroporated with HS RNP or HS RNP+AAV6 on day 3. B) GFP expression within different HSPCs subpopulation (CD34+CD133−; CD34+CD133+ and CD34+CD133+CD90+ cells) edited upon the indicated treatments (DMSO; 2 μM JNKi; 4 μM ERKi) 96 h post-editing (n=2, 2, 1). C) Number of colonies formed at 24 h and 96 h post-editing by HSPCs treated with DMSO, 2 μM JNKi, or 4 μM ERKi before the electroporation with HS RNP or HS/AAV6.

FIG. 25. A) Schematic representation of experimental design: PBMC-derived CD4+ T cells were treated with DMSO or 10 μM p38i on days 1 and 2 post-purification, and on day 3, where indicated, electroporated with HS RNP+AAV6. B) Percentage of edited cells measured by flow cytometry as dNGFR+ cells (n=3). C) Relative quantification of the percentage of T cells subpopulation composition within CD4+ T cells (TEMRA: CD62L-CD45RA+; CM: CD62L+CD45RA−; EM: CD62L-CD45RA−; TSCM: CD62L+CD45RA+) at 96 h post-electroporation. D) Apoptosis analysis performed at 96 h post-electroporation within total CD4+ T cells. Early apoptosis: Annexin V+, 7AAD−; Late apoptosis: Annexin V+, 7AAD+; Necrosis: Annexin V−; 7AAD+. E) Growth curve of CD4+ T cells. Cells from the reported conditions were counted at the indicated time points; total number of cells and the fold increase between untreated (−) and p38 inhibitor cells (p38i) is reported in the graph. F) Quantification of mitochondrial superoxide with MitoSOX in CD4+ T cells purified from 2 different donors not edited or edited after the treatment with 10 μM p38i at 24 h post-editing (n=2).

G, H) Percentage of senescent cells assessed by SPiDER SA-β-Galactosidase (G) or p16 intracellular staining (H) at day 1, 13 and 20 upon gene editing (n=3). Mann-Whitney tests. *p<0.05.

### DETAILED DESCRIPTION OF THE INVENTION

### Cell Survival and Engraftment

In one aspect, the invention provides the use of one or more inhibitor(s) of senescence for increasing the survival and/or engraftment of haematopoietic cells, haematopoietic stem cells, haematopoietic progenitor cells and/or T cells in gene therapy.

In a further aspect, the invention provides one or more inhibitor(s) of senescence for use in haematopoietic cell gene therapy, haematopoietic stem cell gene therapy, haematopoietic progenitor cell gene therapy and/or T cell gene therapy.

In a further aspect, the invention provides one or more inhibitor(s) of senescence for use in gene therapy in increasing the survival and/or engraftment of haematopoietic cells, haematopoietic stem cells, haematopoietic progenitor cells and/or T cells.

In some embodiments, the use is an in vitro or ex vivo use.

In some embodiments, the gene therapy is haematopoietic cell gene therapy, haematopoietic stem cell gene therapy and/or haematopoietic progenitor cell gene therapy.

In some embodiments, the cells are haematopoietic cells, haematopoietic stem cells and/or haematopoietic progenitor cells.

Current protocols for gene transfer and gene editing require prolonged ex-vivo culture, high viral vector doses and nuclease-induced DNA DSBs, that activates the DNA Damage Response (DDR) pathway, leading to cell cycle arrest. Emerging data indicates that cellular detection of viral vectors employed in classical gene therapy settings, instead of eliciting innate immune mediated recognition of viral nucleic acids or proteins, unexpectedly also triggers the DDR.

The DDR pathway is an evolutionary conserved set of actions converging on key decision-making factors such as the tumour suppressor p53 to enforce cell cycle arrest (Piras, F. et al., 2017, EMBO Mol Med 9: 1198-1211). The activation of the DDR pathway leads to DDR-dependent inflammation (Di Micco, R., 2017, Trends Mol. Med. 23: 1067-1070). The present inventors have previously demonstrated that activation of the DDR pathway impairs the haematopoietic reconstitution of gene-modified cells upon transplantation (Schiroli, G. et al., 2019, Cell Stem Cell 24: 551-565; and Conti, A. & Di Micco, R., 2018, Genome Med 10: 66).

In some embodiments, the SASP inhibitor (e.g. the IL-1 inhibitor and/or NF-κB inhibitor) inhibits DDR-dependent inflammation. Suitably, the inhibition of DDR-dependent inflammation increases the survival and/or engraftment of haematopoietic cells, haematopoietic stem cells, haematopoietic progenitor cells and/or T cells in gene therapy.

Protracted DDR signalling has been causatively linked to the establishment of cellular senescence, a condition in which cells, despite being still alive, are unable to further proliferate. Accumulation of cell cycle inhibitors p21 and p16 is associated with senescence. Senescent cells are also characterized by, for example, a senescence-associated secretory phenotype (SASP). Through SASP, mainly characterized by pro-inflammatory cytokines, senescent cells may exert detrimental paracrine functions on bystander cells.

In some embodiments, the one or more inhibitor(s) of senescence inhibits a cellular senescence program. Suitably, the inhibition of a cellular senescence program increases the survival and/or engraftment of haematopoietic cells, haematopoietic stem cells, haematopoietic progenitor cells and/or T cells in gene therapy.

As used herein, the term “survival” refers to the ability of the haematopoietic cells, haematopoietic stem cells, haematopoietic progenitor cells and/or T cells to remain alive (e.g. not die or become apoptotic) during in vitro or ex vivo culture. Haematopoietic stem and/or progenitor cells and/or T cells may, for example, undergo increased apoptosis following transduction with a viral vector during cell culture; thus, the surviving cells may have avoided apoptosis and/or cell death.

Cell survival may be readily analysed by the skilled person. For example, the numbers of live, dead and/or apoptotic cells in a cell culture may be quantified at the beginning of culture and/or following culture for a period of time (e.g. about 6 or 12 hours, or 1, 2, 3, 4, 5, 6, 7 or more days; preferably, the period of time begins with the transduction of the cells with a viral vector). The effect of an inhibitor according to the invention on cell survival may be assessed by comparing the numbers and/or percentages of live, dead and/or apoptotic cells at the beginning and/or end of the culture period between experiments carried out in the presence and absence of the inhibitor, but under otherwise substantially identical conditions.

Cell numbers and/or percentages in certain states (e.g. live, dead or apoptotic cells) may be quantified using any of a number of methods known in the art, including use of haemocytometers, automated cell counters, flow cytometers and fluorescence activated cell sorting machines. These techniques may enable distinguishing between live, dead and/or apoptotic cells. In addition or in the alternative, apoptotic cells may be detected using readily available apoptosis assays (e.g. assays based on the detection of phosphatidylserine (PS) on the cell membrane surface, such as through use of Annexin V, which binds to exposed PS; apoptotic cells may be quantified through use of fluorescently-labelled Annexin V), which may be used to complement other techniques.

As used herein, the term “engraftment” refers to the ability of the haematopoietic stem and/or progenitor cells and/or T cells to populate and survive in a subject following their transplantation, i.e. in the short and/or long term after transplantation. For example, engraftment may refer to the number and/or percentages of haematopoietic cells and/or T cells descended from the transplanted haematopoietic stem cells and/or T cells (e.g. graft-derived cells) that are detected about 1 day to 24 weeks, 1 day to 10 weeks, or 1-30 days or 10-30 days after transplantation. In the xenograft model of human haematopoietic stem and/or progenitor cell engraftment and repopulation, engraftment may be evaluated in the peripheral blood as the percentage of cells deriving from the human xenograft (e.g. positive for the CD45 surface marker), for example. In one embodiment, engraftment is assessed at about 1, 2, 3, 4, 5, 6, 7, 8, 9, 10, 15, 20, 25 or 30 days after transplantation. In another embodiment, engraftment is assessed at about 4, 5, 6, 7, 8, 9, 10, 11, 12, 13, 14, 15, 16, 17, 18, 19, 20, 21, 22, 23 or 24 weeks after transplantation. In another embodiment, engraftment is assessed at about 16-24 weeks, preferably 20 weeks, after transplantation.

Engraftment may be readily analysed by the skilled person. For example, the transplanted haematopoietic stem and/or progenitor cells and/or T cells may be engineered to comprise a marker (e.g. a reporter protein, such as a fluorescent protein), which can be used to quantify the graft-derived cells. Samples for analysis may be extracted from relevant tissues and analysed ex vivo (e.g. using flow cytometry).

Suitably, the inhibitor(s) for use according to the present invention may improve engraftment of gene edited haematopoietic stem and/or progenitor cells and/or T cells compared with gene editing without use of the inhibitor(s). Suitably, engraftment at a given time point may be increased by at least 5%, 10%, 20%, 30%, 40%, 50%, 60%, 70%, 80%, 90% or more when compared with engraftment of untreated gene edited haematopoietic stem and/or progenitor cells and/or untreated gene edited T cells.

Suitably, the inhibitor(s) for use according to the present invention may improve engraftment of transduced haematopoietic stem and/or progenitor cells and/or T cells compared with transduction without use of the inhibitor(s). Suitably, engraftment at a given time point may be increased by at least 5%, 10%, 20%, 30%, 40%, 50%, 60%, 70%, 80%, 90% or more when compared with engraftment of untreated transduced haematopoietic stem and/or progenitor cells and/or untreated transduced T cells.

In a preferred embodiment, an inhibitor (or inhibitors) for use according to the invention does not adversely affect the growth of gene edited and/or transduced haematopoietic cells, haematopoietic stem cells, haematopoietic progenitor cells and/or T cells when compared with untreated gene edited haematopoietic cells, haematopoietic stem cells, haematopoietic progenitor cells and/or T cells.

### Gene Editing Efficiency

In a further aspect, the invention provides the use of one or more inhibitor(s) of senescence for increasing the efficiency of gene editing of haematopoietic cells, haematopoietic stem cells, haematopoietic progenitor cells, and/or T cells.

In a further aspect, the invention provides one or more inhibitor(s) of senescence for use in haematopoietic cell gene therapy, haematopoietic stem cell gene therapy, haematopoietic progenitor cell gene therapy and/or T cell gene therapy. Suitably, the one or more inhibitor(s) of senescence increases the efficiency of gene editing said cells.

In some embodiments, the IL-1 inhibitor and/or NF-κB inhibitor inhibits DDR-dependent inflammation. Suitably, the inhibition of DDR-dependent inflammation increases the efficiency of gene editing of haematopoietic cells, haematopoietic stem cells, haematopoietic progenitor cells, and/or T cells.

In some embodiments, the inhibitor of senescence (e.g. the MAPK inhibitor) inhibits a cellular senescence program. Suitably, the inhibition of a cellular senescence program increases the efficiency of gene editing of haematopoietic cells, haematopoietic stem cells, haematopoietic progenitor cells, and/or T cells.

In some embodiments, the use is an in vitro or ex vivo use.

In some embodiments, the gene therapy is haematopoietic cell gene therapy, haematopoietic stem cell gene therapy and/or haematopoietic progenitor cell gene therapy.

In some embodiments, the cells are haematopoietic cells, haematopoietic stem cells and/or haematopoietic progenitor cells.

Increasing the efficiency of gene editing may refer to an increase in the gene editing of the cells (e.g. haematopoietic cells, haematopoietic stem cells, haematopoietic progenitor cells and/or T cells) using an inhibitor (or combination of inhibitors) according to the invention, in comparison to the gene editing achieved in the absence of the inhibitor but under otherwise substantially identical conditions. Where a viral vector is used to introduce gene editing machinery, an increased efficiency may therefore allow the multiplicity of infection (MOI) and/or the time required to achieve effective transduction to be reduced.

In one embodiment, the percentage of cells which have been edited is increased. Methods for determining the percentage of cells which have been edited are known in the art. Suitable methods include flow cytometry, fluorescence-activated cell sorting (FACS) and fluorescence microscopy. The technique employed is preferably one which is amenable to automation and/or high throughput screening.

For example, a population of cells may be edited with a vector which harbours a reporter gene. Suitably, the reporter gene may be expressed when the cell has been edited. Suitable reporter genes include genes encoding fluorescent proteins, for example green, yellow, cherry, cyan or orange fluorescent proteins. Once the population of cells has been edited, both the number of cells expressing and not-expressing the reporter gene may be quantified using a suitable technique, such as FACS. The percentage of cells which have been edited may then be calculated.

Alternatively, quantitative PCR (qPCR) may be used to determine the percentage of cells which have been gene edited without the use of a reporter gene. For example, single colonies of cells (e.g. CD34+ cells) may be picked from a semi-solid culture and qPCR may be performed on each colony separately to determine the percentage of gene-edited-positive colonies among those analysed.

Methods for determining vector copy number are also known in the art. The technique employed is preferably one which is amenable to automation and/or high throughput screening. Suitable techniques include quantitative PCR (qPCR) and Southern blot-based approaches.

Increasing the efficiency of gene editing may refer to an increase in the number of cells (e.g. haematopoietic cells, haematopoietic stem cells, haematopoietic progenitor cells and/or T cells) in which a target gene or site has been edited (e.g. disrupted, replaced, deleted or had a nucleic acid sequence inserted within or at it) in the intended manner following transduction of a population of cells with a viral vector using an inhibitor (or combination of inhibitors) according to the invention, in comparison to that achieved in the absence of the inhibitor(s) but under otherwise substantially identical conditions. An increased efficiency of gene editing may therefore allow the multiplicity of infection (MOI) and/or the transduction time required to achieve effective gene editing to be reduced. Methods for determining whether a target gene or site has been edited are known in the art.

Increasing the efficiency of gene editing may refer to an increase in the fitness of gene edited cells (e.g. haematopoietic cells, haematopoietic stem cells, haematopoietic progenitor cells and/or T cells) that have been edited using an inhibitor (or combination of inhibitors) according to the invention, in comparison to that achieved in the absence of the inhibitor(s) but under otherwise substantially identical conditions.

Increasing the efficiency of gene editing may refer to an increase in the capacity to survive of gene edited cells (e.g. haematopoietic cells, haematopoietic stem cells, haematopoietic progenitor cells and/or T cells) that have been edited using an inhibitor (or combination of inhibitors) according to the invention, in comparison to that achieved in the absence of the inhibitor(s) but under otherwise substantially identical conditions.

In the context of gene editing, for example using a CRISPR/Cas system, preferably the vector used to transduce the population of cells is a non-integrating vector (e.g. an integration-defective lentiviral vector, IDLV).

In one embodiment, the inhibitor (or combination of inhibitors) for use according to the present invention improves gene editing efficiency compared with gene editing without use of the agent (i.e. standard gene editing). Suitably, gene editing efficiency may be improved by at least 1.1 fold, 1.2 fold, 1.3 fold, 1.4 fold, 1.5 fold, 1.6 fold, 1.7 fold, 1.8 fold, 1.9 fold, 1.8 fold, 1.9 fold, 2.0 fold, 2.1 fold, 2.2 fold, 2.3 fold, 2.4 fold, 2.5 fold, 3 fold or more.

In one embodiment, the inhibitor (or combination of inhibitors) for use according to the present invention improves gene editing efficiency compared with gene editing without use of the agent (i.e. standard gene editing). Suitably, the percentage of cells which have been edited is increased. Suitably, the percentage of cells which have been edited may be increased by 5%, 10%, 15%, 20%, 25%, 30%, 40%, 50%, 60%, 70%, 80%, 90%, 100%, 200%, 300% or more. Suitably, the percentage of the cells which have been edited may be 5%, 10%, 15%, 20%, 25%, 30%, 40%, 50%, 60%, 70%, 80%, 90% or more.

Suitably, the gene editing efficiency may be improved in a particular cell compartment. Suitably, gene editing is improved in a primitive HSPC cell compartment. Suitably, gene editing may be improved in CD34⁺ CD133⁻ cells. Suitably, gene editing may be improved in CD34⁺ CD133⁺ cells. Suitably, gene editing may be improved in CD34⁺ CD133⁺ CD90⁺ cells.

Preferably gene editing efficiency of CD34⁺ CD133⁺ CD90⁺ cells may be improved by at least 1.1 fold, 1.2 fold, 1.3 fold, 1.4 fold, 1.5 fold, 1.6 fold, 1.7 fold, 1.8 fold, 1.9 fold, 1.8 fold, 1.9 fold, 2.0 fold, 2.1 fold, 2.2 fold, 2.3 fold, 2.4 fold, 2.5 fold, 3 fold or more.

### Method of Gene Editing a Population of Cells

In one aspect, the invention provides a method of gene editing a population of haematopoietic cells, haematopoietic stem cells, haematopoietic progenitor cells and/or T cells comprising:
(a) introducing gene editing machinery to the population of haematopoietic cells, haematopoietic stem cells, haematopoietic progenitor cells and/or T cells with one or more vectors; and
(b) editing the genome of said haematopoietic cells, haematopoietic stem cells, haematopoietic progenitor cells and/or T cells;
   wherein prior to, at the same time as, or after the introducing of the gene editing machinery to the population of cells, the population of cells is contacted with one or more inhibitor(s) of senescence.

In a further aspect, the invention provides a method of gene editing a population of haematopoietic cells, haematopoietic stem cells, haematopoietic progenitor cells and/or T cells comprising the steps:
(a) contacting the population of haematopoietic cells, haematopoietic stem cells, haematopoietic progenitor cells and/or T cells with one or more inhibitor(s) of senescence;
(b) introducing gene editing machinery to the population of haematopoietic cells, haematopoietic stem cells, haematopoietic progenitor cells and/or T cells with one or more vectors; and
(c) editing the genome of said haematopoietic cells, haematopoietic stem cells, haematopoietic progenitor cells and/or T cells.

In a further aspect, the invention provides a method of gene editing a population of haematopoietic cells, haematopoietic stem cells, haematopoietic progenitor cells and/or T cells comprising the steps:
(a) introducing gene editing machinery to the population of haematopoietic cells, haematopoietic stem cells, haematopoietic progenitor cells and/or T cells with one or more vectors;
(b) contacting the population of haematopoietic cells, haematopoietic stem cells, haematopoietic progenitor cells and/or T cells with one or more inhibitor(s) of senescence; and
(c) editing the genome of said haematopoietic cells, haematopoietic stem cells, haematopoietic progenitor cells and/or T cells.

In a further aspect, the invention provides a method of gene editing a population of haematopoietic cells, haematopoietic stem cells, haematopoietic progenitor cells and/or T cells comprising the steps:
(a) introducing gene editing machinery to the population of haematopoietic cells, haematopoietic stem cells, haematopoietic progenitor cells and/or T cells with one or more vectors and simultaneously contacting the population of haematopoietic cells, haematopoietic stem cells, haematopoietic progenitor cells and/or T cells with one or more inhibitor(s) of senescence; and
(b) editing the genome of said haematopoietic cells, haematopoietic stem cells, haematopoietic progenitor cells and/or T cells.

In some embodiments, the method is a method of gene editing a population of haematopoietic cells, haematopoietic stem cells and/or haematopoietic progenitor cells.

In some embodiments, the steps of introducing gene editing machinery to the population of cells and of contacting the population of cells with one or more inhibitor(s) of senescence are carried out ex vivo or in vitro.

In some embodiments, the population of haematopoietic cells, haematopoietic stem cells and/or haematopoietic progenitor cells is contacted with the MAPK/ERK signalling inhibitor (e.g. the MAPK inhibitor) prior to or concurrently with the step of introducing gene editing machinery to said cells, preferably prior to the step of introducing gene editing machinery to said cells.

In some embodiments, the population of haematopoietic cells, haematopoietic stem cells and/or haematopoietic progenitor cells is contacted with the IL-1 inhibitor and/or NF-κB inhibitor prior to, concurrently with or following the step of introducing gene editing machinery to said cells.

Thus, in one embodiment of the method of gene editing a population of haematopoietic cells, haematopoietic stem cells, haematopoietic progenitor cells and/or T cells, steps (a) and (b) may be carried out simultaneously. In another embodiment, steps (a) and (b) are carried out sequentially, either step (a) before step (b) or step (b) before step (a).

In one embodiment, the gene editing machinery may comprise a nuclease such as a zinc finger nuclease (ZFNs), a transcription activator like effector nucleases (TALENs), meganucleases, or the clustered regularly interspaced short palindromic repeats (CRISPR)/Cas system.

The gene editing machinery (e.g. CRISPR/Cas system) may comprise one or more guide RNAs complementary to at least one target gene in a cell, an RNA-guided DNA endonuclease enzyme or nucleotide sequence encoding said endonuclease (e.g. Cas9 protein or a nucleotide sequence encoding a Cas9). In one embodiment, the gene editing machinery may be a CRISPR/Cas system.

In one embodiment, the gene editing machinery may be provided by one or more nucleotide sequences. Suitably, the nucleotide sequences encoding the gene editing machinery may be introduced to the cell sequentially or simultaneously. In one embodiment, the inhibitor(s) may be contacted with the cell simultaneously with the introduction of gene editing machinery to the cell. In one embodiment, one or more nucleotide sequences encoding gene editing machinery is introduced to the cell by electroporation. In one embodiment, one or more nucleotide sequences is introduced to the cell by transduction. Suitably, the nucleotide sequence may be introduced by transduction of a viral vector. For example, a Cas9 ribonucleoprotein may be introduced to a cell by electroporation before AAV6 transduction for the delivery of the donor DNA template.

As used herein, the term “introducing” refers to methods for inserting foreign DNA or RNA into a cell. As used herein, the term “introducing” includes both transduction and transfection methods. Transfection is the process of introducing nucleic acids into a cell by non-viral methods. Transduction is the process of introducing foreign DNA or RNA into a cell via a viral vector.

In one embodiment, AAV transduction is used to deliver the donor DNA template.

In one embodiment, AAV6 transduction is used to deliver the donor DNA template.

### Method of Transducing a Population of Cells

In a further aspect, the invention provides a method of transducing a population of haematopoietic cells, haematopoietic stem cells, haematopoietic progenitor cells and/or T cells with a viral vector comprising transducing the population of haematopoietic cells, haematopoietic stem cells, haematopoietic progenitor cells and/or T cells with one or more viral vectors the steps, and wherein prior to, at the same time as or following transducing the population of cells, the population of cells are contacted with one or more inhibitor(s) of senescence.

In a further aspect, the invention provides a method of transducing a population of haematopoietic cells, haematopoietic stem cells, haematopoietic progenitor cells and/or T cells with a viral vector comprising the steps:
(a) contacting the population of haematopoietic cells, haematopoietic stem cells, haematopoietic progenitor cells and/or T cells with one or more inhibitor(s) of senescence; and
(b) transducing the population of haematopoietic cells, haematopoietic stem cells, haematopoietic progenitor cells and/or T cells with one or more viral vectors.

In a further aspect, the invention provides a method of transducing a population of haematopoietic cells, haematopoietic stem cells, haematopoietic progenitor cells and/or T cells with a viral vector comprising the steps:
(a) transducing the population of haematopoietic cells, haematopoietic stem cells, haematopoietic progenitor cells and/or T cells with one or more viral vectors; and
(b) contacting the population of haematopoietic cells, haematopoietic stem cells, haematopoietic progenitor cells and/or T cells with one or more inhibitor(s) of senescence.

In a further aspect, the invention provides a method of transducing a population of haematopoietic cells, haematopoietic stem cells, haematopoietic progenitor cells and/or T cells with a viral vector comprising contacting the population of haematopoietic cells, haematopoietic stem cells, haematopoietic progenitor cells and/or T cells with one or more inhibitor(s) of senescence, and simultaneously transducing the population of haematopoietic cells, haematopoietic stem cells, haematopoietic progenitor cells and/or T cells with one or more viral vectors.

In some embodiments, the method is a method of gene editing a population of haematopoietic cells, haematopoietic stem cells and/or haematopoietic progenitor cells.

In some embodiments, steps (a) and (b) are carried out ex vivo or in vitro.

In some embodiments, the population of haematopoietic cells, haematopoietic stem cells and/or haematopoietic progenitor cells is contacted with the MAPK/ERK signalling inhibitor (e.g. MAPK inhibitor prior to or at the same time as the step of transducing said cells, preferably prior to the step of transducing said cells.

In some embodiments, the population of haematopoietic cells, haematopoietic stem cells and/or haematopoietic progenitor cells is contacted with the IL-1 inhibitor and/or NF-κB inhibitor prior to, concurrently with or following the step of transducing said cells.

### Haematopoietic Stem and Progenitor Cells

A stem cell is able to differentiate into many cell types. A cell that is able to differentiate into all cell types is known as totipotent. In mammals, only the zygote and early embryonic cells are totipotent. Stem cells are found in most, if not all, multicellular organisms. They are characterised by the ability to renew themselves through mitotic cell division and differentiate into a diverse range of specialised cell types. The two broad types of mammalian stem cells are embryonic stem cells that are isolated from the inner cell mass of blastocysts, and adult stem cells that are found in adult tissues. In a developing embryo, stem cells can differentiate into all of the specialised embryonic tissues. In adult organisms, stem cells and progenitor cells act as a repair system for the body, replenishing specialised cells, but also maintaining the normal turnover of regenerative organs, such as blood, skin or intestinal tissues.

Haematopoietic stem cells (HSCs) are multipotent stem cells that may be found, for example, in peripheral blood, bone marrow and umbilical cord blood. HSCs are capable of self-renewal and differentiation into any blood cell lineage. They are capable of recolonising the entire immune system, and the erythroid and myeloid lineages in all the haematopoietic tissues (such as bone marrow, spleen and thymus). They provide for life-long production of all lineages of haematopoietic cells.

Haematopoietic progenitor cells have the capacity to differentiate into a specific type of cell. In contrast to stem cells however, they are already far more specific: they are pushed to differentiate into their “target” cell. A difference between stem cells and progenitor cells is that stem cells can replicate indefinitely, whereas progenitor cells can only divide a limited number of times. Haematopoietic progenitor cells can be rigorously distinguished from HSCs only by functional in vivo assay (i.e. transplantation and demonstration of whether they can give rise to all blood lineages over prolonged time periods).

The haematopoietic stem and progenitor cells of the invention comprise the CD34 cell surface marker (denoted as CD34⁺).

In one embodiment, the cells for use in the present invention are haematopoietic cells, haematopoietic stem cells and/or haematopoietic progenitor cells which have been transduced with one or more viral vectors.

In one embodiment, the cells for use in the present invention are HSPCs.

In one embodiment, the cells for use in the present invention are primitive HSPCs. In one embodiment, a primitive subset of HSPCs refers to a population of HSCs which is CD90⁺. In one embodiment, a primitive subset of HSPCs refers to a population of cells which is CD34⁺ CD133⁺ and CD90⁺.

In one embodiment, the cells for use in the present invention are HSCs.

### Haematopoietic Stem and Progenitor Cell (HSPC) Sources

A population of haematopoietic stem and/or progenitor cells may be obtained from a tissue sample.

For example, a population of haematopoietic stem and/or progenitor cells may be obtained from peripheral blood (e.g. adult and foetal peripheral blood), umbilical cord blood, bone marrow, liver or spleen. Preferably, these cells are obtained from peripheral blood or bone marrow. They may be obtained after mobilisation of the cells in vivo by means of growth factor treatment.

Mobilisation may be carried out using, for example, G-CSF, plerixaphor or combinations thereof. Other agents, such as NSAIDs and dipeptidyl peptidase inhibitors, may also be useful as mobilising agents.

With the availability of the stem cell growth factors GM-CSF and G-CSF, most haematopoietic stem cell transplantation procedures are now performed using stem cells collected from the peripheral blood, rather than from the bone marrow. Collecting peripheral blood stem cells provides a bigger graft, does not require that the donor be subjected to general anaesthesia to collect the graft, results in a shorter time to engraftment and may provide for a lower long-term relapse rate.

Bone marrow may be collected by standard aspiration methods (either steady-state or after mobilisation), or by using next-generation harvesting tools (e.g. Marrow Miner).

In addition, haematopoietic stem and progenitor cells may also be derived from induced pluripotent stem cells.

### HSC Characteristics

HSCs are typically of low forward scatter and side scatter profile by flow cytometric procedures. Some are metabolically quiescent, as demonstrated by Rhodamine labelling which allows determination of mitochondrial activity. HSCs may comprise certain cell surface markers such as CD34, CD45, CD133, CD90 and CD49f. They may also be defined as cells lacking the expression of the CD38 and CD45RA cell surface markers. However, expression of some of these markers is dependent upon the developmental stage and tissue-specific context of the HSC. Some HSCs called “side population cells” exclude the Hoechst 33342 dye as detected by flow cytometry. Thus, HSCs have descriptive characteristics that allow for their identification and isolation.

### Negative Markers

CD38 is the most established and useful single negative marker for human HSCs.

Human HSCs may also be negative for lineage markers such as CD2, CD3, CD14, CD16, CD19, CD20, CD24, CD36, CD56, CD66b, CD271 and CD45RA. However, these markers may need to be used in combination for HSC enrichment.

By “negative marker” it is to be understood that human HSCs lack the expression of these markers.

### Positive Markers

CD34 and CD133 are the most useful positive markers for HSCs.

Some HSCs are also positive for lineage markers such as CD90, CD49f and CD93. However, these markers may need to be used in combination for HSC enrichment.

By “positive marker” it is to be understood that human HSCs express these markers.

In one embodiment, the haematopoietic stem and progenitor cells are CD34+CD38− cells.

### Differentiated Cells

A differentiated cell is a cell which has become more specialised in comparison to a stem cell or progenitor cell. Differentiation occurs during the development of a multicellular organism as the organism changes from a single zygote to a complex system of tissues and cell types. Differentiation is also a common process in adults: adult stem cells divide and create fully-differentiated daughter cells during tissue repair and normal cell turnover. Differentiation dramatically changes a cell's size, shape, membrane potential, metabolic activity and responsiveness to signals. These changes are largely due to highly-controlled modifications in gene expression. In other words, a differentiated cell is a cell which has specific structures and performs certain functions due to a developmental process which involves the activation and deactivation of specific genes. Here, a differentiated cell includes differentiated cells of the haematopoietic lineage such as monocytes, macrophages, neutrophils, basophils, eosinophils, erythrocytes, megakaryocytes/platelets, dendritic cells, T cells, B-cells and NK-cells. For example, differentiated cells of the haematopoietic lineage can be distinguished from stem cells and progenitor cells by detection of cell surface molecules which are not expressed or are expressed to a lesser degree on undifferentiated cells. Examples of suitable human lineage markers include CD33, CD13, CD14, CD15 (myeloid), CD19, CD20, CD22, CD79a (B), CD36, CD71, CD235a (erythroid), CD2, CD3, CD4, CD8 (T) and CD56 (NK).

In one embodiment, the haematopoietic cells referred to herein is a T-cell.

### T Cells

T-cells or T lymphocytes are a type of lymphocyte that play a central role in cell-mediated immunity. They can be distinguished from other lymphocytes, such as B cells and natural killer cells (NK cells), by the presence of a TCR on the cell surface. There are various types of T-cell, as summarised below.

Cytolytic T-cells (TC cells, or CTLs) destroy virally infected cells and tumour cells, and are also implicated in transplant rejection. CTLs express the CD8 at their surface. These cells recognize their targets by binding to antigen associated with MHC class I, which is present on the surface of all nucleated cells. Through IL-10, adenosine and other molecules secreted by regulatory T-cells, the CD8+ cells can be inactivated to an anergic state, which prevent autoimmune diseases such as experimental autoimmune encephalomyelitis.

Regulatory T-cells (Treg cells), formerly known as suppressor T-cells, are crucial for the maintenance of immunological tolerance. Their major role is to shut down T-cell-mediated immunity toward the end of an immune reaction and to suppress auto-reactive T-cells that escaped the process of negative selection in the thymus.

Naturally occurring Treg cells (also known as CD4⁺CD25⁺FoxP3⁺ Treg cells) arise in the thymus and have been linked to interactions between developing T-cells with both myeloid (CD11c⁺) and plasmacytoid (CD123⁺) dendritic cells that have been activated with TSLP. Naturally occurring Treg cells can be distinguished from other T-cells by the presence of an intracellular molecule called FoxP3. Mutations of the FOXP3 gene can prevent regulatory T-cell development, causing the fatal autoimmune disease IPEX.

Adaptive Treg cells (also known as Tr1 cells or Th3 cells) may originate during a normal immune response.

Helper T helper cells (TH cells) assist other white blood cells in immunologic processes, including maturation of B cells into plasma cells and memory B cells, and activation of cytotoxic T-cells and macrophages. TH cells express CD4 on their surface. TH cells become activated when they are presented with peptide antigens by MHC class II molecules on the surface of antigen presenting cells (APCs). These cells can differentiate into one of several subtypes, including TH1, TH2, TH3, TH17, TH9, or TFH, which secrete different cytokines to facilitate different types of immune responses.

Memory T-cells are a subset of antigen-specific T-cells that persist long-term after an infection has resolved. They quickly expand to large numbers of effector T-cells upon re-exposure to their cognate antigen, thus providing the immune system with “memory” against past infections. Memory T-cells comprise three subtypes: central memory T-cells (TCM cells) and two types of effector memory T-cells (TEM cells and TEMRA cells). Memory cells may be either CD4⁺ or CD8⁺. Memory T-cells typically express the cell surface protein CD45RO.

Two major classes of CD4⁺ Treg cells have been described—naturally occurring Treg cells and adaptive Treg cells.

Natural killer T-cells (NKT-cells) are a subset of CD1d-restricted T-cells at the interface between the innate and adaptive immune system. NKT-cells recognize lipids and glycolipids presented by CD1d molecules, a member of the CD1 family of antigen-presenting molecules, rather than peptide/MHC complexes. Naturally occurring NKT-cells co-express an αβ TCR and also a variety of molecular markers that are typically associated with NK cells, such as NK1.1, CD16 and CD56 expression and granzyme production. Thus, these cells feature characteristics of both conventional T-cells and NK cells and include both NK1.1⁺ and NK1.1⁻, as well as CD4⁺, CD4⁻, CD8⁺ and CD8⁻ cells.

NKT-cells can be subdivided into functional subsets that respond rapidly to a wide variety of glycolipids and stress-related proteins using T- or natural killer (NK) cell-like effector mechanisms. Because of their major modulating effects on immune responses via secretion of cytokines, NKT-cells are also considered important players in tumor immunosurveillance.

The cells according to the invention may be any of the cell types mentioned above.

T or NK cells, for example, may be activated and/or expanded, for example by treatment with an anti-CD3 monoclonal antibody, prior to being transduced and/or edited as described herein.

Alternatively, the cell may be derived from ex vivo differentiation of inducible progenitor cells or embryonic progenitor cells to T-cells. Alternatively, an immortalized T-cell line which retains its lytic function may be used.

In one embodiment, the cells for use in the present invention are T cells which have been transduced with one or more viral vectors.

### Inhibitors of Senescence

Senescence is a process by which a cell permanently stops dividing but is still metabolically active and does not die. Therefore, senescent cells may gradually accumulate in a cell population. Cell senescence occurs due to aging and serious DNA damage. As discussed above, protracted DDR signalling has been causatively linked to the establishment of cellular senescence which also results in the activation of a senescence-associated secretory phenotype (SASP).

Inhibitors of senescence can be grouped into two main categories: (i) senosuppressors which are a class of drugs that slow down the rate at which senescent cells form (for example, a MAPK/ERK signalling pathway inhibitor such as a p38 inhibitor) and (ii) SASP inhibitors which are inhibitors of senescence-associated inflammation (for example, IL-1 inhibitors and NF-κB inhibitors). Therefore, senosuppressors act prior to the formation of senescent cells whereas SASP inhibitors act downstream (i.e. after senescent cells have formed) and inhibit a phenotype associated with senescence (i.e. SASP). Hence, a senosuppressor and a SASP inhibitor would be expected to act prior to, during and after gene editing and/or transduction of a cell. Moreover, it is preferable to add a senosuppressor prior to gene editing and/or transduction of the cell, whereas a SASP inhibitor is preferably added during and/or after gene editing and/or transduction of the cell.

In some embodiments, the inhibitor of senescence is a senosuppressor or a Senescence Associated Secretory Phenotype (SASP) inhibitor.

In some embodiments, the inhibitor of senescence is a senosuppressor.

In some embodiments, the inhibitor of senescence is a Senescence Associated Secretory Phenotype (SASP) inhibitor. Suitably, the SASP inhibitor is an IL-1 inhibitor as described herein. Suitably, the SASP inhibitor is an NF-κB inhibitor as described herein.

Suitably, multiple inhibitors of senescence may be used in combination. Suitably, a senosuppressor and a SASP inhibitor may be used in combination. Suitably, multiple senosuppressors and/or multiple SASP inhibitors may be used in combination. Thus, advantageously, slowing of the rate at which senescent cells form along with inhibition of the SASP phenotype can be achieved.

Accordingly, in some embodiments, the methods and uses of the invention comprise the use of a plurality of inhibitors of senescence. In one embodiment, one or more senosuppressors and one or more SASP inhibitors are used in combination. Suitably, the plurality of inhibitors of senescence are distinct from one another. In other words, when multiple inhibitors of senescence are used, each inhibitor has a different target. Thus, the use of multiple inhibitors may provide an additive or synergistic effect.

In some embodiments, the methods and uses of the invention comprise the use of a senosupressor (e.g. a MAPK/ERK signalling pathway inhibitor such as a MAPK inhibitor), an IL-1 inhibitor and/or an inhibitor of NF-κB. Suitably, a senosupressor (e.g. a MAPK/ERK signalling pathway inhibitor such as a MAPK inhibitor) is used. Suitably, an IL-1 inhibitor is used. Suitably, an NF-κB inhibitor is used. Suitably, a senosupressor (e.g. a MAPK/ERK signalling pathway inhibitor such as a MAPK inhibitor) is used in combination with an IL-1 inhibitor. Suitably, a senosupressor (e.g. a MAPK/ERK signalling pathway inhibitor such as a MAPK inhibitor) used in combination with an NF-κB inhibitor. Suitably, an IL-1 inhibitor is used in combination with an NF-κB inhibitor. Suitably, a senosupressor (e.g. a MAPK/ERK signalling pathway inhibitor such as a MAPK inhibitor), an IL-1 inhibitor and an NF-κB inhibitor are used in combination.

The term “combination”, or terms “in combination”, “used in combination with” or “combined preparation” as used herein may refer to the combined administration of two or more entities simultaneously, sequentially or separately.

In one embodiment, the senosuppressor (e.g. a MAPK/ERK signalling pathway inhibitor such as a MAPK inhibitor), IL-1 inhibitor and/or NF-κB inhibitor are administered simultaneously, sequentially or separately.

In one embodiment, the senosuppressor (e.g. a MAPK/ERK signalling pathway inhibitor such as a MAPK inhibitor), IL-1 inhibitor and/or NF-κB inhibitor are administered simultaneously.

In one embodiment, the senosuppressor (e.g. a MAPK/ERK signalling pathway inhibitor such as a MAPK inhibitor), IL-1 inhibitor and/or NF-κB inhibitor are administered sequentially.

In one embodiment, the senosuppressor (e.g. a MAPK/ERK signalling pathway inhibitor such as a MAPK inhibitor), IL-1 inhibitor and/or NF-κB inhibitor are administered separately.

The term “simultaneous” as used herein means that the entities are administered concurrently, i.e. at the same time.

The term “sequential” as used herein means that the entities are administered one after the other.

The term “separate” as used herein means that the entities are administered independently of each other but within a time interval that allows the entities to show a combined, preferably synergistic, effect. Thus, administration “separately” may permit one entity to be administered, for example, within 1 minute, 5 minutes or 10 minutes after the other.

In some embodiments, the inhibition of senescence (e.g. the MAPK/ERK signalling pathway inhibition such as the MAPK inhibition), inhibition of IL-1 and/or inhibition of NF-κB in the haematopoietic cells, haematopoietic stem cells, haematopoietic progenitor cells and/or T cells is transient.

In one embodiment, the senosuppressor (e.g. a MAPK/ERK signalling pathway inhibitor such as a MAPK inhibitor), IL-1 inhibitor and/or NF-κB inhibitor is a transient inhibitor (e.g. has an inhibitory action lasting less than about 1, 2, 3, 4, 5, 6, 7 or 14 days), such as a reversible inhibitor. Suitably, the senosuppressor (e.g. a MAPK/ERK signalling pathway inhibitor such as a MAPK inhibitor) is a transient inhibitor. Suitably, the IL-1 inhibitor is a transient inhibitor. Suitably, the NF-κB inhibitor of is a transient inhibitor. Preferably, the cells are exposed to the inhibitor(s) for about 1-48 or 1-24 hours, preferably 1-24 hours. The cells may be, for example, exposed to the inhibitor(s) at the same time as the viral vector or before the viral vector.

In some embodiments, the haematopoietic cells, haematopoietic stem cells, haematopoietic progenitor cells and/or T cells are exposed to the one or more inhibitor(s) of senescence prior to, at the same time as and/or after the gene editing machinery is introduced into the cell, preferably at the same time as the gene editing machinery is introduced to the cell.

In some embodiments, the inhibition of senescence (e.g. MAPK inhibition), IL-1 and/or NF-κB occurs during gene editing of the haematopoietic cells, haematopoietic stem cells, haematopoietic progenitor cells and/or T cells.

In some preferred embodiments, the haematopoietic cells, haematopoietic stem cells, haematopoietic progenitor cells and/or T cells are exposed to the inhibitor of senescence (in particular a senosuppressor, e.g. a MAPK inhibitor) prior to the gene editing machinery being introduced to the cell.

In some preferred embodiments, the MAPK/ERK signalling inhibition (e.g. the MAPK inhibition) occurs prior to and/or during gene editing of the haematopoietic cells, haematopoietic stem cells, haematopoietic progenitor cells and/or T cells.

In some embodiments, the haematopoietic cells, haematopoietic stem cells, haematopoietic progenitor cells and/or T cells are exposed to the one or more inhibitor(s) of senescence prior to, at the same time as and/or after the cell is transduced with one or more viral vectors, preferably at the same time as the cell is transduced with one or more viral vectors.

In some embodiments, the inhibition of senescence (e.g. MAPK inhibition), IL-1 and/or NF-κB occurs during transduction of the haematopoietic cells, haematopoietic stem cells, haematopoietic progenitor cells and/or T cells with one or more viral vectors.

In some preferred embodiments, the haematopoietic cells, haematopoietic stem cells, haematopoietic progenitor cells and/or T cells are exposed to the inhibitor of senescence (in particular a senosuppressor, e.g. a MAPK inhibitor) prior to the cell being transduced with one or more viral vectors.

In some preferred embodiments, the MAPK/ERK signalling inhibition (e.g. the MAPK inhibition) occurs prior to and/or during transduction of the haematopoietic cells, haematopoietic stem cells, haematopoietic progenitor cells and/or T cells with one or more viral vectors.

In some embodiments, the MAPK inhibitor, IL-1 inhibitor and/or NF-κB inhibitor is added to the haematopoietic cell, haematopoietic stem cell, haematopoietic progenitor cells and/or T cells at a concentration of about 0.5-200 μM or about 0.1-200 ng/μl.

In one embodiment, the MAPK inhibitor, IL-1 inhibitor and/or NF-κB inhibitor is added to the haematopoietic cells, haematopoietic stem cells, haematopoietic progenitor cells and/or T cells (e.g. in an in vitro or ex vivo culture) at a concentration of about 0.5-200, 0.5-150, 0.5-100, 0.5-50, 0.5-40, 0.5-30, 0.5-20 or 0.5-15 μM, preferably about 0.5-30 μM, more preferably about 0.5-15 μM. In one embodiment, the MAPK inhibitor, IL-1 inhibitor and/or NF-κB inhibitor is added to the haematopoietic cells, haematopoietic stem cells, haematopoietic progenitor cells and/or T cells (e.g. in an in vitro or ex vivo culture) at a concentration of about 1-200, 1-150, 1-100, 1-50, 1-40, 1-30, 1-20 or 1-15 μM, preferably about 1-30 μM, more preferably about 1-15 μM. In another embodiment, the inhibitor(s) is added to the haematopoietic cells, haematopoietic stem cells, haematopoietic progenitor cells and/or T cells at a concentration of about 5-200, 5-150, 5-100, 5-50, 5-40, 5-30, 5-20 or 5-15 μM, preferably about 5-30 μM.

In one embodiment, the MAPK inhibitor, IL-1 inhibitor and/or NF-κB inhibitor is added to the haematopoietic cells, haematopoietic stem cells, haematopoietic progenitor cells and/or T cells (e.g. in an in vitro or ex vivo culture) at a concentration of about 1, 2, 3, 4, 5, 6, 7, 8, 9, 10, 11, 12, 13, 14, 15, 20, 25, 30, 35, 40, 45, 50, 60, 70, 80, 90, 100, 125, 150, 175 or 200 μM.

In one embodiment, the MAPK inhibitor, IL-1 inhibitor and/or NF-κB inhibitor is added to the haematopoietic cells, haematopoietic stem cells, haematopoietic progenitor cells and/or T cells (e.g. in an in vitro or ex vivo culture) at a concentration of about 0.1-200, 0.1-150, 0.1-100, 0.1-75, 0.1-60, 0.1-50, 0.1-25, 0.1-20, 0.1-15 or 0.1-10 ng/μl, preferably about 0.1-60 ng/μl. In another embodiment, the inhibitor(s) is added to the haematopoietic cells, haematopoietic stem cells, haematopoietic progenitor cells and/or T cells at a concentration of about 5-200, 5-150, 5-100, 5-75, 5-60, 5-50, 5-25, 5-20, 5-15 or 5-10 ng/μl, preferably about 5-60 ng/μl.

In one embodiment, the MAPK inhibitor, IL-1 inhibitor and/or NF-κB inhibitor is added to the haematopoietic cells, haematopoietic stem cells, haematopoietic progenitor cells and/or T cells (e.g. in an in vitro or ex vivo culture) at a concentration of about 0.1, 1, 2, 3, 4, 5, 6, 7, 8, 9, 10, 11, 12, 13, 14, 15, 20, 25, 30, 35, 40, 45, 50, 55, 60, 65, 70, 75, 80, 90, 100, 125, 150, 175, or 200 ng/μl, preferably about 50 ng/μl.

It will be understood that, where the concentration of a combination of inhibitors is indicated herein, the concentration refers to each individual inhibitor within the combination. For example, where it is stated that the MAPK inhibitor, IL-1 inhibitor and NF-κB inhibitor is added to the haematopoietic cell, haematopoietic stem cell, haematopoietic progenitor cells and/or T cells at a concentration of about 0.5-200 μM or about 0.1-200 ng/μl, this means that the MAPK inhibitor is added at a concentration of about 0.5-200 μM or about 0.1-200 ng/μl, the IL-1 inhibitor is added at a concentration of about 0.5-200 μM or about 0.1-200 ng/μl an the NF-κB inhibitor is added at a concentration of about 0.5-200 μM or about 0.1-200 ng/μl.

In one embodiment, the one or more inhibitors of senescence comprise or consist of a p38 inhibitor, an IL-1 inhibitor and/or an NF-κB inhibitor. Suitably, the one or more inhibitors of senescence comprise or consist of SB203580 or a derivative thereof, anakinra and/or SC514 or a derivative thereof.

In one embodiment, the one or more inhibitors of senescence comprise or consist of a JNK inhibitor, an IL-1 inhibitor and/or an NF-κB inhibitor. Suitably, the one or more inhibitors of senescence comprise or consist of SP600125 or a derivative thereof, anakinra and/or SC514 or a derivative thereof.

In one embodiment, the one or more inhibitors of senescence comprise or consist of an ERK inhibitor), an IL-1 inhibitor and/or an NF-κB inhibitor. Suitably, the one or more inhibitors of senescence comprise or consist of FR180204 or a derivative thereof, anakinra and/or SC514 or a derivative thereof.

A candidate inhibitor may be analysed for its ability to increase cell survival and/or engraftment using a method as disclosed herein. For example, a candidate inhibitor may be analysed for its ability to increase clonogenic potential using a method as disclosed herein. A candidate inhibitor may also be analysed for its ability to increase gene editing efficiency using a method as disclosed herein. A candidate inhibitor may be analysed for its ability to decrease p21 levels as described herein. A candidate inhibitor may be analysed for its ability to dampen proinflammatory programmes as a result of gene editing as described herein.

Senosuppressor The mammalian Mitogen-Activated Protein Kinase (MAPK) family of kinases includes three subfamilies: Extracellular signal-Regulated Kinases (ERKs), c-Jun N-terminal Kinases (JNKs) and p38 mitogen-activated protein kinases (p38s). Generally, ERKs are activated by growth factors and mitogens, whereas JNKs and p38s are activated by cellular stresses and inflammatory cytokines. “Classical” MAPKs are activated by phosphorylation events in their activation loops (typically, the activation is dependent on two phosphorylation events) and form a three-tiered signalling pathway. The tandem MAPK activation loop phosphorylation is performed by members of the Ste7 protein kinase family, also known as MAP2 kinases (MAP2Ks). MAP2Ks in turn, are activated by phosphorylation performed by a number of different upstream serine-threonine kinases termed MAP3 kinases (MAP3Ks). Most MAP2Ks display very little activity on substrates other than their cognate MAPK, such that classical MAPK pathways are multi-tiered but relatively linear. “Atypical” MAPKs do not have dual phosphorylation sites and only form two-tiered pathways.

In some embodiments, the inhibitor of senescence (e.g., the senosuppressor) is an inhibitor of the Mitogen-Activated Protein Kinase (MAPK)/Extracellular-Signal-Regulated Kinases (ERK) signalling pathway. Inhibition of the MAPK/ERK signalling pathway can be determined using methods known in the art. Suitably, the inhibitor is a MAP3K inhibitor, a MAK2K inhibitor, a MAPK inhibitor, preferably wherein the inhibitor is an MKK7 inhibitor, an MKK4 inhibitor, an MKK3/6 inhibitor, an MEK1/2 inhibitor, a JNK inhibitor, a p38 inhibitor or an ERK inhibitor.

In some preferred embodiments, the MAPK/ERK signalling inhibitor is a MAPK inhibitor.

The activity of a MAPK may be analysed directly, for example by analysing the enzymatic activity of the MAPK in vitro.

The ability of a candidate agent to inhibit (e.g. reduce the activity) of a MAPK may be expressed in terms of an IC50 value, which is the concentration of an agent that is required to give rise to a 50% reduction in the activity of the kinase. Preferably, the inhibitors of the invention have an IC50 value for inhibition (e.g. of MAPK) of less than 100 μM, more preferably less than 10 μM, for example less than 1 μM, less than 100 nM or less than 10 nM.

A number of techniques are known in the art for measuring kinase activity. Preferably, the kinase activity assays are carried out on a kinase (e.g. a MAPK) that has been isolated from a cell. The kinase may have been expressed using recombinant techniques, and preferably has been purified. For example, kinase activity may be determined by monitoring the incorporation of radiolabelled phosphate from [γ-³²P]-labelled ATP into a substrate. Such assay techniques are described in, for example, Hastie et al. (Hastie, C. J. et al. (2006) Nat. Protocols 1: 968-971).

Preferably, the MAPK inhibitor is an inhibitor of p38 phosphorylation, an inhibitor of JNK phosphorylation or an inhibitor of ERK phosphorylation, preferably an inhibitor of p38 phosphorylation.

In some preferred embodiments, the MAPK inhibitor is a JNK inhibitor, a p38 inhibitor or an ERK inhibitor, preferably a p38 inhibitor.

In some embodiments, the MAPK inhibitor is FR180204, SP600125, SB203580, SB202190, LY2228820, BIRB 796, TAT-TN13, SB203580 hydrochloride, AMG548, SB239063, CMPD-1, JX 401, EO 1428, RWJ 67657, SCIO 469 hydrochloride, VX 745, TAK 715, ML 3403, AL 8697, SB 706504, DBM 1285 dihydrochloride, PH 797804, Org 48762-0, TMCB, XMD 8-92, Pluripotin, TCS ERK 11e, ERK5-IN-1, DEL 22379, AX 15836, TCS JNK 6o, SU 3327, CEP 1347, c-JUN peptide, AEG 3482, TCS JNK 5a, BI 78D3, IQ 3, SR 3576, CC 401 dihydrochloride, or a variant or derivative thereof.

In some embodiments, the MAPK inhibitor is FR180204, SP600125, SB203580, SB202190, LY2228820, BIRB 796; SB203580 hydrochloride, SCIO 469 hydrochloride, TMCB, XMD 8-92, TCS JNK 6o, SU 3327, CC 401 dihydrochloride, or a variant or derivative thereof.

In some embodiments, the MAPK inhibitor is a JNK inhibitor.

In some embodiments, the JNK inhibitor is SP600125, TCS JNK 6o, SU 3327, CEP 1347, c-JUN peptide, AEG 3482, TCS JNK 5a, BI 78D3, IQ 3, SR 3576, CC 401 dihydrochloride, or a derivative thereof.

In some embodiments, the JNK inhibitor is SP600125, TCS JNK 6o, SU 3327, CC 401 dihydrochloride, or a derivative thereof.

In some preferred embodiments, the MAPK inhibitor is SP600125 or a derivative thereof.

SP600125 (C₁₄H₈N₂O; Anthra[1-9-cd]pyrazol-6(2H)-one) is a selective JNK inhibitor. SP600125 (CAS No. 129-56-6), also known as JNK Inhibitor II, is a cell-permeable, potent, selective, ATP-competitive, and reversible inhibitor of JNK. SP600125 is active in vivo and competitively and reversibly inhibits JNK1, 2 and 3 (IC₅₀=40-90 nM) with negligible activity against ERK2, p383 and a range of other enzymes. This inhibitor protects renal tubular epithelial cells against ischemia/reperfusion-induced apoptosis.

In one embodiment, SP600125 has the following structure:

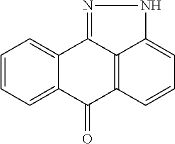 [SMILES: O=C1c2ccccc2-c2n[nH]c3cccc1c23]

TCS JNK 60 (C₁₈H₂ON₄O₄; N-(4-Amino-5-cyano-6-ethoxy-2-pyridinyl)-2,5-dimethoxybenzeneacetamide) is an ATP-competitive JNK inhibitor (IC₅₀ values are 2, 4 and 52 nM for JNK1, JNK2 and JNK3, respectively). TCS JNK 6o displays >1000-fold selectivity over other kinases, including ERK2 and p38. TCS JNK 60 inhibits c-Jun phosphorylation (EC₅₀=920 nM) and prevents collagen-induced platelet aggregation in vitro.

SU 3327 (C₅H₃N₅O₂S₃; 5-[(5-Nitro-2-thiazolyl)thio]-1,3,4thiadiazol-2-amine) is a selective inhibitor of JNK (IC₅₀=0.7 μM). This inhibitor displays selectivity over p38 MAPK and Akt and inhibits the protein-protein interaction between JNK and JIP (IC₅₀=239 nM).

CC 401 dihydrochloride (C₂₂H₂₄N₆O·2HCl; 3-[3-[2-(1-Piperidinyl)ethoxy]phenyl]-5-(1H-1,2,4-triazol-5-yl)-1H-indazole dihydrochloride) is a high affinity JNK inhibitor (Kᵢ values are 25-50 nM). CC 401 dihydrochloride inhibits JNK via competitive binding of the ATP-binding site of active, phosphorylated JNK. This inhibitor exhibits >40-fold selectivity for JNK over p38, ERK, IKK2, protein kinase C, Lck and ZAP70. CC 401 dihydrochloride is hepatoprotective and also inhibits HCMV replication.

Other JNK inhibitors include the following: CEP 1347, an inhibitor of JNK signalling; c-JUN peptide, a peptide inhibitor of JNK/c-Jun interaction; AEG 3482, an inhibitor of JNK signalling; TCS JNK 5a, a selective inhibitor of JNK2 and JNK3; BI 78D3, a selective, competitive JNK inhibitor; IQ 3, a selective JNK3 inhibitor; and SR 3576a highly potent and selective JNK3 inhibitor.

In some preferred embodiments, the MAPK inhibitor is a p38 inhibitor.

In some embodiments, the p38 inhibitor is SB203580, SB202190, LY2228820, BIRB 796; TAT-TN13, SB203580 hydrochloride, AMG548, SB239063, CMPD-1, JX 401, EO 1428, RWJ 67657, SCIO 469 hydrochloride, VX 745, TAK 715, ML 3403, AL 8697, SB 706504, DBM 1285 dihydrochloride, PH 797804, Org 48762-0, or a variant or derivative thereof.

In some embodiments, the p38 inhibitor is SB203580, SB203580 hydrochloride, SB202190, LY2228820, BIRB 796, SCIO 469 hydrochloride, or a derivative thereof.

In some preferred embodiments, the MAPK inhibitor is SB203580, SB203580 hydrochloride, or a derivative thereof.

In some preferred embodiments, the MAPK inhibitor is SB203580 or a derivative thereof.

SB203580 (CAS No. 152121-47-6) is a highly specific, potent, cell-permeable, selective, reversible, and ATP-competitive inhibitor of p38 MAP kinase. SB 203580 hydrochloride is water-soluble. SB 203580 (C₂₀H₁₄N₃OF; 4-[4-(4-Fluorophenyl)-5-(4-pyridinyl)-1H-imidazol-2-yl]phenol), and SB 203580 hydrochloride, is a pyridinyl imidazole that suppresses the activation of MAPKAP kinase-2 and inhibits the phosphorylation of heat shock protein (HSP) 27 in response to IL-1, cellular stresses and bacterial endotoxin in vivo. It does not inhibit JNK or p42 MAP kinase and, therefore, is useful for studying the physiological roles and targets of p38 MAPK and MAPKAP kinase-2. It has been shown to induce the activation of the serine/threonine kinase Raf-1 and has been reported to inhibit cytokine production.

In one embodiment, SB203580 has the following structure:

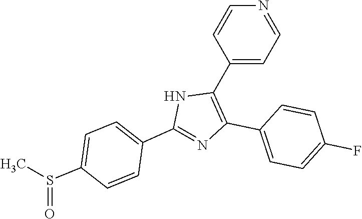 [SMILES: CS(=O)c1ccc(-c2nc(-c3ccc(F)cc3)c(-c3ccncc3)[nH]2)cc1]

SB 202190 (C₂₀H₁₄N₃OF; 4-[4-(4-Fluorophenyl)-5-(4-pyridinyl)-1H-imidazol-2-yl]phenol): SB 202190 is a selective p38 MAP kinase inhibitor with IC₅₀s of 50 nM and 100 nM for p38a and p3832, respectively. SB 202190 binds to the ATP pocket of the active recombinant human p38 kinase with a K_{d} of 38 nM. SB 202190 has anti-cancer activity and rescued memory deficits.

LY2228820 (C₂₄H₂₉FNs·2CH₃SO₃H) (also known as Ralimetinib) is a trisubstituted imidazole derivative and a potent inhibitor of the α- and β-isoforms of p38 MAP kinase (MAPK) in vitro (IC₅₀=5.3 and 3.2 nM, respectively) with anti-inflammatory and anti-neoplastic activities.

BIRB 796 (C₃₁H₃₇N₅O₃; N-[3-(1,1-Dimethylethyl)-1-(4-methylphenyl)-1H-pyrazol-5-yl]-N′-[4-[2-(4-morpholinyl)ethoxy]-1-naphthalenyl]urea) (also known as Doramapimod) is an orally active, highly potent p38 MAPK inhibitor. Doramapimod (BIRB 796) is usually associated with inflammation because of its role in T-cell proliferation and cytokine production. Doramapimod (BIRB 796) blocks the stress-induced phosphorylation of the scaffold protein SAP97, further establishing that this is a physiological substrate of SAPK3/p38γ. The binding of Doramapimod to the p38 MAPKs or JNK1/2 impairs their phosphorylation by the upstream kinase MKK6 or MKK4.

SCIO 469 hydrochloride (C₂₇H₃₀ClFN₄O₃·HCl; 6-Chloro-5-[[(2R,5S)-4-[(4-fluorophenyl)methyl]-2,5-dimethyl-1-piperazinyl]carbonyl]-N,N,1-trimethyl-α-oxo-1H-Indole-3-acetamide hydrochloride) (CAS No. 309913-83-5) (also known as Talmapimod) is an orally active, selective, and ATP-competitive p38a inhibitor with an IC₅₀ of 9 nM. Talmapimod shows about 10-fold selectivity over p38p, and at least 2000-fold selectivity over a panel of 20 other kinases, including other MAPKs. It specifically blocks the cytokine induced phosphorylation of p38 leading to a decrease in apoptosis of CD34⁺ HSPCs and to an increase in colony formation capacity.

Other p38 inhibitors include the following: TAT-TN13, a selective p38 kinase inhibitor; AMG 548, a potent and selective p38α inhibitor; SB 239063 a potent and selective inhibitor of p38 MAPK which is orally active; CMPD-1, a selective inhibitor of p38α-mediated MK2a phosphorylation and is also a tubulin polymerization inhibitor; JX 401, a potent and reversible p38α inhibitor; EO 1428, a selective inhibitor of p38α and p3832; RWJ 67657, a potent and selective p38α and p383 inhibitor; VX 745, a potent and selective p38α inhibitor; TAK 715, a potent p38 MAPK inhibitor which is also anti-inflammatory; ML 3403, a p38 inhibitor; AL 8697, a potent and selective p38α inhibitor; SB 706504, a p38 MAPK inhibitor; DBM 1285 dihydrochloride, a p38 MAPK inhibitor which is also anti-inflammatory; PH 797804, a potent and selective p38α/p inhibitor; and Org 48762-0, a selective p38α/3 inhibitor which is orally bioavailable.

In some embodiments, the MAPK inhibitor is an ERK inhibitor.

In some embodiments, the ERK inhibitor is FR180204, TMCB, XMD 8-92, Pluripotin, TCS ERK 11e, ERK5-IN-1, DEL 22379, AX 15836 or a variant or derivative thereof.

In some embodiments, the ERK inhibitor is FR180204, TMCB, XMD 8-92, or a derivative thereof.

In some preferred embodiments, the MAPK inhibitor is FR180204 or a derivative thereof.

FR180204 (CAS No. 865362-74-9) is a cell-permeable, potent, ATP-competitive inhibitor of ERK1 and ERK2. FR 180204 (C₁₈H₁₃N₇; 5-(2-Phenyl-pyrazolo[1,5-a]pyridin-3-yl)-1H-pyrazolo[3,4-c]pyridazin-3-ylamine) is a selective ERK inhibitor (IC₅₀ values are 0.14 and 0.31 μM for ERK2 and ERK1 respectively). FR 180204 displays 30-fold selectivity for ERK over p38α (IC₅₀=10 μM); displays no activity against human recombinant MEK1, MKK4, IKKα, PKCα, Src, Syc and PDGFα at concentrations less than 30 μM. FR 180204 also inhibits TGFβ-induced AP-1 activation in Mv1Lu cells (IC₅₀=3.1 μM).

In some embodiments, FR180204 has the following structure:

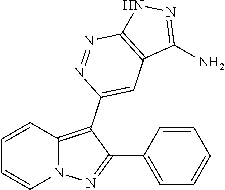 [SMILES: Nc1n[nH]c2nnc(-c3c(-c4ccccc4)nn4ccccc34)cc12]

TMCB (C₁₁H₉Br₄N₃O₂; 2-(4,5,6,7-Tetrabromo-2-(dimethylamino)-1H-benzo[d]imidazol-1-yl)acetic acid) is a dual-kinase inhibitor which inhibits both casein kinase 2 (CK2) and extracellular-signal-regulated kinase 8 (ERK8) (IC₅₀=0.50 μM for both CK2 and ERK8). TMCB displays selectivity for CK2 over protein kinases normally susceptible to CK2 inhibitors (Kᵢ values are 0.25, 8.65, 11.90 and 15.25 μM for CK2, PIM1, DYRK1a and HIPK2 respectively).

XMD 8-92 (C₂₆H₃₀N₈O₃; 2-[[2-Ethoxy-4-(4-hydroxy-1-piperidinyl)phenyl]amino]-5,11-dihydro-5,11-dimethyl-6H-pyrimido[4,5-b][1,4]benzodiazepin-6-one) is an ERK5 (BMK1) and BRD4 inhibitor (K_{d} values are 80 and 190 nM, respectively). XMD 8-92 also inhibits DCAMKL2, PLK4 and TNK1 (K_{d} values are 190, 600 and 890 nM). XMD 8-92 blocks growth factor-induced activation of cellular BMK1 and reduces BMK1 activity in in vitro kinase assays. This inhibitor also reduces BMK1-dependent transactivating activity of MEF2C. XMD 8-92 inhibits proliferation in a variety of cancer cell lines, blocks tumor cell proliferation and tumor-associated angiogenesis.

Other ERK inhibitors include the following: Pluripotin, a Dual ERK1/RasGAP inhibitor which maintains ESC self-renewal; TCS ERK 11e, a potent and selective ERK2 inhibitor; ERK5-IN-1, a potent and selective ERK5 inhibitor; DEL 22379, an ERK dimerization inhibitor; AX 15836, a potent and selective ERK5 inhibitor.

In some embodiments, the MAPK inhibitor is SB203580, FR180204, SP600125 or a derivative thereof.

In some embodiments, the inhibitor of senescence is not an IL-1 inhibitor.

In some embodiments, the inhibitor of senescence is not an NF-κB inhibitor.

In some embodiments, the inhibitor of senescence is not an inhibitor of p53 activation.

Suitably, the inhibitor of senescence is not GSE56. Suitably, the inhibitor of senescence is not GSE56 or a variant thereof.

In some embodiments, the inhibitor of senescence is not an IL-1 inhibitor, an NF-κB inhibitor or an inhibitor of p53 activation.

In one embodiment, the MAPK inhibitor (e.g. SB203580, FR180204, SP600125 or a derivative thereof) is added to the haematopoietic cells, haematopoietic stem cells, haematopoietic progenitor cells and/or T cells (e.g. in an in vitro or ex vivo culture) at a concentration of about 1-200, 1-150, 1-100, 1-50, 1-40, 1-30, 1-20 or 1-15 μM, preferably about 1-15 μM.

In one embodiment, the MAPK inhibitor (e.g. SB203580, FR180204, SP600125 or a derivative thereof) is added to the haematopoietic cells, haematopoietic stem cells, haematopoietic progenitor cells and/or T cells (e.g. in an in vitro or ex vivo culture) at a concentration of about 1, 2, 3, 4, 5, 6, 7, 8, 9, 10, 11, 12, 13, 14, 15 or 20 μM.

In another embodiment, the MAPK inhibitor (e.g. FR180204 or a derivative thereof) is added to the haematopoietic cells, haematopoietic stem cells, haematopoietic progenitor cells and/or T cells (e.g. in an in vitro or ex vivo culture) at a concentration of about 12 μM.

In another embodiment, the MAPK inhibitor (e.g. SP600125 or a derivative thereof) is added to the haematopoietic cells, haematopoietic stem cells, haematopoietic progenitor cells and/or T cells (e.g. in an in vitro or ex vivo culture) at a concentration of about 2 μM.

In another embodiment, the MAPK inhibitor (e.g. SB203580 or a derivative thereof) is added to the haematopoietic cells, haematopoietic stem cells, haematopoietic progenitor cells and/or T cells (e.g. in an in vitro or ex vivo culture) at a concentration of about 4 μM.

In one embodiment, the haematopoietic cells, haematopoietic stem cells, haematopoietic cell progenitor cells and/or T cells are contacted with the MAPK inhibitor about 15 minutes, 30 minutes, 1 hour, 1.5 hours, 2 hours, 2.5 hours, 3 hours, 3.5 hours, 4 hours, 12 hours, 24 hours, 36 hours, 48 hours, 60 hours, or 72 hours, preferably about 24 hours or 48 hours, before transducing the population of cells with the viral vector and/or before introducing the gene editing machinery into the cell.

In one embodiment, the haematopoietic cells, haematopoietic stem cells, haematopoietic progenitor cells and/or T cells are contacted with the MAPK inhibitor about 15 minutes to about 4 hours; about 15 minutes to about 3 hours; or about 15 minutes to about 2 hours after transducing the population of cells with the one or more viral vectors and/or after introducing the gene editing machinery into the cell. In another embodiment, the cells are contacted with the inhibitor about 1-4 hours; 1-3 hours; or 1-2 hours after transducing the population of cells with the viral vector and/or after introducing the gene editing machinery into the cell (e.g. after electroporating the cell).

In one embodiment, the haematopoietic cells, haematopoietic stem cells, haematopoietic cell progenitor cells and/or T cells are contacted with the MAPK inhibitor about 15 minutes, 30 minutes, 1 hour, 1.5 hours, 2 hours, 2.5 hours, 3 hours, 3.5 hours or 4 hours, preferably about 15 minutes, after transducing the population of cells with the viral vector and/or after introducing the gene editing machinery into the cell (e.g. after electroporating the cell).

Suitably, the MAPK inhibitor may be active during gene editing.

Suitably, the MAPK inhibitor may be active during transduction.

In one embodiment, the cells are contacted with the MAPK inhibitor for about 12-96 h, such as 12-60 h, 24-60 h, 36-60 h or 42-54 h, preferably about 42-54 h, after introducing the gene editing machinery and/or transducing the viral vector into the cell. In one embodiment, the contacting is carried out for about 12, 18, 24, 30, 36, 42, 48, 54, 60, 66, 72, 78, 84, 90, 96, 102 h, preferably about 48 h or about 96 h, after introducing and/or transducing the cells is started.

In one embodiment, the cells are contacted with the MAPK inhibitor about 12-60 h, such as 24-60 h, 36-60 h or 42-54 h, preferably about 42-54 h, after beginning culture of the population of haematopoietic cells, haematopoietic stem cells, haematopoietic progenitor cells and/or T cells (e.g. beginning culture after the cells are thawed from a frozen state). In one embodiment, the cells are contacted with the MAPK inhibitor for about 12, 18, 24, 30, 36, 42, 48, 54 or 60 h, preferably about 48 h, after beginning culture of the population of haematopoietic cells, haematopoietic stem cells, haematopoietic progenitor cells and/or T cells.

In one embodiment, the cells are contacted with the MAPK inhibitor for about 12-60 h, such as 24-60 h, 36-60 h or 42-54 h, preferably about 42-54 h, after thawing the population of haematopoietic cells, haematopoietic stem cells, haematopoietic progenitor cells and or T cells (e.g. which had been stored in a frozen state). In one embodiment, the cells are contacted with the MAPK inhibitor for about 12, 18, 24, 30, 36, 42, 48, 54 or 60 h, preferably about 48 h, after thawing the population of haematopoietic cells, haematopoietic stem cells, haematopoietic progenitor cells and/or T cells.

In one embodiment, the cells are contacted with the MAPK inhibitor for about 12-60 h, such as 24-60 h, 36-60 h or 42-54 h, preferably about 42-54 h, before introducing the gene editing machinery and/or of transducing the viral vector to the cell is started. In one embodiment, the contacting is carried out for about 12, 18, 24, 30, 36, 42, 48, 54 or 60 h, preferably about 48 h, before introducing and/or transducing the cells is started.

In one embodiment, the cells are contacted with the MAPK inhibitor for about 12-96 h, such as 12-60 h, 24-60 h, 36-60 h or 42-54 h, preferably about 42-54 h, after t introducing the gene editing machinery and/or transducing the viral vector to the cell is started. In one embodiment, the contacting is carried out for about 12, 18, 24, 30, 36, 42, 48, 54, 60, 66, 72, 78, 84, 90, 96, 102 h, preferably about 48 h or about 96 h, after the introducing and/or transducing step is started.

In one embodiment, the cells are contacted with the MAPK inhibitor about 12-96 h, such as 12-72 h, 12-60 h, 24-60 h, 36-60 h or 42-54 h, preferably about 72-96 h, after beginning culture of the population of haematopoietic cells, haematopoietic stem cells, haematopoietic progenitor cells and/or T cells (e.g. beginning culture after the cells are thawed from a frozen state). In one embodiment, the cells are contacted with the MAPK inhibitor about 12, 18, 24, 30, 36, 42, 48, 54, 60, 66, 72, 78, 84, 90, 96, 102 h, preferably about 72 h, after beginning culture of the population of haematopoietic cells, haematopoietic stem cells, haematopoietic progenitor cells and/or T cells.

In one embodiment, the cells are contacted with the MAPK inhibitor about 12-96 h, such as 12-60 h, 24-60 h, 36-60 h or 42-54 h, preferably about 72-96 h, after thawing the population of haematopoietic cells, haematopoietic stem cells, haematopoietic progenitor cells and or T cells (e.g. which had been stored in a frozen state). In one embodiment, the cells are contacted with the MAPK inhibitor about 12, 18, 24, 30, 36, 42, 48, 54, 60, 66, 72, 78, 84, 90, 96 or 102 h, preferably about 72 h, after thawing the population of haematopoietic cells, haematopoietic stem cells, haematopoietic progenitor cells and/or T cells.

### IL-1 Inhibitor

In some embodiments, the IL-1 inhibitor is an anti-IL-1α antibody, an anti-IL-1β antibody, an IL-1 antagonist, an IL-1 receptor antagonist, an IL-1α converting enzyme inhibitor, an IL-1β converting enzyme inhibitor, or a soluble decoy IL-1 receptor.

In some embodiments, the IL-1 inhibitor is IL-1Ra, anakinra, canakinumab, rilonacept, gevokizumab or a variant thereof; or LY2189102, MABpI, MEDI-8968, CYT013, sIL-1 RI, sIL-1 RII, EBI-005, CMPX-1023, VX-76, or a variant or derivative thereof.

In some embodiments, the IL-1 inhibitor is IL-1Ra, anakinra, canakinumab, rilonacept, gevokizumab or a variant thereof.

In some embodiments, the IL-1 inhibitor is anakinra, canakinumab, rilonacept, gevokizumab or a variant thereof.

In one embodiment, the IL-1 inhibitor is canakinumab or a variant thereof.

In one embodiment, the IL-1 inhibitor is rilonacept or a variant thereof.

In one embodiment, the IL-1 inhibitor is gevokizumab or a variant thereof.

In a preferred embodiment, the IL-1 inhibitor is anakinra or a variant thereof.

The IL-1 inhibitor may be an antagonist of IL-1α or IL-1β. An agonist is a chemical that binds to a receptor and activates the receptor to produce a biological response, for example Interleukin-1. In contrast, an antagonist blocks the action of the agonist.

The Interleukin-1 (IL-1) family is a group of 11 cytokines, which induce a complex network of proinflammatory cytokines and (via expression of integrins on leukocytes and endothelial cells) regulates and initiates inflammatory responses. IL-1α and IL-1β are the most studied members. IL-1α and IL-1β include a beta trefoil fold and bind IL-1 receptor (IL-1R). IL-1α and IL-1β binding to the IL-1 receptor (IL-1R) promotes the recruitment of the IL-1 receptor accessory protein (IL-1 RAcP) and further signalling via MyD88 adaptor.

IL-1α and IL-1β have a natural antagonist: IL-1 receptor antagonist (IL-1Ra). IL-1Ra also includes a beta trefoil fold and binds to IL-1R. IL1Ra binding to IL-1R prevents the recruitment of IL-1RAcP. Thus, IL-1Ra regulates (and inhibits) IL-1α and IL-1β pro-inflammatory activity by competing with them for binding sites of the receptor. IL-1Ra functions as a competitive inhibitor of the IL-1 receptor in vivo and in vitro. It counteracts the effects of both IL-1α and IL-1β. Upon binding of IL-1Ra, the IL-1 receptor does not transmit a signal to the cell.

In one embodiment, the IL-1 inhibitor is Interleukin-1 receptor antagonist (IL-1Ra).

A recombinant and slightly modified form of the Human interleukin-1 receptor antagonist (IL-1Ra) known as Anakinra (C₇₅₉H₁₁₈₆N₂₀₈O₂₃₂S₁₀) is commercially available as the product Kineret® and marketed by Swedish Orphan Biovitrum. Anakinra may be produced in Escherichia coli cells by recombinant DNA technology. Anakinra differs from the sequence of the human IL-1 Ra by the addition of one methionine at its N-terminus; it may also differ from the human protein in that it is not glycosylated, for example, when it is manufactured in E. coli. Anakinra is a biopharmaceutical medication used to treat rheumatoid arthritis, cryopyrin-associated periodic syndromes, familial Mediterranean fever, and Still's disease. Anakinra is administered by subcutaneous injection.

An illustrative sequence of Anakinra is shown in SEQ ID NO: 3:

(SEQ ID NO: 3)
MRPSGRKSSKMQAFRIWDVNQKTFYLRNNQLVAGYLQGPNVNLEEKIDV
VPIEPHALFLGIHGGKMCLSCVKSGDETRLQLEAVNITDLSENRKQDKR
FAFIRSDSGPTTSFESAACPGWFLCTAMEADQPVSLTNMPDEGVMVTKF
YFQEDE

In one embodiment, the IL-1 inhibitor is Anakinra or an amino acid sequence having at least 80% (suitably, at least 85%, at least 90%, at least 95%, or at least 99%) sequence identity to the amino acid sequence according to SEQ ID NO: 3, optionally wherein the inhibitor consists of the amino acid sequence according to SEQ ID NO: 3.

Canakinumab (C₆₄₅₂H₉₉₅₈N₁₇₂₂O₂₀₁₀S₄₂) binds to human IL-1β and neutralizes its inflammatory activity by blocking its interaction with IL-1 receptors, but it has no cross-reactivity with other members of the IL-1 family, including IL-1α or IL-1Ra. Canakinumab is commercially available under the brand name Ilaris® from Novartis. Canakinumab, also known as ACZ885, is a recombinant, human monoclonal antibody targeted at human IL-1β. Canakinumab belongs to the IgG1/κ isotype subclass. Canakinumab is a medication for the treatment of systemic juvenile idiopathic arthritis (SJIA) and active Still's disease, including adult-onset Still's disease (AOSD). Canakinumab comprises two heavy chains and two light chains. Both heavy chains of canakinumab contain oligosaccharide chains linked to the protein backbone at asparagine 298 (Asn 298).

An illustrative sequence of the heavy chain of canakinumab is shown in SEQ ID NO: 4:

(SEQ ID NO: 4)
QVQLVESGGGVVQPGRSLRLSCAASGFTFSVYGMNWVRQAPGKGLEWVA
IIWYDGDNQYYADSVKGRFTISRDNSKNTLYLQMNGLRAEDTAVYYCAR
DLRTGPFDYWGQGTLVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKRVEPKSCDKTHTCPPCPAPELLGGPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKS
LSLSPGK

An illustrative sequence of the light chain of canakinumab is shown in SEQ ID NO: 5:

(SEQ ID NO: 5)
EIVLTQSPDFQSVTPKEKVTITCRASQSIGSSLHWYQQKPDQSPKLLIK
YASQSFSGVPSRFSGSGSGTDFTLTINSLEAEDAAAYYCHQSSSLPFTF
GPGTKVDIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

In one embodiment, the IL-1 inhibitor is canakinumab or an antibody comprising two heavy and two light chains, wherein each heavy chain has at least 80% (suitably, at least 85%, at least 90%, at least 95%, or at least 99%) sequence identity to the amino acid sequence according to SEQ ID NO: 4, optionally consisting of SEQ ID NO: 4; and each light chain has at least 80% (suitably, at least 8, at least 90%, at least 95%, or at least 99%) sequence identity to the amino acid sequence according to SEQ ID NO: 5, optionally consisting of SEQ ID NO: 5.

Gevokizumab (C₆₄₄₂H₉₉₆₂N₁₇₁₀O₂₀₁₀S₅₂) is a monoclonal antibody which binds to IL-1β, a pro-inflammatory cytokine, and downmodulates the cellular signaling events that produce inflammation. IL-1β has been implicated in cardiovascular conditions, lung cancer, and auto-inflammatory diseases. Gevokizumab is an experimental monoclonal antibody, developed by XOMA Corporation, with allosteric modulating properties.

Rilonacept (C₉₀₃₀H₁₃₉₃₂N₂₄₀₀₀₂₆₇₀S₇₄) is a dimeric fusion protein consisting of the ligand-binding domains of the extracellular portions of the human interleukin-1 receptor component (IL-1R1) and IL-1 receptor accessory protein (IL-1RAcP) linked in-line to the fragment-crystallizable portion (Fc region) of human IgG1 that binds and neutralizes IL-1. Rilonacept is available commercially as ARCALYST® from Regeneron. Rilonacept is a soluble decoy receptor.

An illustrative sequence of rilonacept is shown in SEQ ID NO: 6:

(SEQ ID NO: 6)
SERCDDWGLDTMRQIQVFEDEPARIKCPLFEHFLKFNYSTAHSAGLTLI
WYWTRQDRDLEEPINFRLPENRISKEKDVLWERPTLINDTGNYTCMLRN
TTYCSKVAFPLEVVQKDSCFNSPMKLPVHKLYIEYGIQRITCPNVDGYF
PSSVKPTITWYMGCYKIQNFNNVIPEGMNLSFLIALISNNGNYTCVVTY
PENGRTFHLTRTLTVKVVGSPKNAVPPVIHSPNDHVVYEKEPGEELLIP
CTVYFSFLMDSRNEVWWTIDGKKPDDITIDVTINESISHSRTEDETRTQ
ILSIKKVTSEDLKRSYVCHARSAKGEVAKAAKVKQKVPAPRYTVEKCKE
REEKIILVSSANEIDVRPCPLNPNEHKGTITWYKDDSKTPVSTEQASRI
HQHKEKLWFVPAKVEDSGHYYCVVRNSSYCLRIKISAKFVENEPNLCYN
AQAIFKQKLPVAGDGGLVCPYMEFFKNENNELPKLQWYKDCKPLLLDNI
HFSGVKDRLIVMNVAEKHRGNYTCHASYTYLGKQYPITRVIEFITLEEN
KPTRPVIVSPANETMEVDLGSQIQLICNVTGQLSDIAYWKWNGSVIDED
DPVLGEDYYSVENPANKRRSTLITVLNISEIESRFYKHPFTCFAKNTHG
IDAAYIQLIYPVTNSGDKTHTCPPCPAPELLGGPSVFLFPPKPKDTLMI
SRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGK.

In one embodiment, the IL-1 inhibitor is rilonacept or an amino acid sequence having at least 80% (suitably, at least 85%, at least 90%, at least 95%, at least 99%) sequence identity to the amino acid sequence according to SEQ ID NO: 6, optionally wherein the inhibitor consists of the amino acid sequence according to SEQ ID NO: 6.

In one embodiment, the IL-1 inhibitor (e.g. anakinra) is added to the haematopoietic cells, haematopoietic stem cells, haematopoietic progenitor cells and/or T cells (e.g. in an in vitro or ex vivo culture) at a concentration of about 0.1-200, 0.1-150, 0.1-100, 0.1-75, 0.1-60, 0.1-50, 0.1-25, 0.1-20, 0.1-15 or 0.1-10 ng/μl, preferably about 0.1-60 ng/μl. In another embodiment, the IL-1 inhibitor (e.g. anakinra) is added to the haematopoietic cells, haematopoietic stem cells, haematopoietic progenitor cells and/or T cells at a concentration of about 5-200, 5-150, 5-100, 5-75, 5-60, 5-50, 5-25, 5-20, 5-15 or 5-10 ng/μl, preferably about 5-60 ng/μl.

In one embodiment, the IL-1 inhibitor (e.g. anakinra) is added to the haematopoietic cells, haematopoietic stem cells, haematopoietic progenitor cells and/or T cells (e.g. in an in vitro or ex vivo culture) at a concentration of about 0.1, 1, 2, 3, 4, 5, 6, 7, 8, 9, 10, 11, 12, 13, 14, 15, 20, 25, 30, 35, 40, 45, 50, 55, 60, 65, 70, 75, 80, 90 or 100 ng/μl, preferably about 50 ng/μl.

### NF-κB Inhibitor

Nuclear factor kappa-light-chain-enhancer of activated B cells (NF-κB) is a protein complex that controls transcription of DNA, cytokine production and cell survival. NF-κB is found in almost all animal cell types and is involved in cellular responses to a wide range of stimuli such as stress, cytokines, free radicals, TNFα, IL-1β and bacterial or viral antigens.

In unstimulated cells, NF-κB dimers are sequestered in an inactive state in the cytoplasm by a family of inhibitors, called Inhibitor of κB (IκB), which contain multiple copies of ankyrin repeats. IκB proteins mask the nuclear localization signals (NLS) of NF-κB proteins via the ankyrin repeat domains. The IκB family consists of IκBa, IκBP, IκBε, and Bcl-3.

Activation of the NF-κB is initiated by the signal-induced degradation of IκB proteins. This occurs primarily via activation of a kinase called the IκB kinase (IKK). IKK comprises a heterodimer of the catalytic IKKα and IKKβ subunits and a regulatory protein termed NF-κB essential modulator (NEMO) or IKKγ. When activated by signals (usually extracellular signals), IKK phosphorylates two serine residues located in an IκB regulatory domain. The phosphorylated IκB proteins are ubiquitinated, leading to their degradation by the proteasome. With the degradation of IκB, the NF-κB complex is released and able to translocate to the nucleus and act as a transcription factor for many NF-κB target genes.

An NF-κB inhibitor may act at any stage of NF-κB activation and/or at any point within the NF-κB signalling pathway. Suitably, the NF-κB inhibitor is an IL-1 inhibitor.

In one embodiment, the NF-κB inhibitor is not an IL-1 inhibitor.

In some embodiments, the NF-κB inhibitor is an IL-1 inhibitor, an IL-1 receptor inhibitor, a TLR4 inhibitor, a TAK1 inhibitor, an Akt inhibitor, an IKK inhibitor, an inhibitor of IκB phosphorylation, an inhibitor of IκB degradation, an inhibitor of the proteasome, an inhibitor of IκBα upregulation, an inhibitor of NF-κB nuclear translocation, an inhibitor of NF-κB expression, an inhibitor of NF-κB DNA binding, or an inhibitor of NF-κB transactivation.

The activity of a kinase may be analysed directly, for example by analysing the enzymatic activity of the kinase in vitro as described herein. The ability of a candidate agent to inhibit (e.g. reduce the activity) of a kinase may be expressed in terms of an IC50 value, which is the concentration of an agent that is required to give rise to a 50% reduction in the activity of the kinase, as described herein.

In some embodiments, the NF-κB inhibitor is SC514 or a derivative thereof; IL-1Ra, anakinra, canakinumab, rilonacept, gevokizumab or a variant thereof; or LY2189102, MABpI, MEDI-8968, CYT013, sIL-1 RI, sIL-1 RII, EBI-005, CMPX-1023, VX-76 or a derivative thereof; or metformin, apigenin, kaempferol, BAY 11-7082, simvastatin or a derivative thereof; or an siRNA, shRNA, miRNA or antisense DNA/RNA.

In some embodiments, the NF-κB inhibitor is SC514 or a derivative thereof; anakinra, canakinumab, rilonacept, gevokizumab or a variant thereof; or metformin, apigenin, kaempferol, BAY 11-7082, or a derivative thereof.

In some embodiments, the NF-κB inhibitor is SC514 or derivatives thereof; IL-1Ra, anakinra, canakinumab, rilonacept, gevokizumab or variants thereof; or an siRNA, shRNA, miRNA or antisense DNA/RNA.

In some embodiments, the NF-κB inhibitor is an IKK inhibitor. In a preferred embodiment, the NF-κB inhibitor is SC514 or a derivative thereof.

SC514 (C₉H8N₂OS₂) (CAS No. 354812-17-2) is a selective and reversible inhibitor of IKKβ (IKK-2) (IC₅₀=3-12 μM) that displays >10-fold selectivity over 28 other kinases, including JNK, p38, MK2, and ERK. This compound attenuates NF-κB-induced gene expression of IL-6, IL-8 and Cox-2 (IC₅₀=20, 20, and 8 μM, respectively). Improper activation of NF-κB has been linked to cancer, immune and inflammatory diseases, and viral infection.

In some embodiments, SC514 has the following structure:

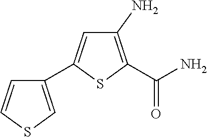 [SMILES: NC(=O)c1sc(-c2ccsc2)cc1N]

Metformin (C₄H₁₁N₅) is an IKK and/or NF-κB inhibitor. Metformin is approved for treatment of type 2 diabetes.

Apigenin (C₁₅H₁₀O₅) is an inhibitor of the NF-κB p65 subunit and IκB. Apigenin is a naturally occurring flavonoid.

Kaempferol (C₁₅H₁₀O₆) is an inhibitor of NF-κB p65 subunit and IκB. Kaempferol is a naturally occurring flavonoid.

BAY 11-7082 (C₁₀H₉NO₂S) is an inhibitor of NF-κB p65 subunit and IκB. BAY 11-7082 has been used in preclinical models of senescence in vitro. Bay 11-7082 acts as a selective inhibitor for the nod-like receptor family pyrin domain containing 3 (NLRP3) inflammasome pathway. In addition to the inhibition of nuclear factor-kappa B (NF-kB), BAY 11-7082 also triggers apoptosis in anucleated erythrocytes, human T-cell leukemia virus type I (HTLV-I)-infected T-cell lines and primary adult T-cell leukemia cells. Bay 11-7082 is an inhibitor of cytokine-induced IκB-α phosphorylation.

In one embodiment, the NF-κB inhibitor (e.g. SC514 or derivatives thereof) is added to the haematopoietic cells, haematopoietic stem cells, haematopoietic progenitor cells and/or T cells (e.g. in an in vitro or ex vivo culture) at a concentration of about 1-200, 1-150, 1-100, 1-50, 1-40, 1-30, 1-20 or 1-15 μM, preferably about 1-30 μM. In another embodiment, the NF-κB inhibitor (e.g. SC514 or derivatives thereof) is added to the haematopoietic cells, haematopoietic stem cells, haematopoietic progenitor cells and/or T cells at a concentration of about 5-200, 5-150, 5-100, 5-50, 5-40, 5-30, 5-20 or 5-15 μM, preferably about 5-30 μM.

In one embodiment, the NF-κB inhibitor (e.g. SC514 or derivatives thereof) is added to the haematopoietic cells, haematopoietic stem cells, haematopoietic progenitor cells and/or T cells (e.g. in an in vitro or ex vivo culture) at a concentration of about 1, 2, 3, 4, 5, 6, 7, 8, 9, 10, 11, 12, 13, 14, 15, 20, 25, 30, 35, 40, 45 or 50 μM, preferably 25 μM.

An Agent which Promotes Homology Directed DNA Repair

The present inventors have previously determined that improving the efficiency of HDR in HSC increases cell survival and engraftment of gene edited HSCs (see WO 2020002380, incorporated by reference herein in its entirety). Suitable agents which promote HDR for use according to the present invention are described in WO 2020002380 and are incorporated herein by reference.

Gene editing in primary cells and the HSPC in particular may be hampered by gene transfer efficiency and limited HDR, likely due to low levels of expression of the HDR machinery and high activity of NHEJ pathway.

As used herein “an agent which promotes homology directed DNA repair” refers to an agent which enhances and/or improves the efficiency of HDR relative to the level of HDR in a cell which has not been treated with the agent.

Suitably, HDR may be increased by 5%, 10%, 20%, 30%, 40%, 50%, 60%, 70%, 80%, 90% or more. Suitably, HDR may be increased by 1.1, 1.2, 1.3, 1.4, 1.5, 1.6, 1.7, 1.8, 1.9, 2.0, 2.5, 3.0 fold or more.

HDR efficiency may be measured using any method in the art. Suitably, HDR efficiency may be determined by FACS measuring the presence of a marker in donor template vector which is introduced to the cell by HDR-mediated integration at the targeted locus. For example, an AAV6 donor template may comprise a PGK.GFP reporter cassette. An increase in HDR efficiency may be obtained via transient p53 inhibition, as measured by determining the percentage of GFP+ cells.

Targeting integration (“on target” HDR) can be determined by digital PCR by using primers and probes which were designed on the junction between the vector sequence and the targeted locus and on control sequences used for normalization (human TTC5 genes).

The percentage of insertions and deletion (indels) introduced by the Non Homologous End Joining (NHEJ) repair pathway on the nucleases target site may be measured by a mismatch-sensitive endonuclease assay (PCR-based amplification of the targeted locus followed by digestion with T7 Endonuclease I; digested DNA fragments were resolved and quantified by capillary electrophoresis on LabChip GX Touch HT, Perkin Elmer). The levels of NHEJ-induced mutations are used as surrogate readout for scoring nucleases activity.

In some embodiments, the one or more inhibitor(s) of senescence is used in combination with an agent which promotes homology directed DNA repair (preferably, an inhibitor of p53 activation). Preferably, the inhibitor of p53 activation is GSE56 or a variant thereof.

In some embodiments, the MAPK inhibitor, IL-1 inhibitor and/or NF-κB inhibitor is used in combination with an agent which promotes homology directed DNA repair (preferably, an inhibitor of p53 activation). Preferably, the inhibitor of p53 activation is GSE56 or a variant thereof.

In some embodiments, the MAPK inhibitor is used in combination with an agent which promotes homology directed DNA repair (preferably, an inhibitor of p53 activation). Preferably, the inhibitor of p53 activation is GSE56 or a variant thereof.

In some embodiments, the IL-1 inhibitor is used in combination with an agent which promotes homology directed DNA repair (preferably, an inhibitor of p53 activation). Preferably, the inhibitor of p53 activation is GSE56 or a variant thereof.

In some preferred embodiments, the NF-κB inhibitor is used in combination with an agent which promotes homology directed DNA repair (preferably, an inhibitor of p53 activation). Preferably, the inhibitor of p53 activation is GSE56 or a variant thereof.

Preferably, the agent has low cellular toxicity.

Preferably, the agent does not significantly change the composition of the gene edited cells.

Preferably, the agent does not significantly induce differentiation of the gene edited haematopoietic stem and/or progenitor cells. Therefore, the gene edited haematopoietic stem and/or progenitor cells and/or T cells according to the present invention retain their long-term re-population capacity.

Suitably, the change in composition or differentiation of the gene edited cells is less than 5%, less than 4%, less than 3% less than 2% or less than 1% when compared with an untreated control.

### Inhibitors of p53 Activation

In a preferred embodiment, the agent which promotes HDR is an inhibitor of p53 activation.

Accordingly, in one aspect, the invention provides the use of (i) one or more inhibitor(s) of senescence and (ii) an inhibitor of p53 activation for increasing the survival and/or engraftment of haematopoietic cells, haematopoietic stem cells, haematopoietic progenitor cells and/or T cells in gene therapy.

In a further aspect, the invention provides the use of (i) one or more inhibitor(s) of senescence and (ii) an inhibitor of p53 activation for increasing the efficiency of gene editing of haematopoietic cells, haematopoietic stem cells, haematopoietic progenitor cells, and/or T cells.

In a further aspect, the invention provides a combination comprising (i) one or more inhibitor(s) of senescence and (ii) an inhibitor of p53 activation for use in haematopoietic cell gene therapy, haematopoietic stem cell gene therapy, haematopoietic progenitor cell gene therapy and/or T cell gene therapy.

In a further aspect, the invention provides a combination comprising (i) one or more inhibitor(s) of senescence and (ii) an inhibitor of p53 activation for use in increasing the survival and/or engraftment of haematopoietic cells, haematopoietic stem cells, haematopoietic progenitor cells and/or T cells in gene therapy.

In some embodiments, the inhibitor of p53 activation is an inhibitor of p53 phosphorylation, more preferably an inhibitor of p53 Serine 15 phosphorylation.

In some embodiments, the inhibitor of p53 activation is a p53 dominant negative peptide, an ataxia telangiectasia mutated (ATM) kinase inhibitor or an ataxia telangiectasia and Rad3-related protein (ATR) inhibitor.

In some embodiments, the inhibitor of p53 activation is pifithrin-α or a derivative thereof; KU-55933 or a derivative thereof; GSE56 or a variant thereof; KU-60019, BEZ235, wortmannin, CP-466722, Torin 2, CGK 733, KU-559403, AZD6738 or derivatives thereof; or an siRNA, shRNA, miRNA or antisense DNA/RNA.

### Dominant Negative Peptides

In a preferred embodiment, the inhibitor of p53 activation is a mutant p53 peptide.

In a preferred embodiment, the inhibitor of p53 activation is a dominant negative peptide (e.g. a dominant negative p53 peptide).

Suitably, a dominant negative peptide may comprise mutations in the homo-oligomerisation domain. Suitably, dominant negative peptides comprising mutations in the homo-oligomerisation domain may dimerise with wild-type p53 and prevent wild-type p53 from activating transcription.

In a preferred embodiment, the dominant negative peptide is GSE56 or a variant thereof.

In one embodiment, the nucleotide sequence for mRNA translation of GSE56 is set forth in SEQ ID No. 8.

(SEQ ID NO. 8)
ATGGATGGATGGTGTCCTGGGAGAGACCGTCGGACAGAGGAAGAAAATT
TCCGCAAAAAAGAAGAGCATTGCCCGGAGCTGCCCCCAGGGAGTGCAAA
GAGAGCACTGCCCACCAGCACAAGCTCCTCTCCCCAGCAAAAGAAAAAA
CCACTCGATGGAGAATATTTCACCCTTAAGATCCGTGGGCGTGAGCGCT
TCGAGATGTTCCGAGAGCTGAATGAGGCCTTGGAATTAAAGGATGCCCG
TGCTGCCGAGGAGTCAGGAGACAGCAGGGCTCACTCCAGCTACCCGAAG
ATAGTTAGTTAG

In one embodiment, the amino acid sequence of GSE56 is set forth in SEQ ID No. 9.

(SEQ ID NO. 9)
MDGWCPGRDRRTEEENFRKKEEHCPELPPGSAKRALPTSTSSSPQQKKK
PLDGEYFTLKIRGRERFEMFRELNEALELKDARAAEESGDSRAHSSYPK
IVS

Suitably, the inhibitor of 53 activation may be a nucleotide sequence which encodes GSE56.

Suitably, the inhibitor of p53 activation may be an amino acid sequence encoding GSE56.

Suitably, the inhibitor of p53 activation may be GSE56 mRNA.

### Kinase Inhibitors

In some embodiments, the agent which promotes HDR may be an ataxia telangiectasia mutated (ATM) kinase inhibitor or an ataxia telangiectasia and Rad3-related protein (ATR) inhibitor.

The activity of ATM kinase and ATR may be analysed directly, for example by analysing the enzymatic activity of the ATM kinase or ATR in vitro.

The ability of a candidate agent to inhibit (e.g. reduce the activity) ATM kinase or ATR may be expressed in terms of an IC50 value, which is the concentration of an agent that is required to give rise to a 50% reduction in the activity of the kinase. Preferably, the inhibitors of the invention have an IC50 value for inhibition (e.g. of ATM kinase or ATR) of less than 100 μM, more preferably less than 10 μM, for example less than 1 μM, less than 100 nM or less than 10 nM (e.g. KU-55933 has an IC50 value of about 13 nM for ATM kinase).

A number of techniques are known in the art for measuring kinase activity. Preferably, the kinase activity assays are carried out on a kinase (e.g. ATM kinase or ATR) that has been isolated from a cell. The kinase may have been expressed using recombinant techniques, and preferably has been purified. For example, kinase activity may be determined by monitoring the incorporation of radiolabelled phosphate from [γ-³²P]-labelled ATP into a substrate. Such assay techniques are described in, for example, Hastie et al. (Hastie, C. J. et al. (2006) Nat. Protocols 1: 968-971).

Preferably, the inhibitors are of low toxicity for mammals, such as humans, and in particular are of low toxicity for haematopoietic stem and/or progenitor cells.

A candidate inhibitor may be further analysed for its ability to increase cell survival and/or engraftment using a method as disclosed herein.

Preferably, the inhibitor is a transient inhibitor (e.g. has an inhibitory action lasting less than about 1, 2, 3, 4, 5, 6, 7 or 14 days).

Preferably, the inhibitor is a pharmacological inhibitor.

In a preferred embodiment, the inhibitor is KU-55933 or a derivative thereof.

KU-55933 (CAS No. 587871-26-9) is a selective, competitive ATM kinase inhibitor having the following structure:

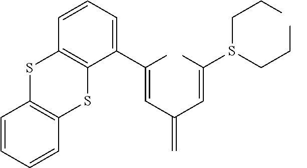

Solutions of KU-55933 for use in the invention may be prepared using routine methods known in the art, for example KU-55933 is known to be soluble in DMSO and ethanol.

The concentration at which KU-55933 or a derivative thereof is applied to a population of haematopoietic stem and/or progenitor cells may be adjusted for different vector systems to optimise cell survival (e.g. during in vitro or ex vivo culture) and/or engraftment.

The invention encompasses the use of KU-55933 and derivatives of KU-55933. The KU-55933 derivatives for use according to the invention are those which increase the survival (e.g. during in vitro or ex vivo culture) and/or engraftment of haematopoietic cells, haematopoietic stem cells and/or haematopoietic progenitor cells, in particular cells transduced by a viral vector.

KU-55933 derivatives for use according to the invention may have been developed, for example, for increased solubility, increased stability and/or reduced toxicity.

KU-55933 derivatives for use according to the invention are preferably of low toxicity for mammals, in particular humans. Preferably, KU-55933 derivatives for use according to the invention are of low toxicity for haematopoietic cells, haematopoietic stem cells and/or haematopoietic progenitor cells and/or T cells.

Suitable KU-55933 derivatives may be identified using methods known in the art for determining cell survival in culture and/or engraftment. Examples of such methods have been described above. The method employed is preferably one which is amenable to automation and/or high throughput screening of candidate KU-55933 derivatives. The candidate KU-55933 derivatives may form part of a library of KU-55933 derivatives.

Further kinase inhibitors that may be used in the invention include:

KU-60019, which is an improved analogue of KU-55933 and has an IC50 of 6.3 nM for ATM kinase in cell-free assays. KU-60019 has the structure:

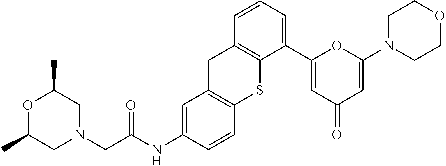 [SMILES: C[C@@H]1CN(CC(=O)Nc2ccc3c(c2)Cc2cccc(-c4cc(=O)cc(N5CCOCC5)o4)c2S3)C[C@H](C)O1]

BEZ235 (NVP-BEZ235, Dactolisib), which is a dual ATP-competitive PI3K and mTOR inhibitor for p110α/γ/δ/β and mTOR(p70S6K) and inhibits ATR with an IC50 of about 21 nM in 3T3TopBP1-ER cells. BEZ235 has the structure:

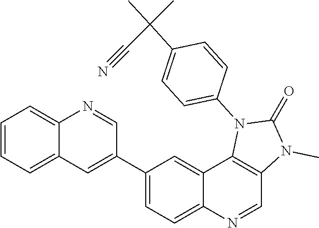 [SMILES: Cn1c(=O)n(-c2ccc(C(C)(C)C#N)cc2)c2c3cc(-c4cnc5ccccc5c4)ccc3ncc21]

Wortmannin, which has the structure:

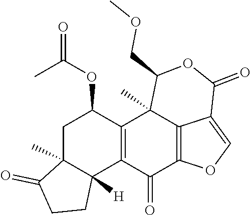 [SMILES: COC[C@H]1OC(=O)c2coc3c2[C@@]1(C)C1=C(C3=O)[C@@H]2CCC(=O)[C@@]2(C)C[C@H]1OC(C)=O]

CP-466722, which is a potent and reversible ATM kinase inhibitor, but does not affect ATR. CP-466722 has the structure:

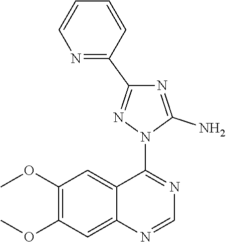 [SMILES: COc1cc2ncnc(-n3nc(-c4ccccn4)nc3N)c2cc1OC]

Torin 2, which ATM kinase and ATR with EC50 values of 28 nM and 35 nM, respectively. Torin 2 has the structure:

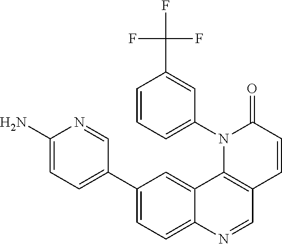 [SMILES: Nc1ccc(-c2ccc3ncc4ccc(=O)n(-c5cccc(C(F)(F)F)c5)c4c3c2)cn1]

CGK 733 (CAS No. 905973-89-9), which is a potent and selective inhibitor of ATM kinase and ATR with IC50 values of about 200 nM. CGK 733 has the structure:

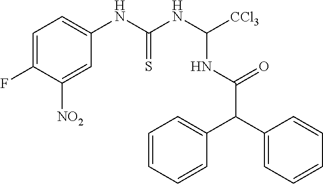

KU-559403 (Weber et al. (2015) Pharmacology & Therapeutics 149: 124-138). KU-559403 has the structure:

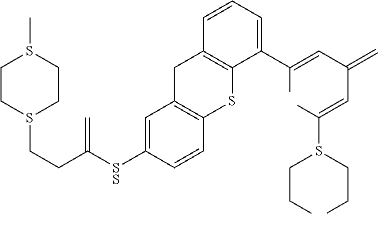

AZD6738, which has the structure:

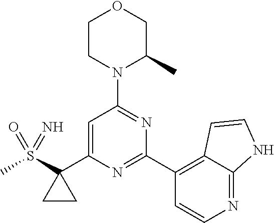 [SMILES: C[C@@H]1COCCN1c1cc(C2([S@](C)(=N)=O)CC2)nc(-c2ccnc3[nH]ccc23)n1]

Derivatives of these inhibitors, possessing characteristics as described for the KU-55933 derivatives, may also be used in the invention, and may be identified using analogous methods to those described for the KU-55933 derivatives.

In a preferred embodiment, the p53 inhibitor may be pifithrin-α, pifithrin-α cyclic and pifithrin-α p-nitro or a derivative thereof. Pifithrin-α has the structure:

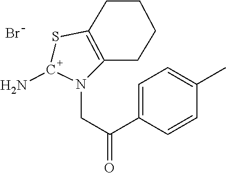 [SMILES: Br ; Cc1ccc(C(=O)CN2C3=C(CCCC3)SC2N)cc1]

siRNAs, shRNAs, miRNAs and Antisense DNAs/RNAs

Inhibition (e.g. of the kinase) may be achieved using post-transcriptional gene silencing (PTGS). Post-transcriptional gene silencing mediated by double-stranded RNA (dsRNA) is a conserved cellular defence mechanism for controlling the expression of foreign genes. It is thought that the random integration of elements such as transposons or viruses causes the expression of dsRNA which activates sequence-specific degradation of homologous single-stranded mRNA or viral genomic RNA. The silencing effect is known as RNA interference (RNAi) (Ralph et al. (2005) Nat. Medicine 11: 429-433). The mechanism of RNAi involves the processing of long dsRNAs into duplexes of about 21-25 nucleotide (nt) RNAs. These products are called small interfering or silencing RNAs (siRNAs) which are the sequence-specific mediators of mRNA degradation. In differentiated mammalian cells, dsRNA >30 bp has been found to activate the interferon response leading to shut-down of protein synthesis and non-specific mRNA degradation (Stark et al. (1998) Ann. Rev. Biochem. 67: 227-64). However, this response can be bypassed by using 21 nt siRNA duplexes (Elbashir et al. (2001) EMBO J. 20: 6877-88; Hutvagner et al. (2001) Science 293: 834-8) allowing gene function to be analysed in cultured mammalian cells.

shRNAs consist of short inverted RNA repeats separated by a small loop sequence. These are rapidly processed by the cellular machinery into 19-22 nt siRNAs, thereby suppressing the target gene expression.

Micro-RNAs (miRNAs) are small (22-25 nucleotides in length) noncoding RNAs that can effectively reduce the translation of target mRNAs by binding to their 3′ untranslated region (UTR). Micro-RNAs are a very large group of small RNAs produced naturally in organisms, at least some of which regulate the expression of target genes. Founding members of the micro-RNA family are let-7 and lin-4. The let-7 gene encodes a small, highly conserved RNA species that regulates the expression of endogenous protein-coding genes during worm development. The active RNA species is transcribed initially as an ˜70 nt precursor, which is post-transcriptionally processed into a mature ˜21 nt form. Both let-7 and lin-4 are transcribed as hairpin RNA precursors which are processed to their mature forms by Dicer enzyme.

The antisense concept is to selectively bind short, possibly modified, DNA or RNA molecules to messenger RNA in cells and prevent the synthesis of the encoded protein.

Methods for the design of siRNAs, shRNAs, miRNAs and antisense DNAs/RNAs to modulate the expression of a target protein, and methods for the delivery of these agents to a cell of interest are well known in the art.

### Adenoviral Proteins

Adenoviruses are natural co-helpers of AAV infection and provide a set of genes: E1a, E1b, E2a and E4 which optimize AAV infection.

It has previously been demonstrated that delivery of adenoviral proteins during gene editing improves the efficiency of HDR in HSC (and enhances the long-term repopulating activity of HSC). Without wishing to be bound by theory, the adenoviral proteins may provide helper functions to the AAV infection during gene editing.

In some embodiments, the one or more inhibitor(s) of senescence (e.g. a MAPK inhibitor, an IL-1 inhibitor and/or an NF-κB inhibitor) is used in combination with at least one adenoviral protein.

In one embodiment, the one or more inhibitor(s) of senescence (e.g. a MAPK inhibitor, an IL-1 inhibitor and/or an NF-κB inhibitor) comprises at least one adenoviral protein.

In one embodiment, the agent which promotes homology directed DNA repair comprises at least one adenoviral protein.

In one embodiment, one or more inhibitor(s) of senescence (e.g. a MAPK inhibitor, an IL-1 inhibitor and/or an NF-κB inhibitor) comprises a nucleic acid sequence encoding at least one adenoviral protein.

In one embodiment, the agent which promotes homology directed DNA repair comprises a nucleic acid sequence encoding at least one adenoviral protein.

The adenoviral protein is not limited to a particular Adenovirus serotype. For example, in one embodiment, the at least one adenoviral proteins is from an Adenovirus of serotype 4, Adenovirus of serotype 5, Adenovirus of serotype 7 and/or Adenovirus of serotype 9.

In one embodiment, the at least one adenoviral protein is selected from the group comprising E1a, E1b, E2a and E4.

In a preferred embodiment, the at least one adenoviral protein is an open reading frame of the E4 gene.

In a preferred embodiment, the at least one adenoviral protein is E4orf1 or a variant thereof.

An example of a nucleotide sequence encoding Ad5-E4orf1 is set forth in SEQ ID No. 10. Suitably, the at least one adenoviral protein may comprise a nucleotide sequence for mRNA translation as set forth in SEQ ID No. 10 or a variant thereof.

(SEQ ID No. 10)
ATGGCCGCTGCTGTGGAAGCCCTGTACGTGGTGCTTGAAAGAGAGGGCG
CCATCCTGCCTAGACAAGAGGGCTTTTCTGGCGTGTACGTGTTCTTCAG
CCCCATCAACTTCGTGATCCCTCCAATGGGCGCCGTGATGCTGAGCCTG
AGACTGAGAGTGTGTATCCCTCCTGGCTACTTCGGCCGGTTTCTGGCCC
TGACCGATGTGAACCAGCCTGACGTGTTCACCGAGAGCTACATCATGAC
CCCTGACATGACCGAGGAACTGAGCGTGGTGCTGTTCAACCACGGCGAC
CAGTTCTTTTATGGCCACGCCGGAATGGCCGTCGTGCGGCTGATGCTGA
TCAGAGTGGTGTTTCCCGTCGTCCGGCAGGCCAGCAATGTTTGA.

An example of an amino acid sequence of Ad5-E4orf1 is set forth in SEQ ID No. 1. Suitably, the at least one adenoviral protein may comprise an amino acid sequence as set forth in SEQ ID No. 1 or a variant thereof.

(SEQ ID No. 1)
MAAAVEALYVVLEREGAILPRQEGFSGVYVFFSPINFVIPPMGAVMLSL
RLRVCIPPGYFGRELALTDVNQPDVFTESYIMTPDMTEELSVVLFNHGD
QFFYGHAGMAVVRLMLIRVVFPVVRQASNV

Other examples of an amino acid sequence of E4orf1 are set forth in SEQ ID No. 57 to SEQ ID No. 76. Suitably, the at least one adenoviral protein may comprise an amino acid sequence as set forth in SEQ ID No. 57 to SEQ ID No. 76 or a variant thereof.

| | | |
|---|---|---|
| SEQ ID | Ad1-E4orf1 | MAAAVEALYVVLEREGAILPRQEGFSGVYVFFSPINFVIPPMGAVM |
| No. 57 | | LPLRLRVCIPPGYFGRFLALTDVNQPDVFTESYIMTPDMTEELSVV |
| | | LFNHGDQFFYGHAGMAVVRIMLIRVVFPVVRQASNV |
| SEQ ID | Ad2-E4orf1 | MAAAVEALYVVLEREGAILPRQEGESGVYVFFSPINEVIPPMGAVM |
| No. 58 | | LSLRLRVCIPPGYFGRFLALTDVNQPDVFTESYIMTPDMIEELSVV |
| | | LFNHGDQFFYGHAGMAVVRIMLIRVVFPVVRQASNV |
| SEQ ID | Ad3-E4orf1 | MADEALYVYLEGPGATLPEQQQRNNYIFYSPVPFTLYPRGVALLYL |
| No. 59 | | KLSIIIPRGYVGCFFSLTDANMSGLYASSRIIHAGHREELSVLLFN |
| | | HNDRFYEGRAGDPVACLVMERLIFPPVRQATMI |
| SEQ ID | Ad4-E4orf1 | MDAQVLYVFLEGAGALLPVQKGSNYIFYAPANFVLHPHGVALLELR |
| No. 60 | | LSIVVPQGFIGRFFSLTDANVPGVYASSRIIHAGHREGLSVMLFNH |
| | | NVSFYNGRAGDPVACLVLERVIYPPVRQASMV |
| SEQ ID | Ad6-E4orf1 | MAAAVEALYVVLEREGAILPRQEGFSGVYVFFSPINFVIPPMGAVM |
| No. 61 | | LSLRLRVCIPPGYFGRFLALTDVSQPDVFTESYIMTPDMTEELSVV |
| | | LFNHGDQFFYGHAGMAVVRIMLIRVVFPVVRQASNV |
| SEQ ID | Ad7-E4orf1 | MADEALYVYLEGPGATLPEQQQRNNYIFYSPVPFTLYPRGVALLYL |
| No. 62 | | RLSIIIPRGYVGCFFSLTDANMSGLYASSRIIHAGHREELSVLLFN |
| | | HDDRFYEGRAGDPVACLVMERLIYPPVRQATMI |
| SEQ ID | Ad9-E4orf1 | MAESLYAFIDSPGGIAPVQEGTSNRYTFFCPESFHIPPHGVVLLHL |
| No. 63 | | KVSVLVPTGYQGRFMALNDYHARDILTQSDVIFAGRRQELTVLLFN |
| | | HTDRFLYVRKGHPVGTLLLERVIFPSVKIATLV |
| SEQ ID | Ad14-E4orf1 | MADEALYVYLEGPGATLPEQQQRNNYIFYSPVPFTLYPRGVALLYL |
| No. 64 | | RLSIIIPRGYIGCFLSLTDANMEGLYASSRIIHAGHREELSVLLFN |
| | | HDDRFYEGRAGDPVACLVMERLIYPPVRQATLI |
| SEQ ID | Ad16-E4orf1 | MADEALYVYFRGPGATLPEQQQQRNNYIFYSPVPFTLYPRGVALLY |
| No. 65 | | LRLSIIIPRGYVGCFFSLTDANMSGLYASSRIIHAGHREELSVLLF |
| | | NHDDRFYEGRAGDPVACLVMERLIYPPVRQATMI |
| SEQ ID | Ad18-E4orf1 | MAALQALYVYFKGPGAMLPEQEGYSNAYVLFSPANFVIPPHGVVLL |
| No. 66 | | YLHIAVDIPPGYLGTLFSLSDMNARGVFVGAETLYPGSRMELSVLL |
| | | FNHSDVFCDVRAKQPVARLLLSRVIFPPVRQASLL |
| SEQ ID | Ad20-E4orf1 | MAESLYAFIDSPGGIAPVQEGTSNRYNFFCPQSFHIPPHGVVLLHL |
| No. 67 | | KVSVLVPTGYQGREMALNDYHARDILTQSDVIFAGRRQELTVLLFN |
| | | HTDRFLYVRKGHPVGTLLLERVIFPSVKIATLV |
| SEQ ID | Ad21-E4orf1 | MAEVLYVILEGPGARLPVQEGNNYIFYAPVDFTLHPRGVALLHLRL |
| No. 68 | | SIIVPRCYIGRFFSLTDTNTSGLYASSQIIFAAHQQPLSVMLFNHT |
| | | DRFYEGRVGDPVACLVLERVIYPSVRQASMM |
| SEQ ID | Ad23-E4orf1 | MAESLYAFIDSPGGIAPVQEGSSNRYNFFCPESFHIPPHGVVLLHL |
| No. 69 | | RVSVLIPTGYQGRFMALNDYHARGILTQSDVIFAGRRHELTVLLFN |
| | | HTDRFLYVREGHPVGTLLLERVIFPSVRLATLV |
| SEQ ID | Ad25-E4orf1 | MAESLYAFIDSPGGIAPVQEGTSNRYTFFCPESFHIPPHGVVLVHL |
| No. 70 | | RVSVLIPNGYQGRFMALNDYHSRGILTQSDVIFAGRRQELTVLLFN |
| | | HTDRFLYVREGHPVGTLLLERVIFPSVRLATLV |
| SEQ ID | Ad30-E4orf1 | MAESLYAFIDSPGGIAPVQEGASNRYTFFCPESFHIPPHGVILLHL |
| No. 71 | | RVSVLVPTGYQGRFMALNDYHARGILTQSDVIFAGRRHDLSVLLFN |
| | | HTDRFLYVREGHPVGTLLLERVIFPSVRLATLV |
| SEQ ID | Ad36-E4orf1 | MAESLYAFIDSPGGIAPVQEGASNRYIFFCPESFHIPPHGVILLHL |
| No. 72 | | RVSVLVPTGYQGRFMALNDYHARGILTQSDVIFAGRRHDLSVLLFN |
| | | HTDRFLYVREGHPVGTLLLERVIFPSVRIATLV |
| SEQ ID | Ad50-E4orf1 | MADEALYVYLDGPGATLPEQQQRNNYIFYSPVPFTLYPRGVALLYL |
| No. 73 | | RLSIIIPRGYVGCFFSLTDANMSGLYASSRIIHAGHREELSVLLFN |
| | | HDDRFYEGRAGDPVACLVMERLIYPPVRQATMI |
| SEQ ID | Ad55-E4orf1 | MAHEALYVYLEGPGATLPEQQQRNNYIFYSPVPFTLYPRGVALLYL |
| No. 74 | | RLSIIIPRGYIGCFLSLTDANMFGLYASSRIIHAGHREELSVLLFN |
| | | HDDRFYEGRAGDPVACLVMERLIYPPVRQATLI |
| SEQ ID | Ad62-E4orf1 | MAESLYAFIDSPGGIAPVQEGASNRYIFFCPESFHIPPHGVILLHL |
| No. 75 | | RVSVMVPTGYQGREMALNDYHARGILTQSDVIFAGRRHDLSVLLFN |
| | | HTDRFLYVREGHPVGTLLLERVIFPSVRIATLV |
| SEQ ID | Ad71-E4orf1 | MAESLYAFIDSPGGIAPVQEGTSNRYDFFCPESFHIPPHGVVLLHL |
| No. 76 | | RVSVLIPTGYQGREMALNDYHARGILTQSDVIFAGRRHELTVLLFN |
| | | HTDRFLYVREGHPVGTLLLERVIFPSVRLATLV |

In a preferred embodiment, the at least one adenoviral protein is E4orf6/7 or a variant thereof.

An example of a nucleotide sequence encoding Ad5-E4orf6/7 is set forth in SEQ ID No. 11. Suitably, the at least one adenoviral protein may comprise a nucleotide sequence for mRNA translation as set forth in SEQ ID No. 11 or a variant thereof.

(SEQ ID NO. 11)
ATGACCACCAGCGGCGTGCCCTTCGGCATGACACTCAGACCTACCAGAA
GCCGGCTGAGCAGAAGAACCCCTTACAGCAGAGACAGGCTGCCTCCATT
CGAGACAGAGACACGGGCCACCATCCTGGAAGATCACCCTCTGCTGCCC
GAGTGTAACACCCTGACCATGCACAACGCCTGGACAAGCCCATCTCCTC
CAGTGAAACAGCCCCAAGTGGGACAGCAGCCTGTTGCTCAGCAGCTGGA
CAGCGACATGAACCTGTCTGAACTGCCCGGCGAGTTCATCAACATCACC
GACGAGAGACTGGCCCGGCAAGAGACAGTGTGGAACATCACCCCTAAGA
ACATGAGCGTGACCCACGACATGATGCTGTTCAAGGCCAGCAGAGGCGA
GCGGACAGTGTACAGCGTTTGTTGGGAAGGCGGCGGACGGCTGAATACC
AGAGTGCTGTAA

An example of an amino acid sequence of Ad5-E4orf6/7 is set forth in SEQ ID No. 2. Suitably, the at least one adenoviral protein may comprise an amino acid sequence as set forth in SEQ ID No. 2 or a variant thereof.

(SEQ ID NO. 2)
MTTSGVPFGMTLRPTRSRLSRRTPYSRDRLPPFETETRATILEDHPLLP
ECNTLTMHNAWTSPSPPVKQPQVGQQPVAQQLDSDMNLSELPGEFINIT
DERLARQETVWNITPKNMSVTHDMMLFKASRGERTVYSVCWEGGGRLNT
RVL

Other examples of an amino acid sequence of E4orf6/7 are set forth in SEQ ID NO. 7, SEQ ID No. 39 to SEQ ID NO. 56 and SEQ ID No. 77 to SEQ ID No. 88. Suitably, the at least one adenoviral protein may comprise an amino acid sequence as set forth in SEQ ID NO. 7, SEQ ID No. 39 to SEQ ID NO. 56 and SEQ ID No. 77 to SEQ ID No. 88 or a variant thereof.

| | | |
|---|---|---|
| SEQ ID | Ad1-E4orf6/7 | MTTSGVPFGMTLRPTRSRLSRRTPYSRDRLPPFETETRATI |
| No. 77 | | LEDHPLLPECNTLTMHNAWTSPSPPVEQPQVGQQPVAQQLD |
| | | SNMNLRELPGEFINITDERLARQETVWNITPKNMSVTHDMM |
| | | LFKASRGERTVYSVCWEGGGRLNTRVL* |
| SEQ ID | Ad2-E4orf6/7 | MTTSGVPFGMTLRPTRSRLSRRTPYSRDRLPPFETETRATI |
| No. 78 | | LEDHPLLPECNTLTMHNAWTSPSPPVEQPQVGQQPVAQQLD |
| | | SDMNLSELPGEFINITDERLARQETVWNITPKNMSVTHDMM |
| | | LFKASRGERTVYSVCWEGGGRLNTRVL* |
| SEQ ID | Ad3-E4orf6/7 | MSGSNSIMTRLRARSTSCARHHPYTRAQLPRCEENETRASM |
| No. 79 | | TEDHPLLPDCDTMTMHSMTVIQTPESHPQQLDCESALKDYP |
| | | DAFLCITDPRLARSETVWNVESKTMSISNGIQMFKAVRGER |
| | | LVYSVKWEGGGKITTRIL* |
| SEQ ID | Ad4-E4orf6/7 | MSGNSSIMTRSRTRLALSRHHPYQPPATLPRCEETESRASL |
| No. 80 | | VEDHPVLPDCDTLSMHNITVIPITEDSPQLLNCEVQMQECP |
| | | EGFISLTDPRLSRSETVWNVEIKTMSITNSIQMEKAVRGER |
| | | IVYSMRWEGGGKITTRIL* |
| SEQ ID | Ad6-E4orf6/7 | MTTSGVPFGMTLRPTRSRLSRRTPYSRDRLPPFETETRATI |
| No. 81 | | LEDHPLLPECNTLTMHNAWTSPSPSVKRPQVGQQPVAQQLD |
| | | SDMNLSELPGEFINITDERLARQETVWNITPKNMSVTHDMM |
| | | LFKASRGERTVYSVCWEGGGRLNTRVL* |
| SEQ ID | Ad7-E4orf6/7 | MSGSNSIMTRLRARSTSCARHHPYTRAQLPRCEENETRASM |
| No. 82 | | TEDHPLLPDCDTMTMHSMTVIQTPESHPQQLDCESALKDYP |
| | | DAFLCITDPRLARFETVWNVESKTMSICNGIQMFKAVRGER |
| | | LVYSVKWEGGGKITTRIL* |
| SEQ ID | Ad11-E4orf6/7 | MSGSNSIMTRLRARSTSCARHHPYTRAQLPRCEENETRASM |
| No. 83 | | TEDHPLLPDCDTMTMHSMTVIQTPESQPQQLDCESALKDYP |
| | | DAFLSITDPRLARSETVWNVESKTMSISNGIQMFKAVRGER |
| | | LVYSVKWEGGGKITTRIL* |
| SEQ ID | Ad14-E4orf6/7 | MSGSNSIMTRLCARSTSCARHHPYTRAQLPRCEENETRASM |
| No. 84 | | TEDHPLLPDCDTMTMHSMTVIQTPESHPQQLDCESALKDYP |
| | | AGFLSITDPRLARYETVWNVESKTMSISNGIQMFKAVRGER |
| | | LVYSVKWEGGGKITTRSL* |
| SEQ ID | Ad18-E4orf6/7 | MQRNRRYPYRLAPYGKYPLPPCEKEMRASLFGPENSSVSEC |
| No. 85 | | NSLTLHNVINMDLVLDGESYLSDCVGEGFVSIIDHRFARKE |
| | | TIWTVTPKNLSRNMHMQLFSAIKGERVVYKIKWEGGGSLTT |
| | | RIV* |
| SEQ ID | Ad19-E4orf6/7 | MQTEDQSSLLRHHPYRRARLPRSDEETRASLTEQHPLLPDC |
| No. 86 | | DHADYHNTVTLDCEARLDDESEDGFISITDPRLARQETVWI |
| | | IDPKLSSRTNQNIPLEKATRAERTVYTVKWAGGGRLTTRAG |
| | | VKINKDT* |
| SEQ ID | Ad20-E4orf6/7 | MQTEIQSSSLRHHPYRRARLPRSDEETRASLTEQHPLLPDC |
| No. 87 | | DHADYHNTVTLDCEARLDDFSEDGFISITDPRLARQETVWI |
| | | IDPKSSSRTNQNISLFKATRAERTVYTVKWAGGGRLTTRAG |
| | | VKINKDT* |
| SEQ ID | Ad23-E4orf6/7 | MSTEEQSSSLRHHPYRRARLPRCEEETRASLTEQHPLLPDC |
| No. 88 | | DHADYHNTVTLDCEARLDDFSEDGFISITDPRLARQETVWI |
| | | IDTKTSSRTNNNIPLFKATRAERIVYTVKWAGGGRLTTRAG |
| | | VKINKDT* |
| SEQ ID | Ad25-E4orf6/7 | MSTEEQSSSLRHHPYRRARLPRCEEETRASLTEQHPLLPDC |
| No. 7 | | DHADYHNTVTLDCEARLDDFSEDGFISITDPRLARQETVWI |
| | | IDPKSSSRTNQNISLFKATRAERTVYTVKWAGGGRLTTRAG |
| | | VKINKDT* |
| SEQ ID | Ad27-E4orf6/7 | MQTEDQSSLLRHHPYRRARLPRSDEETRASLTEQHPLLPDC |
| No. 39 | | DHADYHNTVTLDCEARLDDFSEDGFISITDPRLARQETVWI |
| | | IDPKFSSRTNQNIPLFKATRAERIVYTVKWAGGGRLTTRAG |
| | | VKINKDT* |
| SEQ ID | Ad29-E4orf6/7 | MQTEDQSSLLRHHPYRRARLPRSDEETRASLTEQHPLLPDC |
| No. 40 | | DHAEYHNTVTLDCEARLDDESEDGFISITDPRLARQETVWL |
| | | IDTKSSSRANQNIPLFKATRAERIVYTVKWAGGGRLTTRAG |
| | | VKINKDT* |
| SEQ ID | Ad30-E4orf6/7 | MSTEEQSSSLRHHPYRRARLPRCEEETRASLTEQHPLLPDC |
| No. 41 | | DHADYHNTVTLDCEARLDDFSEDGFISITDPRLARQETVWI |
| | | IDSKSSSRTNQNIPLFKATRAERIVYTVKWAGGGRLTTRAG |
| | | VKINKDP* |
| SEQ ID | Ad32-E4orf6/7 | MSTEEQSSSLRHHPYRRARLPRCDEETRASLTEQHPLLPDC |
| No. 42 | | DHADYHNTVTLDCEARLDDFSEDGFISITDPRLARQETVWI |
| | | IDPKSSSRTNQNIFLFKATRAERIVYTVKWAGGGRLTTRAG |
| | | VKINKDT* |
| SEQ ID | Ad34-E4orf6/7 | MSGSNSIMTRLRARSTSCARHHPYTRAQLPRCEENETRASM |
| No. 43 | | TEDHPLLPDCDTMTMHSMTVIQTPESHPQQLDCESALKDYP |
| | | DAFLSITDPRLARSETVWNVESKTMSISNGIQMFKAVRGER |
| | | LVYSVKWEGGGKITTRIL* |
| SEQ ID | Ad36-E4orf6/7 | MSTEEQSTSLRHHPYRRARLPRSEEETRASLTEQHPLLPDC |
| No. 44 | | DHAEYHNTVTLDCEARLEDFSEDGFISITDPRLARQETVWI |
| | | IDTKSSSRTNQNIPLFKATRAERIVYTVKWAGGGRLTTRAG |
| | | VKINKDT* |
| SEQ ID | Ad38-E4orf6/7 | MSTEEQSSLLRHHPYRRARLPRCEEETRASLTEQHPLLPDC |
| No. 45 | | DHADYHNTVTLDCEARLDDFSEDGFISITDPRLARQETVWI |
| | | IDTKSSSRSNNNIPLFKATRAERIVYTVKWAGGGRLTTRAG |
| | | VKINKDS* |
| SEQ ID | Ad39-E4orf6/7 | MSTEEQSSLLRHHPYRRARLPRCEEETRASLTEQHPLLPDC |
| No. 46 | | DHADYHNTVTLDCEARLEDFSEDGFISITDPRLARQETVWI |
| | | IDTKSSSRTNQNIPLEKATRAERIVYTVKWAGGGRLTTRAG |
| | | VKINKDT* |
| SEQ ID | Ad43-E4orf6/7 | MSTEEQSSLLRHHPYRRARLPRCEQETRASLTEQHPLLPDC |
| No. 47 | | DHAEYHNTVTLDCEPRLEDFSEDGFISITDPRLARQETVWL |
| | | IDTKTSSRTNQNIPLEKATRAERTVYTVKWAGGGRLTTRAG |
| | | VKINKDT* |
| SEQ ID | Ad44-E4orf6/7 | MQTEDQSSLLRHHPYRRARLPRCEEETRASLTEQHPLLPDC |
| No. 48 | | DHADYHNTVTLDCEARLEDFSEDGFISITDPRLARQETVWL |
| | | IDTKSSSRTNQNIPLEKATRAERTVYTVKWAGGGRLTTRAG |
| | | VKINKDT* |
| SEQ ID | Ad45-E4orf6/7 | MSTEEQSSLLRHHPYRRARLPRCEEETRASLTEQHPLLPDC |
| No. 49 | | DHADYHNTVTLDCEARLDDFSEDGFISITDPRLARQETVWI |
| | | IDPKSSSRTNQNISLFKATRAERIVYTVKWAGGGRLTTRAG |
| | | VKINKDT* |
| SEQ ID | Ad47-E4orf6/7 | MSTEEQSSLLRHHPYRRARLPRSDEETRASLTEQHPLLPDC |
| No. 50 | | DHADYHNTVTLDCEARLEDESEDGFISITDPRLARQETVWI |
| | | IDPKSSSRTNQNIPLFKATRAERIVYTVKWAGGGRLTTRAG |
| | | VKINKDT* |
| SEQ ID | Ad50-E4orf6/7 | MSGNNSIMTRLRARSTSCARHHPYTRAQLPRCEENETRASM |
| No. 51 | | TEDHPLLPDCDTMTMHSMTVIQTPESHPQQLDCESALKDYP |
| | | DAFLCITDPRLARSETVWNVETKTMSISNGIQMFKAVRGER |
| | | LVYSVKWEGGGKITTRIL* |
| SEQ ID | Ad53-E4orf6/7 | MQTEIQSSSLRHHPYRRARLPRSDEETRASLTEQHPLLPDC |
| No. 52 | | DHADYHNTVTLDCEARLDDFSEDGFISITDPRLARQETVWI |
| | | INPKSSSRTNENFPLFKATRAERIVYTVKWAGGGRMCTRAG |
| | | VKINKDT* |
| SEQ ID | Ad54-E4orf6/7 | MSTEEQSTLLRHHPYRRARLSRYDKETRASLTEQHPLLPDC |
| No. 53 | | DHADYHNTVTLDCEARLDDLSKDGFISITDPRLARQETVWI |
| | | IDPKSSSRTNENFPLFKATRTERIVYTVKWAGGGRMCTRAG |
| | | VKINKDT* |
| SEQ ID | Ad58-E4orf6/7 | MQTEIQSSLLRHHPYRRARLPRSDEETRASLTEQHPLLPDC |
| No. 54 | | DHADYHNTVTLDCEARLDDESEDGFISITDPRLARQETVWL |
| | | IDTKSSSRANQNIPLFKATRAERIVYTVKWAGGGRLTTRAG |
| | | VKINKDT* |
| SEQ ID | Ad64-E4orf6/7 | MQTEIQSSSLRHHPYRRARLPRSDEETRASLTEQHPLLPDC |
| No. 55 | | DHADYHNTVTLDCEARLDDFSEDGFISITDPRLARQETVWI |
| | | IDPKSNSRTNENISLFKATRAERTVYTVKWAGGGRLSTRAG |
| | | VKINKDT* |
| SEQ ID | Ad69-E4orf6/7 | MQTEIQSSSLRHHPYRRARLPRSDEETRASLTEQHPLLPDC |
| No. 56 | | DHADYHNTVTLDCEARLDDESEDGFISITDPRLARQETVWI |
| | | IDPKSSSRTNQNISLFKATRAERIVYTVKWAGGGRLTTRAG |
| | | VKINKDT* |

An example of a nucleotide sequence encoding E4orf6 is set forth in SEQ ID No. 12.

(SEQ ID NO. 12)
ATGACAACCAGCGGCGTGCCCTTCGGCATGACACTGAGGCCTACAAGAA
GCCGGCTGAGCAGAAGAACCCCTTACAGCAGAGACAGACTGCCTCCATT
CGAGACAGAGACACGGGCCACCATCCTGGAAGATCACCCTCTGCTGCCC
GAGTGCAACACCCTGACAATGCACAACGTGTCCTACGTGCGGGGCCTGC
CTTGTAGCGTTGGCTTCACACTGATCCAAGAGTGGGTCGTGCCCTGGGA
CATGGTGCTGACCAGAGAGGAACTGGTCATCCTGCGGAAGTGTATGCAC
GTGTGCCTGTGCTGCGCCAACATCGACATCATGACCAGCATGATGATCC
ACGGCTACGAGAGCTGGGCCCTGCACTGTCACTGTTCTAGCCCTGGCAG
CCTGCAGTGTATTGCTGGTGGACAGGTTCTGGCCAGCTGGTTCAGAATG
GTGGTGGACGGCGCCATGTTCAACCAGAGATTCATCTGGTACAGAGAGG
TGGTCAACTACAACATGCCCAAAGAAGTGATGTTCATGAGCAGCGTTTT
CATGCGGGGCAGACACCTGATCTACCTGCGGCTTTGGTACGATGGCCAC
GTGGGATCTGTGGTGCCTGCCATGAGCTTTGGCTACAGCGCCCTGCATT
GCGGCATCCTGAACAACATCGTGGTGCTGTGCTGCAGCTACTGCGCCGA
TCTGAGCGAGATCAGAGTGCGGTGTTGTGCCAGACGGACCAGACGGCTG
ATGCTGAGAGCCGTGCGGATCATTGCCGAAGAGACAACCGCCATGCTGT
ACTCTTGCCGGACCGAGAGAAGAAGGCAGCAGTTCATCAGAGCCCTGCT
CCAGCACCACCGGCCTATCCTGATGCACGACTACGACAGCACCCCTATG
TAG

An example of an amino acid sequence of E4orf6 is set forth in SEQ ID No. 13.

(SEQ ID NO. 13)
MTTSGVPFGMTLRPTRSRLSRRTPYSRDRLPPFETETRATILEDHPLLP
ECNTLTMHNVSYVRGLPCSVGFTLIQEWVVPWDMVLTREELVILRKCMH
VCLCCANIDIMTSMMIHGYESWALHCHCSSPGSLQCIAGGQVLASWFRM
VVDGAMENQRFIWYREVVNYNMPKEVMEMSSVEMRGRHLIYLRLWYDGH
VGSVVPAMSFGYSALHCGILNNIVVLCCSYCADLSEIRVRCCARRTRRL
MLRAVRIIAEETTAMLYSCRTERRRQQFIRALLQHHRPILMHDYDSTPM

An example of a nucleotide sequence encoding E1B55K is set forth in SEQ ID No. 14.

(SEQ ID No. 14)
ATGGAAAGACGGAACCCCAGCGAGAGAGGCGTGCCAGCTGGATTTTCTG
GACACGCCAGCGTGGAAAGCGGCTGCGAGACACAAGAAAGCCCTGCCAC
CGTGGTGTTCAGACCACCTGGCGATAATACCGATGGCGGAGCTGCTGCT
GCAGCTGGTGGATCTCAGGCAGCAGCTGCAGGCGCTGAACCTATGGAAC
CTGAGAGCAGACCTGGACCTAGCGGCATGAATGTGGTGCAGGTCGCCGA
GCTGTATCCCGAGCTGAGAAGAATCCTGACCATCACCGAGGATGGCCAG
GGACTGAAGGGCGTGAAGAGAGAAAGAGGCGCCTGCGAGGCCACAGAGG
AAGCCAGAAATCTGGCCTTCAGCCTGATGACCAGACACAGACCCGAGTG
CATCACCTTCCAGCAGATCAAGGACAACTGCGCCAACGAGCTGGACCTG
CTGGCCCAGAAGTACAGCATCGAGCAGCTGACCACCTACTGGCTGCAAC
CCGGCGACGATTTCGAAGAGGCCATCAGAGTGTACGCCAAGGTGGCCCT
CAGACCTGACTGCAAGTACAAGATCAGCAAGCTGGTCAACATCCGGAAC
TGCTGCTACATCAGCGGCAATGGCGCCGAGGTGGAAATCGACACAGAGG
ACAGAGTGGCCTTCCGGTGCAGCATGATCAACATGTGGCCTGGCGTGCT
CGGCATGGATGGCGTGGTCATTATGAACGTGCGGTTCACAGGCCCCAAC
TTCAGCGGCACAGTGTTTCTGGCCAACACCAACCTGATCCTGCACGGCG
TGTCCTTCTACGGCTTCAACAATACCTGCGTGGAAGCCTGGACCGACGT
TCGCGTTAGAGGCTGCGCCTTCTACTGCTGTTGGAAGGGCGTCGTGTGC
AGACCCAAGAGCAGAGCCAGCATCAAGAAGTGCCTGTTCGAGAGATGCA
CCCTGGGCATCCTGAGCGAGGGCAACAGCAGAGTCAGACACAACGTGGC
CAGCGACTGCGGCTGCTTCATGCTGGTTAAGAGCGTGGCCGTGATCAAG
CACAACATGGTCTGCGGCAACTGCGAGGATAGAGCCAGCCAGATGCTGA
CCTGCAGCGACGGCAATTGTCATCTGCTGAAAACCATCCACGTGGCCTC
TCACAGCAGAAAGGCCTGGCCTGTGTTCGAGCACAATATCCTGACACGG
TGCTCCCTGCACCTGGGCAATAGACGGGGAGTGTTCCTGCCTTACCAGT
GCAACCTGAGCCACACCAAGATCCTGCTGGAACCCGAGTCCATGAGCAA
AGTGAACCTGAATGGCGTGTTCGACATGACCATGAAGATCTGGAAAGTG
CTGCGCTACGACGAGACACGGACCAGATGTAGACCTTGCGAGTGTGGCG
GCAAGCACATCAGAAACCAGCCTGTGATGCTGGACGTGACCGAGGAACT
GAGGCCTGATCATCTGGTGCTGGCCTGTACCAGAGCCGAGTTTGGCAGC
TCCGACGAGGATACCGAT

An example of an amino acid sequence of E1B55K is set forth in SEQ ID No. 15.

(SEQ ID NO. 15)
MERRNPSERGVPAGESGHASVESGCETQESPATVVERPPGDNTDGGAAA
AAGGSQAAAAGAEPMEPESRPGPSGMNVVQVAELYPELRRILTITEDGQ
GLKGVKRERGACEATEEARNLAFSLMTRHRPECITFQQIKDNCANELDL
LAQKYSIEQLTTYWLQPGDDFEEAIRVYAKVALRPDCKYKISKLVNIRN
CCYISGNGAEVEIDTEDRVAFRCSMINMWPGVLGMDGVVIMNVRFTGPN
FSGTVFLANTNLILHGVSFYGFNNTCVEAWTDVRVRGCAFYCCWKGVVC
RPKSRASIKKCLFERCTLGILSEGNSRVRHNVASDCGCEMLVKSVAVIK
HNMVCGNCEDRASQMLTCSDGNCHLLKTIHVASHSRKAWPVFEHNILTR
CSLHLGNRRGVFLPYQCNLSHTKILLEPESMSKVNLNGVEDMTMKIWKV
LRYDETRTRCRPCECGGKHIRNQPVMLDVTEELRPDHLVLACTRAEFGS
SDEDTD

In one embodiment, the at least one adenoviral protein is not E4orf6. In one embodiment, the at least one adenoviral protein is not E1B55K. In one embodiment, the at least one adenoviral protein does not comprise E4orf6 or E1B55K.

Preferably, the at least one adenoviral protein is E40RF1, preferably wherein the amino acid sequence of E40RF1 is set forth in SEQ ID No. 1, or SEQ ID Nos. 57-76. The at least one adenoviral protein may be a variant or fragment of E40RF1, when the variant or fragment substantially retains the biological activity of the full length E40RF1, e.g. the ability to increase the survival and/or engraftment of gene edited haematopoietic stem/progenitor cells defined herein.

Preferably, the at least one adenoviral protein is E40RF6/7, preferably wherein the amino acid sequence of E40RF6/7 is set forth in SEQ ID No. 2, or SEQ ID Nos. 77-107. The at least one adenoviral protein may be a variant or fragment of E40RF6/7, when the variant or fragment substantially retains the biological activity of the full length E40RF6/7, e.g. the ability to increase the survival and/or engraftment of gene edited haematopoietic stem/progenitor cells defined herein.

In one embodiment, the agent comprises adenoviral protein E40RF1, preferably wherein the amino acid sequence of E40RF1 is set forth in SEQ ID No. 1, or SEQ ID Nos. 57-76; and adenoviral protein E40RF6/7, preferably wherein the amino acid sequence of E40RF6/7 is set forth in SEQ ID No. 2, or SEQ ID Nos. 77-107.

In one embodiment, the inhibitor according to the invention or the agent for use according to the invention comprises a nucleic acid sequence encoding adenoviral protein E40RF1, preferably wherein the amino acid sequence of E40RF1 is set forth in SEQ ID No. 1, or SEQ ID Nos. 57-76; and a nucleic acid sequence adenoviral protein E40RF6/7, preferably wherein the amino acid sequence of E40RF6/7 is set forth in SEQ ID No. 2, or SEQ ID Nos. 77-107.

In one embodiment, the agent comprises two compounds as defined herein that promote homology directed repair. In one embodiment, the agent comprises the inhibitor of p53 activation and the adenoviral protein, or a nucleotide sequence encoding therefor.

Preferably, the inhibitor of p53 activation is GSE56 or a variant thereof.

In one embodiment, the agent comprises:
(a) the inhibitor of p53 activation and adenoviral protein E40RF1 (or a nucleotide sequence encoding therefor). In one embodiment, the E40RF1 has the amino acid sequence of E40RF1 set forth in SEQ ID No. 1, or SEQ ID Nos. 57-76.;
(b) the inhibitor of p53 activation and adenoviral protein E40RF6/7 (or a nucleotide sequence encoding therefor). In one embodiment, the E40RF6/7 has the amino acid sequence of E40RF6/7 set forth in SEQ ID No. 2; or SEQ ID Nos. 77-107.
(c) the inhibitor of p53 activation, adenoviral protein E40RF1 (or a nucleotide sequence encoding therefor), preferably wherein the amino acid sequence of E40RF1 is set forth in SEQ ID No. 1, or SEQ ID Nos. 57-76, and adenoviral protein E40RF6/7 (or a nucleotide sequence encoding therefor), preferably wherein the amino acid sequence of E40RF6/7 is set forth in SEQ ID No. 2; or SEQ ID Nos. 77-107. Preferably, the inhibitor of p53 activation is GSE56 or a variant thereof.

In one embodiment, the agent is a composition comprising the inhibitor of p53 activation and the adenoviral protein, or a nucleotide sequence encoding therefor.

In one embodiment, the inhibitor of p53 activation is administered simultaneously, sequentially or separately in combination with the adenoviral protein, or a nucleotide sequence encoding therefor.

In one embodiment, the adenoviral protein, or a nucleotide sequence encoding therefor, is administered simultaneously, sequentially or separately in combination with the inhibitor of p53 activation.

In one embodiment, the nucleic acid encoding the adenoviral protein is an mRNA.

In one embodiment, the adenoviral protein is expressed transiently in the haematopoietic cell, haematopoietic stem cell, haematopoietic progenitor cell and/or T cell. Preferably, the transient expression occurs during gene editing and/or transduction of the haematopoietic cell, haematopoietic stem cell, progenitor cell and/or T cell.

### Combinations

The invention contemplates all possible combinations of embodiments described herein, irrespective of whether the embodiments are specifically described in combination.

In one embodiment the invention provides one or more inhibitor(s) of senescence in combination with one or more of:
(i) one or more adenoviral proteins, or a nucleic acid encoding one or more adenoviral proteins, as described herein;
(ii) one or more transduction enhancers as described herein;
(iii) one or more p53 inhibitors as described herein; and/or
(iv) an integrase-defective-lentiviral vector (IDLV).

In another embodiment the invention provides one or more inhibitor(s) of senescence in combination with:
(i) one or more adenoviral proteins, or a nucleic acid encoding one or more adenoviral proteins, as described herein;
(ii) one or more transduction enhancers as described herein;
(iii) one or more p53 inhibitors as described herein; and
(iv) an integrase-defective-lentiviral vector (IDLV).

In a preferred embodiment the p53 inhibitor is GSE56.

In another preferred embodiment the transduction enhancer is CsH.

In a preferred embodiment the invention provides one or more inhibitor(s) of senescence in combination with:
(i) (one or more adenoviral proteins, or a nucleic acid encoding one or more adenoviral proteins, as described herein;
(ii) CsH;
(iii) GSE56; and
(iv) an integrase-defective-lentiviral vector (IDLV).

These combinations are specifically disclosed in relation to all of the inhibitors of senescence, uses, methods, pharmaceutical compositions, and populations of gene edited and/or transduced haematopoietic cells of the invention.

### Isolation and Enrichment of Populations of Cells

The term “isolated population” of cells as used herein may refer to the population of cells having been previously removed from the body. An isolated population of cells may be cultured and manipulated ex vivo or in vitro using standard techniques known in the art. An isolated population of cells may later be reintroduced into a subject. Said subject may be the same subject from which the cells were originally isolated or a different subject.

A population of cells may be purified selectively for cells that exhibit a specific phenotype or characteristic, and from other cells which do not exhibit that phenotype or characteristic, or exhibit it to a lesser degree. For example, a population of cells that expresses a specific marker (such as CD34) may be purified from a starting population of cells. Alternatively, or in addition, a population of cells that does not express another marker (such as CD38) may be purified.

By “enriching” a population of cells for a certain type of cells it is to be understood that the concentration of that type of cells is increased within the population. The concentration of other types of cells may be concomitantly reduced.

Purification or enrichment may result in the population of cells being substantially pure of other types of cell.

Purifying or enriching for a population of cells expressing a specific marker (e.g. CD34 or CD38) may be achieved by using an agent that binds to that marker, preferably substantially specifically to that marker.

An agent that binds to a cellular marker may be an antibody, for example an anti-CD34 or anti-CD38 antibody.

The term “antibody” refers to complete antibodies or antibody fragments capable of binding to a selected target, and including Fv, ScFv, F(ab′) and F(ab′)₂, monoclonal and polyclonal antibodies, engineered antibodies including chimeric, CDR-grafted and humanised antibodies, and artificially selected antibodies produced using phage display or alternative techniques.

In addition, alternatives to classical antibodies may also be used in the invention, for example “avibodies”, “avimers”, “anticalins”, “nanobodies” and “DARPins”.

The agents that bind to specific markers may be labelled so as to be identifiable using any of a number of techniques known in the art. The agent may be inherently labelled, or may be modified by conjugating a label thereto. By “conjugating” it is to be understood that the agent and label are operably linked. This means that the agent and label are linked together in a manner which enables both to carry out their function (e.g. binding to a marker, allowing fluorescent identification or allowing separation when placed in a magnetic field) substantially unhindered. Suitable methods of conjugation are well known in the art and would be readily identifiable by the skilled person.

A label may allow, for example, the labelled agent and any cell to which it is bound to be purified from its environment (e.g. the agent may be labelled with a magnetic bead or an affinity tag, such as avidin), detected or both. Detectable markers suitable for use as a label include fluorophores (e.g. green, cherry, cyan and orange fluorescent proteins) and peptide tags (e.g. His tags, Myc tags, FLAG tags and HA tags).

A number of techniques for separating a population of cells expressing a specific marker are known in the art. These include magnetic bead-based separation technologies (e.g. closed-circuit magnetic bead-based separation), flow cytometry, fluorescence-activated cell sorting (FACS), affinity tag purification (e.g. using affinity columns or beads, such biotin columns to separate avidin-labelled agents) and microscopy-based techniques.

It may also be possible to perform the separation using a combination of different techniques, such as a magnetic bead-based separation step followed by sorting of the resulting population of cells for one or more additional (positive or negative) markers by flow cytometry.

Clinical grade separation may be performed, for example, using the CliniMACS® system (Miltenyi). This is an example of a closed-circuit magnetic bead-based separation technology.

It is also envisaged that dye exclusion properties (e.g. side population or rhodamine labelling) or enzymatic activity (e.g. ALDH activity) may be used to enrich for haematopoietic stem cells.

Suitably, the agent does not reduce the fraction CD34⁺ CD133⁺ CD90⁺ cells in population of gene edited cells compared with a population of untreated gene edited cells.

### Gene Editing

The term “gene editing” refers to a type of genetic engineering in which a nucleic acid is inserted, deleted or replaced in a cell. Gene editing may be achieved using engineered nucleases, which may be targeted to a desired site in a polynucleotide (e.g. a genome). Such nucleases may create site-specific double-strand breaks at desired locations, which may then be repaired through non-homologous end-joining (NHEJ) or homologous recombination (HR), resulting in targeted mutations.

Such nucleases may be delivered to a target cell using viral vectors. The present invention provides methods of increasing the efficiency of the gene editing process.

Examples of suitable nucleases known in the art include zinc finger nucleases (ZFNs), transcription activator like effector nucleases (TALENs), and the clustered regularly interspaced short palindromic repeats (CRISPR)/Cas system (Gaj, T. et al. (2013) Trends Biotechnol. 31: 397-405; Sander, J. D. et al. (2014) Nat. Biotechnol. 32: 347-55). Meganucleases (Silve, G. et al. (2011) Cur. Gene Ther. 11: 11-27) may also be employed as suitable nucleases for gene editing.

The CRISPR/Cas system is an RNA-guided DNA binding system (van der Oost et al. (2014) Nat. Rev. Microbiol. 12: 479-92), wherein the guide RNA (gRNA) may be selected to enable a Cas9 domain to be targeted to a specific sequence. Methods for the design of gRNAs are known in the art. Furthermore, fully orthogonal Cas9 proteins, as well as Cas9/gRNA ribonucleoprotein complexes and modifications of the gRNA structure/composition to bind different proteins, have been recently developed to simultaneously and directionally target different effector domains to desired genomic sites of the cells (Esvelt et al. (2013) Nat. Methods 10: 1116-21; Zetsche, B. et al. (2015) Cell pii: S0092-8674(15)01200-3; Dahlman, J. E. et al. (2015) Nat. Biotechnol. 2015 Oct. 5. doi: 10.1038/nbt.3390. [Epub ahead of print]; Zalatan, J. G. et al. (2015) Cell 160: 339-50; Paix, A. et al. (2015) Genetics 201: 47-54), are suitable for use in the invention.

### Pre-Treatment

The method of the invention may further comprise a pre-culturing step. As used herein, a “pre-culturing step” may refer to a culturing step which occurs prior to introduction of gene editing machinery to the population of cells and/or transduction of the population of cells. As used herein, a “pre-activating step” may refer to an activation step or stimulation step which occurs prior to introduction of gene editing machinery to the population of cells and/or transduction of the population of cells. As used herein, a “pre-expansion step” may refer to an expansion step which occurs prior to introduction of gene editing machinery to the population of cells and/or transduction of the population of cells.

In some embodiments, the method further comprises a pre-culturing step before the contacting of the population of cells with the one or more inhibitor(s) of senescence. In some embodiments, the method further comprises a pre-culturing step before and/or during the contacting of the population of cells with the one or more inhibitor(s) of senescence. In some embodiments, the method further comprises a pre-culturing step before the introducing gene editing machinery to the population of cells. In some embodiments, the method further comprises a pre-culturing step before the transducing the population of cells.

The pre-culturing step (e.g. pre-activation step and/or pre-expansion step) may be carried out using any suitable conditions.

During the pre-culturing step (e.g. pre-activation step and/or pre-expansion step) the population of cells may be seeded at a concentration of about 1×10⁵ cells/ml to about 10×10⁵ cells/ml, e.g. about 2×10⁵ cells/ml, or about 5×10⁵ cells/ml.

Suitably, the pre-culturing step (e.g. pre-activation step and/or pre-expansion step) is at least 1 day, at least 2 days, or at least 3 days. Suitably, the population of cells are pre-cultured (e.g. pre-activated and/or pre-expanded) for about 3 days. Suitably, the population of cells are pre-cultured in a 5% CO₂ humidified atmosphere at 37° C.

Any suitable culture medium may be used. For example, commercially available medium such as StemSpan medium may be used, which contains bovine serum albumin, insulin, transferrin, and supplements in Iscove's MDM. The culture medium may be supplemented with one or more antibiotic (e.g. penicillin, streptomycin).

The pre-culturing step (e.g. pre-activation step and/or pre-expansion step) may be carried out in the presence in of one or more cytokines and/or growth factors. As used herein, a “cytokine” is any cell signalling substance and includes chemokines, interferons, interleukins, lymphokines, and tumour necrosis factors. As used herein, a “growth factor” is any substance capable of stimulating cell proliferation, wound healing, or cellular differentiation. The terms “cytokine” and “growth factor” may overlap.

The pre-culturing step (e.g. pre-activation step and/or pre-expansion step) may be carried out in the presence of one or more early-acting cytokine, one or more transduction enhancer, and/or one or more expansion enhancer.

### Early-Acting Cytokines

As used herein, an “early-acting cytokine” is a cytokine which stimulates cells such as HSCs or HPCs. Early-acting cytokines include thrombopoietin (TPO), stem cell factor (SCF), Flt3-ligand (FLT3-L), interleukin (IL)-3, and IL-6. In some embodiments, the pre-culturing step (e.g. pre-activation step and/or pre-expansion step) is carried out in the presence of at least one early-acting cytokine. Any suitable concentration of early-acting cytokine may be used. For example, 1-1000 ng/ml, or 10-1000 ng/ml, or 10-500 ng/ml.

In some embodiments, the pre-culturing step (e.g. pre-activation step and/or pre-expansion step) is carried out in the presence of SCF. The concentration of SCF may be about 10-1000 ng/ml, about 50-500 ng/ml, or about 100-300 ng/ml.

In some embodiments, the pre-culturing step (e.g. pre-activation step and/or pre-expansion step) is carried out in the presence of FLT3-L. The concentration of FLT3-L may be about 10-1000 ng/ml, about 50-500 ng/ml, or about 100-300 ng/ml.

In some embodiments, the pre-culturing step (e.g. pre-activation step and/or pre-expansion step) is carried out in the presence of TPO. The concentration of TPO may be about 5-500 ng/ml, about 10-200 ng/ml, or about 20-100 ng/ml.

In some embodiments, the pre-culturing step (e.g. pre-activation step and/or pre-expansion step) is carried out in the presence of IL-3. The concentration of IL-3 may be about 10-200 ng/ml, about 20-100 ng/ml, or about 60 ng/ml.

In some embodiments, the pre-culturing step (e.g. pre-activation step and/or pre-expansion step) is carried out in the presence of IL-6. The concentration of IL-6 may be about 5-100 ng/ml, about 10-50 ng/ml, or about 20 ng/ml.

In some embodiments, the pre-culturing step (e.g. pre-activation step and/or pre-expansion step) is carried out in the presence of SCF (e.g. in a concentration of about 100 ng/ml), FLT3-L (e.g. in a concentration of about 100 ng/ml), TPO (e.g. in a concentration of about 20 ng/ml) and IL-6 (e.g. in a concentration of about 20 ng/ml), in particular when the population of cells are cord-blood CD34+ cells.

In some embodiments, the pre-culturing step (e.g. pre-activation step and/or pre-expansion step) is carried out in the presence of SCF (e.g. in a concentration of about 300 ng/ml), FLT3-L (e.g. in a concentration of about 300 ng/ml), TPO (e.g. in a concentration of about 100 ng/ml) and IL-3 (e.g. in a concentration of about 60 ng/ml), in particular when the population of cells are (mobilised) peripheral blood CD34+ cells.

### Transduction Enhancers

As used herein, a “transduction enhancer” is a substance that is capable of improving viral transduction of cells such as HSCs or HPCs. Suitable transduction enhancers include LentiBOOST, prostaglandin E2 (PGE2), protamine sulfate (PS), Vectofusin-1, ViraDuctin, RetroNectin, staurosporine (Stauro), 7-hydroxy-stauro, human serum albumin, polyvinyl alcohol, and cyclosporin H (CsH). In some embodiments, the pre-culturing step (e.g. pre-activation step and/or pre-expansion step) is carried out in the presence of at least one transduction enhancer. Any suitable concentration of transduction enhancer may be used, for example as described in Schott, J. W., et al., 2019. Molecular Therapy-Methods & Clinical Development, 14, pp. 134-147 or Yang, H., et al., 2020. Molecular Therapy-Nucleic Acids, 20, pp. 451-458.

In some embodiments, the pre-culturing step (e.g. pre-activation step and/or pre-expansion step) is carried out in the presence of PGE2. Suitably, the PGE2 is 16,16-dimethyl prostaglandin E2 (dmPGE2). The concentration of PGE2 may be about 1-100 μM, about 5-20 μM, or about 10 μM.

In some embodiments, the pre-culturing step (e.g. pre-activation step and/or pre-expansion step) is carried out in the presence of CsH. The concentration of CsH may be about 1-50 μM, 5-50 μM, about 10-50 μM, or about 10 μM.

### Expansion Enhancers

As used herein, an “expansion enhancer” is a substance that is capable of improving expansion of cells such as HSCs or HPCs. Suitable expansion enhancers include UM171, UM729, StemRegenin1 (SR1), diethylaminobenzaldehyde (DEAB), LG1506, BIO (GSK3β inhibitor), NR-101, trichostatin A (TSA), garcinol (GAR), valproic acid (VPA), copper chelator, tetraethylenepentamine, and nicotinamide. In some embodiments, the pre-culturing step (e.g. pre-activation step and/or pre-expansion step) is carried out in the presence of at least one expansion enhancer. Any suitable concentration of expansion enhancer may be used, for example as described in Huang, X., et al., 2019. F1000Research, 8, 1833.

In some embodiments, the pre-culturing step (e.g. pre-activation step and/or pre-expansion step) is carried out in the presence of UM171 or UM729. The concentration of UM171 may be about 10-200 nM, about 20-100 nM, or about 50 nM.

In some embodiments, the pre-culturing step (e.g. pre-activation step and/or pre-expansion step) is carried out in the presence of SR1. The concentration of SR1 may be about 0.1-10 μM, about 0.5-5 μM, or about 1 μM.

In some embodiments, the pre-culturing step (e.g. pre-activation step and/or pre-expansion step) is carried out in the presence of UM171 (e.g. in a concentration of about 50 nM) or UM729 and SR1 (e.g. in a concentration of about 1 μM).

In some embodiments, the pre-culturing step (e.g. pre-activation step and/or pre-expansion step) is carried out in the presence of SCF (e.g. in a concentration of about 100 ng/ml), FLT3-L (e.g. in a concentration of about 100 ng/ml), TPO (e.g. in a concentration of about 20 ng/ml), IL-6 (e.g. in a concentration of about 20 ng/ml), PGE2 (e.g. in a concentration of about 10 μM), UM171 (e.g. in a concentration of about 50 nM), and SR1 (e.g. in a concentration of about 1 μM), in particular when the population of cells are cord-blood CD34+ cells.

In some embodiments, the pre-culturing step (e.g. pre-activation step and/or pre-expansion step) is carried out in the presence of SCF (e.g. in a concentration of about 300 ng/ml), FLT3-L (e.g. in a concentration of about 300 ng/ml), TPO (e.g. in a concentration of about 100 ng/ml), IL-3 (e.g. in a concentration of about 60 ng/ml), PGE2 (e.g. in a concentration of about 10 μM), UM171 (e.g. in a concentration of about 50 nM), and SR1 (e.g. in a concentration of about 1 μM), in particular when the population of cells are (mobilised) peripheral blood CD34+ cells.

### Vectors

A vector is a tool that allows or facilitates the transfer of an entity from one environment to another. The vectors used to transduce haematopoietic stem and/or progenitor cells in the invention are viral vectors.

Preferably, the viral vector is in the form of a viral vector particle.

The viral vector may be, for example, an adeno-associated viral (AAV), adenoviral, a retroviral or lentiviral vector. Preferably, the viral vector is an AAV vector or a retroviral or lentiviral vector, more preferably an AAV vector. Preferably, the retroviral vector is not a γ-retroviral vector.

By “vector derived from” a certain type of virus, it is to be understood that the vector comprises at least one component part derivable from that type of virus.

### Adeno-Associated Viral (AAV) Vectors

Adeno-associated virus (AAV) is an attractive vector system for use in the invention as it has a high frequency of integration and it can infect non-dividing cells. This makes it useful for delivery of genes into mammalian cells in tissue culture.

AAV has a broad host range for infectivity. Details concerning the generation and use of AAV vectors are described in U.S. Pat. Nos. 5,139,941 and 4,797,368.

Recombinant AAV vectors have been used successfully for in vitro and in vivo transduction of marker genes and genes involved in human diseases.

Preferred vectors are those which are able to achieve a high transduction efficiency in human primary cells, such as HSPC cells.

In one embodiment, the vector is an AAV6 vector or a vector derived from an AAV6 vector. Preferably the vector is an AAV6 vector.

### Adenoviral Vectors

The adenovirus is a double-stranded, linear DNA virus that does not go through an RNA intermediate. There are over 50 different human serotypes of adenovirus divided into 6 subgroups based on the genetic sequence homology. The natural targets of adenovirus are the respiratory and gastrointestinal epithelia, generally giving rise to only mild symptoms. Serotypes 2 and 5 (with 95% sequence homology) are most commonly used in adenoviral vector systems and are normally associated with upper respiratory tract infections in the young.

Adenoviruses have been used as vectors for gene therapy and for expression of heterologous genes. The large (36 kb) genome can accommodate up to 8 kb of foreign insert DNA and is able to replicate efficiently in complementing cell lines to produce very high titres of up to 10¹². Adenovirus is thus one of the best systems to study the expression of genes in primary non-replicative cells.

The expression of viral or foreign genes from the adenovirus genome does not require a replicating cell. Adenoviral vectors enter cells by receptor mediated endocytosis. Once inside the cell, adenovirus vectors rarely integrate into the host chromosome. Instead, they function episomally (independently from the host genome) as a linear genome in the host nucleus. Hence the use of recombinant adenovirus alleviates the problems associated with random integration into the host genome.

### Retroviral and Lentiviral Vectors

A retroviral vector may be derived from or may be derivable from any suitable retrovirus. A large number of different retroviruses have been identified. Examples include murine leukaemia virus (MLV), human T-cell leukaemia virus (HTLV), mouse mammary tumour virus (MMTV), Rous sarcoma virus (RSV), Fujinami sarcoma virus (FuSV), Moloney murine leukaemia virus (Mo-MLV), FBR murine osteosarcoma virus (FBR MSV), Moloney murine sarcoma virus (Mo-MSV), Abelson murine leukaemia virus (A-MLV), avian myelocytomatosis virus-29 (MC29) and avian erythroblastosis virus (AEV). A detailed list of retroviruses may be found in Coffin, J. M. et al. (1997) Retroviruses, Cold Spring Harbour Laboratory Press, 758-63.

Retroviruses may be broadly divided into two categories, “simple” and “complex”. Retroviruses may be even further divided into seven groups. Five of these groups represent retroviruses with oncogenic potential. The remaining two groups are the lentiviruses and the spumaviruses. A review of these retroviruses is presented in Coffin, J. M. et al. (1997) Retroviruses, Cold Spring Harbour Laboratory Press, 758-63.

The basic structure of retrovirus and lentivirus genomes share many common features such as a 5′ LTR and a 3′ LTR. Between or within these are located a packaging signal to enable the genome to be packaged, a primer binding site, integration sites to enable integration into a host cell genome, and gag, pol and env genes encoding the packaging components—these are polypeptides required for the assembly of viral particles. Lentiviruses have additional features, such as rev and RRE sequences in HIV, which enable the efficient export of RNA transcripts of the integrated provirus from the nucleus to the cytoplasm of an infected target cell.

In the provirus, these genes are flanked at both ends by regions called long terminal repeats (LTRs). The LTRs are responsible for proviral integration and transcription. LTRs also serve as enhancer-promoter sequences and can control the expression of the viral genes.

The LTRs themselves are identical sequences that can be divided into three elements: U3, R and U5. U3 is derived from the sequence unique to the 3′ end of the RNA. R is derived from a sequence repeated at both ends of the RNA. U5 is derived from the sequence unique to the 5′ end of the RNA. The sizes of the three elements can vary considerably among different retroviruses.

In a defective retroviral vector genome gag, pol and env may be absent or not functional.

In a typical retroviral vector, at least part of one or more protein coding regions essential for replication may be removed from the virus. This makes the viral vector replication-defective. Portions of the viral genome may also be replaced by a library encoding candidate modulating moieties operably linked to a regulatory control region and a reporter moiety in the vector genome in order to generate a vector comprising candidate modulating moieties which is capable of transducing a target host cell and/or integrating its genome into a host genome.

Lentivirus vectors are part of the larger group of retroviral vectors. A detailed list of lentiviruses may be found in Coffin, J. M. et al. (1997) Retroviruses, Cold Spring Harbour Laboratory Press, 758-63. In brief, lentiviruses can be divided into primate and non-primate groups. Examples of primate lentiviruses include but are not limited to human immunodeficiency virus (HIV), the causative agent of human acquired immunodeficiency syndrome (AIDS); and simian immunodeficiency virus (SIV). Examples of non-primate lentiviruses include the prototype “slow virus” visna/maedi virus (VMV), as well as the related caprine arthritis-encephalitis virus (CAEV), equine infectious anaemia virus (EIAV), and the more recently described feline immunodeficiency virus (FIV) and bovine immunodeficiency virus (BIV).

The lentivirus family differs from retroviruses in that lentiviruses have the capability to infect both dividing and non-dividing cells (Lewis, P et al. (1992) EMBO J. 11: 3053-8; Lewis, P. F. et al. (1994) J. Virol. 68: 510-6). In contrast, other retroviruses, such as MLV, are unable to infect non-dividing or slowly dividing cells such as those that make up, for example, muscle, brain, lung and liver tissue.

A lentiviral vector, as used herein, is a vector which comprises at least one component part derivable from a lentivirus. Preferably, that component part is involved in the biological mechanisms by which the vector infects cells, expresses genes or is replicated.

The lentiviral vector may be a “primate” vector. The lentiviral vector may be a “non-primate” vector (i.e. derived from a virus which does not primarily infect primates, especially humans). Examples of non-primate lentiviruses may be any member of the family of lentiviridae which does not naturally infect a primate.

As examples of lentivirus-based vectors, HIV-1- and HIV-2-based vectors are described below.

The HIV-1 vector contains cis-acting elements that are also found in simple retroviruses. It has been shown that sequences that extend into the gag open reading frame are important for packaging of HIV-1. Therefore, HIV-1 vectors often contain the relevant portion of gag in which the translational initiation codon has been mutated. In addition, most HIV-1 vectors also contain a portion of the env gene that includes the RRE. Rev binds to RRE, which permits the transport of full-length or singly spliced mRNAs from the nucleus to the cytoplasm. In the absence of Rev and/or RRE, full-length HIV-1 RNAs accumulate in the nucleus. Alternatively, a constitutive transport element from certain simple retroviruses such as Mason-Pfizer monkey virus can be used to relieve the requirement for Rev and RRE. Efficient transcription from the HIV-1 LTR promoter requires the viral protein Tat.

Most HIV-2-based vectors are structurally very similar to HIV-1 vectors. Similar to HIV-1-based vectors, HIV-2 vectors also require RRE for efficient transport of the full-length or singly spliced viral RNAs.

In one system, the vector and helper constructs are from two different viruses, and the reduced nucleotide homology may decrease the probability of recombination. In addition to vectors based on the primate lentiviruses, vectors based on FIV have also been developed as an alternative to vectors derived from the pathogenic HIV-1 genome. The structures of these vectors are also similar to the HIV-1 based vectors.

Preferably, the viral vector used in the present invention has a minimal viral genome.

By “minimal viral genome” it is to be understood that the viral vector has been manipulated so as to remove the non-essential elements and to retain the essential elements in order to provide the required functionality to infect, transduce and deliver a nucleotide sequence of interest to a target host cell. Further details of this strategy can be found in WO 1998/017815.

Preferably, the plasmid vector used to produce the viral genome within a host cell/packaging cell will have sufficient lentiviral genetic information to allow packaging of an RNA genome, in the presence of packaging components, into a viral particle which is capable of infecting a target cell, but is incapable of independent replication to produce infectious viral particles within the final target cell. Preferably, the vector lacks a functional gag-pol and/or env gene and/or other genes essential for replication.

However, the plasmid vector used to produce the viral genome within a host cell/packaging cell will also include transcriptional regulatory control sequences operably linked to the lentiviral genome to direct transcription of the genome in a host cell/packaging cell. These regulatory sequences may be the natural sequences associated with the transcribed viral sequence (i.e. the 5′ U3 region), or they may be a heterologous promoter, such as another viral promoter (e.g. the CMV promoter).

The vectors may be self-inactivating (SIN) vectors in which the viral enhancer and promoter sequences have been deleted. SIN vectors can be generated and transduce non-dividing cells in vivo with an efficacy similar to that of wild-type vectors. The transcriptional inactivation of the long terminal repeat (LTR) in the SIN provirus should prevent mobilisation by replication-competent virus. This should also enable the regulated expression of genes from internal promoters by eliminating any cis-acting effects of the LTR.

The vectors may be integration-defective. Integration defective lentiviral vectors (IDLVs) can be produced, for example, either by packaging the vector with catalytically inactive integrase (such as an HIV integrase bearing the D64V mutation in the catalytic site; Naldini, L. et al. (1996) Science 272: 263-7; Naldini, L. et al. (1996) Proc. Natl. Acad. Sci. USA 93: 11382-8; Leavitt, A. D. et al. (1996) J. Virol. 70: 721-8) or by modifying or deleting essential att sequences from the vector LTR (Nightingale, S. J. et al. (2006) Mol. Ther. 13: 1121-32), or by a combination of the above.

### Target Locus

In one embodiment, the gene editing targets a haematopoietic stem cell and/or progenitor locus. In one embodiment, the donor template targets a haematopoietic stem cell and/or progenitor locus.

In one embodiment, the gene editing targets a T cell locus. In one embodiment, the donor template targets a T cell locus.

### AAVS1 Locus

In one embodiment, the gene editing targets the adeno-associated virus integration site 1 (AAVS1) locus. In one embodiment, the donor template targets the adeno-associated virus integration site 1 (AAVS1) locus.

An example of an AAV donor cassette for AAVS1 may comprise the following nucleotide sequences:

3′-ITR
(SEQ ID No. 16)
cctgcaggcagctgcgcgctcgctcgctcactgaggccgcccgggcaaagc
ccgggcgtcgggcgacctttggtcgcccggcctcagtgagcgagcgagcgc
gcagagagggagtggccaactccatcact
Homologous arm-left
(SEQ ID No. 17)
tgctttctctgaccagcattctctcccctgggcctgtgccgctttctgtct
gcagcttgtggcctgggtcacctctacggctggcccagatccttccctgcc
gcctccttcaggttccgtcttcctccactccctcttccccttgctctctgc
tgtgttgctgcccaaggatgctctttccggagcacttccttctcggcgctg
caccacgtgatgtcctctgagcggatcctccccgtgtctgggtcctctccg
ggcatctctcctccctcacccaaccccatgccgtcttcactcgctgggttc
ccttttccttctccttctggggcctgtgccatctctcgtttcttaggatgg
ccttctccgacggatgtctcccttgcgtcccgcctccccttcttgtaggcc
tgcatcatcaccgtttttctggacaaccccaaagtaccccgtctccctggc
tttagccacctctccatcctcttgctttctttgcctggacaccccgttctc
ctgtggattcgggtcacctctcactcctttcatttgggcagctcccctacc
ccccttacctctctagtctgtgctagctcttccagccccctgtcatggcat
cttccaggggtccgagagctcagctagtcttcttcctccaacccgggcccc
tatgtccacttcaggacagcatgtttgctgcctccagggatcctgtgtccc
cgagctgggaccaccttatattcccagggccggttaatgtggctctggttc
tgggtacttttatctgtcccctccaccccac
hPGK promoter (without SA)
(SEQ ID No. 18)
ccacggggttggggttgcgccttttccaaggcagccctgggtttgcgcagg
gacgcggctgctctgggcgtggttccgggaaacgcagcggcgccgaccctg
ggtctcgcacattcttcacgtccgttcgcagcgtcacccggatcttcgccg
ctacccttgtgggccccccggcgacgcttcctgctccgcccctaagtcggg
aaggttccttgcggttcgcggcgtgccggacgtgacaaacggaagccgcac
gtctcactagtaccctcgcagacggacagcgccagggagcaatggcagcgc
gccgaccgcgatgggctgtggccaatagcggctgctcagcagggcgcgccg
agagcagcggccgggaaggggcggtgcgggaggcggggtgtggggcggtag
tgtgggccctgttcctgcccgcgcggtgttccgcattctgcaagcctccgg
agcgcacgtcggcagtcggctccctcgttgaccgaatcaccgacctctctc
ccca
eGFP
(SEQ ID No. 19)
atggtgagcaagggcgaggagctgttcaccggggtggtgcccatcctggtc
gagctggacggcgacgtaaacggccacaagttcagcgtgtccggcgagggc
gagggcgatgccacctacggcaagctgaccctgaagttcatctgcaccacc
ggcaagctgcccgtgccctggcccaccctcgtgaccaccctgacctacggc
gtgcagtgcttcagccgctaccccgaccacatgaagcagcacgacttcttc
aagtccgccatgcccgaaggctacgtccaggagcgcaccatcttcttcaag
gacgacggcaactacaagacccgcgccgaggtgaagttcgagggcgacacc
ctggtgaaccgcatcgagctgaagggcatcgacttcaaggaggacggcaac
atcctggggcacaagctggagtacaactacaacagccacaacgtctatatc
atggccgacaagcagaagaacggcatcaaggtgaacttcaagatccgccac
aacatcgaggacggcagcgtgcagctcgccgaccactaccagcagaacacc
cccatcggcgacggccccgtgctgctgcccgacaaccactacctgagcacc
cagtccgccctgagcaaagaccccaacgagaagcgcgatcacatggtcctg
ctggagttcgtgaccgccgccgggatcactctcggcatggacgagctgtac
aagtaa
bGH-pA
(SEQ ID No. 20)
actgtgccttctagttgccagccatctgttgtttgcccctcccccgtgcct
tccttgaccctggaaggtgccactcccactgtcctttcctaataaaatgag
gaaattgcatcgcattgtctgagtaggtgtcattctattctggggggtggg
gtggggcaggacagcaagggggaggattgggaagacaatagcaggcatgct
ggggatgcggtgggctctatgg
Homologous arm-right
(SEQ ID No. 21)
tagggacaggattggtgacagaaaagccccatccttaggcctcctccttcc
tagtctcctgatattgggtctaacccccacctcctgttaggcagattcctt
atctggtgacacacccccatttcctggagccatctctctccttgccagaac
ctctaaggtttgcttacgatggagccagagaggatcctgggagggagagct
tggcagggggtgggagggaagggggggatgcgtgacctgcccggttctcag
tgg
5′-ITR
(SEQ ID No. 22)
agtgatggagttggccactccctctctgcgcgctcgctcgctcactgaggc
cgggcgaccaaaggtcgcccgacgcccgggcggcctcagtgagcgagcgag
cgcgcagctgcctgcagg

### IL2RG Locus

In another embodiment, the gene editing targets Interleukin 2 Receptor Subunit Gamma (IL2RG), preferably targeting intron 1 of IL2RG. In another embodiment, the donor template targets Interleukin 2 Receptor Subunit Gamma (IL2RG), preferably targeting intron 1 of IL2RG.

An example of an AAV donor cassette for IL2RG may comprise the following nucleotide sequences:

3′-ITR
(SEQ ID No. 23)
cctgcaggcagctgcgcgctcgctcgctcactgaggccgcccgggcaaagc
ccgggcgtcgggcgacctttggtcgcccggcctcagtgagcgagcgagcgc
gcagagagggagtggccaactccatcact
Homologous arm-left
(SEQ ID No. 24)
agaggaaacgtgtgggtggggggggtagtgggtgagggacccaggttcctg
acacagacagactacacccagggaatgaagagcaagcgccatgttgaagcc
atcattaccattcacatccctcttattcctgcagctgcccctgctgggagt
ggggctgaacacgacaattctgacgcccaatgggaatgaagacaccacagc
tggtgggaaatctgggactggagggggctggtgagaagggtggctgtggga
aggggccgtacagagatctggtgcctgccactgg
Exon2
(SEQ ID No. 25)
atttctttctgaccaccatgcccaccgacagcctgagcgtgagcaccctgc
ccctgcccgaggtgcagtgcttcgtgttcaacgtggagtacatgaactgca
cctggaacagcagcagcgagccccagcccaccaatctgaccctgcactact
g
Exon3
(SEQ ID No. 26)
gtacaagaacagcgacaacgacaaggtgcagaagtgcagccactacctgtt
cagcgaggaaatcaccagcggctgccagctgcagaagaaagagatccacct
gtaccagaccttcgtggtgcagctgcaggacccccgggagccccgcaggca
ggccacccagatgctgaagctgcagaacctgg
Exon4
(SEQ ID No. 27)
tgatcccctgggcccctgagaacctgacactgcacaagctgtccgagagcc
agctggaactgaactggaacaaccgcttcctgaaccactgcctggaacacc
tggtgcagtaccggaccgactgggaccacagctggacc
Exon5
(SEQ ID No. 28)
gagcagagcgtggactaccggcacaagttcagcctgcccagcgtggacggc
cagaagcggtacaccttcagagtgcggagccggttcaaccccctgtgcggc
agcgcccagcactggtccgagtggagccaccccatccactggggcagcaac
accagcaaag
Exon6
(SEQ ID No. 29)
agaaccccttcctgttcgccctggaagccgtggtgatcagcgtgggcagca
tgggcctgatcatctccctgctgtgcgtgtacttctggctggaacg
Exon7
(SEQ ID No. 30)
gaccatgcccagaatccccaccctgaagaacctggaagatctggtgaccga
gtaccacggcaacttcagc
Exon8
(SEQ ID No. 31)
gcctggtccggcgtgagcaagggcctggccgagagcctgcagcccgactac
agcgagcggctgtgcctggtgtccgagatcccccccaaaggcggagccctg
ggcgaaggccctggcgccagcccctgcaaccagcacagcccctactgggcc
cctccttgctacaccctgaagcccgagacctga
3′-UTR
(SEQ ID No. 32)
tgaaccccaatcctctgacagaagaaccccagggtcctgtagccctaagtg
gtactaactttccttcattcaacccactgcgtctcatactcacctcacccc
actgtggctgatttggaattttgtgcccccatgtaagcaccccttcatttg
gcattccccacttgagaattacccttttgccccgaacatgtttttcttctc
cctcagtctggcccttccttttcgcaggattcttcctccccccctctttcc
ctcccttcctctttccatctaccctccgattgttcctgaaccgatgagaaa
taaagtttctgttgataatcatcg
bGH-pA
(SEQ ID No. 33)
actgtgccttctagttgccagccatctgttgtttgcccctcccccgtgcct
tccttgaccctggaaggtgccactcccactgccctttcctaataaaatgag
gaaattgcatcgcattgtctgagtaggtgtcattctattctggggggtggg
gggggcaggacagcaagggggaggattgggaagacaatagcaggcatgctg
gggatgcggtgggctctatgg
hPGK promoter
(SEQ ID No. 34)
ccacggggttggggttgcgccttttccaaggcagccctgggtttgcgcagg
gacgcggctgctctgggcgtggttccgggaaacgcagcggcgccgaccctg
ggtctcgcacattcttcacgtccgttcgcagcgtcacccggatcttcgccg
ctacccttgtgggccccccggcgacgcttcctgctccgcccctaagtcggg
aaggttccttgcggttcgcggcgtgccggacgtgacaaacggaagccgcac
gtctcactagtaccctcgcagacggacagcgccagggagcaatggcagcgc
gccgaccgcgatgggctgtggccaatagcggctgctcagcagggcgcgccg
agagcagcggccgggaaggggcggtgcgggaggcggggtgtggggcggtag
tgtgggccctgttcctgcccgcgcggtgttccgcattctgcaagcctccgg
agcgcacgtcggcagtcggctccctcgttgaccgaatcaccgacctctctc
cccagg
eGFP
(SEQ ID No. 35)
atggtgagcaagggcgaggagctgttcaccggggggtgcccatcctggtcg
agctggacggcgacgtaaacggccacaagttcagcgtgtccggcgagggcg
agggcgatgccacctacggcaagctgaccctgaagttcatctgcaccaccg
gcaagctgcccgtgccctggcccaccctcgtgaccaccctgacctacggcg
tgcagtgcttcagccgctaccccgaccacatgaagcagcacgacttcttca
agtccgccatgcccgaaggctacgtccaggagcgcaccatcttcttcaagg
acgacggcaactacaagacccgcgccgaggtgaagttcgagggcgacaccc
tggtgaaccgcatcgagctgaagggcatcgacttcaaggaggacggcaaca
tcctggggcacaagctggagtacaactacaacagccacaacgtctatatca
tggccgacaagcagaagaacggcatcaaggtgaacttcaagatccgccaca
acatcgaggacggcagcgtgcagctcgccgaccactaccagcagaacaccc
ccatcggcgacggccccgtgctgctgcccgacaaccactacctgagcaccc
agtccgccctgagcaaagaccccaacgagaagcgcgatcacatggtcctgc
tggagttcgtgaccgccgccgggatcactctcggcatggacgagctgtaca
agtaa
SV40-pA
(SEQ ID No. 36)
tgagatccagacatgataagatacattgatgagtttggacaaaccaaaact
agaatgcagtgaaaaaaatgccttatttgtgaaatttgtgatgctattgcc
ttatttgtaaccattataagctgcaataaacaagttaacaacaacaattgc
attcattttatgtttcaggttcagggggaggtgtgggaggttttttaaagc
aagtaaaacctctacaaatgtggtatggctgattatgatc
Homologous arm-right
(SEQ ID No. 37)
tacaatcatgtgggcagaattgaaaagtggagtgggaagggcaagggggag
ggttccctgcctcacgctacttcttctttctttcttgtttgtttgtttctt
tctttcttttgaggcagggtctcactatgttgcctaggctggtctcaaact
cctggcctctagtgatcctcctgcctcagcctttcaaagcaccaggattac
agacatgagccaccgtgcttggcctcctccttctgaccatcatttctcttt
ccctccctgcct
5′-ITR
(SEQ ID No. 38)
agtgatggagttggccactccctctctgcgcgctcgctcgctcactgaggc
cgggcgaccaaaggtcgcccgacgcccgggcggcctcagtgagcgagcgag
cgcgcagctgcctgcagg

In another embodiment, the gene editing targets RAG-1. In another embodiment, the donor template targets RAG-1.

### Nucleotide of Interest

The vector used in the present invention preferably comprises one or more nucleotides of interest.

Preferably, the nucleotide of interest gives rise to a therapeutic effect.

Suitably, the one or more NOIs for use in the present invention may be selected from: a guide RNA, a nucleotide encoding a Cas9 ribonucleoprotein, nucleotide sequences encoding one or more adenoviral proteins, nucleotide sequences encoding an agent which promotes homology directed DNA repair (such as an inhibitor of p53 activation or nucleotide sequences encoding one or more adenoviral proteins).

Suitable NOIs include, but are not limited to sequences encoding enzymes, cytokines, chemokines, hormones, antibodies, anti-oxidant molecules, engineered immunoglobulin-like molecules, single chain antibodies, fusion proteins, immune co-stimulatory molecules, immunomodulatory molecules, anti-sense RNA, microRNA, shRNA, siRNA, guide RNA (gRNA, e.g. used in connection with a CRISPR/Cas system), ribozymes, miRNA target sequences, a transdomain negative mutant of a target protein, toxins, conditional toxins, antigens, tumour suppressor proteins, growth factors, transcription factors, membrane proteins, surface receptors, anti-cancer molecules, vasoactive proteins and peptides, anti-viral proteins and ribozymes, and derivatives thereof (such as derivatives with an associated reporter group). The NOIs may also encode pro-drug activating enzymes. Preferably, the NOI is a guide RNA (gRNA).

### Pharmaceutical Composition

In one embodiment, the cells of the present invention may be formulated for administration to subjects with a pharmaceutically acceptable carrier, diluent or excipient. Suitable carriers and diluents include isotonic saline solutions, for example phosphate-buffered saline, and potentially contain human serum albumin.

Handling of the cell therapy product is preferably performed in compliance with FACT-JACIE International Standards for cellular therapy.

Haematopoietic Cell, Haematopoietic Stem Cell, Haematopoietic Progenitor Cell and/or T Cell Transplantation

The present invention provides a population of haematopoietic cells, haematopoietic stem cells, haematopoietic progenitor cells and/or T cells, prepared according to a method of the invention for use in therapy, for example for use in gene therapy.

The use may be as part of a cell transplantation procedure, for example a haematopoietic stem cell transplantation procedure.

Haematopoietic stem cell transplantation (HSCT) is the transplantation of blood stem cells derived from the bone marrow (in this case known as bone marrow transplantation) or blood. Stem cell transplantation is a medical procedure in the fields of haematology and oncology, most often performed for people with diseases of the blood or bone marrow, or certain types of cancer.

Many recipients of HSCTs are multiple myeloma or leukaemia patients who would not benefit from prolonged treatment with, or are already resistant to, chemotherapy. Candidates for HSCTs include paediatric cases where the patient has an inborn defect such as severe combined immunodeficiency or congenital neutropenia with defective stem cells, and also children or adults with aplastic anaemia who have lost their stem cells after birth. Other conditions treated with stem cell transplants include sickle-cell disease, myelodysplastic syndrome, neuroblastoma, lymphoma, Ewing's Sarcoma, Desmoplastic small round cell tumour and Hodgkin's disease. More recently non-myeloablative, or so-called “mini transplant”, procedures have been developed that require smaller doses of preparative chemotherapy and radiation. This has allowed HSCT to be conducted in the elderly and other patients who would otherwise be considered too weak to withstand a conventional treatment regimen.

In one embodiment, a population of haematopoietic stem cells prepared according to a method of the invention is administered as part of an autologous stem cell transplant procedure.

In another embodiment, a population of haematopoietic stem cells prepared according to a method of the invention is administered as part of an allogeneic stem cell transplant procedure.

In one embodiment, a population of T cells prepared according to a method of the invention is administered as part of an autologous T cell transplant procedure.

In another embodiment, a population of T cells prepared according to a method of the invention is administered as part of an allogeneic T cell transplant procedure.

The term “autologous cell transplant procedure” as used herein refers to a procedure in which the starting population of cells (which are then transduced according to a method of the invention) is obtained from the same subject as that to which the transduced cell population is administered. Autologous transplant procedures are advantageous as they avoid problems associated with immunological incompatibility and are available to subjects irrespective of the availability of a genetically matched donor.

The term “allogeneic m cell transplant procedure” as used herein refers to a procedure in which the starting population of cells (which are then transduced according to a method of the invention) is obtained from a different subject as that to which the transduced cell population is administered. Preferably, the donor will be genetically matched to the subject to which the cells are administered to minimise the risk of immunological incompatibility.

Suitable doses of transduced cell populations are such as to be therapeutically and/or prophylactically effective. The dose to be administered may depend on the subject and condition to be treated, and may be readily determined by a skilled person.

Haematopoietic progenitor cells provide short term engraftment. Accordingly, gene therapy by administering transduced haematopoietic progenitor cells would provide a non-permanent effect in the subject. For example, the effect may be limited to 1-6 months following administration of the transduced haematopoietic progenitor cells.

Such haematopoietic progenitor cell gene therapy may be suited to treatment of acquired disorders, for example cancer, where time-limited expression of a (potentially toxic) anti-cancer nucleotide of interest may be sufficient to eradicate the disease.

The present invention may be useful in the treatment of the disorders listed in WO 1998/005635. For ease of reference, part of that list is now provided: cancer, inflammation or inflammatory disease, dermatological disorders, fever, cardiovascular effects, haemorrhage, coagulation and acute phase response, cachexia, anorexia, acute infection, HIV infection, shock states, graft-versus-host reactions, autoimmune disease, reperfusion injury, meningitis, migraine and aspirin-dependent anti-thrombosis; tumour growth, invasion and spread, angiogenesis, metastases, malignant, ascites and malignant pleural effusion; cerebral ischaemia, ischaemic heart disease, osteoarthritis, rheumatoid arthritis, osteoporosis, asthma, multiple sclerosis, neurodegeneration, Alzheimer's disease, atherosclerosis, stroke, vasculitis, Crohn's disease and ulcerative colitis; periodontitis, gingivitis; psoriasis, atopic dermatitis, chronic ulcers, epidermolysis bullosa; corneal ulceration, retinopathy and surgical wound healing; rhinitis, allergic conjunctivitis, eczema, anaphylaxis; restenosis, congestive heart failure, endometriosis, atherosclerosis or endosclerosis.

In addition, or in the alternative, the invention may be useful in the treatment of the disorders listed in WO 1998/007859. For ease of reference, part of that list is now provided: cytokine and cell proliferation/differentiation activity; immunosuppressant or immunostimulant activity (e.g. for treating immune deficiency, including infection with human immune deficiency virus; regulation of lymphocyte growth; treating cancer and many autoimmune diseases, and to prevent transplant rejection or induce tumour immunity); regulation of haematopoiesis, e.g. treatment of myeloid or lymphoid diseases; promoting growth of bone, cartilage, tendon, ligament and nerve tissue, e.g. for healing wounds, treatment of burns, ulcers and periodontal disease and neurodegeneration; inhibition or activation of follicle-stimulating hormone (modulation of fertility); chemotactic/chemokinetic activity (e.g. for mobilising specific cell types to sites of injury or infection); haemostatic and thrombolytic activity (e.g. for treating haemophilia and stroke); anti-inflammatory activity (for treating e.g. septic shock or Crohn's disease); as antimicrobials; modulators of e.g. metabolism or behaviour; as analgesics; treating specific deficiency disorders; in treatment of e.g. psoriasis, in human or veterinary medicine.

In addition, or in the alternative, the invention may be useful in the treatment of the disorders listed in WO 1998/009985. For ease of reference, part of that list is now provided: macrophage inhibitory and/or T cell inhibitory activity and thus, anti-inflammatory activity; anti-immune activity, i.e. inhibitory effects against a cellular and/or humoral immune response, including a response not associated with inflammation; inhibit the ability of macrophages and T cells to adhere to extracellular matrix components and fibronectin, as well as up-regulated of receptor expression in T cells; inhibit unwanted immune reaction and inflammation including arthritis, including rheumatoid arthritis, inflammation associated with hypersensitivity, allergic reactions, asthma, systemic lupus erythematosus, collagen diseases and other autoimmune diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, eclampsia, pre-eclampsia and other immune and/or inflammatory-related gynaecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of stokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, myasthenia gravis, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery, bone marrow transplantation or other transplantation complications and/or side effects, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukaemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue.

In addition, or in the alternative, the invention may be useful in the treatment of β-thalassemia, chronic granulomatous disease, metachromatic leukodystrophy, mucopolysaccharidoses disorders and other lysosomal storage disorders.

### Kit

In one aspect, the invention provides a kit comprising one or more inhibitors according to the invention and/or cell populations of the invention.

In another aspect, the present invention provides a kit comprising one or more inhibitors according to the invention, one or more nucleotide sequences encoding gene editing machinery and means for selecting haematopoietic stem cells.

The one or more inhibitors according to the invention and/or cell populations may be provided in suitable containers.

Suitably, the kit may comprise a MAPK inhibitor. Suitably, the kit may comprise an IL-1 inhibitor. Suitably, the kit may comprise an NF-κB inhibitor. Suitably, the kit may comprise an MAPK inhibitor and an IL-1 inhibitor. Suitably, the kit may comprise a MAPK inhibitor and an NF-κB inhibitor. Suitably, the kit may comprise an IL-1 inhibitor and an NF-κB inhibitor. Suitably, the kit may comprise a MAPK inhibitor, an IL-1 inhibitor and an NF-κB inhibitor.

Suitably, the kit may comprise a nucleic acid sequence encoding at least one adenoviral protein

The kit may also include instructions for use.

### Method of Treatment

In a further aspect, the invention provides a method of gene therapy comprising the steps:
(a) gene editing a population of haematopoietic cells, haematopoietic stem cells, haematopoietic progenitor cells and/or T cells according to the method of the invention; and
(b) administering the gene edited haematopoietic cells, haematopoietic stem cells, haematopoietic progenitor cells and/or T cells to a subject.

In some embodiments, the gene edited cells are administered to a subject as part of an autologous stem cell transplant procedure or an allogeneic stem cell transplant procedure.

In a further aspect, the invention provides a method of gene therapy comprising the steps:
(a) transducing a population of haematopoietic cells, haematopoietic stem cells, haematopoietic progenitor cells and/or T cells according to the method of the invention; and
(b) administering the transduced population of haematopoietic cells, haematopoietic stem cells, haematopoietic progenitor cells and/or T cells to a subject.

In some embodiments, step (b) comprises administering the transduced cells to a subject as part of an autologous stem cell transplant procedure or an allogeneic stem cell transplant procedure.

The method of gene therapy may be, for example, a method of treatment of a disease selected from the group consisting of mucopolysaccharidosis type I (MPS-1), chronic granulomatous disorder, Fanconi anaemia (FA), sickle cell disease, metachromatic leukodystrophy (MLD), globoid cell leukodystrophy (GLD), GM2 gangliosidosis, thalassemia and cancer.

The method of gene therapy may be, for example, a method of treatment of diseases caused by Rag-1 mutations, eg SCID, atypical SCID and Omenn syndrome

In one embodiment, the subject is a mammalian subject, preferably a human subject.

In a further aspect, the invention provides a gene edited population of haematopoietic cells, haematopoietic stem cells, haematopoietic progenitor cells and/or T cells prepared according to the method of the invention.

In a further aspect, the invention provides a transduced population of haematopoietic cells, haematopoietic stem cells, haematopoietic progenitor cells and/or T cells prepared according to the method of the invention.

In a further aspect, the invention provides a pharmaceutical composition comprising the population of gene edited haematopoietic cells, haematopoietic stem cells, haematopoietic progenitor cells and/or T cells of the invention.

In a further aspect, the invention provides a pharmaceutical composition comprising the population of transduced haematopoietic cells, haematopoietic stem cells, haematopoietic progenitor cells and/or T cells of the invention.

In a further aspect, the invention provides the population of gene edited haematopoietic cells, haematopoietic stem cells, haematopoietic progenitor cells and/or T cells of the invention for use in therapy, preferably gene therapy.

In a further aspect, the invention provides the population of transduced haematopoietic cells, haematopoietic stem cells, haematopoietic progenitor cells and/or T cells of the invention for use in therapy, preferably gene therapy.

In some embodiments, the population is administered as part of an autologous stem cell transplant procedure or an allogeneic stem cell transplant procedure.

It is to be appreciated that all references herein to treatment include curative, palliative and prophylactic treatment; although in the context of the invention references to preventing are more commonly associated with prophylactic treatment. In one embodiment, the treatment of mammals, particularly humans, is preferred. Both human and veterinary treatments are within the scope of the invention.

The skilled person will understand that they can combine all features of the invention disclosed herein without departing from the scope of the invention as disclosed.

### Administration

Although the inhibitors and/or cells for use in the invention (in particular, the populations of cells produced by a method of the invention) can be administered alone, they will generally be administered in admixture with a pharmaceutical carrier, excipient or diluent, particularly for human therapy.

### Dosage

The skilled person can readily determine an appropriate dose of one of the agents (e.g. inhibitors and/or cells) of the invention to administer to a subject without undue experimentation. Typically, a physician will determine the actual dosage which will be most suitable for an individual patient and it will depend on a variety of factors including the activity of the specific agent employed, the metabolic stability and length of action of that agent, the age, body weight, general health, sex, diet, mode and time of administration, rate of excretion, drug combination, the severity of the particular condition, and the individual undergoing therapy. There can of course be individual instances where higher or lower dosage ranges are merited, and such are within the scope of the invention.

### Subject

A “subject” refers to either a human or non-human animal.

Examples of non-human animals include vertebrates, for example mammals, such as non-human primates (particularly higher primates), dogs, rodents (e.g. mice, rats or guinea pigs), pigs and cats. The non-human animal may be a companion animal.

Preferably, the subject is a human.

### Variants, Derivatives, Homologues and Fragments

In addition to the specific proteins and nucleotides mentioned herein, the present invention also encompasses the use of variants, derivatives, analogues, homologues and fragments thereof.

In the context of the present invention, a variant of any given sequence is a sequence in which the specific sequence of residues (whether amino acid or nucleic acid residues) has been modified in such a manner that the polypeptide or polynucleotide in question retains at least one of its endogenous functions. A variant sequence can be obtained by addition, deletion, substitution, modification, replacement and/or variation of at least one residue present in the naturally-occurring protein.

Variant sequences of SEQ ID NOs recited herein may have at least 80%, 85%, 90%, 95%, 98% or 99% sequence identity to the reference sequence SEQ ID NOs. Preferably, the variant sequence retains one or more functions of the reference sequence (i.e. is a functional variant).

Variant sequences may comprise one or more conservative substitutions. Conservative amino acid substitutions may be made on the basis of similarity in polarity, charge, solubility, hydrophobicity, hydrophilicity, and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

The present invention also encompasses homologous substitution (substitution and replacement are both used herein to mean the interchange of an existing amino acid residue, with an alternative residue) variants i.e. like-for-like substitution such as basic for basic, acidic for acidic, polar for polar etc.

Unless otherwise explicitly stated herein by way of reference to a specific, individual amino acid, amino acids may be substituted using conservative substitutions as recited below.

An aliphatic, non-polar amino acid may be a glycine, alanine, proline, isoleucine, leucine or valine residue.

An aliphatic, polar uncharged amino may be a cysteine, serine, threonine, methionine, asparagine or glutamine residue.

An aliphatic, polar charged amino acid may be an aspartic acid, glutamic acid, lysine or arginine residue.

An aromatic amino acid may be a histidine, phenylalanine, tryptophan or tyrosine residue.

Suitably, a conservative substitution may be made between amino acids in the same line in the Table above.

As used herein, “sequence identity” is determined by comparing the sequence of the reference amino acid sequence to that portion of another amino acid sequence so aligned so as to maximize overlap between the two sequences while minimizing sequence gaps, wherein any overhanging sequences between the two sequences are ignored.

Homology comparisons can be conducted by eye or, more usually, with the aid of readily available sequence comparison programs. These commercially available computer programs can calculate percentage homology or identity between two or more sequences.

Percentage homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the nucleotide sequence may cause the following codons to be put out of alignment, thus potentially resulting in a large reduction in percent homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum percentage homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al. (1984) Nucleic Acids Res. 12: 387). Examples of other software that can perform sequence comparisons include, but are not limited to, the BLAST package (see Ausubel et al. (1999) ibid—Ch. 18), FASTA (Atschul et al. (1990) J. Mol. Biol. 403-410) and the GENEWORKS suite of comparison tools. Both BLAST and FASTA are available for offline and online searching (see Ausubel et al. (1999) ibid, pages 7-58 to 7-60). However, for some applications, it is preferred to use the GCG Bestfit program. Another tool, called BLAST 2 Sequences is also available for comparing protein and nucleotide sequences (see FEMS Microbiol. Lett. (1999) 174: 247-50; FEMS Microbiol. Lett. (1999) 177: 187-8).

Although the final percent homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see the user manual for further details). For some applications, it is preferred to use the public default values for the GCG package, or in the case of other software, the default matrix, such as BLOSUM62.

Once the software has produced an optimal alignment, it is possible to calculate percent homology, preferably percent sequence identity. The software typically does this as part of the sequence comparison and generates a numerical result.

The term “homology” may be equated with the term “identity”.

Preferably, reference to a sequence which has a percent identity to any one of the SEQ ID NOs detailed herein refers to a sequence which has the stated percent identity over the entire length of the SEQ ID NO referred to.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The publications discussed herein are provided solely for their disclosure prior to the filing date of the present application. Nothing herein is to be construed as an admission that such publications constitute prior art to the claims appended hereto.

The invention will now be further described by way of Examples, which are meant to serve to assist one of ordinary skill in the art in carrying out the invention and are not intended in any way to limit the scope of the invention.

### EXAMPLES

### Example 1—Anakinra Treatment at the Time of Gene Editing Improves HSPC Clonogenic Potential

The present inventors previously reported the induction of the human hematopoietic stem and progenitors cells (HSPCs) DNA Damage Response (DDR)-dependent pro-inflammatory programs and its downstream impact on edited HSPC function (Schiroli et al., 2019, Cell Stem Cell 24: 551-565). The inventors hypothesised that DDR-dependent inflammation would also impact gene editing (GE) of HSPCs. In order to counteract the reported and/or potential effects of DDR-dependent inflammation, the present inventors employed Anakinra, the receptor antagonist of IL-1 (Cavalli and Dinarello, 2018, Front. Pharmacol. 9: 1157). IL-1 is reportedly an upstream mediator of DDR-dependent inflammation (Pietras, 2017, Blood 130: 1693-1698; and Gnani et al., 2019, Aging Cell 18: e12933).

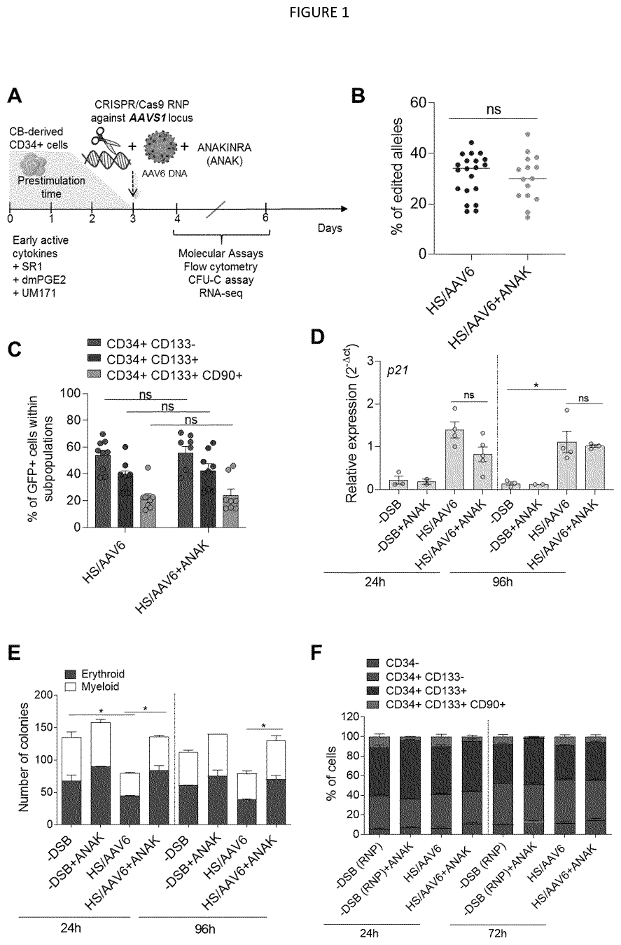

We edited the Adeno-Associated Virus Site 1 (AAVS1) locus as a model safe harbor for targeted transgene insertion (Lombardo, A. et al., 2011, Nat. Methods 8: 861-869) in human cord blood (CB) HSPCs by electroporating CRISPR/SpCas9 ribonucleoprotein (RNP) with a highly specific chemically modified guide RNA (gRNA) (Schiroli et al., 2019, Cell Stem Cell 24: 551-565). Moreover, we added Anakinra immediately after the transduction of the GFP-expressing repair template AAV6 (HS/AAV6 hereafter for gene-edited cells and HS/AAV6+ANAK for edited HSPC in presence of Anakinra). As negative controls, we employed a RNP with a guide RNA with no predicted activity in the human genome in presence (−DSB+ANAK) or absence of Anakinra (−DSB) (FIG. 1A). We first verified that Anakinra treatment maintained the efficiency of GE across all HSPC subpopulations, from the more committed to the more primitive subfractions, as demonstrated by equal percentage of HDR-edited alleles comparing Anakinra-treated edited cells to cells treated with our standard protocol (FIG. 1B, C). We next measured the expression of the DDR downstream effector CDKN1A (hereafter named p21), as surrogate marker for DDR activation as previously reported (Schiroli et al., 2019, Cell Stem Cell 24: 551-565). p21 levels were upregulated upon GE compared to negative controls independently from Anakinra treatment (FIG. 1D) and this effect was maintained 24 and 96 hours (h) upon treatment. In addition, significantly fewer colonies were generated in methylcellulose assays from gene-edited HSPCs (FIG. 1E), while we found a significant increase in HSPC clonogenic potential upon Anakinra treatment which was maintained late upon GE (FIG. 1E). No treatment skewed culture composition over time (FIG. 1F).

Overall, these data indicate that Anakinra treatment at the time of gene editing did not affect HSPC ability to repair via HDR and p21 levels but strongly improved HSPC clonogenic potential in vitro.

### Example 2—Anakinra Dampens Pro-Inflammatory Programs Consequent to GE Procedure in HSPCs

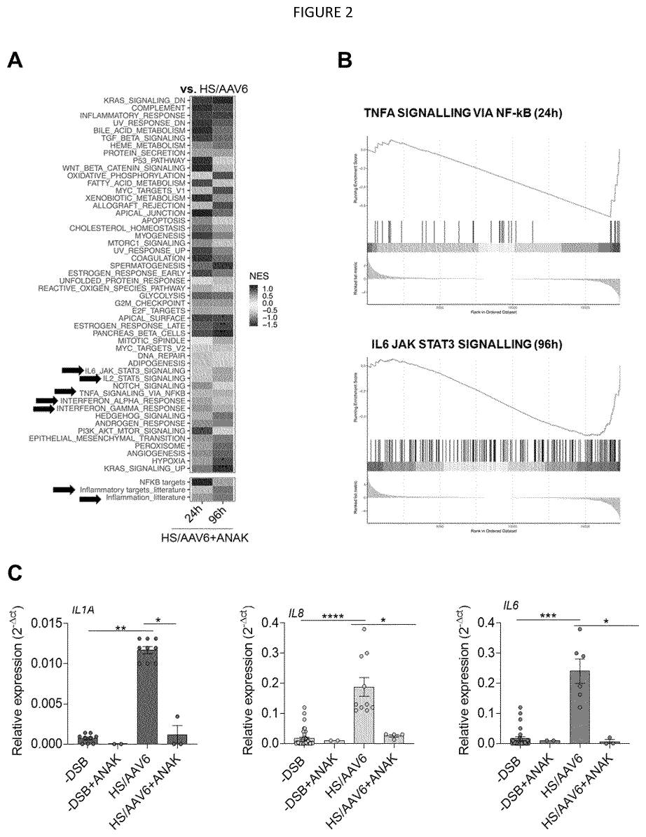

We next assessed whether Anakinra might have an impact on the transcriptional changes occurring upon GE, and we performed whole transcriptomic analysis on CB-derived HSPCs 24 and 96 h after AA VS locus editing, in order to investigate the early and late effects that Anakinra could exert on edited HSPCs. We tested editing in presence or absence of Anakinra in edited HSPC and their respective negative controls. We performed GSEA on gene lists ranked based on log 2FC to identify the pathways modulated by Anakinra. We found negative and significantly high normalized enrichment scores (NES) for inflammatory/TNFα dependent pathways (TNF-α signaling via nuclear factor kB [NFkB]; IL2-STAT5 signalling; IL-6/JAK/STAT3 signaling) and interferon responses in cells edited in presence of Anakinra in respect to not treated counterpart (FIG. 2A,B). Accordingly, we observed a significant upregulation of key inflammatory genes of the IL-1 signaling pathway in edited HSPCs, which are downregulated 96 h upon Anakinra treatment (FIG. 2C).

Altogether, these studies suggest that the induction of pro-inflammatory programs early and late upon gene editing are downregulated early and late upon Anakinra treatment.

### Example 3—Blocking IL-1 Signalling Pathway is a Way to Preserve In Vivo Long-Term Reconstitution of HDR-Edited Cells

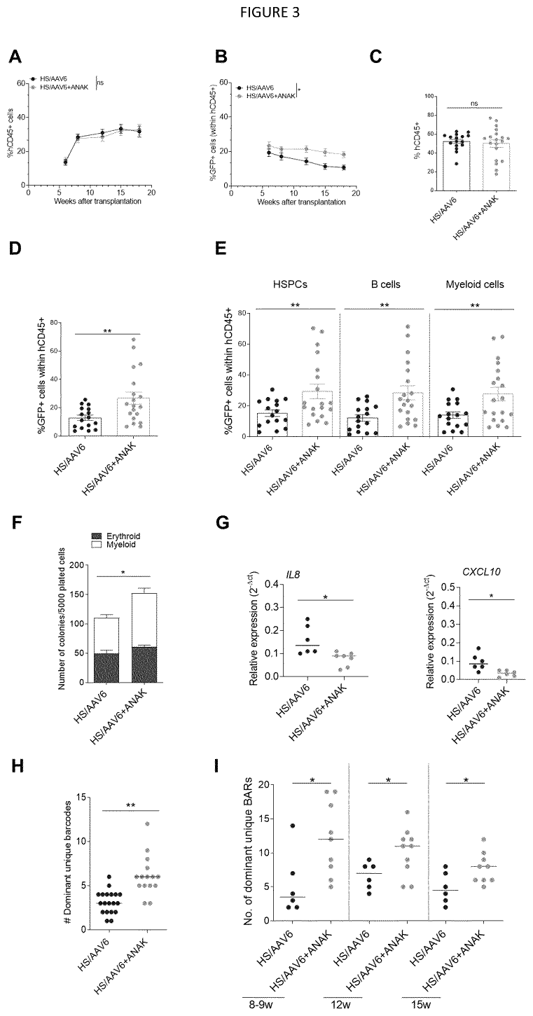

To investigate the repopulating potential of HSPCs edited in presence or absence of Anakinra, we transplanted matched saturating cell doses into NOD Prkdcscid II2rg−/− (NSG) mice. Moreover, we used as positive control cells edited in presence of p53 inhibition (co-electroporating an mRNA encoding for a dominant negative p53 truncated form (GSE56) as previously reported (Schiroli et al., 2019, Cell Stem Cell 24: 551-565; and Ferrari et al., 2020, Nat Biotechnol 38: 1298-1308)) alone or in combination with Anakinra. We observed similar human engraftment across treatments, which reached a plateau of 35-40% circulating cells (FIG. 3A). Similar patterns of engraftment were also found long-term after reconstitution in the hematopoietic organs (FIG. 3C). Considering the human repopulation of HDR-edited HSPCs, we observed higher and stable percentage of GFP+ cells when cells are treated with Anakinra compared to standard protocol (FIG. 3B). The percentage of GFP+ cells within the human graft, sorted progenitors and individual lineages were consistent with the levels observed in the blood, with Anakinra treated HDR-edited cells showing an improved long-term reconstitution compared to standard protocol edited cells (FIG. 3D,E).

In addition, when we re-challenged human CD34+ cells retrieved from the bone marrow of primary recipients into semisolid medium, we found that Anakinra-treated edited HSPCs displayed higher clonogenic potential compared to standard protocol treated cells (FIG. 3F) which correlates with a decrease in pro-inflammatory cytokines expression (FIG. 3G).

In order to assess the clonal composition of host repopulation by Anakinra-edited HSPCs, we embedded a 22-basepair (bp) degenerated heritable ‘barcode’ sequence (BAR) in the repair template downstream of a green fluorescent protein (GFP) reporter cassette. We then generated a plasmid library and an AAV6 pool of high and comparable complexity (7.5×10⁵ and 5.9×10⁵ unique BARs, respectively) and nearly homogeneous representation of degenerated consensus sequences (Ferrari et al., 2020, Nat Biotechnol 38: 1298-1308). We found that Anakinra-treated edited HSPCs show a higher polyclonal reconstitution compared to standard gene edited cells, with increasing number of dominant BARs compared to respective control (FIG. 3H). Polyclonal reconstitution after Anakinra treatment was also confirmed by analyzing PBMCs in the saturating-dose experiment (FIG. 3G).

Overall, these data suggest that HDR-edited HSPCs in presence of Anakinra displayed a higher long-term polyclonal reconstitution, which is associated to a dampening of pro-inflammatory cytokines.

### Example 4—Anakinra Reduces NF-κB Nuclear Translocation Upon GE

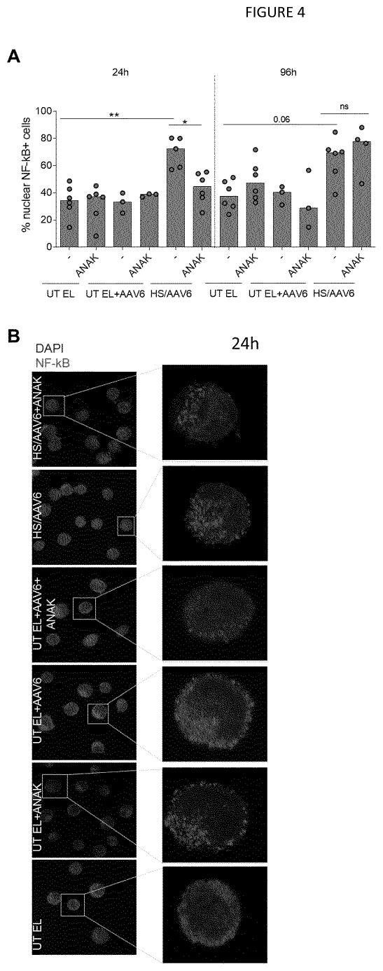

To study the molecular mechanism at the basis of IL-1 mediated inflammation, we measured the nuclear factor-kappa Beta (NF-kB) localization, as a surrogate marker for its activation, as previously reported. We treated edited cells with Anakinra during the gene editing procedure, and we used additional controls to discriminate Anakinra effect. We performed the sole electroporation of the cells (hereafter UT electro sample), the transduction of the cells after their electroporation (hereafter UT electro+ AAV6 sample) and the standard gene editing procedure (HS/AAV6 sample) in presence or absence of Anakinra. We found an increase in NF-kB nuclear translocation upon gene editing compared to negative controls (UT electro and UT electro+AAV6) and a decrease upon Anakinra treatment (FIG. 4A-B), suggesting that IL-1 signaling pathway is upstream NF-kB activation upon GE. According to the transient nature of Anakinra treatment, we no longer observed NF-kB activation 96 h post GE.

### Example 5—NF-kB Inhibition Improves HSPC Long-Term Reconstitution and Synergizes with p53 Inhibition

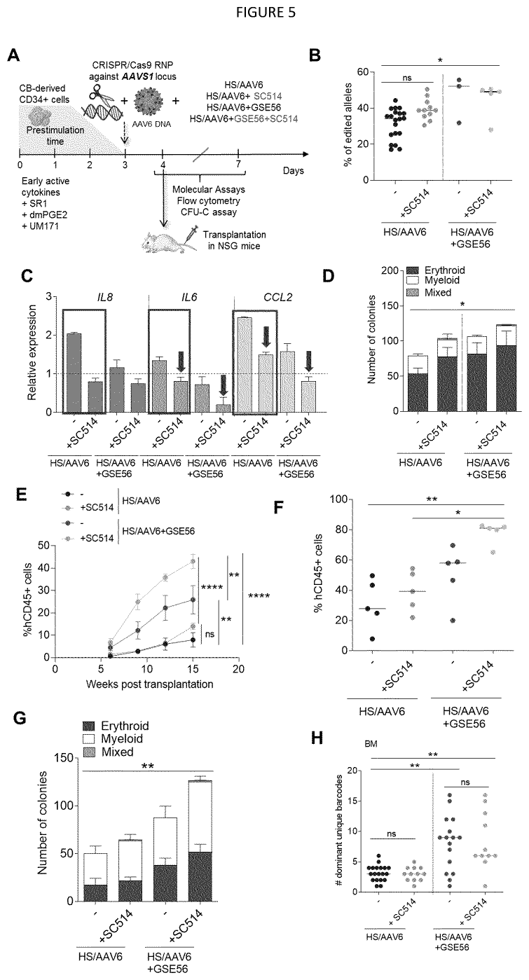

In order to more broadly inhibit NF-kB pathway to have a higher improvement in edited HSPC functionality, we employed an IKK-2 Inhibitor (SC514), a suppressor of the NF-kB-dependent inflammatory transcriptional program (Kishore, N. et al., 2003, Journal of Biological Chemistry 278: 32861-32871; and Roman-Blas, J. A., & Jimenez, S. A., 2006, Osteoarthritis Cartilage 14: 839-848). We treated cells with SC514 at the time of GE of the AAVS1 locus and we performed p53 inhibition alone or in combination to NF-kB inhibitor, collecting cells 24 and 96 h upon GE (FIG. 5A). We observed no negative impact on the efficiency of genome editing in terms of HDR-edited alleles comparing edited HSPC in presence or absence of NF-kB inhibition. On the contrary, we found that GSE56 tended to increase the percentage of HDR editing and this effect was maintained upon NF-kB inhibition (FIG. 5B). Moreover, since NF-kB is an upstream mediator of pro-inflammatory program, we observed a decrease in the expression levels of some upregulated DDR-related cytokines (such as IL-6, IL-8 and CCL2) in SC514-treated edited HSPCs. In addition, we also observed a downregulation of the cytokines in presence of GSE56, which was even exacerbated when performing GSE56 and SC514 simultaneous (i.e. combination) treatments (FIG. 5C). Colony-forming potential was similarly increased for editing treatment in presence of SC514 or GSE56 as compared to standard protocol edited HSPCs, with a trend toward more colonies for GSE56/SC514 combination, without detectable difference in erythroid to myeloid ratio (FIG. 5D). To investigate the repopulation potential of HSPCs edited in presence or absence of SC514 and GSE56, we transplanted matched limiting cell doses into NSG mice and we confirmed that GSE56 addition allowed fourfold higher engraftment than the standard protocol, while its combination with SC514 even potentiate this increase, reaching nearly the 40% of human engraftment (FIG. 5E). Similar patterns of engraftment were found long-term after reconstitution in the hematopoietic organs (FIG. 5F). To further investigate long-term functionality of edited HSPCs, we performed a colony-forming assay by purifying human CD34+ cells from the bone marrow of primary recipients (FIG. 5G). We showed a small increase in the clonogenic potential of the cells upon SC514 treatment that became greater upon combination with p53 inhibition. Moreover, we observed a significant increase in the fitness of edited HSPC when combining NF-kB and p53 inhibition, suggesting a synergistic effect. Finally, by performing clonal tracking of HDR-edited HSPC by a barcoding-based strategy, we also found that NF-kB inhibition improved polyclonal reconstitution and preserved self-renewal and multi-potency of individual edited HSPC in combination with GSE56.

### Example 6—Edited HSPCs Pre-Treated with p38 Inhibitor Show Increased Clonogenic Capacity and Decreased ROS Levels

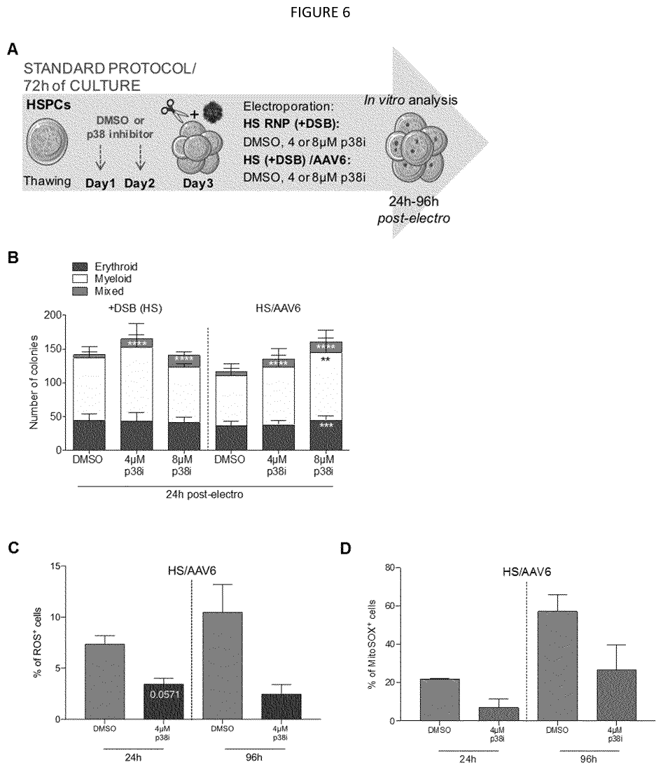

In order to ameliorate culture conditions and eventually achieve more functional edited HSPC, we reasoned to inhibit p38-MAPK signalling by employing SB203580, a highly specific ATP-competitive inhibitor of its kinase activity, that does not affect protein phosphorylation. Specifically, we decided to treat HSPCs during the first 2 days of culture, since the standard gene editing protocol requires to perform gene-editing at day 3 post thawing. In detail, HSPCs were treated with DMSO or p38i (4 μM) at Day 1 and Day 2. At Day 3, HSPCs were electroporated with the HS RNP alone or in addition of the AAV6 GFP-expressing donor template as required for the standard gene editing protocol (Schiroli et al., 2019, Cell Stem Cell 24: 551-565) (FIG. 6A).

We next evaluated HSPC functionality testing their clonogenic capacity, we observed an increase in colony numbers after p38 inhibitor treatments in both HS RNP and HS/AAV6 conditions. Surprisingly, the higher number of colonies was mostly due to an increase in the number of mixed colonies, the progeny of more primitive and multipotent HSCs (Carow, C. E., Hangoc, G. and Broxmeyer, H. E, 1993, Blood, 81: 942-949) (FIG. 6B). Interestingly, we found that the 2 doses of p38 inhibitor 4 μM at day 1 and day 2 after thawing, were able to diminish ROS and mitochondrial superoxides accumulation in HSPCs upon editing (FIG. 6C).

### Example 7—Edited HSPCs Pre-Treated with p38 Inhibitor Display a Higher Engraftment and Number of Edited Clones Post-Transplant in NSG Mice

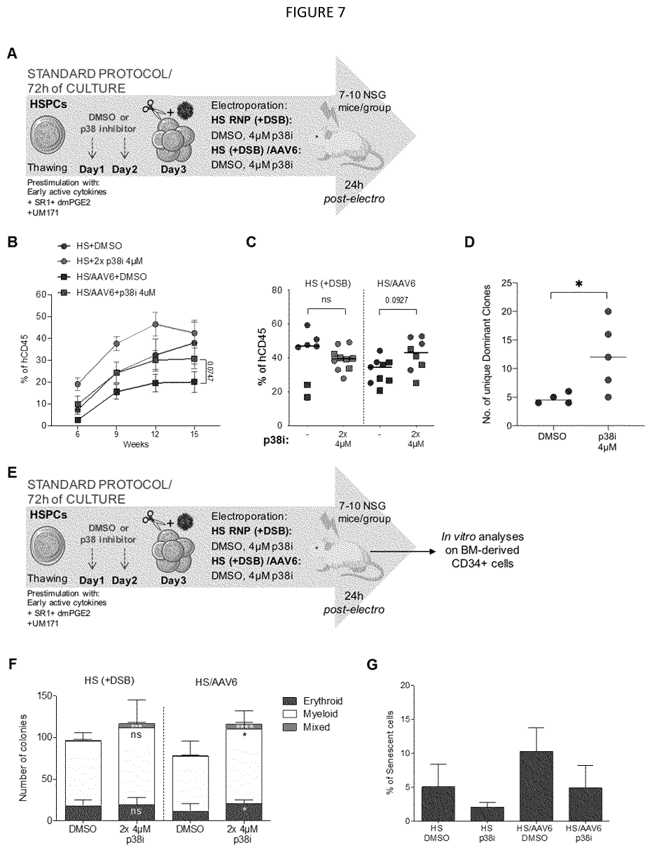

The increase in mixed colonies observed when we edited HSPCs upon p38 inhibition, suggested that the treatment could preserve the fitness of more primitive HSCs. Thus, we tested the in vivo functionality of HSPCs upon a single DSB (HS) or full editing (HS/AAV6) in presence or absence of p38 inhibition. We treated HSPCs at day 1 and day 2 with DMSO (as a control) or with 4 μM p38i and we transplanted CB-derived CD34⁺ HSPCs treated with the HS RNP alone or in combination with the AAV6 donor template into NOD Prkdcscid//2rg−/− (NSG) mice (FIG. 7A). We followed HSPC repopulating capacity by performing serological analysis at different time points upon transplantation (from 6 to 15 weeks), and by measuring the percentage of human CD45⁺ (hCD45⁺) cells in peripheral blood. We found that the percentage of hCD45⁺ cells of mice transplanted with HSPCs that experienced only the DSB (HS condition) pre-treated with p38 inhibitor was effectively increased up to 12 w but stabilized at 15 w and, the engraftment returned similar to DMSO control. Instead, the percentage of engrafting HS/AAV6+p38i treated HSPCs was, from the beginning, higher compared to DMSO control group (FIG. 7B). Data obtained in the Bone Marrow (BM) at the time of euthanasia (15 w), mirrored the findings in PB: the engraftment of HS condition was similar across the 2 treatments, while p38 inhibition improved HS/AAV6 engraftment (FIG. 7C). Given the promising results on the engraftment and the improved functionality of edited HSPCs upon p38 inhibitor treatment, we asked whether p38 inhibition could impact on the number of edited HSPC clones. Indeed, we selected for BAR-seq analysis mice transplanted with HSPCs edited with DMSO or p38 inhibitor with detectable engraftment (>0.1%) of GFP+ cells in the BM at 15 w (Ferrari et al., 2020, Nat Biotechnol 38: 1298-1308). We included 5 out of 6 mice of the HS/AAV6+DMSO group and 5 out of 6 of HS/AAV6+p38i 4 μM group. Interestingly, we found that the number of dominant BARs was significantly increased upon p38 inhibition, suggesting an enhanced polyclonal reconstitution of HDR-edited cells upon p38i treatment (FIG. 7D).

### Example 8—p38 Inhibition Increases HSPC Functionality at Long-Term Post-Transplantation and Reduces the Percentage of Senescent Cells

At time of euthanasia (15 w), we purified CD34+ cells from the bone marrow and tested their clonogenic potential in methylcellulose assay (FIG. 7E). Interestingly, we observed a higher number of colonies generated by BM-derived HS and HS/AAV6 HSPCs that were treated with p38 inhibitor, even if the differences were more evident in HS/AAV6 group. As previously stated, this increase in colony number was due to an increase in mixed colonies in both conditions upon p38i treatment (FIG. 7F).

In line with these results, we also observed a reduction in the presence of senescent HSPCs when pre-treated with the p38 inhibitor, corroborating the idea that p38 inhibition better preserves edited HSPC functionality (FIG. 7F).

### Example 9—Inhibition of Different MAPKs Increases HDR within Different Subpopulations and Ameliorates HSPC Clonogenic Potential

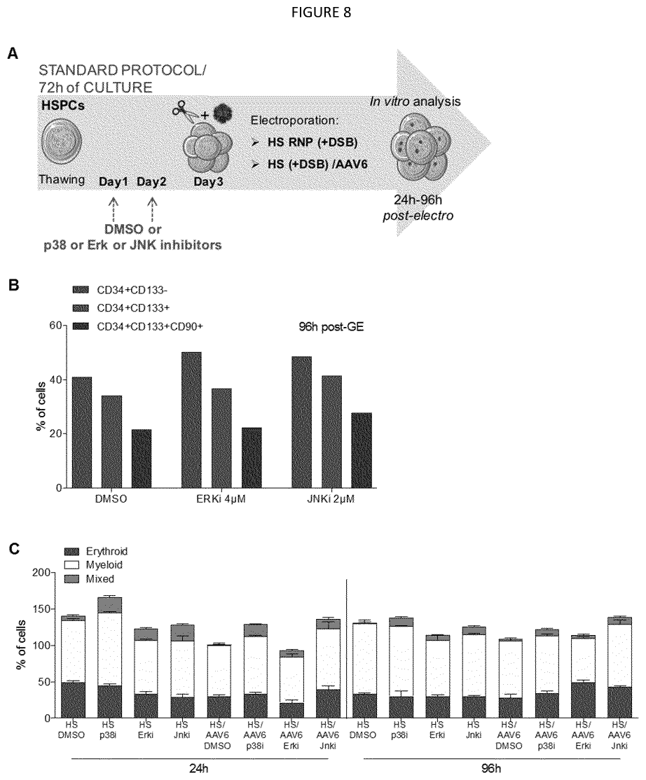

Given the promising results obtained by p38 inhibition, we reasoned that the activation of other mitogen-activated protein MAP kinases (MAPKs) could contribute to the loss of HSPCs functionality upon ex vivo expansion and genome editing procedures. Indeed, the family members of the MAPKs are evolutionarily conserved and are involved in the control of physiological cellular processes including cell proliferation, survival, differentiation, apoptosis and tumorigenesis. In mammalian cells, three distinct subgroups within the MAPK family have been described: namely classical MAPK (also known as ERK), C-Jun N-terminal kinase/stress-activated protein kinase (JNK/SAPK) and p38 kinase (Wei, Z. and Liu, H. T., 2002, Cell Research. Science Press 12: 9-18; and Cicenas et al., 2018, Cancers 10: 63). In detail, HSPCs were treated with DMSO, p38 (4 μM), Erk (4 μM) or Jnk (2 μM) inhibitors at Day 1 and Day 2. At Day 3 HSPCs were electroporated with the HS RNP alone or in addition of the AAV6 GFP-expressing donor template as required for the standard gene editing protocol (FIG. 8A). We investigated GE efficiency in HSPC subpopulations, from the more committed to the more primitive subfractions and we observed an increase in GFP levels upon Erk and Jnk inhibition and, interestingly, inhibition of the Jnk pathways resulted in a higher percentage of more primitive edited cells (FIG. 8B).

Moreover, we evaluated HSPC functionality testing their clonogenic capacity and we observed an increase in mixed colonies after all inhibitor treatments in both HS RNP and HS/AAV6 conditions (FIG. 8C).

### Example 10—p38 Inhibitor Treatment Reduces the Percentage of Mitochondrial Superoxides and Senescence

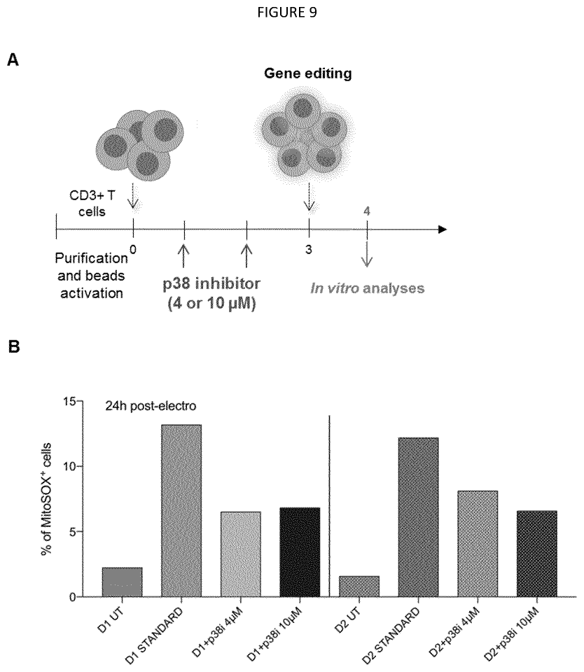

Since p38 inhibition ameliorated HSPC functionality upon gene editing, we thought to test it also in other cellular types clinically relevant in the contest of gene and cellular therapies. We started from CD3+ T cells purified from 2 different donors and we tested 2 concentrations of p38 inhibitor. In particular, T cells were treated at day 1 and day 2 with 4 or 10 μM p38i and edited at day 3 (FIG. 9A). 24 h post-editing we measured mitochondrial superoxides levels: we observed an increase of their levels upon gene editing compared to the non-edited control (UT) but, importantly, both doses of p38i reduced their levels (FIG. 9B).

Furthermore, we also observed an increase in cell growth and a decrease in senescence of edited T cells upon p38 treatments (data not shown).

### Example 11—Gene Editing Leads to the Activation of Senescence Programs Impairing HSPC Functionality Long-Term

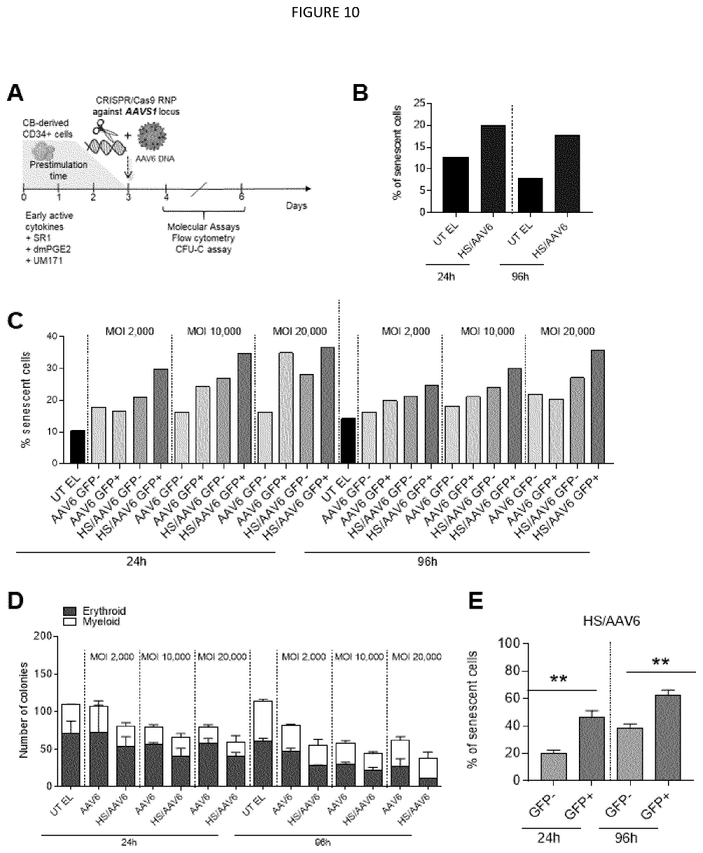

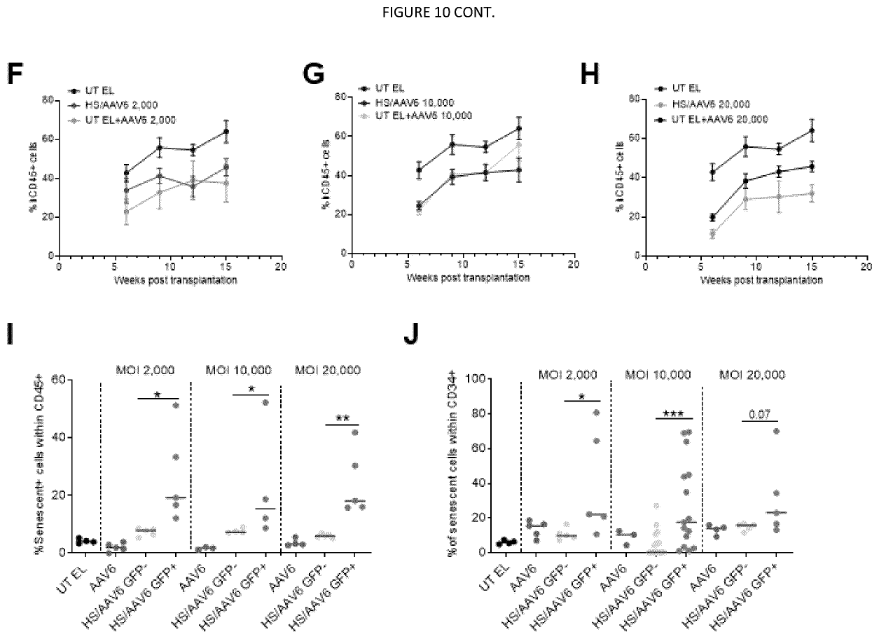

To investigate the unintended effects of the gene editing procedure on human HSC functions, we employed our optimized protocol consisting in culturing CB-derived CD34+ for 3 days in an early active cytokines and stem-preserving activity medium (Schiroli, G. et al., 2019, Cell Stem Cell 24: 551-565). Then, we performed the gene editing of the safe harbor AAVS1 locus with highly purified and clinical grade CRISPR-Cas9 ribonucleoprotein complex and an AAV6 delivering a GFP-expressing DNA repair template for HDR (FIG. 10A). We collected cells 24 hours (h) and 96 h upon gene editing and we found the increase of the senescence-associated marker p16 in edited cells (HS/AAV6) compared to cells experiencing the sole electroporation (untreated electro, UT EL) (FIG. 10B). In addition, we also measured the accumulation of the senescence markers lysosomal enzyme senescence-associated-β-galactosidase (SA-β-gal) comparing side by side all the reagents involved in the GE procedure and employing increasing doses of AAV6 as DNA donor template for HDR. We reported that transduction with increasing doses of AAV6 donor template triggers a senescence-like phenotype compared to only electroporated control cells 24 h upon GE, but this response was transient and resolved at 94 h post-editing. On the contrary, GE led to higher levels of senescence induction compared to the sole electroporation and AAV6 transduction and such a response was maintained over time especially in cells that have undergone HDR (GFP positive) (FIG. 10C). Moreover, while we reported HSPC impaired clonogenic potential upon increasing doses of AAV6 transduction, this impairment became more evident in the context of the full GE procedure (FIG. 10D). Interestingly, we also observed that the HDR-edited cells displayed senescence features in their clonogenic output, indeed we found increased levels of SA-β-gal in colonies derived from GFP+ cells compared to GFP− ones (FIG. 10E). Consistently, we found impaired long term hematopoietic reconstitution when edited cells were transplanted into NSG mice compared to UT EL controls, especially in cells experiencing GE with the highest AAV6 dose (FIGS. 10F, G, H).

Then, looking at the senescence burden in the human graft outgrown from the transplanted edited cells into NOD Prkdcscid II2rg−/− (NSG) mice, we reported an increase in senescence induction in HDR edited cells compared to not edited GFP− ones (light grey) and cells experiencing the sole electroporation (black) or AAV6 transduction (dark grey) in all human CD45+ cells (FIG. 101) as well as in BM-derived CD34+ cells (FIG. 1J). Overall, these data indicate that GE in HSPCs led to the establishment of a senescence program affecting HSPC functionality in the long-term.

### Example 12—Transient DDR Inhibition Prevents the Accumulation of Senescence in HDR-Edited HSPCs

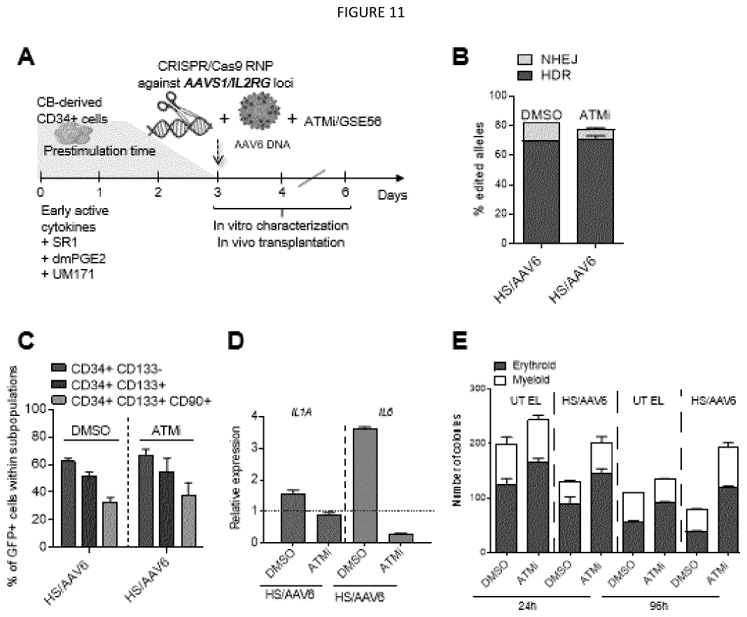

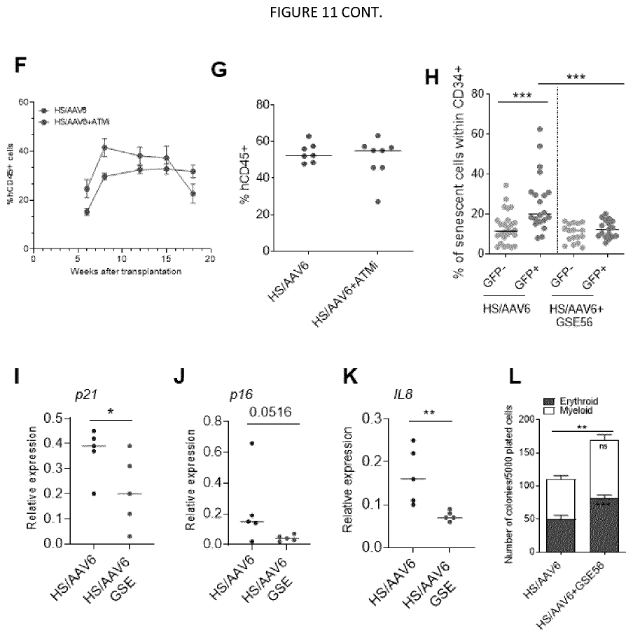

To assess the functional role of cell senescence in edited HSPCs, we temporarily inhibited the DDR pathway acting on the DDR apical kinase ATM and employing a p53 inhibitor (GSE56) at the time of ex-vivo editing (FIG. 11A). We reported that transient ATM inhibition within 15 minutes upon GE did not impair HSPC ability to repair via HDR (FIG. 11B). Indeed the percentage of GFP+ cells within the most primitive (CD34+CD133+CD90+ cells) and more committed progenitors (CD34+CD133−, CD34+CD133+) was similar in ATM inhibitor (ATMi)-treated edited HSPCs compared to the not treated counterpart (FIG. 11C).

The activation of inflammatory pathways is a key feature of senescent cells. Given that ATM kinase is reported to directly activate NF-kB (Fang, L. et al. Nucleic Acids Res 2014. 42: 8416-8432), the major transcription factor playing a crucial role in the transcriptional activation of pro-inflammatory cytokines, we reasoned that ATM could be also involved in GE-related inflammation. By performing a gene expression analysis of some key inflammatory cytokines, previously reported to be regulated by DDR, we found that edited HSPCs showed an upregulation of the reported IL1A, IL6 cytokines 24 h upon GE and ATM inhibition dampened this induction (FIG. 11D). Moreover, testing the functional properties of the cells, we found that edited HSPCs have a reduced clonogenic potential compared to negative control (UT EL DMSO) overtime that is improved upon ATM inhibition. However, we also reported a modest improvement in the clonogenic potential also for only electroporated cells upon ATM inhibition (FIG. 11E). In addition, we found a trend of increased engraftment early upon transplantation in vivo (FIG. 11F), confirming that ATMi improves edited HSPC repopulating potential and suggesting that ATMi mainly exerts its beneficial effects on HSPC progenitors involved in short-term repopulation. Indeed, we did not report any differences in terms of engraftment in BM-derived HSPCs in presence or absence of ATM inhibition (FIG. 11G). Since we previously reported that p53 inhibition is valuable strategy to increase HSPC engraftment and clonality (Schiroli, G. et al., 2019, Cell Stem Cell 24: 551-565, Ferrari, S. et al., 2020, Nat Biotech 38(11):1298-1308), we reasoned that GSE56 administration at the time of GE could also help us in reducing GE-mediated cellular senescence establishment. We found that p53 inhibition prevented the accumulation of the senescence marker SA-β-gal in HDR-edited BM-derived CD34+ cells (FIG. 11H). Finally, HSPCs harvested from the bone marrow of mice transplanted with edited cells (HS/AAV6) displayed increased mRNA levels of p21, p16 and IL8 that are mitigated by the administration of GSE56 at the time of ex-vivo editing (FIGS. 11I, 11J, 11K) and this was consistent with an improvement of HSPC clonogenic potential upon p53 inhibition when BM-derived cells were re-challenged in semisolid medium (FIG. 11L).

Altogether these results support the notion that gene editing may inadvertently trigger a DDR-dependent senescence program in transplanted HSPCs.

### Example 13—Anakinra Treatment at the Time of Gene Editing Improves HSPC Clonogenic Potential

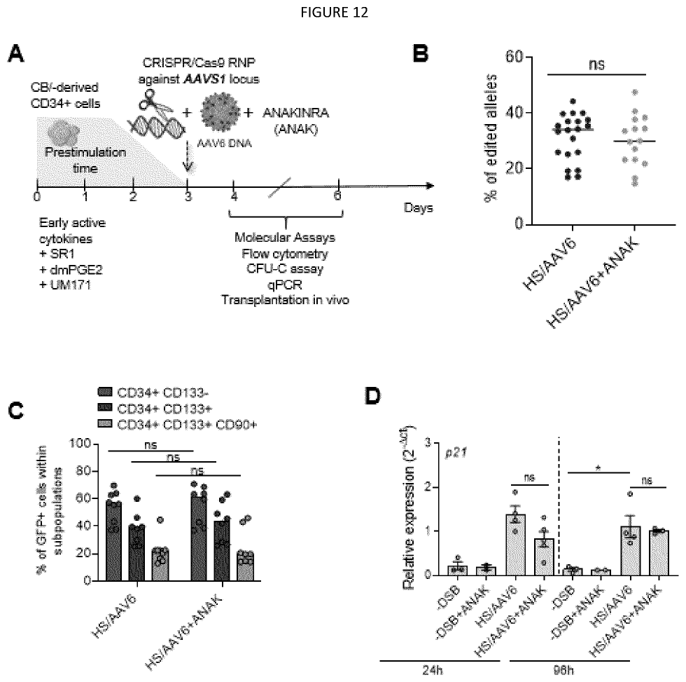

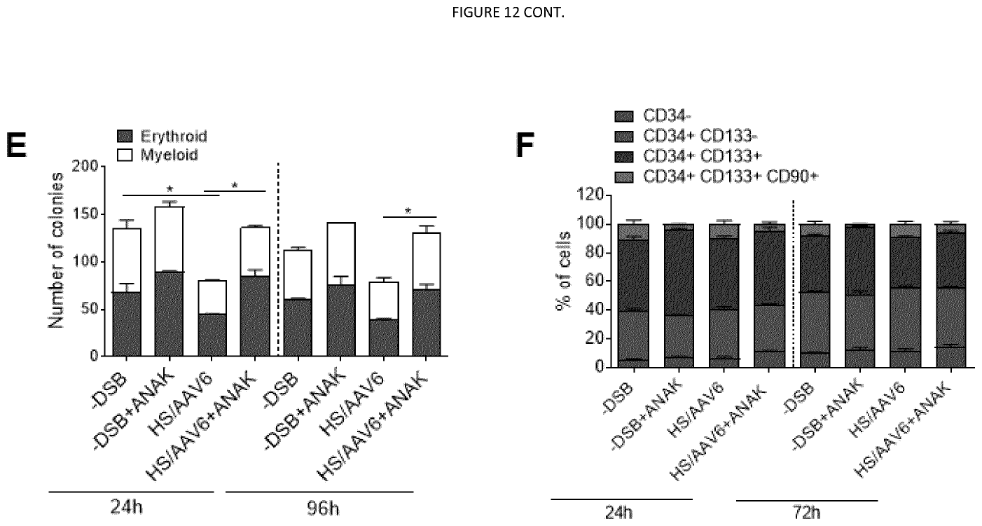

Given that p53 inhibition may aggravate the genotoxicity risks of gene editing procedure, we decided to modulate the downstream GE-related inflammation. In order to counteract the previously reported human hematopoietic stem and progenitors cells (HSPCs) pro-inflammatory and senescence-related programs induction and their downstream impact on edited HSPC function (Schiroli et al., 2019, Cell Stem Cell 24: 551-565), we employed Anakinra, the receptor antagonist of IL1 (Cavalli and Dinarello, 2018, Front. Pharmacol. 9: 1157), an extensively reported upstream mediator of HSC exhaustion, DDR-dependent inflammation, and a key component of the senescence-associated inflammatory programs (Pietras, 2017, Blood 130: 1693-1698; and Gnani et al., 2019, Aging Cell 18: e12933). Indeed, we reasoned that inhibiting IL1 signalling pathway within 15 minutes upon GE with Anakinra, a recombinant IL1 receptor antagonist, might help in preserving the long-term functionality of corrected HSPCs. We edited the Adeno-Associated Virus Site 1 (AAVS1) locus as a model safe harbor for targeted transgene insertion (Lombardo, A. et al., 2011, Nat. Methods 8: 861-869) in human cord blood (CB)-derived HSPCs by electroporating CRISPR/SpCas9 ribonucleoprotein (RNP) with a highly specific chemically modified guide RNA (gRNA) (Schiroli et al., 2019, Cell Stem Cell 24: 551-565). Moreover, we added Anakinra within 15 minutes after the transduction of the GFP-expressing repair template AAV6 (HS/AAV6 hereafter for gene-edited cells and HS/AAV6+ANAK for edited HSPC in presence of Anakinra). As negative controls, we employed an RNP with a guide RNA with no predicted activity in the human genome in presence (−DSB+ANAK) or absence of Anakinra (−DSB) (FIG. 12A). We first verified that Anakinra treatment did not affect the efficiency of GE, as demonstrated by equal percentage of HDR-edited alleles comparing Anakinra-treated edited cells to cells treated with our standard protocol (FIG. 12B). Furthermore, we found no significant differences in GE efficiency in all HSPC subpopulations, from the more committed to the more primitive subfractions (FIG. 12C). We next measured the expression of DDR downstream effector CDKN1A (hereafter named p21), as surrogate marker for DDR activation as previously reported (Schiroli et al., 2019, Cell Stem Cell 24: 551-565). p21 levels were upregulated upon GE compared to negative controls independently from Anakinra treatment (FIG. 12D) and this effect was maintained 24 and 96 h upon treatment. In addition, significantly fewer colonies were generated in methylcellulose assays from gene-edited HSPCs, while we found a significant increase in HSPC clonogenic potential upon Anakinra treatment, which was maintained late upon GE (FIG. 12E). No treatment skewed culture composition over time (FIG. 12F).

Overall, these data indicate that Anakinra treatment at the time of gene editing did not affect HSPC ability to repair via HDR and p21 levels but strongly improved HSPC clonogenic potential ex-vivo.

### Example 14—Anakinra Dampens Inflammation and Senescence Consequent to GE Procedure in HSPCs

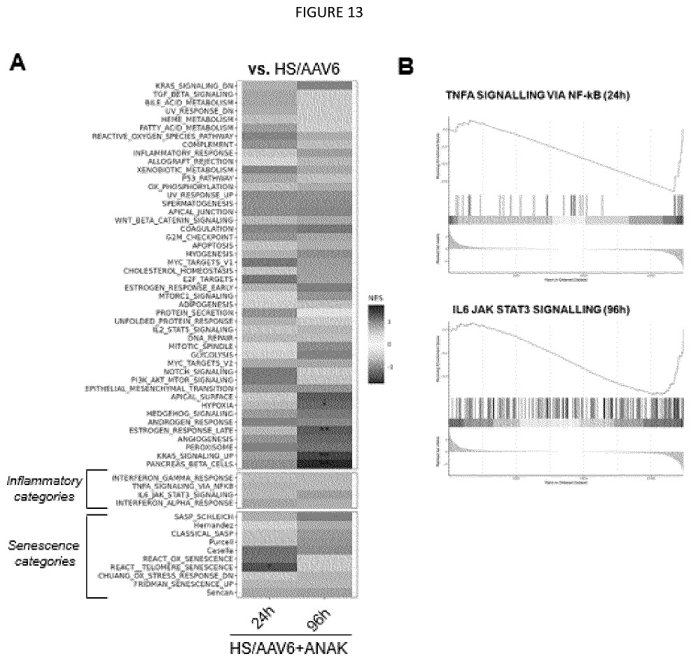

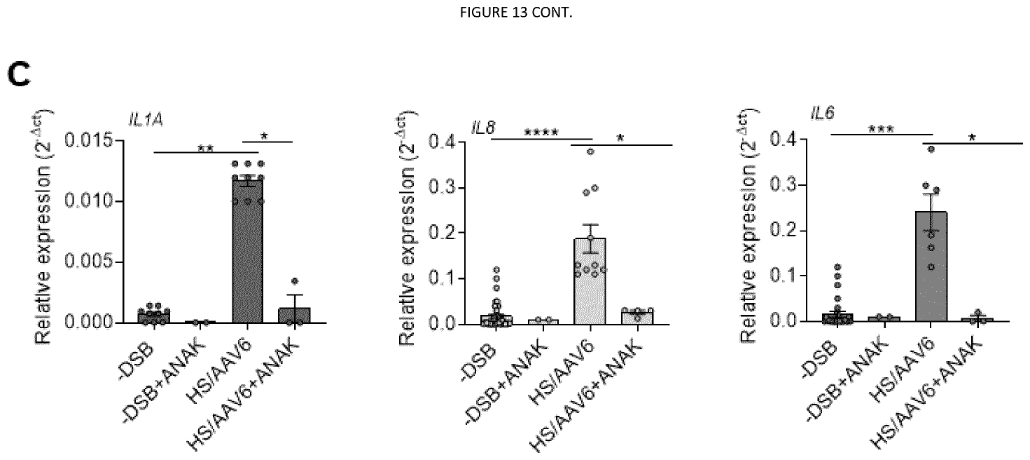

We next assessed whether Anakinra might have an impact on the transcriptional changes occurring upon GE, and we performed whole transcriptomic analysis on CB-derived HSPCs 24 and 96 h after AAVS locus editing, in order to investigate the early and late effects that Anakinra could exert on edited HSPCs. We tested editing in presence or absence of Anakinra in edited HSPC and their respective negative controls. We performed GSEA on gene lists ranked based on log 2FC to identify the pathways modulated by Anakinra. We found negative and significantly high normalized enrichment scores (NES) for inflammatory/TNFα dependent pathways (e.g. TNF-a signalling via nuclear factor kB [NFkB]; IL2-STAT5 signalling; IL-6/JAK/STAT3 signalling), interferon responses and senescence gene categories in cells edited in presence of Anakinra in respect to not treated counterpart mainly 24 h upon GE (FIGS. 13A, 13B). Accordingly, we reported a significant upregulation of key inflammatory genes of the IL1 signalling pathway in edited HSPCs, which are downregulated 96 h upon Anakinra treatment (FIG. 13C). Indeed, qPCR showed significant induction of IL1A, CXCL8 (interleukin-8 [IL8]) and IL6 in response to GE which is modulated in Anakinra-treated edited cells.

Altogether, these studies uncover that the induction of pro-inflammatory and senescence programs early and late upon gene editing are downregulated upon Anakinra treatment.

### Example 15—Anakinra Reduces NF-KB Nuclear Translocation Upon GE

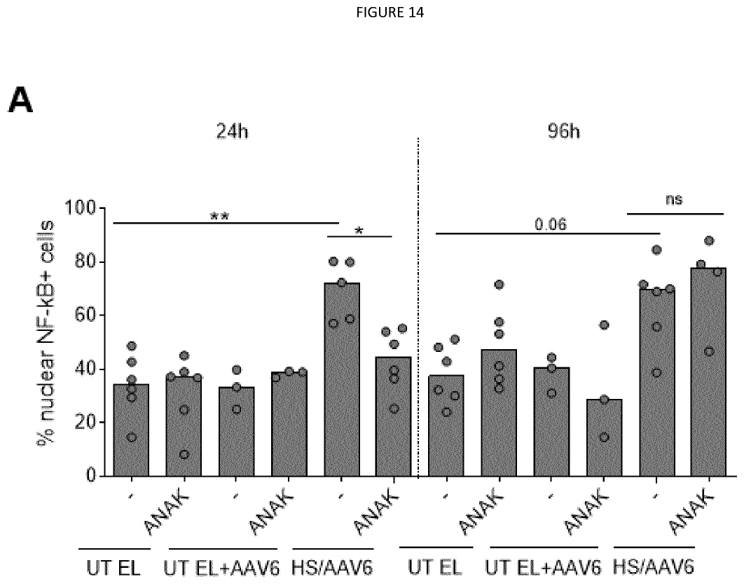

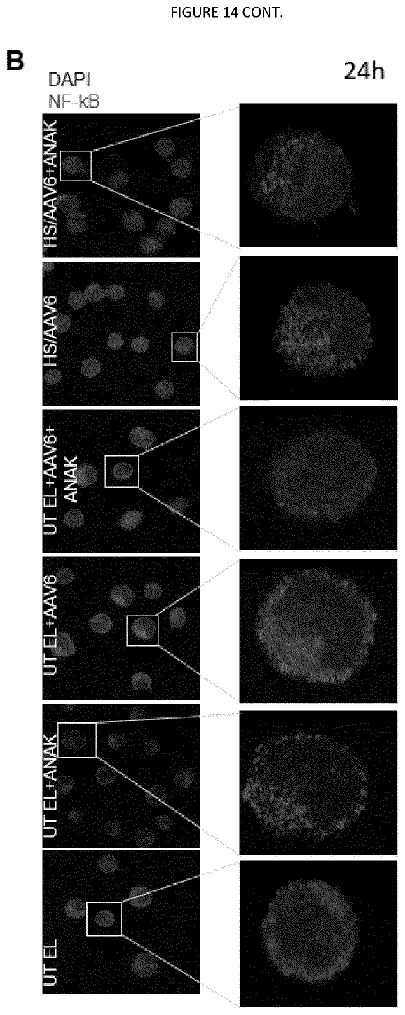

To study the molecular mechanism at the basis of IL-1 mediated inflammation, we measured the nuclear factor-kappa Beta (NF-kB) localization, as a surrogate marker for its activation. We treated edited cells with Anakinra during the gene editing procedure, and we used additional controls to discriminate Anakinra effect. We performed the sole electroporation of the cells (hereafter UT electro sample), the AAV6 transduction of the cells after their electroporation (hereafter UT electro+ AAV6 sample) and the standard gene editing procedure (HS/AAV6 sample) in presence or absence of Anakinra. We found an increase in NF-kB nuclear translocation upon gene editing compared to negative controls (UT electro and UT electro+AAV6) and a decrease upon Anakinra treatment (FIGS. 14A, 14B), suggesting that IL-1 signaling pathway is upstream NF-kB activation upon GE. According to the transient nature of Anakinra treatment, we no longer reported NF-kB activation 96 h post GE. Altogether, these studies highlight that IL1 regulates GE-related inflammation and senescence establishment acting upstream NF-kB.

### Example 16—Blocking IL1 Signalling Pathway is a Way to Preserve In Vivo Long-Term Reconstitution of HDR-Edited Cells

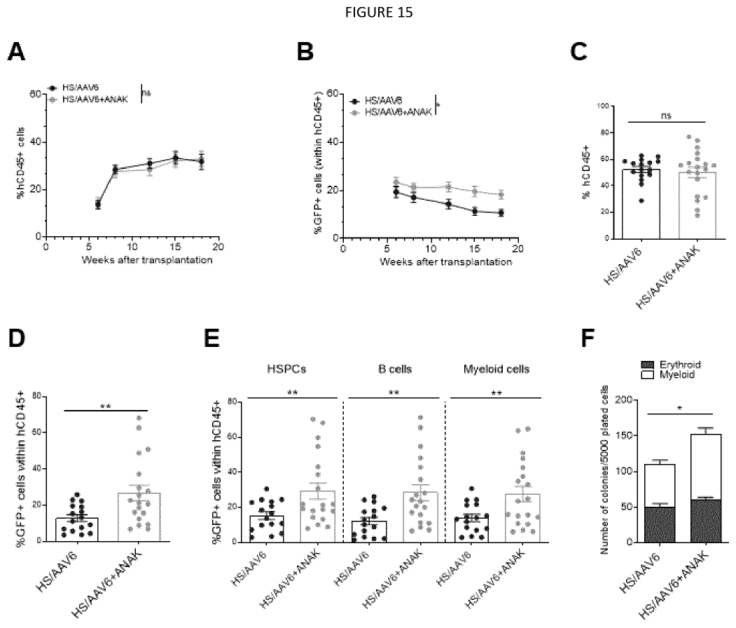

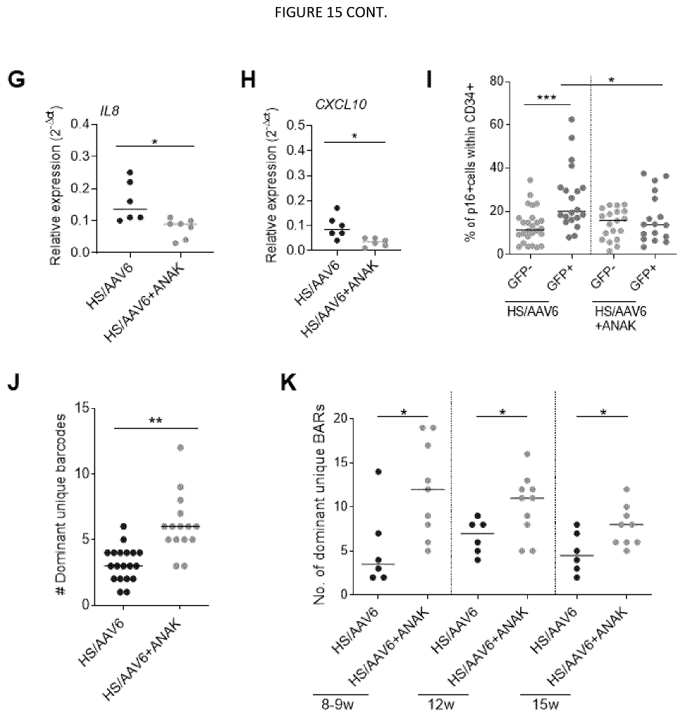

To investigate the repopulating potential of HSPCs edited in presence or absence of Anakinra, we transplanted matched saturating doses of GE-cell into NSG mice. We observed comparable human engraftment independently of the Anakinra treatment, which reached a plateau of 35-40% circulating cells (FIG. 15A), but reproducible higher and stable levels of HDR-edited cells edited in presence of Anakinra when compared to standard controls in peripheral blood (PB) overtime (FIG. 15B). Similar patterns of engraftment and % of GFP+HDR edited cells were also found within the human graft long-term after reconstitution in the bone marrow (FIGS. 15C, 15D). The percentages of GFP+ cells in BM-derived HSPC, B cells and myeloid cells lineages at the endpoint were consistent with the levels observed in the blood and BM, with Anakinra treated HDR-edited cells showing an improved long-term reconstitution compared to standard protocol edited cells (FIG. 15E). In addition, when we re-challenged human CD34+ cells retrieved from the bone marrow of primary recipients into semisolid medium, we found that Anakinra-treated edited HSPCs displayed higher clonogenic potential compared to standard protocol treated cells (FIG. 15F). The higher engraftment of anakinra-treated HDR-edited cells was in line with their decrease in pro-inflammatory cytokines expression (FIGS. 15G, 15H) and in the percentage of senescence cells in the human graft long-term (FIG. 151). To assess the clonal composition of host repopulation by Anakinra-edited HSPCs, we embedded a 22-basepair (bp) degenerated heritable ‘barcode’ sequence (BAR) in the repair template downstream of a green fluorescent protein (GFP) reporter cassette. We then generated a plasmid library and an AAV6 pool of high and comparable complexity (7.5×105 and 5.9×105 unique BARs, respectively) and nearly homogeneous representation of degenerated consensus sequences (Ferrari et al., 2020, Nat Biotechnol 38: 1298-1308). We found that Anakinra-treated edited HSPCs show a higher polyclonal reconstitution compared to standard gene edited cells, with increasing number of dominant BARs compared to respective control (FIG. 15J). Polyclonal reconstitution of HDR-edited clones in the human graft short and long term upon transplantation after Anakinra treatment was also confirmed by analyzing PBMCs in the saturating-dose experiment (FIG. 15K).

Overall, these data reveal that HDR-edited HSPCs in presence of Anakinra displayed a higher long-term polyclonal reconstitution, which is associated to a dampening of pro-inflammatory cytokines and senescence markers.

### Example 17—Anakinra Treatment Improves mPB-Derived HSPC Clonogenicity without Affecting HSPC Ability to Repair Via HDR

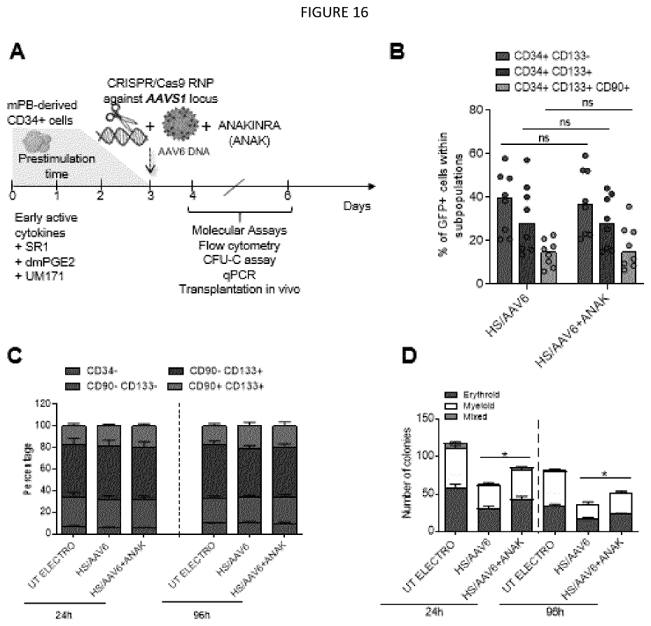

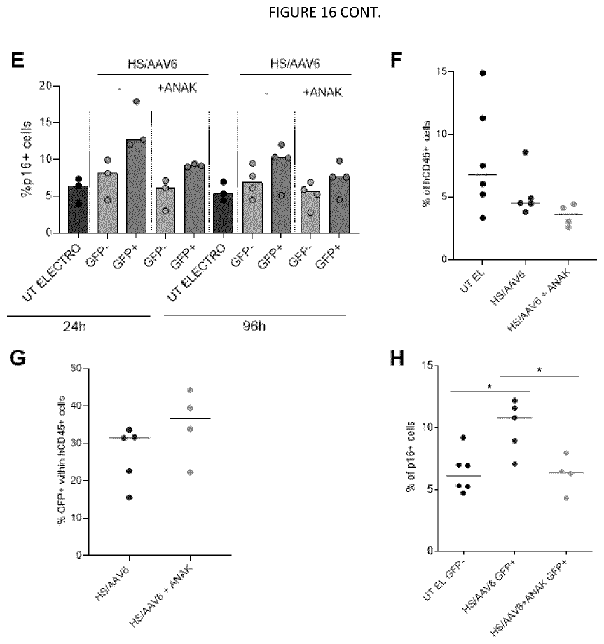

To extend our data to a more clinically relevant source of HSPC, we performed the same experiments reported in CB-derived HSPCs on mPB-derived HSPC. We treated cells within 15 minutes upon gene editing of the AAVS1 locus with Anakinra (FIG. 16A) and we showed that Anakinra treatment did not affect HSPC ability to repair via HDR (FIG. 16B), did not alter HSPC culture composition and differentiation (FIG. 16C) but significantly improved the clonogenic potential of edited cells (FIG. 16D). Moreover, GFP+ HDR-edited cells displayed high accumulation of senescence marker p16 early and late upon GE which was partially mitigated by Anakinra treatment (FIG. 16E). Consistently, when we transplanted matching low doses of edited cells into NSG mice, we observed comparable human engraftment independently of Anakinra treatment (FIG. 16F), but a trend of higher levels of HDR-edited cells edited in presence of Anakinra when compared to standard controls (FIG. 16G) and a reduction in the senescence burden in the human graft (FIG. 16H).

Overall, these data suggest that Anakinra dampened GE-related senescence establishment also in mPB-derived HSPCs improving their long-term hematopoietic reconstitution.

### Example 18—Anakinra Treatment Improves mPB-Derived HSPC Clonogenicity without Affecting HSPC Ability to Repair Via HDR

To dissect the role of senescence and inflammation in the context of GE exploiting an alternative DNA donor template for HDR different from AAV6, we performed AAVS1 editing in mPB HSPCs using and Integrase-defective-lentiviral vector (IDLV) as template for HDR.

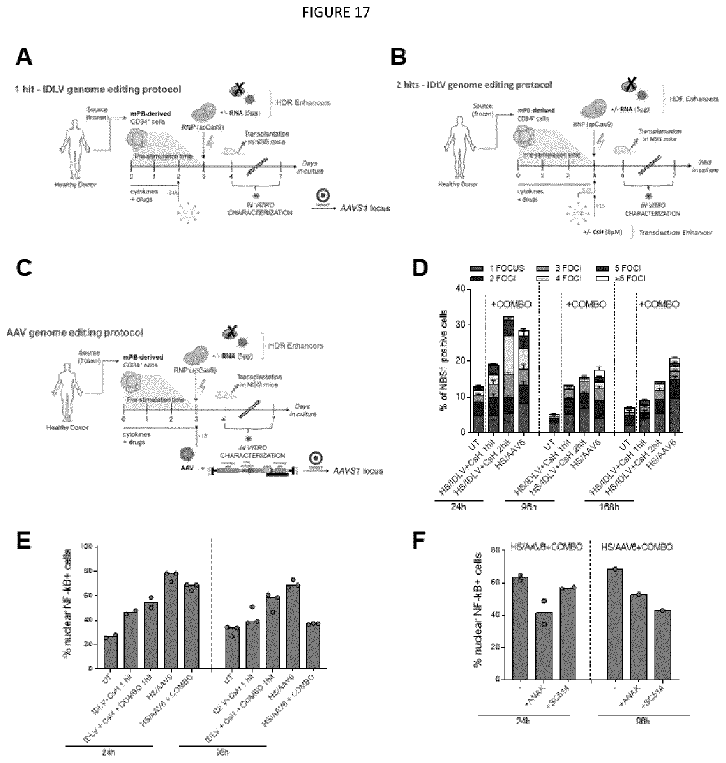

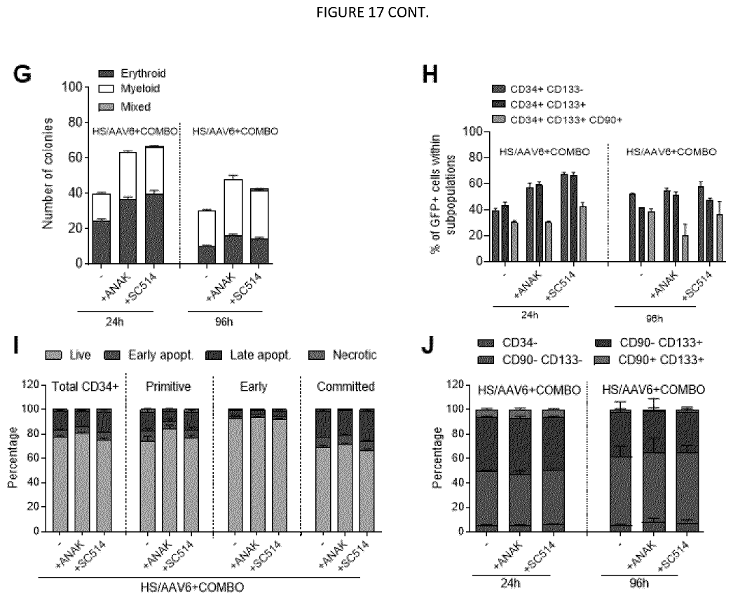

To reach higher efficiency of IDLV transduction, we employed the transduction enhancer CsH as previously reported (Petrillo, C. et al. 2018, Cell Stem Cell 23, 820-832.e9). In addition, we also performed AAVS1 editing in mPB HSPCs by combining CsH, GSE56 and E4orf6/7 adenoviral proteins (GSE56+E4orf6/7 hereafter named COMBO for optimized protocol with HDR enhancers) to reach higher levels of HDR (Ferrari, S. et al., 2020, Nat Biotech 38(11):1298-1308). To identify the optimal conditions for maximal DNA donor availability during DSB repair, we tested different timings (12 or 24 hours before RNP electroporation) and one or two hits of IDLV delivery compared to AAV6 one (see the employed protocols in FIGS. 17A, 17B, 17C). We reported a persistent increase of the DDR marker NBS1 when cells were edited in presence of the AAV6 donor template compared to 1-hit or 2-hit IDLV templates with the optimized protocols for all the treatments (FIG. 17D). Notably, this was consistent with NF-kB activation, which was higher upon AAV6 GE compared to 1-hit IDLV GE early and late upon genetic manipulation (FIG. 17E). To dampen the AAV6 NF-kB higher nuclear activation compared to IDLV, we performed anti-inflammatory treatments were performed 15 minutes upon GE with Anakinra or the NF-kB inhibitor SC-514 (Kishore, N. et al., 2003, Journal of Biological Chemistry 278: 32861-32871; and Roman-Blas, J. A., & Jimenez, S. A., 2006, Osteoarthritis Cartilage 14: 839-848) in CB-derived HSPCs electroporated with HDR enhancers (COMBO). We reported a further decrease in NF-kB nuclear localization upon both anti-inflammatory treatments compared to cells experiencing GE in the sole presence of GSE56 and E4orf6/7 (FIG. 17F). The decrease in NF-kB induction upon Anakinra or SC514 treatments was associated with an increase in the clonogenic potential of the cells (FIG. 17G) without impairing HSPC ability to repair via HDR (FIG. 17H), without overt apoptosis (FIG. 17I) and culture composition skewing (FIG. 17J).

Overall, these findings reveal that also GE with IDLV donor template led to a DDR and NF-kB induction in mPB-derived HSPCs but with a lower extend compared to AAV6 treatment for higher transduction efficiency. Moreover, when performing GE with AAV6 in presence of HDR enhancers, NF-kB induction can be mitigated by temporary inhibition of inflammatory pathways leading to an increase in the clonogenic potential of the edited HSPCs.

### Example 19—NF-kB Inhibition Improves HSPC Long-Term Reconstitution and Synergize with p53 Inhibition

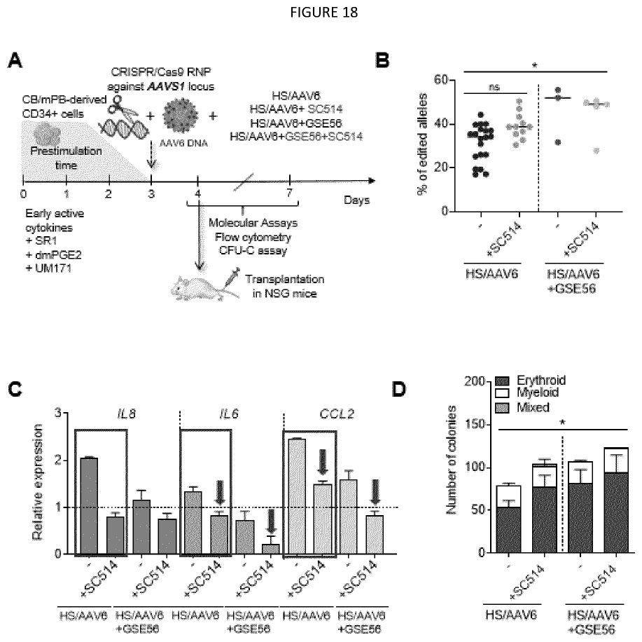

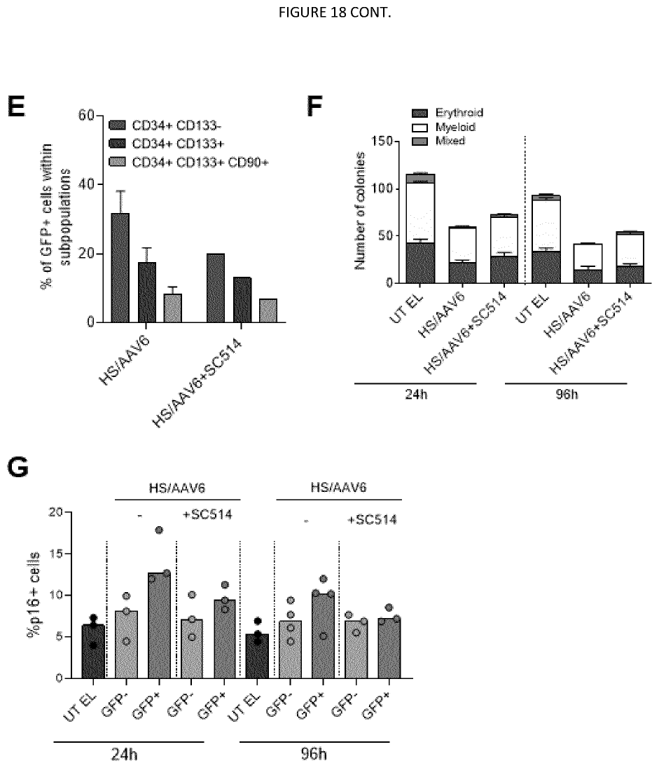

In order to more broadly inhibit NF-kB pathway to have a higher improvement in edited HSPC functionality, we employed the above mentioned IKK-2 Inhibitor (SC514), a suppressor of the NF-kB-dependent inflammatory transcriptional program (Kishore, N. et al., 2003, Journal of Biological Chemistry 278: 32861-32871; and Roman-Blas, J. A., & Jimenez, S. A., 2006, Osteoarthritis Cartilage 14: 839-848). We treated cells with SC514 within 15 minutes upon GE of the AAVS1 locus in CB-derived HSPCs and we performed p53 inhibition alone or in combination to NF-kB inhibitor, collecting cells 24 and 96 h upon GE (FIG. 18A). We reported no differences in terms of HDR-edited alleles comparing edited HSPC in presence or absence of NF-kB inhibition. On the contrary, we found that GSE56 tended to increase the percentage of HDR editing and this effect was maintained upon NF-kB inhibition (FIG. 18B). Moreover, since NF-kB is an upstream mediator of inflammation, we reported a decrease in the expression levels of some upregulated DDR-related cytokines (such as IL6, IL8 and CCL2) in SC514-treated edited HSPCs. In addition, we also reported a downregulation of the cytokines in presence of GSE56, which was even exacerbated performing GSE56 and SC514 simultaneous treatments (FIG. 18C). Colony-forming potential was similarly increased for editing treatment in presence of SC514 or GSE56 as compared to standard protocol edited HSPCs, with a trend toward more colonies for GSE56/SC514 combination, without detectable difference in erythroid to myeloid ratio (FIG. 18D).

We also performed NF-kB inhibition experiments in mPB-derived HSPCs, and we reported no differences in GFP+ cells, especially in the most primitive subpopulation (FIG. 18E), but an improved clonogenicity of the edited cells experiencing NF-kB inhibition respect to non-treated controls overtime (FIG. 18F) correlated with a decrease in senescence establishment in HDR-edited cells (FIG. 18G). Altogether, these data suggest that NF-kB transient inhibition allows edited HSPC higher clonogenic potential due to a decrease in senescence markers accumulation that is further improved when employed in combination with GSE56.

### Example 20—NF-kB Inhibition During GE Reduces the Percentage of HDR-Edited Senescent Cells in the Human Graft

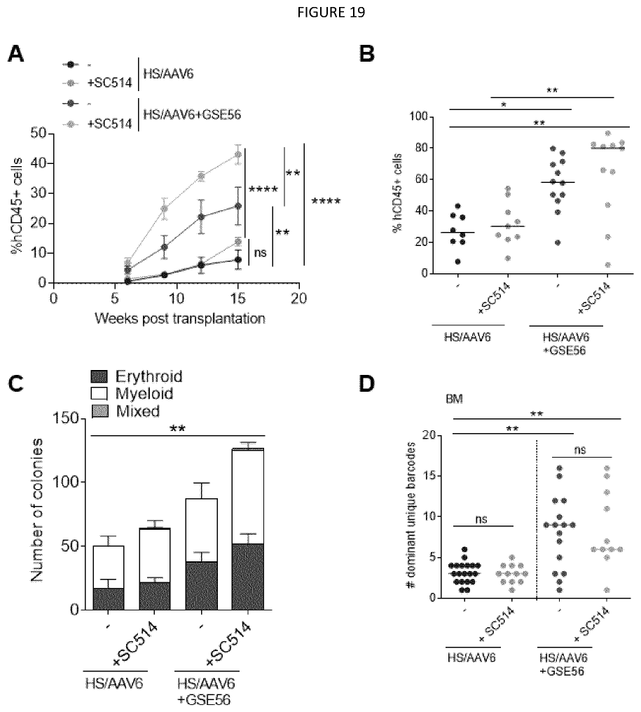

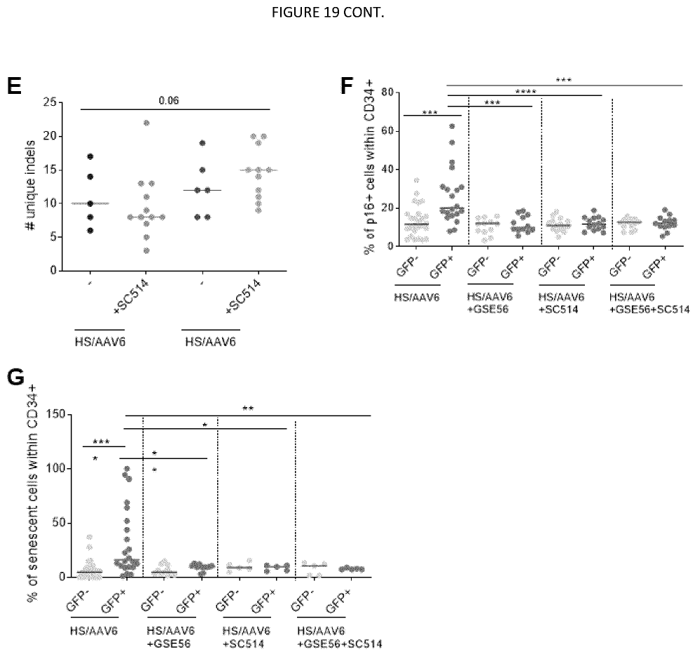

To investigate the repopulation potential of HSPCs edited in the presence or absence of SC514 and GSE56, we transplanted matched limiting cell doses into NSG mice, and we confirmed that GSE56 addition allowed fourfold higher engraftment than the standard protocol, while it was combined with SC514 even potentiate this increase, reaching nearly the 40% of human engraftment (FIG. 19A). Similar patterns of engraftment were found long-term after reconstitution in the hematopoietic organs (FIG. 19B). To further investigate long-term functionality of edited HSPCs, we performed a colony-forming assay by purifying human CD34+ cells from the bone marrow of primary recipients. We showed a slight trend of increase in the clonogenic potential of the cells upon SC514 treatment that became higher upon p53 inhibition (FIG. 19C). Moreover, we reported a significant increase in the fitness of edited HSPC when combining NF-kB and p53 inhibition, suggesting that they might have a synergistic effect.

Finally, by performing clonal tracking of HDR-edited HSPC by a barcoding-based strategy (FIG. 19D), as well as the clonal tracking of unique indels-bearing alleles in the human graft (FIG. 19E), we found that NF-kB inhibition improved polyclonal reconstitution and preserved self-renewal and multi-potency of individual edited HSPCs in combination of GSE56. In addition, we reported that NF-kB inhibition reduces the percentage of senescence HDR-edited cells (FIGS. 19F, 19G) and this reduction was observed also upon p53 inhibition in presence or absence of NFKB inhibition.

Overall, these findings reveal that NF-kB inhibition at the time of GE can be a valuable strategy, mainly in combination to p53 inhibition, to reach higher level of hematopoietic reconstitution and decrease in senescence establishment in the human graft.

### Example 21—Chemical Inhibition of p38-MAPK Reduces ROS Levels, Cellular Proliferation, DNA Damage Accumulation and DDR Activation in HSPCs after Ex Vivo Culture

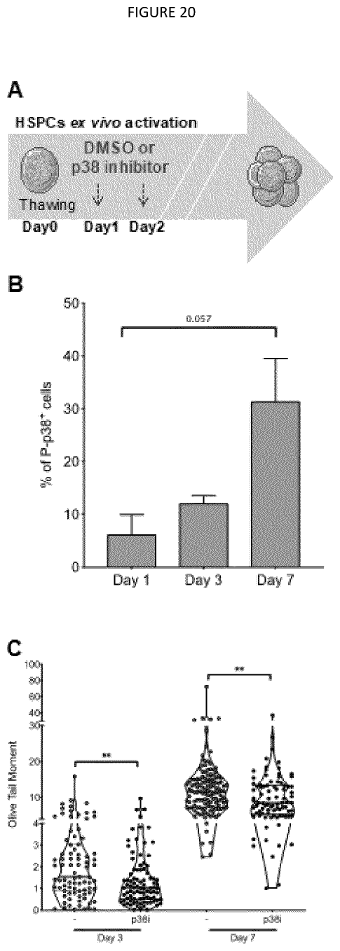

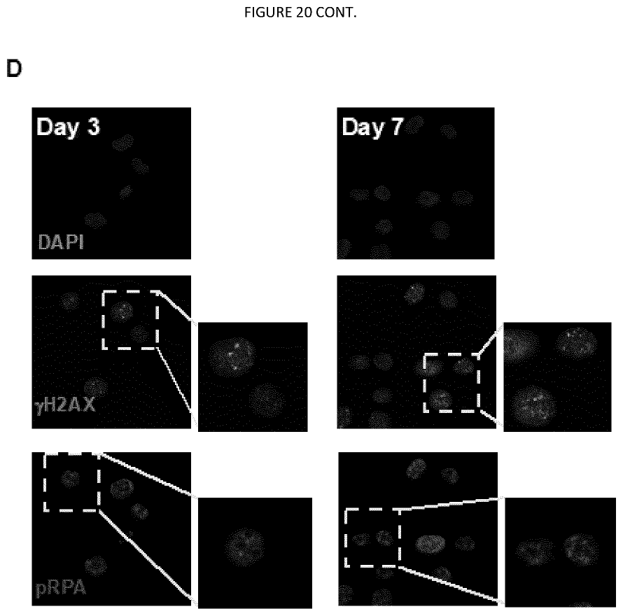

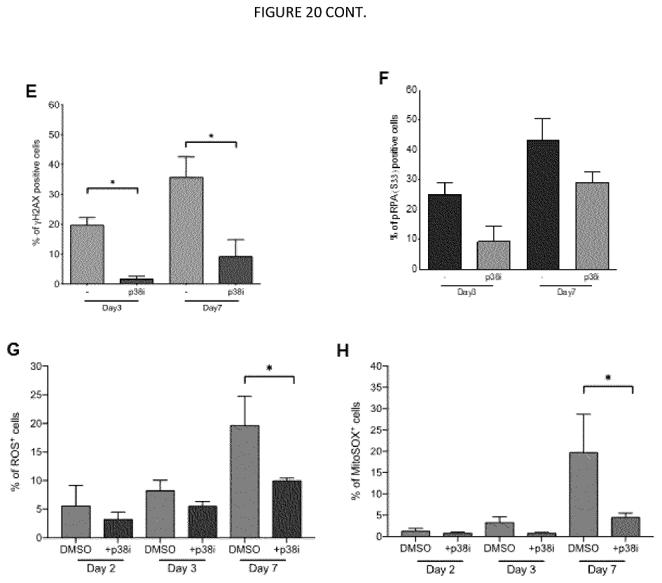

Current culture conditions for ex vivo activation have been tailored over the past decade to ensure high gene editing efficiency and preserve HSPC long-term repopulating capacity upon ex vivo gene manipulation (Hoggatt et al., 2009, Blood 122: 5444-5455; Boitano et al., 2010, Science 329: 1345-1348; Fares et al., 2014, Science 345: 1509-1512; Zonari et al. 2017, Stem Cell Reports 8: 977-990). However, we reported that activated stem cells, when exiting from their quiescent status, displayed features of stress, ultimately resulting in stem cell dysfunctions. Indeed, by alkaline comet assay, we found that human HSPCs accumulate physical DNA damage in the form of both single- and double-strand breaks (SSBs and DSBs) (FIG. 20C). Consistently with the presence of DNA breaks, HSPCs also showed increasing numbers of γH2Ax and pRPA foci, indicating DDR activation (FIGS. 20D, E, F). Moreover, we noticed an increase in oxidative stress, assessed by flow cytometry for cytosolic Reactive Oxygen Species (ROS) and mitochondrial superoxides (FIG. 20G, H) in HSPCs. Interestingly, it has been reported that HSCs with high levels of ROS also showed increased activation of the p38 mitogen-activated protein kinase (MAPK) and it is becoming clearer that p38-MAPK plays an important role in hematopoiesis and its activation induces HSPCs proliferation, differentiation, and exhaustion (Jang and Sharkis, 2007, Blood 110: 3056-3063; Geest and Coffer, 2009, Journal of Leukocyte Biology 86: 237-250; Lu et al., 2016, Int J Mol Sci 17: 905). p38 is a stress-responsive MAPK involved in several aspects, including senescence, aging, and metabolism, and can be activated by several stressors such as ROS, osmotic stress, and DNA damage (Canovas and Nebreda, 2021, Nature Reviews Molecular Cell Biology 22: 346-366). Indeed, we reported increasing levels of the phosphorylated form of p38 upon time in culture (FIG. 20B). Thus, in order to ameliorate culture conditions and eventually achieve more functional HSPCs, we reasoned to inhibit p38-MAPK signaling by employing SB203580, a highly specific ATP-competitive inhibitor of its kinase activity, that does not affect protein phosphorylation. Specifically, we decided to treat HSPCs during the first 2 days of culture. (FIG. 20A). Interestingly, chemical inhibition of p38 reduced the production of cytosolic ROS and mitochondrial superoxides (FIG. 20G, H), improved proliferative stress diminishing proliferation-induced DNA damage, and DDR activation (FIGS. 20D, E, F).

Overall, these data suggest that p38-MAPK is a key mediator of culture-induced stress and its inhibition ameliorates proliferative stress accumulated with time in culture by HSPCs.

### Example 22—Pre-Treatment of Edited HSPCs with p38 Inhibitor Decreased ROS Levels and Increased Primitive Cells Clonogenic Capacity and HDR Efficiency

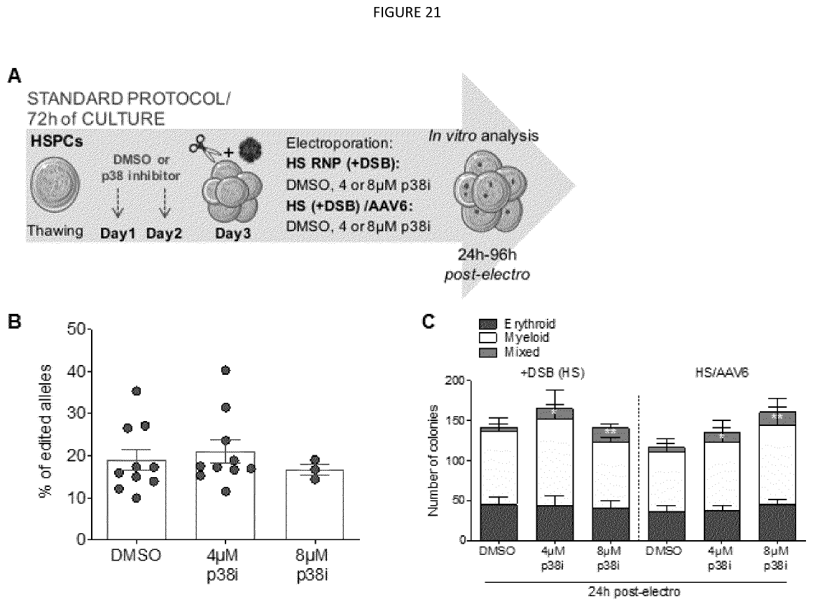

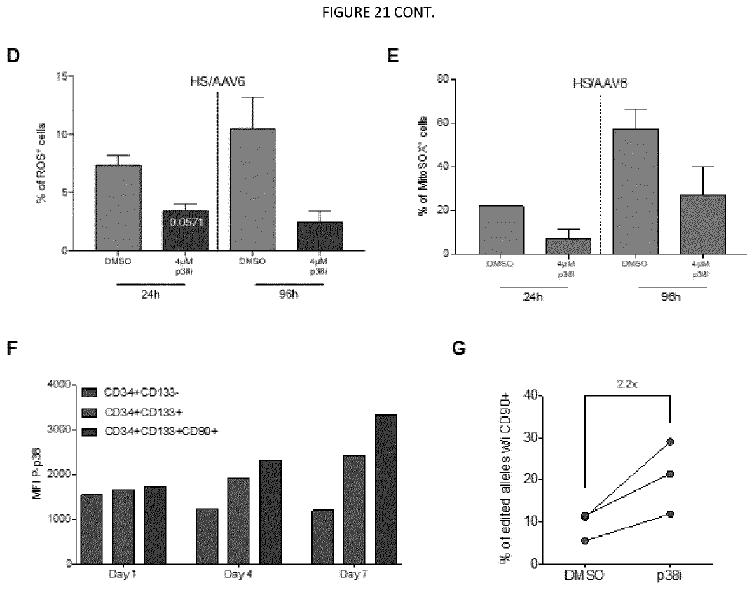

Given these results, we thought that decreasing the detrimental accumulation of ROS and DNA damage with a p38 inhibitor during HSPC ex vivo activation previous to genetic manipulation would better preserve HSPC long-term functionality. In detail, we decided to culture HSPCs in the presence of two different doses (4 μM or 8 μM) of p38 inhibitor for the 2 days before the editing procedure. On day 3, HSPCs were electroporated with a Cas9 loaded with gRNAs highly specific for the AAVS1 locus (HS RNP) alone or in combination with the delivery of the GFP-expressing AAV6 donor template (HS/AAV6) (FIG. 21A) (Schiroli et al., 2019; Cell Stem Cell 24: 551-565.e8). p38 inhibition did not impact HDR efficiency measured molecularly by ddPCR in the bulk CD34+ cells at 96 h post-electroporation (FIG. 21B). Evaluating HSPC functionality by testing their clonogenic capacity, we observed an increase in colony numbers after p38 inhibitor treatments in both HS RNP and HS/AAV6 conditions. Surprisingly, the higher number of colonies was mostly due to an increase in the number of mixed colonies (CFU-GEMM), the progeny of more primitive and multipotent HSCs (Carow, Hangoc and Broxmeyer, 1993, Blood 81: 942-949) (FIG. 21C). Interestingly, we found that p38 inhibitor was able to diminish ROS and mitochondrial Superoxides accumulation in HSPCs upon editing (FIGS. 21D, E). Furthermore, the most primitive subpopulation (CD34+CD133+CD90+) revealed to be the compartment with the highest p38 activation (FIG. 12F), and surprisingly, we detected more than 2-fold increase of edited alleles in sorted HSC treated with p38 inhibitor prior to gene editing (FIG. 21G).

Altogether these results indicate that p38 inhibition mitigates ROS and superoxide accumulation upon gene editing, improves in vitro functionality and boosts HDR-efficiency in primitive HSCs.

### Example 23—Edited HSPC Pre-Treated with p38 Inhibitor Display an Higher Engraftment, Number of Edited Clones Post-Transplant in NSG Mice and Improved Peripheral Blood Composition

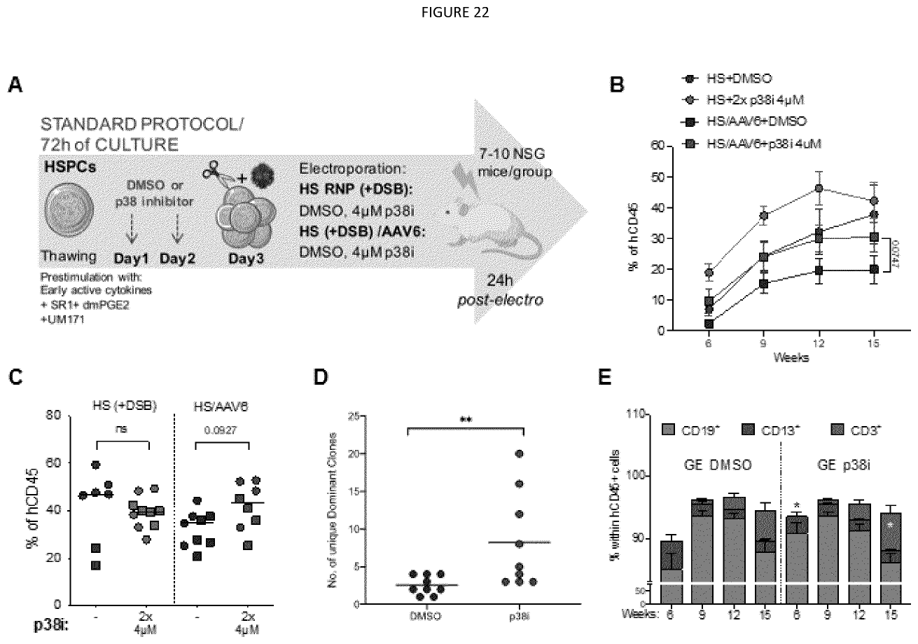

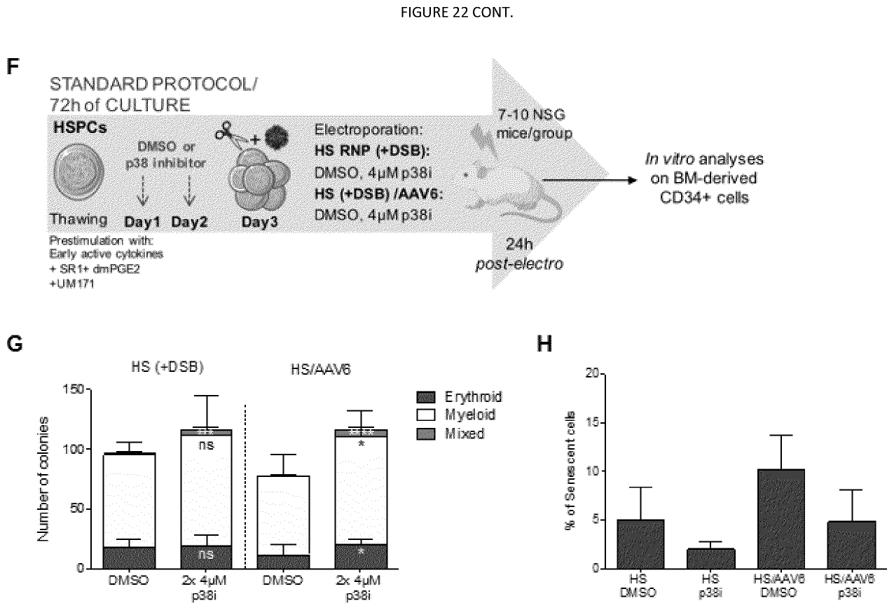

The increase in mixed colonies observed when we edited HSPCs upon p38 inhibition, suggested that the treatment could preserve the fitness of more primitive HSC subsets. Thus, we tested the in vivo functionality of HSPCs upon a single DSB (HS) or full editing (HS/AAV6) in the presence or absence of p38 inhibition and transplanted edited CB-derived CD34+ into NSG mice (FIG. 22A). We followed HSPC repopulating capacity by performing serological analysis at different time points upon transplantation (from 6 to 15 weeks), and measuring the percentage of human CD45+ (hCD45+) cells in peripheral blood. We found that the percentage of hCD45+ cells of mice transplanted with HSPCs that experienced only the DSB (HS condition) pre-treated with p38 inhibitor was effectively increased up to 12 w but stabilized at 15 w, and the engraftment returned similar to DMSO control. Instead, the percentage of engrafting HS/AAV6+p38i treated HSPCs was, from the beginning, higher compared to the DMSO control group (FIG. 22B). Data obtained in the Bone Marrow (BM) at the time of euthanasia (15 w), mirrored the findings in PB: the engraftment of HS condition was similar across treatments, while p38 inhibition improved HS/AAV6 engraftment (FIG. 22C). Given the promising results on the engraftment and the improved functionality of edited HSPCs upon p38 inhibitor treatment, we asked whether p38 inhibition could influence the number of edited HSPC clones. Indeed, we selected for BAR-seq analysis mice transplanted with HSPCs edited with DMSO or p38 inhibitor with detectable engraftment (>0.1%) of GFP+ cells in the BM at 15 w (Ferrari et al., 2020; Nature Biotechnology 38: 1298-1308). Interestingly, we found that the number of dominant BARs was significantly increased upon p38 inhibition, suggesting an enhanced polyclonal reconstitution of HDR-edited cells upon p38i treatment (FIG. 22D). However, when analysing the impact of p38i in the different subpopulations within the human graft, we found an early decrease in myeloid cells and a late expansion of T cells in the PB when HSPCs were edited upon p38 inhibitor treatment (FIG. 22E).

Overall, these findings reveal that upon in vivo transplantation, p38 inhibitor treated gene edited-HSPCs displayed better repopulating capacity and differentiation, together with an enhanced polyclonal reconstitution.

### Example 24—p38 Inhibition Increases HSPC Functionality at Long-Term Post-Transplantation and Reduces the Percentage of Senescent Cells

At the time of euthanasia (15 w), we purified CD34+ cells from the BM and tested their clonogenic potential in CFU-C assay (FIG. 22F). Interestingly, we reported a higher number of colonies generated by BM-derived HS and HS/AAV6 HSPCs treated with p38 inhibitor; however, differences were more evident in HS/AAV6 group. As previously reported, we confirmed an increase of mixed colonies in both conditions upon p38i treatment (FIG. 22G). In line with these results, we also reported a reduction of senescent cells when edited HSPCs were pre-treated with the p38 inhibitor, indicating that p38 inhibition better preserves edited HSPC functionality (FIG. 22H).

Overall, these data suggest that p38 inhibition dampened GE-related senescence establishment, improving their long-term hematopoietic reconstitution.

### Example 25—p38 Inhibitor Treated HSPCs have Higher Clonogenic Potential and Improved Differentiation Toward Megakaryocyte and Erythroid Lineages

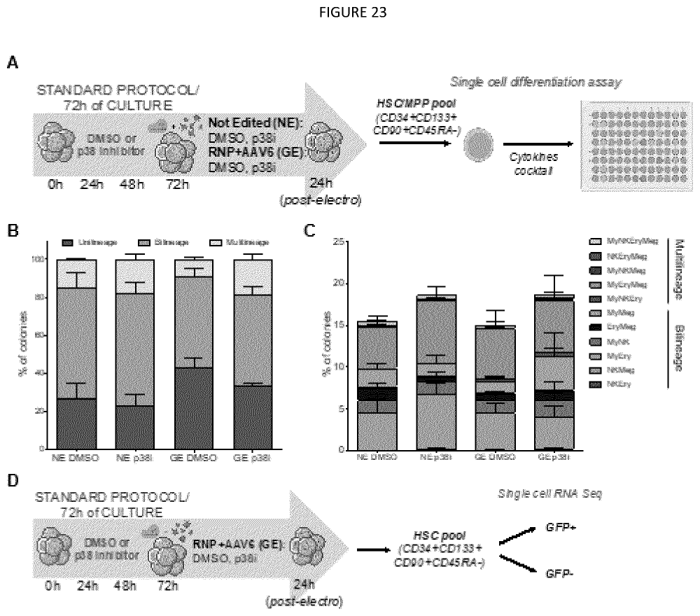

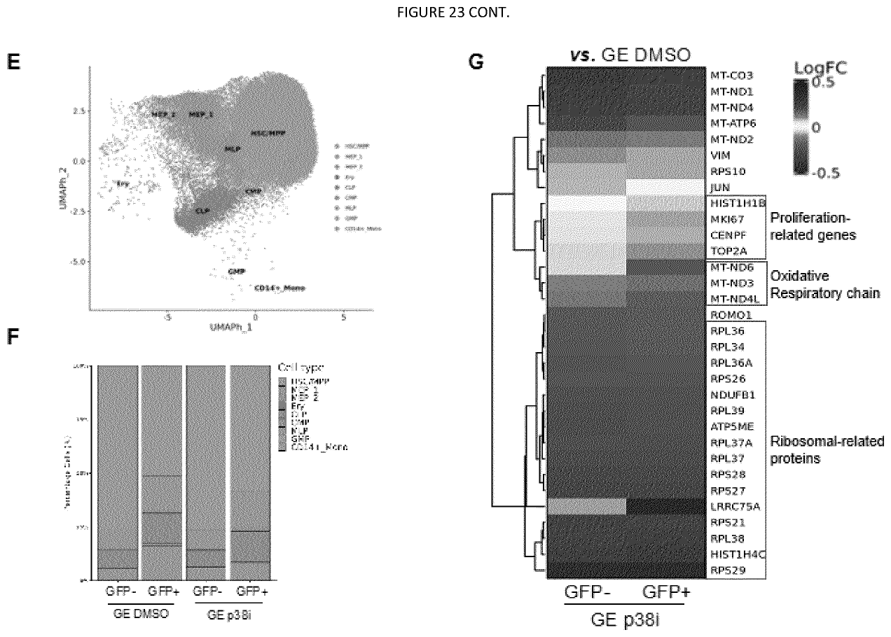

Since HSPCs edited upon p38 inhibitor pre-treatment showed increased in vitro clonogenicity and in vivo repopulation capacity (higher engraftment, improved composition, and clonality), we wondered whether p38 inhibition was specifically preserving the functionality of the most primitive subset of CD34+ cells. To address this point, we characterised the differentiation potential of single human phenotypic HSPCs along the My, Ly, Meg, and Ery lineages (Belluschi et al., 2018, Nature Communication 9). 24 h post-editing or upon 4 days of culture, we sorted more than 500 single HSC/MPP pool cells (CD34+CD133+CD90+CD45RA−) from four individual umbilical cord blood (CB) samples (FIG. 23A). In this assay, around 80% of single cells reproducibly produced colonies of ten different types across individual HSC donors and experiments. In the not edited condition (NE), approximately 70% of the cells gave rise colonies with 2 or 3 lineages, 10% of which were half of the My/Ery and the other half of the My/Ery/Meg type (FIGS. 23B, 23C). Among uni-lineage colonies, around 90% were My-only colonies; My colonies contained either monocytes, granulocytes, or both (data not shown). Interestingly, p38 inhibitor favoured the formation of bi-lineage and, even more, multi-lineage colonies in both not edited and edited HSPCs (FIG. 23B). Furthermore, within the bi- and multi-lineage composition, we observed an expansion of My/Ery and My/Ery/Meg colonies in not edited conditions and My/NK, Ery/Meg, and My/Meg colonies in gene-edited HSPCs when p38 inhibitor was added to the culture prior to genetic engineering (FIG. 23C).

Altogether, these studies highlight that p38 inhibition ameliorates gene edited HSPC clonogenic potential, and importantly, megakaryocyte differentiation, suggesting a better preservation of differentiation potential of more primitive HSCs.

### Example 26—Single-Cell RNA Sequencing Analysis of GE-HSPCs Treated with p38i Revealed Distinct Cluster Composition and Enrichment for Gene Categories Involved in Cell Proliferation, ROS Detoxification and Stemness

To investigate the molecular pathways associated with primitive gene-edited HSCs treated with p38 inhibitor, we performed single-cell RNA sequencing analysis. Specifically, 24 h post-GE, we sorted a cellular population enriched in GFP+ (gene-edited) HSC (CD34+CD133+CD90+CD45RA−GFP+) and GFP− (not edited) HSC (CD34+CD133+CD90+CD45RA−GFP−) (FIG. 23D). To identify cell subpopulations and better enable comparison across datasets, we segregated cell clusters, computing the significant components using a supervised approach based on a list of differentially expressed genes (DEGs) between HSCs and progenitors in culture. We identified 9 clusters with the biggest composed of HSC and multipotent progenitors (MPP) marker genes and depleted in lineage-associated markers (hereafter named “HSC/MPP” cluster), consistently with the enrichment in primitive cells by cell sorting (FIG. 23E). The other clusters were enriched for different sets of lineage-associated genes, hereafter named: Megakaryocyte-erythroid progenitors (MEP_1 and 2), Myeloid-Lymphoid progenitors (MLP), Common myeloid progenitors (CMP), Common Lymphoid progenitors (CLP), Granulocyte-monocyte progenitors (GMP), Erythroid (Ery), and Monocytes (CD14+_Mono) (FIG. 23E). Nevertheless, cluster composition changed across samples: edited (GFP+) cells displayed a remarkable reduction of HSC/MPP cluster compared to not edited (GFP−) cells, independently from the treatment and reflecting less permissiveness of more primitive cells to gene editing manipulation (FIG. 23F). Importantly, p38 inhibitor treatment expanded the proportion of the HSC/MPP cluster of GFP+ cells (FIG. 23F). Moreover, gene expression comparison of the sorted HSC-pool GE cells pre-treated or not with p38 inhibitor confirmed our own functional data. Indeed, genes involved in proliferation, in the oxidative respiratory chain, and genes of ribosomal-related proteins were down-regulated upon p38 inhibition. Interestingly, the latter gene category was associated with cellular quiescence (Signer et al., 2014, Nature Medicine 20: 833-846), further supporting the hypothesis of preserving more primitive cells upon p38i treatment (FIG. 23G).

Overall, transcriptomic analyses revealed an expansion of HSC and MPP compartment and confirmed in vitro results, reporting lower proliferation, ROS responses and protein synthesis of edited HSC treated with p38 inhibitor.

### Example 27—Inhibition of Different MAPKs Increases HDR within Different Subpopulations and Ameliorates HSPC Clonogenic Potential

Given the promising results obtained with p38 inhibition, we reasoned that other mitogen-activated protein MAP kinases (MAPKs) could contribute to the loss of HSPCs functionality upon ex vivo expansion and genome editing procedures. Indeed, the family members of the MAPKs are evolutionarily conserved and are involved in the control of physiological cellular processes including: cell proliferation, survival, differentiation, apoptosis, and tumorigenesis. In mammalian cells, three distinct subgroups within the MAPK family have been described: namely classical MAPK (also known as ERK), C-Jun N-terminal kinase/stress-activated protein kinase (JNK/SAPK), and p38 kinase (Wei and Liu, 2002, Cell Research 12: 9-18; Cicenas, Zalyte, Bairoch, et al., 2018, Cancers 10: 63).

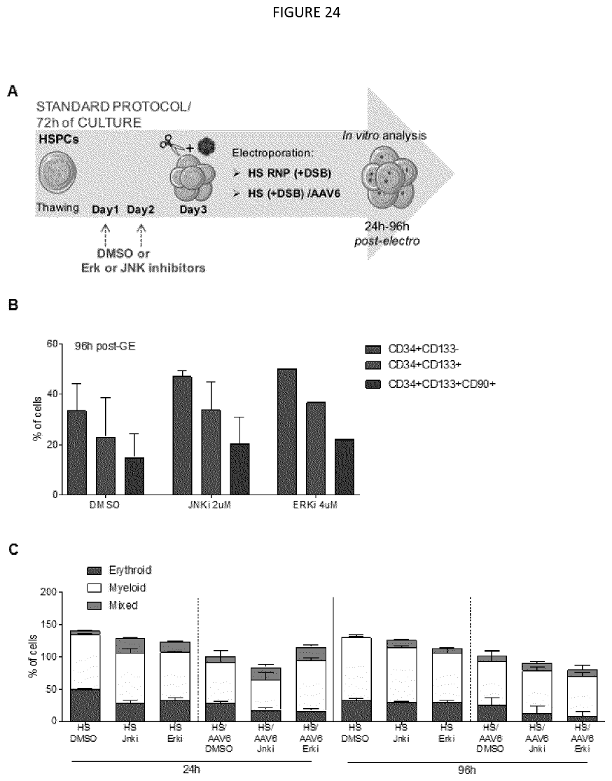

In detail, HSPCs were treated with DMSO, p38 (4 μM), Erk (4 μM), or Jnk (2 μM) inhibitors on Day 1 and Day 2. On Day 3 HSPCs were electroporated with the HS RNP alone or in addition to the AAV6 GFP-expressing donor template as required for the standard gene editing protocol (FIG. 24A). We investigated GE efficiency in HSPC subpopulations, from the more committed to the more primitive subfractions, and we reported an increase in GFP levels upon Erk and Jnk inhibition (FIG. 24B). Moreover, we evaluated HSPC functionality by testing their clonogenic capacity, and 24 h post-electroporation, and observed an increase in mixed colonies after all inhibitor treatments in both HS RNP and HS/AAV6 conditions (FIG. 24C).

Overall, MAPK inhibition improved HSPC editing efficiency and functionality, underlining a central role of MAPK activation in driving HSC dysfunctions.

### Example 28—p38 Inhibitor Treatment Expanses T Stem Cell Compartment and Reduces the Percentage of Mitochondrial Superoxides and Senescence

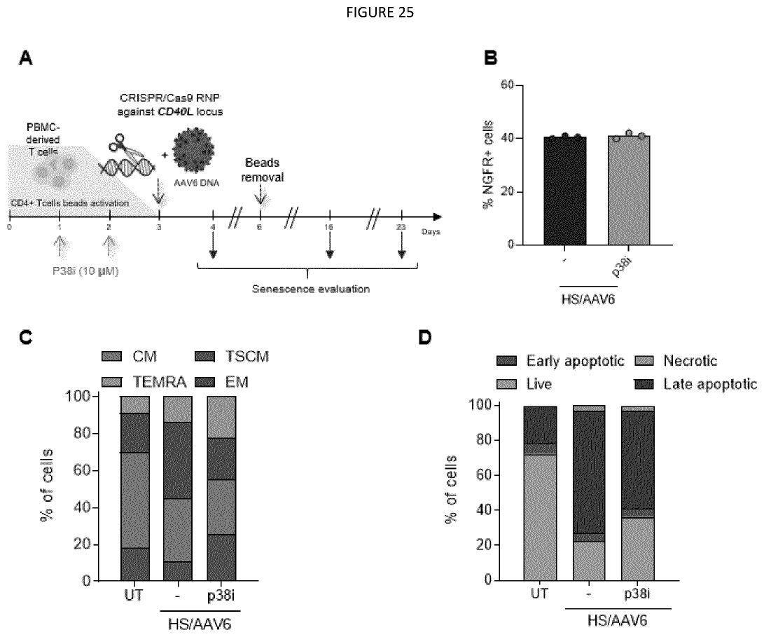

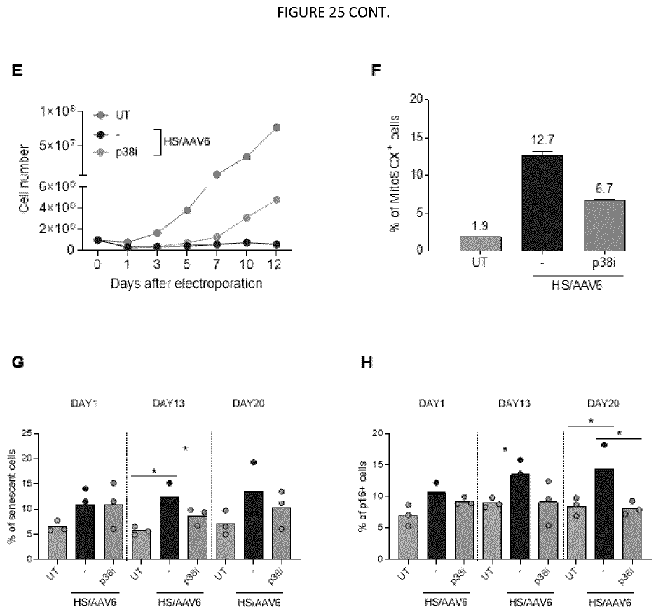

Since p38 inhibition ameliorated HSPC functionality upon gene editing, we thought to also test it in other cellular types clinically relevant in the context of gene and cellular therapies. We started from CD4+ T cells purified from 3 different donors and tested the effect of the p38 inhibitor. In particular, T cells were treated on day 1 and day 2 with 10 μM p38i and edited on day 3, targeting the CD40L locus, as previously reported (Vavassori, V. et al., 2021, EMBO Mol Med. 13(3):e13545) (FIG. 25A). While editing efficiency was not affected by p38i (FIG. 25B), we reported an expansion of the T stem cell memory (TSCM) compartment and T effector memory RA (TEMRA) (FIG. 25C), a decrease in apoptosis (FIG. 25D), and a concomitant increase in the proliferation rate upon gene editing (FIG. 25E). 24 h post-editing, we measured mitochondrial superoxides levels: their levels increased upon gene editing compared to the non-edited control (UT), but more importantly, p38i reduced superoxide accumulation (FIG. 25F). Furthermore, p38i also decreased the accumulation of the senescence markers SA-β-Gal and p16 early and late upon gene editing (FIGS. 25G, 25H). Altogether. these results suggest that p38 inhibition positively regulated T cell responses to gene editing.

### Materials & Methods

### Vectors and Nucleases

AAV6 DNA donor templates were generated from a construct containing AAV2 inverted terminal repeats, produced at the TIGEM Vector Core by a triple-transfection method and purified by ultracentrifugation on a cesium chloride gradient. Design of the barcoded AAV6 vector was obtained by subcloning a degenerated BAR sequence downstream of the GFP reporter cassette in the reference AAV backbone for AA VS1 editing. For molecular cloning of the barcoded AAV, a single-stranded oligonucleotide (ssODN) embedding the 22-bp BAR sequence flanked by unique restriction sites (Bsu36I and SphI, New England Biolabs) was purchased from Sigma Aldrich. Theoretical complexity of the ssODN was estimated in 2.9×1010. A BAR consensus sequence was designed to contain some invariant positions (7, 9, 15) and others limited to few bases (3, 14, 17, 21, 22) to avoid generating Bsu36I and SphI restriction sites. To generate the complementary strand, 50 pmol of the ssODN underwent ten PCR cycles with Easy-A High-Fidelity enzyme (Agilent Technologies) using the appropriate primers (see Ferrari, S. et al., 2020, Nat Biotech 38(11):1298-1308) and according to the manufacturer's instructions. The amplified product was purified with MinElute PCR Purification kit (QIAGEN), digested with the restriction enzymes and verified by capillary electrophoresis. Then 2 μg of this purified product were ligated with the digested reference backbone (molar ratio 7:1) using T4 DNA Ligase (New England Biolabs) by scaling up the manufacturer's protocol. XL-10 Gold Ultracompetent Cells (Agilent Technologies) were transformed with the ligation product, plated and incubated for 12 h at 30° C. to minimize the occurrence of recombination events. Colonies were scraped, mixed, grown in LB medium for additional 6 h and processed with NucleoBond Xtra MaxiPrep (Machery Nagel) according to the manufacturer's instruction. The plasmid prep was screened with MscI and XmaI restriction enzymes (New England Biolabs) for inverted terminal repeats and plasmid integrity. Design of the non barcoded AAV6 donor templates carrying homologies for AAVS1 or IL2RG (both encompassing a PGK.GFP reporter cassette) and CD40L were previously reported (Schiroli, G. et al., 2019, Cell Stem Cell 24: 551-565; Vavassori, V. et al., 2021, EMBO Mol Med. 13(3):e13545). The IDLV donor was generated using HIV-derived, third-generation self-inactivating transfer construct and the IDLV stock was prepared by transient transfection of human embryonic kidney 293T (HEK293T). At 30 h post transfection, vector-containing supernatant was collected, filtered, clarified, DNAse treated and loaded on a DEAE-packed column for Anion Exchange Chromatography. The vector-containing peak was collected, subjected to a second round of DNAse treatment, concentration by Tangential Flow Filtration and a final Size Exclusion Chromatography separation followed by sterilizing filtration and titration of the purified stock as previously described (Ferrari, S. et al., 2020, Nat Biotech 38(11):1298-1308). Sequences of the gRNAs were designed using an online tool54 and selected for predicted specificity score and on-target activity. Genomic sequences recognized by the gRNAs were previously reported (Schiroli, G. et al., 2019, Cell Stem Cell 24: 551-565; Vavassori, V. et al., 2021, EMBO Mol Med. 13(3):e13545). RNP complexes were assembled by incubating at a 1:1.5 molar ratio Streptococcus pyogenes (Sp)Cas9 protein (Aldevron) with pre-annealed synthetic Alt-R crRNA:tracrRNA (Integrated DNA Technologies) for 10 min at 25° C. together with 0.1 nmol of Alt-R Cas9 Electroporation Enhancer (Integrated DNA Technologies) was added before electroporation according to the manufacturer's instructions.

Vector maps were designed with SnapGene software v.5.0.7 (from GSL Biotech, available at snapgene.com) or Vector NTI Express v.1.6.2 (from Thermo Fisher Scientific, available at thermofisher.com).

mRNA In Vitro Transcription

The GSE56 construct was previously described (Schiroli et al., 2019, Cell Stem Cell 24: 551-565; and Ferrari et al., 2020, Nat Biotechnol 38: 1298-1308).

### Primary Cells

CB CD34⁺ HSPCs were purchased frozen from Lonza on approval by the TIGET-HPCT and were seeded at the concentration of 5×10⁵ cells per ml in serum-free StemSpan medium (StemCell Technologies) supplemented with 100 IU ml⁻¹ penicillin, 100 μg ml⁻¹ streptomycin, 2% glutamine, 100 ng ml⁻¹ hSCF (PeproTech), 100 ng ml⁻¹ hFlt3-L (PeproTech), 20 ng ml⁻¹ hTPO (PeproTech) and 20 ng ml⁻¹ hlL-6 (PeproTech) and 10 μM PGE2 (at the beginning of the culture, Cayman). Culture medium was also supplemented with 1 μM SR1 (Biovision) and 50 nM UM171 (STEMCell Technologies), unless otherwise specified.

G-CSF mPB CD34+ HSPCs were purified with the CliniMACS CD34 Reagent System (Miltenyi Biotec) from Mobilized Leukopak (AllCells) on approval by the TIGET-HPCT according to the manufacturer's instructions. HSPCs were seeded at the concentration of 5×105 cells per ml in serum-free StemSpan medium (StemCell Technologies) supplemented with 100 IU ml-1 penicillin, 100 μg ml-1 streptomycin, 2% glutamine, 300 ng ml-1 hSCF, 300 ng ml-1 hFlt3-L, 100 ng ml-1 hTPO and 10 μM PGE2 (at the beginning of the culture). Culture medium was also supplemented with 1 μM SR1 and 35 nM UM171.

Primary T cells were isolated from healthy male donors' PBMCs purified from buffy coats by sequential centrifugations in a Ficoll gradient according to a protocol approved by the Ospedale San Raffaele Scientific Institute Bioethical Committee (TIGET-HPCT). CD3+ T cells were stimulated using magnetic beads (a ratio of one to three cells to beads) conjugated with anti-CD3/anti-CD28 antibodies (Dynabeads human T-activator CD3/CD28, Thermo Fisher). Cells were maintained in Iscove's modified Dulbecco's medium (Corning) supplemented with 10% heat-inactivated FBS, 100 IU ml-1 penicillin, 100 μg ml-1 streptomycin, 2% glutamine, 5 ng ml-1 hlL-7 (PreproTech) and 5 ng ml-1 hIL-15 (PreproTech). Dynabeads were removed after 6 days of culture. In all the experiments, T cells were derived from male healthy donors.

All cells were cultured in a 5% CO2 humidified atmosphere at 37° C.

### Mice

NOD-SCID-IL2Rg−/− (NSG) mice were purchased from The Jackson Laboratory and maintained in specific-pathogen-free (SPF) conditions. The procedures involving animals were designed and performed with the approval of the Animal Care and Use Committee of the San Raffaele Hospital (IACUC #1165) and communicated to the Ministry of Health and local authorities according to Italian law.

### Gene Editing of Human HSPCs and Analyses

CD34⁺ HSPCs either freshly purified from human CB after obtaining informed consent and upon approval by the Ospedale San Raffaele Bioethical Committee, or purchased frozen from Lonza were edited according to a previously optimized protocol (Schiroli et al., 2017). Concerning p38, Erk and Jnk inhibition, CD34⁺ HSPCs were seeded at the concentration of 5×10⁵ CD34⁺ cells/ml and at day 1 and day 2 post-thawing, cells, where indicated, were treated where indicated with 4 μM of p38; 12 μM of Erk or with 2 μM Jnk inhibitors dissolved in DMSO (SB-203580; FR180204; SP600125 from Sigma-Aldrich) or with the highest volume used of DMSO for negative controls. After 3 days of stimulation, cells were washed with PBS and electroporated using P3 Primary Cell 4D-Nucleofector X Kit and program EO-100 (Lonza). Cells were electroporated with 1.25-2.5 mM of RNPs as indicated, performing the editing of the AAVS1, IL2RG and CD40L loci. Transduction with barcoded AAV6 was performed at a dose of 1×10⁴ vg/cell 15′ after electroporation. GSE56 mRNA was utilized when indicated at a dose of 150 mg/ml. Where indicated, Anakinra and SC514 were added in the culture medium 15′ after electroporation at the final concentration of 50 ng/μl and 25 μM, respectively. T cells were expanded for 20 days to perform flow cytometry. Gene editing efficiency was measured from cultured cells in vitro 96 hours after electroporation for CB and mPB-derived HSPCs by flow cytometry measuring the percentage of cells expressing the GFP marker or by digital droplet PCR analysis designing primers and probe on the junction between the vector sequence and the targeted locus and on control sequences utilized as normalizer as previously described (Schiroli et al., 2019, Cell Stem Cell 24: 551-565). T cells gene editing efficiency was measured from cultured cells in vitro 13 days after electroporation by flow cytometry measuring the percentage of cells expressing the NGFR marker.

For one-hit IDLV-based gene editing, after 2 or 2.5 days of stimulation 1×105-5×105 cells were treated with 8 μM cyclosporin H (CsH, Sigma) and then transduced with purified IDLV at MOI of 150, unless otherwise specified. After 24 or 12 h, cells were washed with DPBS and electroporated as described above. For two-hits IDLV-based gene editing, another round of transduction in presence of 8 μM CsH was performed immediately after electroporation with purified IDLV at MOI of 150, unless otherwise specified. When indicated, in vitro transcribed mRNAs were added to the electroporation mixture at the following final concentrations: 150 μg/μl GSE56; 250 μg/μl GSE56/E4orf6/7 (COMBO, see Ferrari, S. et al., 2020, Nat Biotech 38(11):1298-1308).

### CD34⁺ HSPC Xenotransplantation Studies in NSG Mice

For transplantation, 1.5-3×10⁵ CD34+ cells treated for editing at day 4 of culture were injected intravenously into NSG mice after sublethal irradiation (150-180 cGy). Sample size was determined by the total number of available treated cells. Mice were randomly attributed to each experimental group. Human CD45+ cell engraftment and the presence of gene-edited cells were monitored by serial collection of blood from the mouse tail and, at the end of the experiment (15-18 weeks after transplantation), BM and spleen were harvested and analyzed.

### Colony-Forming Unit Cell Assay

CFU-C assay was performed at the indicated timings after electroporation, plating 800 cells in methylcellulose-based medium (MethoCult H4434, StemCell Technologies) supplemented with 100 IU/ml penicillin and 100 mg/ml streptomycin. For selected analysis, the medium was also supplemented with 50 ng/ml IL6 and 20 ng/ml FT3L (for each condition 3 technical replicates were performed). Two weeks after plating, colonies were counted in blinded fashion and identified according to morphological criteria.

### Flow Cytometry

Immunophenotypic analyses were performed on the fluorescence activated cell sorting (FACS) Canto II (BD Pharmingen) or CytoFLEX LX Flow Cytometer (Beckman Coulter). From 0.5×10⁵ to 2×10⁵ cells (either from culture or mouse samples) were analyzed by flow cytometry. Ex vivo treated cells were stained for 15 min at 4° C. with CD34-PE, CD133-PEcy7 and CD90-APC antibodies, while BM-derived cells with CD45-APCCy7, CD34-PECy7, CD19-PE, CD3-APC, CD13 BV421 in a final volume of 50 μl and then washed with DPBS+2% heat-inactivated FBS. Single stained and fluorescence-minus-one-stained cells were used as controls. The Live/Dead Fixable Dead Cell Stain Kit (Thermo Fisher) or 7-aminoactinomycin D (Sigma Aldrich) Annexin V Pacific blue staining were included during sample preparation according to the manufacturer's instructions to identify dead cells. Apoptosis analysis was performed as previously described (Schiroli et al., 2019, Stem Cell 24: 551-565Single stained and fluorescence-minus-one-stained cells were used as controls. Data were analyzed with FlowJo software v. 10.8.1.

### Immunofluorescence Analysis.

Multitest slides (MP Biomedicals, 096041505) were coated with Poly-L-lysine solution (Sigma-Aldrich, P8920-500ML) at 1 mg/ml concentration for 20 min. After three washes with PBS solution, 0.3-0.5×105 cells were seeded on covers for 20 min and fixed with 4% PFA (Santa Cruz Biotechnology, sc-281692) for 20 min. Cells were then permeabilized with 0.1%-0.5% Triton X-100. After blocking with 0.5% BSA and 0.2% fish gelatin in DPBS, cells were stained with the indicated primary antibodies (53BP1 Antibody, Bethyl Laboratories; Anti-phospho Histone H2A.X (Ser139) Antibody, clone JBW301, Merck; NBS1 Antibody, Novus Biologicals; pRPA (S33) Antibody, polyclonal, Bethyl Laboratories, NF-kB total, Cell signalling). Cells were than washed with DPBS and incubated with Alexa Fluor 568- and 647-labeled secondary antibodies (Invitrogen/Thermo Scientific). Nuclear DNA was stained with DAPI at 0.2 μg ml-1 concentration (Sigma-Aldrich, D9542) and covers were mounted with Aqua-Poly/Mount solution (TebuBio, 18606-20) on glass slides (Bio-Optica). Fluorescent images were acquired using Leica SP5 Confocal microscopes. Quantification of DDR foci in immunofluorescence images was conducted using Cell Profiler or ImageJ software.

### CM-H₂DCFDA (General Oxidative Stress Indicator)

CM-H₂DCFDA (Thermo Fisher Scientific) was dissolved in DMSO. This molecule passively diffuses into cells, where its acetate groups are cleaved by intracellular esterases and its thiol-reactive chloromethyl group reacts with intracellular glutathione and other thiols. Subsequent oxidation yields a fluorescent adduct that is trapped inside the cell. Cells were resuspended in pre-warmed 1×DPBS and the probe was added to provide a final working concentration of 10 μM. Cells were the placed for 30 min in the incubator at 37° C., then 1×DPBS was removed and cells were resuspended in pre-warmed PBS-2% FBS. Fluorescence was quickly acquired at FACSCanto II using excitation sources and filters appropriate for fluorescein (FITC) and the collected data were analyzed using the FlowJo software. Because the dye is susceptible to photo-oxidation, all the steps were performed in the dark.

### MitoSOX Red Mitochondrial Superoxide Indicator

MitoSOX Red reagent (Thermo Fisher Scientific) permeates live cells and selectively targets mitochondria. It is rapidly oxidized by superoxide but not by other reactive oxygen species (ROS) and reactive nitrogen species (RNS). The oxidized product become highly fluorescent (Ex/Em 510/580) upon binding to nucleic acid. MitoSOX Red reagent was dissolved in DMSO and 5 μM were directly added to the cell culture. Cells were incubated for 10 min at 37° C. protected from light. After a washing in PBS-2% FBS, fluorescence was quickly acquired at FACSCanto II and the collected data were analyzed using the FlowJo software.

### SPiDER β-Galactosidase

CD34⁺ HSPCs were seeded at the concentration of 5×10⁵ CD34⁺ cells/ml and incubated with Chloroquin at a final working concentration of 150 μM for 1 h at 37° C. Then, CD34⁺ HSPCs were stained with 0.57 ng/μL for 15 min at 37° C. After a washing in PBS, fluorescence was quickly acquired at FACSCanto II and the collected data were analyzed using the FlowJo software.

### Myeloid-Erythroid-Megakaryocyte (MEM) Single-Cell Differentiation Assay.

On the day of the sorting, U-bottom 96-well were filled with 100 μl/well MEM cytokine medium: StemPro medium with nutrients supplement (Life Technologies) supplemented with cytokines (SCF 100 ng/ml, Flt3-L 20 ng/ml, TPO 100 ng/ml, IL-6 50 ng/ml, IL-3 10 ng/ml, IL-11 50 ng/ml, GM-CSF 20 ng/ml, IL-2 10 ng/ml, IL-7 20 ng/ml; all Miltenyi Biotec), erythropoietin (EPO) 3 units/ml (Eprex, Janssen-Cilag), h-LDL 50 ng/ml (Stem Cell Technologies), 1% L-Glutamine (Life Technologies) and 1% Pen/Strep (Life Technologies). CD34+CD133+CD45RA-CD90+population was sorted as single cells (1 cell/well) and cultured for 3 weeks at 37° C. Cell sorting was performed on a BD FACSAria Fusion (BD Biosciences) using BDFACS Diva software and equipped with four lasers: blue (488 nm), yellow/green (561 nm), red (640 nm) and violet (405 nm). Cells were sorted with a 100 mm nozzle.

### MEM Single-Cell Differentiation Assays Analysis

All single-cell-derived colonies were harvested into 96 U-bottom plates. Cells were then stained with 50 μl/well of antibody mix (CD45-PECy7; CD11b-PE; CD41-PECy5; GlyA-APC; CD14-APCCy7; CD56-BV421; CD15-BV510), incubated for 20 min in the dark at RT and then washed with 100 μl/well of PBS+2% FBS. The type (lineage composition) and the size of the colonies formed were assessed by high-throughput flow cytometry. A single cell was defined as giving rise to a colony if the sum of cells detected in the CD45+ gates was 30 cells. Erythroid (Ery) colonies were identified as CD45-GlyA+≥30 cells, Megakaryocytes (Meg) colonies as CD45+CD41+≥30 cells, Myeloid (My) colonies as [Monocytes (Mono: CD45+CD14+)+Granulocytes (Gran: CD45+CD15+)]≥30 cells and NK colonies as CD45+CD56+≥30 cells.

### Molecular Analyses

For molecular analyses, gDNA was isolated with QIAamp DNA Micro Kit (QIAGEN) according to the manufacturer's instructions.

For HDR digital droplet PCR (ddPCR) analysis, 5-30 ng of gDNA were analyzed using the QX200 Droplet Digital PCR System (Bio-Rad) according to the manufacturer's instructions. HDR ddPCR primers and probes were designed on the junction between the vector sequence and the targeted locus. Human TTC5 (Bio-Rad) was used for normalization. The percentage of cells harboring biallelic integration was calculated with the following formula: (no. of AAVS1+ droplets/no. of of TTC5+ droplets×200)−percentage of GFP+ cells. The percentage of monoallelic integration was then calculated with the following formula: percentage of GFP+ cells−percentage of cells with biallelic integration.

For gene expression analyses, total RNA was extracted using either miRNeasy Micro Kit (QIAGEN) according to the manufacturer's instructions and DNase treatment was performed using RNase-free DNase Set (QIAGEN). cDNA was synthetized with iScript cDNA Synthesis Kit (Bio-Rad). cDNA was then pre-amplified using TaqMan PreAmp Master Mix (ThermoFisher) and used for q-PCR in a Viia7 Real-time PCR thermal cycler using both Fast SYBR Green Master Mix (Thermofisher), after standard curve method optimization to reach the 100% primer efficiency for each couple of primers listed in 1. The relative expression of each target gene was first normalized to GUSB housekeeping genes expression and then represented as 2\hat{ }-DCt for each sample or as fold changes (2\hat{ }-DDCt) relative to the indicated control conditions.

TABLE 1
List of primers:

| Gene | Primers |
|---|---|
| CCL2 | FW 5′CTGTGATCTTCAAGACCATTGTG 3′ |
| | REV 5′ AGTTTGGGTTTGCTTGTCCAG 3′ |
| IL8 | FW 5′ CATCTCACTGTGTGTAAACATGAC 3′ |
| | REV 5′ CCTTGGCAAAACTGCACCTTCAC 3′ |
| IL6 | FW 5′GATTCAATGAGGAGACTTGCCTGG 3′ |
| | REV 5′ CTCACTACTCTCAAATCTGTTCTGG 3′ |
| IL1A | FW 5′ GGTTGAGTTTAAGCCAATCCA 3′ |
| | REV 5′ TGCTGACCTAGGCTTGATGA 3′ |
| CDKN1A | FW 5′ CAGCATGACAGATTTCTACCACTC 3′ |
| (p21) | REV 5′ CTCGCGCTTCCAGGACTG 3′ |
| CXCL10 | FW 5′ GCAGAGGAACCTCCAGTCTCAGC 3′ |
| | REV 5′ GAGAGAGGTACTCCTTGAATGCC 3′ |
| GUSB | FW 5′ CTGACACCTCCAAGTATCCCAAG 3′ |
| | REV 5′ GTCGTGTACAGAAGTACAGACCGC 3′ |

### Total RNA-Seq Library Preparation and Analysis

Whole transcriptomic analysis was performed on a pool of HSPCs derived from five CB donors. All conditions were performed in triplicate. Total RNA was isolated at 12 h after editing using miRNeasy Micro Kit (QIAGEN), and DNase treatment was performed using RNase-free DNase Set (QIAGEN), according to the manufacturer's instructions. RNA was quantified with The Qubit 2.0 Fluorometer (ThermoFisher) and its quality was assessed by a 2100 Agilent Bioanalyser (Agilent Technologies). Minimum quality was defined as RNA integrity number (RIN) >8,300 ng of total RNA were used for library preparation with TruSeq Stranded Total RNA with Ribo-Zero Gold kit (Illumina) and sequenced on a NextSeq 500 (Illumina). Read quality was determined using FastQC and low-quality sequences were trimmed using Trimmomatic. RNA-seq data were aligned to the human reference genome (GRCh38/hg38) using both HISAT2 and STAR with standard input parameters. Gene counts were produced using Subread featureCounts against Genecode v26 or v31, considering results produced by both the aligners. After visual inspection of results, we continued the analysis using alignments achieved using HISAT, which provided more conservative alignments. Transcript counts were processed by R/Bioconductor package edgeR (normalizing for library size using trimmed mean of M-values), DeSeq2 (normalizing for library size using Relative Log Expression) and limma-voom (normalizing for library size using trimmed mean of M-values), using standard protocols as reported in the respective manuals. All the predictions tools achieved very similar results in terms of differential expressed genes. We continued the analysis using edgeR results, which provided the more conservative and shared list of genes. Differential expression was determined considering only protein coding genes and correcting p-values using FDR.

### BAR-Seq Library Preparation

PCR amplicons for individual samples were generated by nested PCR using primers listed in Ferrari, S. et al., 2020, Nat Biotech 38(11):1298-1308 and starting from >50-100 ng of purified gDNA. The first PCR step was performed with GoTaq G2 DNA Polymerase (Promega) according to the manufacturer's instructions using the following amplification protocol: 95° C. for 5 min, (95° C. for 0.5 min, 60° C. for 0.5 min, 72° C. for 0.5 min)×20 cycles, 72° C. for 5 min. Forward primer was designed to bind donor template upstream the BAR sequence, while the reverse primer annealed outside the homology arm, thus amplifying 328 bp of the on-target integrated cassette. For targeted deep sequencing of the plasmid and AAV libraries, the reverse primer annealed to the homology arm. The second PCR step was performed with GoTaq G2 DNA Polymerase (Promega) according to the manufacturer's instructions using 5 μl of the first-step PCR product and the following amplification protocol: 95° C. for 5 min, (95° C. for 0.5 min, 60° C. for 0.5 min, 72° C. for 0.5 min)×20 cycles, 72° C. for 5 min. Second-step PCR primers were endowed with tails containing P5/P7 sequences, i5/i7 Illumina tags to allow multiplexed sequencing and R1/R2 primer binding sites (Ferrari, S. et al., 2020, Nat Biotech 38(11):1298-1308). PCR amplicons were separately purified using MinElute PCR Purification kit (QIAGEN) and AmpPure XP beads (Beckman Coulter). Library quality was assessed by Agilent Tapestation (Agilent Technologies). Amplicons were multiplexed and run on MiSeq 2×75 bp or 2×150 bp paired end (Illumina).

### BAR-Seq Analyses

BAR-Seq data were processed with TagDust57 (v.2.33) to identify and extract the BAR from each sample by taking advantage of the structural composition of the reads. Each putative BAR was then examined to filter out those having an incorrect nucleotide at the fixed positions or a BAR length different from the expected one (22 bp). BAR abundance was quantified by summing the number of identical sequences. Since amplification and sequencing errors may produce highly similar barcodes, a graph-based procedure was employed. For each sample, a graph structure was created in which BARs represented nodes and two nodes were linked with an edge if the corresponding sequences had an edit distance of <3. Ego subnetworks, that is, subgraphs focalized on highly abundant BARs, were iteratively identified and collapsed into a single node and, consequently, into a single BAR sequence. More precisely, nodes were ranked based on their counts, and at each iteration the ego network composed of the most abundant BAR and its neighbors were merged into a single BAR (the focal node) and its nodes were removed from the graph. The rationale behind this approach was that, although sequencing errors could produce different sequences, the parental BAR, which constitutes the focal node of the network, would have the highest count. BARs with a read count lower than three were discarded and the remaining set of BARs were identified as the valid BARs of this sample. To verify that all the samples used in the analysis were informative after the filtering process, we employed a previously described approach to estimate the richness of each sample, verifying that such a value was above the threshold of 95% in all the samples. After BAR ranking from the most to the least abundant, a saturation-based approach was implemented. The dominant set of BARs for each sample was defined as the pool of BARs representing >90% of the total abundance of valid BARs, while the remaining <10% comprised rare BARs.

### Alkaline Comet Assay

24 h post electroporation 3×103 cells per condition were mixed with molten Comet LMAgarose (Trevigen, MD) at a ratio of 1:10 (v/v) and immediately pipetted onto CometSlides (Trevigen, MD) and placed at +4° C. for 30′. Once solidified, the slides were immersed in prechilled Lysis Solution (Trevigen, MD) for 1 h at +4° C. Following lysis, slides were immersed in freshly prepared Alkaline Unwinding Solution pH>13 (300 mM NaOH, 1 mM EDTA) for 1 h at +4° C. and then electrophoresed in alkaline electrophoresis solution pH>13 (300 mM NaOH, 1 mM EDTA) at 1V/cm (21V) for 30 min. Slides were washed twice in ddH2O and fixed in 70% ethanol for 5 min. Comets were stained with SYBR Safe for 30′ at RT. All steps were conducted in the dark to prevent additional DNA damage. Comets were analysed using a Nikon Eclipse E600 microscope and a Nikon-DS-R12 camera. Up to 100 nuclei for each individual donor were analysed with CaspLab—Comet assay software project to determine “Olive Tail Moments” of individual nuclei.

### Single-Cell RNA-Sequencing and Analysis

Droplet-based digital 3′-end scRNA-Seq was performed on a Chromium Single-Cell Controller (10× Genomics) using the Chromium Next GEM Single Cell 3′ Reagent Kit v3.1 according to the manufacturer's instructions. 24 h after the editing treatment, viable CD34+cells (negativite to ZombieAqua viable dye) were sorted according to surface expression of CD34+CD133+CD45RA−CD90+GFP+ and CD34+CD133+CD45RA−CD90+GFP−. Cell sorting was performed on a BD FACSAria Fusion (BD Biosciences) using BDFACS Diva software and equipped with four lasers: blue (488 nm), yellow/green (561 nm), red (640 nm) and violet (405 nm). Cells were sorted with a 100 mm nozzle. A highly pure sorting modality (two-way purity sorting) was chosen. Sorted cells were collected in 1.5 ml Eppendorf tubes containing 100 μl of DPBS. Sorted population where stained for 30′ at 4° C. with Total Seq-B hashtag antibodies (H3 for CD34+CD133+CD45RA−CD90+GFP+GE DMSO and GE p38i; H4 for CD34+CD133+CD45RA−CD90+GFP− GE DMSO and H5 for CD34+CD133+CD45RA−CD90+GFP− GEp38i), and after pooling population from same condition, viable cells were counted with Trypan Blue solution 0.4% (GIBCO) and 5×104 viable cells (2,5×104 from each population) were utilized for the subsequent procedure (estimated recovery: 3×104 cells/sample). Briefly, single cells were partitioned in Gel Beads in Emulsion (GEMs) and lysed, followed by RNA barcoding, reverse transcription and PCR amplification (11 cycles). scRNA-Seq libraries were prepared according to the manufacturer's instructions, checked and quantified on LabChip GX Touch HT (Perkin Elmer) and Qubit 3.0 (Invitrogen) instruments. Sequencing was performed on a Nova Seq S2 (Illumina) using the NextSeq 500/550 High Output v2 kit (75 cycles).

Illumina sequencer's base call files (BCLs) were demultiplexed, for each flow cell directory, into FASTQ files using Cellranger mkfastq with default parameters (v. 6.1.1, https://github.com/10× Genomics/cellranger). FASTQ files with the distinct hashtags (HTO) were then processed using Cellranger count with default parameters. Internally, the software relies on STAR for aligning reads to the human reference genome (GRCh38) that was modified to include the insertion of the GFP gene in the AAVS1 locus. The output of Cellranger is a BAM file containing reads aligned to the genome and annotated with barcodes and hashtags information. Furthermore, three filtered matrices are generated, containing barcodes, features and hashtag names and the UMI counts for each gene-barcode information for each cell. Cells and features were retained if the following parameters were satisfied: over than 200 and fewer than 6000 unique genes per cell, and genes expressed in more than 3 cells. Cells with more than 15% of features mapping to mitochondrial genes were excluded. HTO data, of each sample separately, were normalized with a centered log-ratio (CLR) transformation across features. Cell demultiplexing was performed with HTODemux function in Seurat (v 4.1.1), considering an average positive quantile of 0.9 per each sample. Finally, only single cells, with a unique hashtag identifying a sorted population, were retained, and collected within a unique Seurat object. Counts were normalized using Seurat function NormalizeData with default parameters. Cell cycle scores were calculated using the CellCycleScoring function, providing as input a previously reported gene list (Nestorowa et al., 2016). Expression data were then scaled using the ScaleData function, regressing on the cell cycle phases (S, G2M scores as calculated with CellCycleScoring function), on the percentage of mitochondrial genes and the total number of reads per cell.

Principal Component Analysis (PCA) was then performed with all the samples using the RunPCA function, using as input a list of most variable genes (vst method in FindVariableFeatures function). Batch correction by sample/treatment was performed with Harmony (Korsunsky et al., 2019). Cell clusters were defined evaluating the first 55 principal components at resolution r=0.6 and using the FindClusters function, which relies on a hard nearest neighbour clustering algorithm, using the default Louvain implementation. Cells were visualized in 2-dimensions using UMAP (Uniform Manifold Approximation and Projection, McInnes et al., 2018). Genes enriched in cells within each cluster were identified by means of Wilcoxon test (FindAllMarkers), selecting only genes expressed in at least 20% of clustered cells. For the cluster annotation, similarly to what has been published (Wang et al., 2022), we utilized genes differentially express from the identified clusters, and performed GSEA (Subramanian et al., 2005) on signature lists from literature available gene sets from cord blood and bone marrow (Ju et al., 2013; Doulatov et al., 2013; Chen et al., 2014; Tirosh et al., 2016; Fares et al., 2017; Velten et al., 2017; Popescu et al., 2019; Drissen et al., 2019,; Psaila et al., 2020; Mende et al., 2022). The analysis was performed ranking differentially expressed genes by decreased log 2FC multiplied by −log 10 of the adjusted P-value. Once cluster annotation has been obtained with the help of the lists, genes differentially expressed across different conditions/cluster were identified using the FindMarkers function, applying the Wilcoxon test and the Bonferroni correction. For the functional annotation, differentially expressed genes were pre-ranked according to Log 2FC values and GSEA was performed on MSigDB (http://www.gsea-msigdb.org/gsea/msigdb/collections.jsp) and literature available datasets.

### Quantification and Statistical Analysis

Data were expressed as means±SEM or dot plots with median values indicated as a line. Inferential techniques were carried out whenever appropriate sample size was available; otherwise descriptive statistics are reported. Percentage values were transformed into a log-odds scale to perform parametric statistical analyses. Assumptions for the correct application of standard parametric procedures were checked (e.g., normality of the data). t test for paired data was applied to compare dependent observations. For mutational analysis significance was tested using a two-way ANOVA with Tukey's multiple comparison post-test and Bonferroni's correction. Growth curves and cell cycle analyses were analyzed using linear mixed-effects models which account for longitudinal course and inclusion of additional random-effect terms, thus considering sources of heterogeneity among experimental units. Treatment group indicator and time variable, along with their interaction, were included as covariates in the model to identify potential differences in growth dynamics of treatment groups. Logarithmic logit or cubic transformations were used to linearize the relationship between the outcome and the dependent variables. LME were estimated in R (version 3.5.1) by means of the nlme package, while the Ismeans package was used to perform the post hoc analysis and compute all pairwise comparisons of treatment groups at a fixed time point. Adjusted p-values using Bonferroni's correction are also reported. Whenever these assumptions were not met, nonparametric statistical tests were performed. In particular, Mann-Whitney test was performed to compare two independent groups. In presence of more than two independent groups, Kruskal-Wallis test was performed, followed by post hoc pairwise comparisons. This strategy was applied also to analyze growth curves when the interest was in differences among groups at specific time points. For paired observations, Wilcoxon matched-pairs signed rank test was performed. For the analysis of repeated-measures data, a robust rank-based method for the analysis of longitudinal data in factorial designs were used as indicated. For gene expression data, analyses were performed on 2(−DCT) values relative to housekeeping gene (GUSB or HPRT). Spearman's correlation coefficient was calculated to evaluate the presence of a monotonic relationship between two variables. Analyses were performed using GraphPad Prism v8 and R statistical software. Differences were considered statistically significant at *p<0.05, **p<0.01, ***p<0.001, ****p<0.0001, “ns” represents non significance.

Various preferred features and embodiments of the present invention will now be described with reference to the following numbered paragraphs.
1. Use of one or more inhibitor(s) of senescence for increasing the survival and/or engraftment of haematopoietic cells, haematopoietic stem cells, haematopoietic progenitor cells and/or T cells in gene therapy.
2. Use of one or more inhibitor(s) of senescence for increasing the efficiency of gene editing of haematopoietic cells, haematopoietic stem cells, haematopoietic progenitor cells, and/or T cells.
3. One or more inhibitor(s) of senescence for use in haematopoietic cell gene therapy, haematopoietic stem cell gene therapy, haematopoietic progenitor cell gene therapy and/or T cell gene therapy.
4. One or more inhibitor(s) of senescence for use in gene therapy in increasing the survival and/or engraftment of haematopoietic cells, haematopoietic stem cells, haematopoietic progenitor cells and/or T cells.
5. The use according to paragraph 1 or the one or more inhibitor(s) of senescence for use according to paragraph 3 or paragraph 4, wherein the gene therapy comprises gene editing.
6. The use according to any one of paragraphs 1, 2 or 5, or the one or more inhibitor(s) of senescence for use according to any one of paragraphs 3-5, wherein the one or more inhibitor(s) of senescence comprises or consists of a MAPK inhibitor, an IL-1 inhibitor and/or an NF-κB inhibitor.
7. The use according to paragraph 6, or the one or more inhibitor(s) of senescence for use according to paragraph 6, wherein the MAPK inhibitor is an inhibitor of p38 phosphorylation, an inhibitor of JNK phosphorylation or an inhibitor of ERK phosphorylation, optionally an inhibitor of p38 phosphorylation.
8. The use according to paragraph 6 or paragraph 7, or the one or more inhibitor(s) of senescence for use according to paragraph 6 or paragraph 7, wherein the MAPK inhibitor is a JNK inhibitor, a p38 inhibitor or an ERK inhibitor.
9. The use according to any one of paragraphs 6-8, or the one or more inhibitor(s) of senescence for use according to any one of paragraphs 6-8, wherein the MAPK inhibitor is FR180204, SP600125, SB203580 or a derivative thereof.
10. The use according to any one of paragraphs 6-9, or the one or more inhibitor(s) of senescence for use according to any one of paragraphs 6-9, wherein the IL-1 inhibitor is an anti-IL-1α antibody, an anti-IL-1p antibody, an IL-1 antagonist, an IL-1 receptor antagonist, an IL-1α converting enzyme inhibitor, an IL-1p converting enzyme inhibitor, or a soluble decoy IL-1 receptor.
11. The use according to any one of paragraphs 6-10, or the one or more inhibitor(s) of senescence for use according to any one of paragraphs 6-10, wherein the IL-1 inhibitor is IL-1 Ra, anakinra, canakinumab, rilonacept, gevokizumab or a variant thereof.
12. The use according to any one of paragraphs 6-11, or the one or more inhibitor(s) of senescence for use according to any one of paragraphs 6-11, wherein the NF-κB inhibitor is an IL-1 inhibitor, an IL-1 receptor inhibitor, a TLR4 inhibitor, a TAK1 inhibitor, an Akt inhibitor, an IKK inhibitor, an inhibitor of IκB phosphorylation, an inhibitor of IκB degradation, an inhibitor of the proteasome, an inhibitor of IκBα upregulation, an inhibitor of NF-κB nuclear translocation, an inhibitor of NF-κB expression, an inhibitor of NF-κB DNA binding, or an inhibitor of NF-κB transactivation.
13. The use according to any one of paragraphs 6-12, or the one or more inhibitor(s) of senescence for use according to any one of paragraphs 6-12, wherein the NF-κB inhibitor is SC514 or a derivative thereof; IL-1Ra, anakinra, canakinumab, rilonacept, gevokizumab or a variant thereof; or an siRNA, shRNA, miRNA or antisense DNA/RNA.
14. The use according to any one of paragraphs 1, 2 or 5-13, or the one or more inhibitor(s) of senescence for use according to any one of paragraphs 3-13, wherein the use further comprises the use of an agent which promotes homology directed DNA repair.
15. The use according to paragraph 14, or the one or more inhibitor(s) of senescence for use according to paragraph 14, wherein the agent is an inhibitor of p53 activation, optionally wherein the inhibitor is an inhibitor of p53 phosphorylation, optionally an inhibitor of p53 Serine 15 phosphorylation.
16. The use according to paragraph 15, or the one or more inhibitor(s) of senescence for use according to paragraph 15, wherein the inhibitor of p53 activation is a p53 dominant negative peptide, an ataxia telangiectasia mutated (ATM) kinase inhibitor or an ataxia telangiectasia and Rad3-related protein (ATR) inhibitor.
17. The use according to paragraph 15 or paragraph 16, or the one or more inhibitor(s) of senescence for use according to paragraph 15 or paragraph 16, wherein the inhibitor of p53 activation is pifithrin-α or a derivative thereof; KU-55933 or a derivative thereof; GSE56 or a variant thereof; KU-60019, BEZ235, wortmannin, CP-466722, Torin 2, CGK 733, KU-559403, AZD6738 or a derivative thereof; or an siRNA, shRNA, miRNA or antisense DNA/RNA, optionally wherein the inhibitor of p53 activation is GSE56 or a variant thereof.
18. The use according to any one of paragraphs 1, 2 or 5-17, or the one or more inhibitor(s) of senescence for use according to any one of paragraphs 3-17, wherein the inhibition of senescence in the haematopoietic cells, haematopoietic stem cells, haematopoietic progenitor cells and/or T cells is transient, optionally wherein the inhibition of MAPK, inhibition of IL-1 and/or inhibition of NF-κB in the haematopoietic cells, haematopoietic stem cells, haematopoietic progenitor cells and/or T cells is transient.
19. The use according to any one of paragraphs 13-18, or the one or more inhibitor(s) of senescence for use according to any one of paragraphs 13-18, wherein the inhibition of p53 activation in the haematopoietic cells, haematopoietic stem cells, haematopoietic progenitor cells and/or T cells is transient.
20. The use according to any one of paragraphs 6-19, or the one or more inhibitor(s) of senescence for use according to any one of paragraphs 6-19, wherein the haematopoietic cells, haematopoietic stem cells, haematopoietic progenitor cells and/or T cells are exposed to the IL-1 inhibitor and/or NF-κB inhibitor prior to, at the same time as and/or after the gene editing machinery is introduced into the cell, optionally at the same time as the gene editing machinery is introduced to the cell.
21. The use according to any one of paragraphs 6-20, or the one or more inhibitor(s) of senescence for use according to any one of paragraphs 6-20, wherein the inhibition of IL-1 and/or NF-κB occurs during gene editing of the haematopoietic cells, haematopoietic stem cells, haematopoietic progenitor cells and/or T cells.
22. The use according to any one of paragraphs 6-21, or the one or more inhibitor(s) of senescence for use according to any one of paragraphs 6-21, wherein the haematopoietic cells, haematopoietic stem cells, haematopoietic progenitor cells and/or T cells are exposed to the MAPK inhibitor prior to the gene editing machinery being introduced to the cell.
23. The use according to any one of paragraphs 6-22 or the one or more inhibitor(s) of senescence for use according to any one of paragraphs 6-22, wherein the MAPK inhibition occurs prior to and/or during gene editing of the haematopoietic cells, haematopoietic stem cells, haematopoietic progenitor cells and/or T cells.
24. The use according to any one of paragraphs 14-23, or the one or more inhibitor(s) of senescence for use according to any one of paragraphs 14-23, wherein the haematopoietic cells, haematopoietic stem cells, haematopoietic progenitor cells and/or T cells are exposed to the inhibitor of p53 activation prior to, at the same time as and/or after the gene editing machinery is introduced into the cell.
25. The use according to any one of paragraphs 14-24, or the one or more inhibitor(s) of senescence for use according to any one of paragraphs 14-24, wherein the inhibition of p53 occurs during gene editing of the haematopoietic cells, haematopoietic stem cells, haematopoietic progenitor cells and/or T cells.
26. The use according to any one of paragraphs 1, 2 or 5-25, or the one or more inhibitor(s) of senescence for use according to any one of paragraphs 3-25, wherein the one or more inhibitor(s) of senescence is added to the haematopoietic cell, haematopoietic stem cell, haematopoietic progenitor cells and/or T cells at a concentration of about 0.5-200 μM or about 0.1-200 ng/μl.
27. The use according to any one of paragraphs 1, 2 or 5-26, or the one or more inhibitor(s) of senescence for use according to any one of paragraphs 3-26, wherein the one or more inhibitor(s) of senescence is used in combination with a nucleic acid sequence encoding at least one adenoviral protein.
28. The use according to paragraph 27, or the one or more inhibitor(s) of senescence for use according to paragraph 27, wherein the adenoviral protein is expressed transiently in the haematopoietic cell, haematopoietic stem cell, haematopoietic progenitor cell and/or T cell, optionally wherein the transient expression occurs during gene editing of the haematopoietic cell, haematopoietic stem cell, haematopoietic progenitor cell and/or T cell.
29. The use according to any one of paragraphs 2 or 5-28, or the one or more inhibitor(s) of senescence for use according to any one of paragraphs 5-28, wherein the target of the gene editing is selected from the group consisting of FANC-A, CD40L, RAG-1, IL-2RG, CYBA, CYBB, NCF1, NCF2, and NCF4.
30. A method of gene editing a population of haematopoietic cells, haematopoietic stem cells, haematopoietic progenitor cells and/or T cells comprising the steps:
   (a) contacting the population of haematopoietic cells, haematopoietic stem cells, haematopoietic progenitor cells and/or T cells with one or more inhibitor(s) of senescence;
   (b) introducing gene editing machinery to the population of haematopoietic cells, haematopoietic stem cells, haematopoietic progenitor cells and/or T cells with one or more vectors; and
   (c) editing the genome of said haematopoietic cells, haematopoietic stem cells, haematopoietic progenitor cells and/or T cells.
31. A method of transducing a population of haematopoietic cells, haematopoietic stem cells, haematopoietic progenitor cells and/or T cells with a viral vector comprising the steps:
   (a) contacting the population of haematopoietic cells, haematopoietic stem cells, haematopoietic progenitor cells and/or T cells with one or more inhibitor(s) of senescence; and
   (b) transducing the population of haematopoietic cells, haematopoietic stem cells, haematopoietic progenitor cells and/or T cells with one or more viral vectors.
32. The method according to paragraph 30 or paragraph 31, wherein steps (a) and (b) are carried out ex vivo or in vitro.
33. The method according to any one of paragraphs 30-32, wherein the one or more inhibitor(s) of senescence comprises or consists of a MAPK inhibitor, an IL-1 inhibitor and/or an NF-κB inhibitor.
34. The method according to any one of paragraphs 30-33, wherein the population of haematopoietic cells, haematopoietic stem cells and/or haematopoietic progenitor cells is contacted with the MAPK inhibitor prior to or concurrently with the step of introducing gene editing machinery to said cells or to the step of transducing said cells, optionally prior to the step of introducing gene editing machinery to said cells or to the step of transducing said cells.
35. The method according to any one of paragraphs 30-34, wherein the population of haematopoietic cells, haematopoietic stem cells and/or haematopoietic progenitor cells is contacted with the IL-1 inhibitor and/or NF-κB inhibitor prior to, concurrently with or following the step of introducing gene editing machinery to said cells or the step of transducing said cells.
36. The method according to any one of paragraphs 30-35, wherein the method further comprises the step of contacting the population of haematopoietic cells, haematopoietic stem cells, haematopoietic progenitor cells and/or T cells with an agent which promotes homology directed DNA repair, optionally wherein the agent is an inhibitor of p53 activation.
37. The method according to paragraph 36, wherein the agent is an inhibitor of p53 as defined in any one of paragraphs 15-17.
38. The method according to any one of paragraphs 30-37, wherein one or more inhibitor(s) of senescence is added to the haematopoietic cells, haematopoietic stem cells, haematopoietic progenitor cells and/or T cells at a concentration of about 0.5-200 μM or about 0.1-200 ng/μl.
39. The method according to any one of paragraphs 30-38, wherein the method further comprises the step of contacting the population of haematopoietic cells, haematopoietic stem cells, haematopoietic progenitor cells and/or T cells with a nucleic acid sequence encoding at least one adenoviral protein.
40. The method according to paragraph 39, wherein the adenoviral protein is expressed transiently in the haematopoietic cell, haematopoietic stem cell, haematopoietic progenitor cell and/or T cell.
41. The method according to any one of paragraphs 30-40, wherein the population of haematopoietic cells, haematopoietic stem cells, haematopoietic progenitor cells and/or T cells is obtained from mobilised peripheral blood, bone marrow or umbilical cord blood.
42. The method according to any one of paragraphs 30-41, which includes a further step of enriching the population for haematopoietic stem and/or progenitor cells and/or T cells.
43. The method according to any one of paragraphs 30 or 32-42, wherein the target of the gene editing is selected from the group consisting of FANC-A, CD40L, RAG-1, IL-2RG, CYBA, CYBB, NCF1, NCF2, and NCF4.
44. A method of gene therapy comprising the steps:
   (a) gene editing a population of haematopoietic cells, haematopoietic stem cells, haematopoietic progenitor cells and/or T cells according to the method of any one of paragraphs 30 or 32-43; and
   (b) administering the gene edited haematopoietic cells, haematopoietic stem cells, haematopoietic progenitor cells and/or T cells to a subject.
45. The method of paragraph 44, wherein the gene edited cells are administered to a subject as part of an autologous stem cell transplant procedure or an allogeneic stem cell transplant procedure.
46. A method of gene therapy comprising the steps:
   (a) transducing a population of haematopoietic cells, haematopoietic stem cells, haematopoietic progenitor cells and/or T cells according to the method of any one of paragraphs 31-42; and
   (b) administering the population of transduced haematopoietic cells, haematopoietic stem cells, haematopoietic progenitor cells and/or T cells to a subject.
47. The method of paragraph 46, wherein step (b) comprises administering the transduced cells to a subject as part of an autologous stem cell transplant procedure or an allogeneic stem cell transplant procedure.
48. A population of gene edited and/or transduced haematopoietic cells, haematopoietic stem cells, haematopoietic progenitor cells and/or T cells prepared according to the method of any one of paragraphs 30-47.
49. A pharmaceutical composition comprising the population of gene edited and/or transduced haematopoietic cells, haematopoietic stem cells, haematopoietic progenitor cells and/or T cells of paragraph 48.
50. The population of gene edited and/or transduced haematopoietic cells, haematopoietic stem cells, haematopoietic progenitor cells and/or T cells of paragraph 48 for use in therapy.
51. The population of gene edited and/or transduced haematopoietic cells, haematopoietic stem cells, haematopoietic progenitor cells and/or T cells for use according to paragraph 50, wherein the population is administered as part of an autologous stem cell transplant procedure or an allogeneic stem cell transplant procedure.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. Use of one or more inhibitor(s) of senescence for increasing the survival and/or engraftment of haematopoietic cells, haematopoietic stem cells, haematopoietic progenitor cells and/or T cells in gene therapy.

2. Use of one or more inhibitor(s) of senescence for increasing the efficiency of gene editing of haematopoietic cells, haematopoietic stem cells, haematopoietic progenitor cells, and/or T cells.

3. One or more inhibitor(s) of senescence for use in haematopoietic cell gene therapy, haematopoietic stem cell gene therapy, haematopoietic progenitor cell gene therapy and/or T cell gene therapy.

4. One or more inhibitor(s) of senescence for use in gene therapy in increasing the survival and/or engraftment of haematopoietic cells, haematopoietic stem cells, haematopoietic progenitor cells and/or T cells.

5. The use according to claim 1 or the one or more inhibitor(s) of senescence for use according to claim 3 or claim 4, wherein the gene therapy comprises gene editing.

6. The use according to any one of claims 1, 2 or 5, or the one or more inhibitor(s) of senescence for use according to any one of claims 3-5, wherein the one or more inhibitor(s) of senescence comprises or consists of a MAPK inhibitor, an IL-1 inhibitor and/or an NF-κB inhibitor.

7. The use according to claim 6, or the one or more inhibitor(s) of senescence for use according to claim 6, wherein:
a) the MAPK inhibitor is a JNK inhibitor, a p38 inhibitor or an ERK inhibitor; and/or
b) the MAPK inhibitor is FR180204, SP600125, SB203580 or a derivative thereof.

8. The use according to claim 6 or claim 7, or the one or more inhibitor(s) of senescence for use according to claim 6 or claim 7, wherein:
a) the IL-1 inhibitor is an anti-IL-1α antibody, an anti-IL-1p antibody, an IL-1 antagonist, an IL-1 receptor antagonist, an IL-1α converting enzyme inhibitor, an IL-1β converting enzyme inhibitor, or a soluble decoy IL-1 receptor; and/or
b) the IL-1 inhibitor is IL-1Ra, anakinra, canakinumab, rilonacept, gevokizumab or a variant thereof.

9. The use according to any one of claims 6-8, or the one or more inhibitor(s) of senescence for use according to any one of claims 6-8, wherein:
a) the NF-κB inhibitor is an IL-1 inhibitor, an IL-1 receptor inhibitor, a TLR4 inhibitor, a TAK1 inhibitor, an Akt inhibitor, an IKK inhibitor, an inhibitor of IκB phosphorylation, an inhibitor of IκB degradation, an inhibitor of the proteasome, an inhibitor of IκBα upregulation, an inhibitor of NF-κB nuclear translocation, an inhibitor of NF-κB expression, an inhibitor of NF-κB DNA binding, or an inhibitor of NF-κB transactivation; and/or
b) the NF-κB inhibitor is SC514 or a derivative thereof; IL-1 Ra, anakinra, canakinumab, rilonacept, gevokizumab or a variant thereof; or an siRNA, shRNA, miRNA or antisense DNA/RNA.

10. The use according to any one of claims 1, 2 or 5-9, or the one or more inhibitor(s) of senescence for use according to any one of claims 3-9, wherein the use further comprises the use of an agent which promotes homology directed DNA repair, optionally wherein the agent is an inhibitor of p53 activation, optionally wherein the inhibitor of p53 activation is GSE56 or a variant thereof.

11. The use according to any one of claims 1, 2 or 5-10, or the one or more inhibitor(s) of senescence for use according to any one of claims 3-10, wherein the inhibition of senescence in the haematopoietic cells, haematopoietic stem cells, haematopoietic progenitor cells and/or T cells is transient, optionally wherein the inhibition of MAPK, inhibition of IL-1 and/or inhibition of NF-κB in the haematopoietic cells, haematopoietic stem cells, haematopoietic progenitor cells and/or T cells is transient.

12. The use according to any one of claims 6-12, or the one or more inhibitor(s) of senescence for use according to any one of claims 6-12, wherein:
a) the haematopoietic cells, haematopoietic stem cells, haematopoietic progenitor cells and/or T cells are exposed to the IL-1 inhibitor and/or NF-κB inhibitor prior to, at the same time as and/or after the gene editing machinery is introduced into the cell, optionally at the same time as the gene editing machinery is introduced to the cell; and/or
b) the haematopoietic cells, haematopoietic stem cells, haematopoietic progenitor cells and/or T cells are exposed to the MAPK inhibitor prior to the gene editing machinery being introduced to the cell.

13. The use according to any one of claims 1, 2 or 5-12, or the one or more inhibitor(s) of senescence for use according to any one of claims 3-12, wherein the one or more inhibitor(s) of senescence is added to the haematopoietic cell, haematopoietic stem cell, haematopoietic progenitor cells and/or T cells at a concentration of about 0.5-200 μM or about 0.1-200 ng/μl.

14. The use according to any one of claim 2 or 5-13 or the one or more inhibitor(s) of senescence for use according to any one of claims 5-13, wherein the target of the gene editing is selected from the group consisting of FANC-A, CD40L, RAG-1, IL-2RG, CYBA, CYBB, NCF1, NCF2, and NCF4.

15. A method of gene editing a population of haematopoietic cells, haematopoietic stem cells, haematopoietic progenitor cells and/or T cells comprising the steps:
(a) contacting the population of haematopoietic cells, haematopoietic stem cells, haematopoietic progenitor cells and/or T cells with one or more inhibitor(s) of senescence;
(b) introducing gene editing machinery to the population of haematopoietic cells, haematopoietic stem cells, haematopoietic progenitor cells and/or T cells with one or more vectors; and
(c) editing the genome of said haematopoietic cells, haematopoietic stem cells, haematopoietic progenitor cells and/or T cells.

16. A method of transducing a population of haematopoietic cells, haematopoietic stem cells, haematopoietic progenitor cells and/or T cells with a viral vector comprising the steps:
(a) contacting the population of haematopoietic cells, haematopoietic stem cells, haematopoietic progenitor cells and/or T cells with one or more inhibitor(s) of senescence; and
(b) transducing the population of haematopoietic cells, haematopoietic stem cells, haematopoietic progenitor cells and/or T cells with one or more viral vectors.

17. The method according to claim 15 or claim 16, wherein the one or more inhibitor(s) of senescence comprises or consists of a MAPK inhibitor, an IL-1 inhibitor and/or an NF-κB inhibitor.

18. The method according to any one of claims 15-17, wherein:
a) the population of haematopoietic cells, haematopoietic stem cells and/or haematopoietic progenitor cells is contacted with the MAPK inhibitor prior to or concurrently with the step of introducing gene editing machinery to said cells or to the step of transducing said cells, optionally prior to the step of introducing gene editing machinery to said cells or to the step of transducing said cells; and/or
b) the population of haematopoietic cells, haematopoietic stem cells and/or haematopoietic progenitor cells is contacted with the IL-1 inhibitor and/or NF-κB inhibitor prior to, concurrently with or following the step of introducing gene editing machinery to said cells or the step of transducing said cells.

19. The method according to any one of claims 15-18, wherein the method further comprises the step of contacting the population of haematopoietic cells, haematopoietic stem cells, haematopoietic progenitor cells and/or T cells with an agent which promotes homology directed DNA repair, optionally wherein the agent is an inhibitor of p53 activation.

20. The method according to any one of claims 15-19, wherein one or more inhibitor(s) of senescence is added to the haematopoietic cells, haematopoietic stem cells, haematopoietic progenitor cells and/or T cells at a concentration of about 0.5-200 μM or about 0.1-200 ng/μl.

21. The method according to any one of claims 15-20, wherein the population of haematopoietic cells, haematopoietic stem cells, haematopoietic progenitor cells and/or T cells is obtained from mobilised peripheral blood, bone marrow or umbilical cord blood.

22. The method according to any one of claims 15-21, which includes a further step of enriching the population for haematopoietic stem and/or progenitor cells and/or T cells.

23. The method according to any one of claim 15 or 17-22, wherein the target of the gene editing is selected from the group consisting of FANC-A, CD40L, RAG-1, IL-2RG, CYBA, CYBB, NCF1, NCF2, and NCF4.

24. A method of gene therapy comprising the steps:
(a) gene editing a population of haematopoietic cells, haematopoietic stem cells, haematopoietic progenitor cells and/or T cells according to the method of any one of claim 15 or 17-23; and
(b) administering the gene edited haematopoietic cells, haematopoietic stem cells, haematopoietic progenitor cells and/or T cells to a subject.

25. A method of gene therapy comprising the steps:
(a) transducing a population of haematopoietic cells, haematopoietic stem cells, haematopoietic progenitor cells and/or T cells according to the method of any one of claims 16-22; and
(b) administering the population of transduced haematopoietic cells, haematopoietic stem cells, haematopoietic progenitor cells and/or T cells to a subject.

26. A population of gene edited and/or transduced haematopoietic cells, haematopoietic stem cells, haematopoietic progenitor cells and/or T cells prepared according to the method of any one of claims 15-22.

27. A pharmaceutical composition comprising the population of gene edited and/or transduced haematopoietic cells, haematopoietic stem cells, haematopoietic progenitor cells and/or T cells of claim 26.

28. The population of gene edited and/or transduced haematopoietic cells, haematopoietic stem cells, haematopoietic progenitor cells and/or T cells of claim 26 for use in therapy.
